(19) United States
(12) Patent Application Publication
(10) Pub. No.: US 2008/0045537 A1
(43) Pub. Date: Feb. 21, 2008

(54) Chemical Compounds

(76) Inventors: Kristjan Gudmundsson, Durham, NC (US); Paul Richard Sebahar, Durham, NC (US); Leah D'Aurora Richardson, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/573,729

(22) PCT Filed: Aug. 12, 2005
(86) PCT No.: PCT/US05/28811

§ 371 Date: Feb. 15, 2007

Related U.S. Application Data
(60) Provisional application No. 60/601,928, filed on Aug. 16, 2004.

(51) Int. Cl. A61K 31/495; A61K 31/44; C07D 221/02; C07D 241/00

(52) U.S. Cl. 514/252.1; 514/299; 544/336; 546/112

(57) ABSTRACT

The present invention provides compounds that demonstrate protective effects on target cells from HIV infection in a manner as to bind to chemokine receptor, and which affect the binding of the natural ligand or chemokine to a receptor such as CXCR4 of a target cell.

### FIELD OF THE INVENTION

The present invention provides novel compounds that demonstrate protective effects on target cells from HIV infection in a manner as to bind specifically to the chemokine receptor, and which affect the binding of the natural ligand or chemokine to a receptor such as CXCR4 and/or CCR5 of a target cell.

### BACKGROUND OF THE INVENTION

HIV gains entry into host cells by means of the CD4 receptor and at least one co-receptor expressed on the surface of the cell membrane. M-tropic strains of HIV utilize the chemokine receptor CCR5, whereas T-tropic strains of HIV mainly use CXCR4 as the co-receptor. HIV co-receptor usage largely depends on hyper-variable regions of the V3 loop located on the viral envelope protein gp120. Binding of gp120 with CD4 and the appropriate co-receptor results in a conformational change and unmasking of a second viral envelope protein called gp41. The protein gp41 subsequently interacts with the host cell membrane resulting in fusion of the viral envelop with the cell. Subsequent transfer of viral genetic information into the host cell allows for the continuation of viral replication. Thus infection of host cells with HIV is usually associated with the virus gaining entry into the cell via the formation of the ternary complex of CCR5 or CXCR4, CD4, and gp120.

A pharmacological agent that would inhibit the interaction of gp120 with either CCR5/CD4 or CXCR4/CD4 would be a useful therapeutic in the treatment of a disease, disorder, or condition characterized by infection with M-tropic or T-tropic strains, respectively, either alone or in combination therapy.

Evidence that administration of a selective CXCR4 antagonist could result in an effective therapy comes from in vitro studies that have demonstrated that addition of ligands selective for CXCR4 as well as CXCR4-neutralizing antibodies to cells can block HIV viral/host cell fusion. In addition, human studies with the selective CXCR4 antagonist AMD-3100, have demonstrated that such compounds can significantly reduce T-tropic HIV viral load in those patients that are either dual tropic or those where only the T-tropic form of the virus is present.

In addition to serving as a co-factor for HIV entry, it has been recently suggested that the direct interaction of the HIV viral protein gp120 with CXCR4 could be a possible cause of CD8⁺ T-cell apoptosis and AIDS-related dementia via induction of neuronal cell apoptosis.

The signal provided by SDF-1 on binding to CXCR4 may also play an important role in tumor cell proliferation and regulation of angiogenesis associated with tumor growth; the known angiogenic growth factors VEG-F and bFGF up-regulate levels of CXCR4 in endothelial cells and SDF-1 can induce neovascularization in vivo. In addition, leukemia cells that express CXCR4 migrate and adhere to lymph nodes and bone marrow stromal cells that express SDF-1.

The binding of SDF-1 to CXCR4 has also been implicated in the pathogenesis of atherosclerosis, renal allograft rejection asthma and allergic airway inflammation, Alzheimer's disease, and arthritis.

The present invention is directed to compounds that can act as agents that modulate chemokine receptor activity. Such chemokine receptors include, but are not limited to, CCR1, CCR2, CCR3, CCR4, CCR5, CCR6, CCR7, CCR8, CXCR1, CXCR2, CXCR3, CXCR4, and CXCR5.

The present invention provides novel compounds that demonstrate protective effects on target cells from HIV infection in a manner as to bind specifically to the chemokine receptor, and which affect the binding of the natural ligand or chemokine to a receptor, such as CXCR4 and/or CCR5 of a target cell.

### SUMMARY OF THE INVENTION

The present invention includes compounds of formula (I): 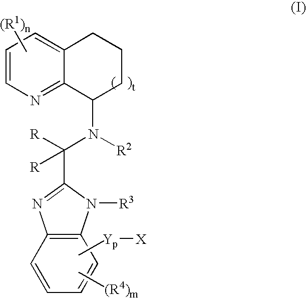
including salts, solvates, and physiologically functional derivatives thereof, wherein:
t is 0, 1, or 2;
each R independently is H, alkyl, alkenyl, alkynyl, haloalkyl, cycloalkyl, —R^{a}Ay, —R^{a}OR⁵, or —R^{a}S(O)_{q}R⁵;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁵R⁷, R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2, and, as shown, R¹ can be substituted throughout the depicted tetrahydroquinoline;
R² is selected from a group consisting of H, optionally substituted alkyl, haloalkyl, cycloalkyl, alkenyl, alkynyl, —R^{a}Ay, —R^{a}OR⁵, —R^{a}S(O)_{q}R⁵; wherein R² is not amine or alkylamine, or substituted with amine or alkylamine;
R³ is H, optionally substituted alkyl, haloalkyl, cycloalkyl, alkenyl, alkynyl, —R^{a}Ay, —R^{a}OR⁵, or —R^{a}S(O)_{q}R⁵, wherein when p is 0, R³ is not amine or alkylamine, or substituted with amine or alkylamine;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, —R^{a}Ay or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, S(O)_{q}—, S(O)_{q}NR¹⁰—, or —NR¹⁰S(O)_{q}—;
X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het, wherein when p is 0 then X is not —N(R¹⁰)₂;
each R^{a} independently is an optionally substituted alkylene, cycloalkylene, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or —R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁵, —R^{a}NR⁸R⁹, -Ay, -Het, —R^{a}Ay, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted 4-, 5-, or 6-membered heterocyclyl or heteroaryl group. Compounds of the present invention also include pharmaceutically acceptable salts or esters thereof.

In one embodiment t is 0.

In one embodiment t is 1 or 2. In yet another embodiment, t is 1.

In one embodiment R is H or alkyl.

In one embodiment R is H. In one embodiment R is alkyl, cycloalkyl, R^{a}Ay, R^{a}OR⁵. In one embodiment R is alkyl or R^{a}OR⁵.

In one embodiment n is 0.

In one embodiment n is 1 and R¹ is halogen, haloalkyl, alkyl, OR¹⁰, NR⁶R⁷, CO₂R¹⁰, CONR⁶R⁷, or cyano.

In one embodiment R² is H, optionally substituted alkyl, haloalkyl, or cycloalkyl. Preferably R² is optionally substituted alkyl, haloalkyl, or cycloalkyl. In one embodiment, R² is alkyl optionally substituted with cycloalkyl. In one embodiment R³ is H, optionally substituted alkyl, haloalkyl, cycloalkyl, alkenyl, or alkynyl. In one embodiment R² is branched alkyl. In one embodiment R² is alkyl substituted with cycloalkyl, hydroxyl or oxo.

In one embodiment R³ is R^{a}OR⁵ or R^{a}Ay. Preferably R³ is H, alkyl, haloalkyl, or cycloalkyl. In one embodiment, R³ is R^{a}OR⁵ or R^{a}Ay. More preferably R³ is H or optionally substituted alkyl. More preferably R³ is H. In one embodiment, R³ is R^{a}OR⁵ or alkyl. In one embodiment R³ is optionally substituted alkyl wherein when p is 0, R³ is not substituted with amine or alkylamine. In one embodiment R³ is optionally substituted alkyl, haloalkyl or cycloalkyl, and wherein when p is 0, R³ is not substituted with amine or alkylamine. In one embodiment R³ is branched alkyl. In one embodiment R³ is alkyl substituted with cycloalkyl, hydroxyl or oxo.

In one embodiment m is 0.

In one embodiment m is 1 or 2. Preferably m is 1.

When m is not 0, R⁴ preferably is one or more of halogen, haloalkyl, alkyl, OR¹⁰, NR⁶R⁷, CO₂R¹⁰, CONR⁶R⁷, or cyano.

In one embodiment, R^{a} is alkylene or cycloalkylene, optionally substituted with at least one of alkyl, hydroxyl, or oxo.

In one embodiment p is 0 and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂. Preferably X is —R^{a}N(R¹⁰)₂, -Het, —R^{a}Het, -HetN(R¹⁰)₂, —R^{a}HetN(R¹⁰)₂, or -HetR^{a}N(R¹⁰)₂. More preferably X is -Het, —R^{a}Het or -HetN(R¹⁰)₂. Most preferred is X is -Het. In one embodiment, X is -Het, optionally substituted with alkyl, (C═O)alkyl, alkoxy or hydroxyl. In one embodiment X is -Het or —R^{a}Het and -Het is optionally substituted with at least one alkyl.

In one embodiment p is 1; Y is —N(R¹⁰)—, —O—, —S—, —CONR¹⁰—, —NR¹⁰CO—, or —S(O)_{q}NR¹⁰—; and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂. More preferably p is 1; Y is —N(R¹⁰)—, —CONR¹⁰— or —NR¹⁰CO— and X is -Het, —R^{a}Het or -HetN(R¹⁰)₂— Most preferred is Y is —N(R¹⁰)— and X is Het.

In one embodiment -Het is optionally substituted with at least one alkyl, —(C═O)alkyl, alkoxy, hydroxyl, halogen, cycloalkyl, cycloalkoxy, cyano, amide, amino or alkylamino. In one embodiment -Het is substituted with a branched chain alkyl.

Preferably Het is piperidine, piperazine, azetidine, pyrrolidine, imidazole, pyridine, and the like. The Het can be optionally substituted on carbon or nitrogen.

In one embodiment each R is H; R² is alkyl, haloalkyl, or cycloalkyl; R³ is alkyl, haloalkyl, or cycloalkyl; n is 0; m is 0; p is 0; X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂; R^{a} is an optionally substituted alkylene, cycloalkylene, alkenylene, cycloalkenylene, or alkynylene; and R¹⁰ is H or alkyl.

In one embodiment p is 1; Y is C(O), —N(R¹⁰)—, —O—, —S—, —C(O)NR¹⁰—, —NR¹⁰CO—, or —S(O)_{q}NR¹⁰—; X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂; and -Het is optionally substituted with at least one of alkyl, —(C═O)alkyl, alkoxy, hydroxyl.

In one embodiment p is 1; Y is —C(O) or —C(O)NR10; X is —R^{a}Het or -Het; and -Het is optionally substituted with at least one alkyl.

Preferably the substituent —(Y)ₚ—X is located on the depicted benzimidazole ring as in formula (I-A): 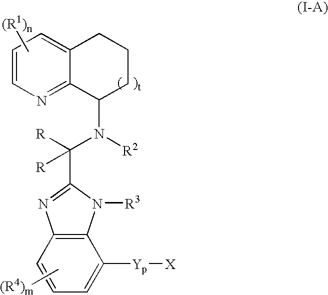
wherein all variables are as defined with respect to formula (I); or a pharmaceutically acceptable salt or ester thereof.

The present invention features a compound of formula (I-A) wherein p is 0 and X is Het and all other variables are as defined with respect to formula (I). Most preferably the Het is alkyl substituted piperazine.

The present invention features a compound of formula (I-A) wherein each R is H; R² is alkyl or cycloalkyl; R³ is alkyl or cycloalkyl; n is 0; m is 0; p is 0; X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂; R^{a} is alkylene, cycloalkylene, alkenylene, cycloalkenylene, or alkynylene; and R¹⁰ is H or alkyl, or a pharmaceutically acceptable salt or ester thereof.

Preferred compounds of the present invention include:
N-[2-(1H-Imidazol-4-yl)ethyl]-1-methyl-2-{[methyl(5,6,7,8-tetrahydro-8-quinolinyl)amino]methyl}-1H-benzimidazole-7-carboxamide;
N-Methyl-N-{[1-methyl-7-(1-piperazinylcarbonyl)-1H-benzimidazol-2-yl]methyl}-5,6,7,8-tetrahydro-8-quinolinamine;
N-[2-(1H-Imidazol-4-yl)ethyl]-1-methyl-2-{[methyl(5,6,7,8-tetrahydro-8-quinolinyl)amino]methyl}-1H-benzimidazole-4-carboxamide;
N-Methyl-N-{[1-methyl-4-(1-piperazinylcarbonyl)-1H-benzimidazol-2-yl]methyl}-5,6,7,8-tetrahydro-8-quinolinamine;
N-(4-Aminobutyl)-2-{[methyl(5,6,7,8-tetrahydro-8-quinolinyl)amino]methyl}-1H-benzimidazole-4-carboxamide;
2-{[Ethyl(5,6,7,8-tetrahydro-8-quinolinyl)amino]methyl}-N-[2-(1H-imidazol-4-yl)ethyl]-1H-benzimidazole-5-carboxamide;
N-(3-Aminopropyl)-2-{[ethyl(5,6,7,8-tetrahydro-8-quinolinyl)amino]methyl}-1H-benzimidazole-5-carboxamide;
N-(2-Aminoethyl)-2-{[ethyl(5,6,7,8-tetrahydro-8-quinolinyl)amino]methyl}-1H-benzimidazole-5-carboxamide;
N-(3-Aminopropyl)-2-{[ethyl(5,6,7,8-tetrahydro-8-quinolinyl)amino]methyl}-1H-benzimidazole-4-carboxamide;
N-(4-Aminobutyl)-2-{[ethyl(5,6,7,8-tetrahydro-8-quinolinyl)amino]methyl}-1H-benzimidazole-4-carboxamide;
N-{[5-(Aminomethyl)-1H-benzimidazol-2-yl]methyl}-N-methyl-5,6,7,8-tetrahydro-8-quinolinamine;
N-[(2-{[Methyl(5,6,7,8-tetrahydro-8-quinolinyl)amino]methyl}-1H-benzimidazol-5-yl)methyl]-1,3-propanediamine;
N-Methyl-N-[(5-{[(4-piperidinylmethyl)amino]methyl}-1H-benzimidazol-2-yl)methyl]-5,6,7,8-tetrahydro-8-quinolinamine;
N-{[4-(Aminomethyl)phenyl]methyl}-2-{[methyl(5,6,7,8-tetrahydro-8-quinolinyl)amino]methyl}-1H-benzimidazole-5-carboxamide;
N-Methyl-N-{[4-(1-piperazinylcarbonyl)-1H-benzimidazol-2-yl]methyl}-5,6,7,8-tetrahydro-8-quinolinamine;
N-Methyl-N-({4-[(4-methyl-1-piperazinyl)carbonyl]-1H-benzimidazol-2-yl}methyl)-5,6,7,8-tetrahydro-8-quinolinamine;
N-[2-(1H-Imidazol-4-yl)ethyl]-2-{[methyl(5,6,7,8-tetrahydro-8-quinolinyl)amino]methyl}-1H-benzimidazole-4-carboxamide;
N-[2-(1-Methyl-1H-imidazol-4-yl)ethyl]-2-{[methyl(5,6,7,8-tetrahydro-8-quinolinyl)amino]methyl}-1H-benzimidazole-4-carboxamide;
2-{[Methyl(5,6,7,8-tetrahydro-8-quinolinyl)amino]methyl}-1H-benzimidazole-4-carboxamide;
N-(2-Aminoethyl)-2-{[methyl(5,6,7,8-tetrahydro-8-quinolinyl)amino]methyl}-1H-benzimidazole-4-carboxamide;
2-{[Methyl(5,6,7,8-tetrahydro-8-quinolinyl)amino]methyl}-N-[2-(1-piperidinyl)propyl]-1H-benzimidazole-4-carboxamide;
2-{[Methyl(5,6,7,8-tetrahydro-8-quinolinyl)amino]methyl}-N-[3-(1-pyrrolidinyl)propyl]-1H-benzimidazole-4-carboxamide;
N-[3-(Dimethylamino)propyl]-2-{[methyl(5,6,7,8-tetrahydro-8-quinolinyl)amino]methyl}-1H-benzimidazole-4-carboxamide;
N-({4-[(4-Amino-1-piperidinyl)carbonyl]-1H-benzimidazol-2-yl}methyl)-N-methyl-5,6,7,8-tetrahydro-8-quinolinamine;
N-({4-[(3-Amino-1-pyrrolidinyl)carbonyl]-1H-benzimidazol-2-yl}methyl)-N-methyl-5,6,7,8-tetrahydro-8-quinolinamine;
N-{[4-({[2-(1H-Imidazol-4-yl)ethyl]amino}methyl)-1H-benzimidazol-2-yl]methyl}-N-methyl-5,6,7,8-tetrahydro-8-quinolinamine;
N-Methyl-N-{[4-(1-piperazinylmethyl)-1H-benzimidazol-2-yl]methyl}-5,6,7,8-tetrahydro-8-quinolinamine;
N-methyl-N-{[4-(1-piperazinyl)-1H-benzimidazol-2-yl]methyl}-5,6,7,8-tetrahydro-8-quinolinamine;
N-methyl-N-{[4-(4-methyl-1-piperazinyl)-1H-benzimidazol-2-yl]methyl}-5,6,7,8-tetrahydro-8-quinolinamine; and
N-methyl-N-({4-[4-(2-methylpropyl)-1-piperazinyl]-1H-benzimidazol-2-yl}methyl)-5,6,7,8-tetrahydro-8-quinolinamine; and pharmaceutically acceptable salts and esters thereof.

Particularly preferred compounds of the present invention include:
N-[2-(1H-Imidazol-4-yl)ethyl]-1-methyl-2-{[methyl(5,6,7,8-tetrahydro-8-quinolinyl)amino]methyl}-1H-benzimidazole-7-carboxamide;
N-Methyl-N-{[1-methyl-7-(1-piperazinylcarbonyl)-1H-benzimidazol-2-yl]methyl}-5,6,7,8-tetrahydro-8-quinolinamine;
N-[2-(1H-Imidazol-4-yl)ethyl]-1-methyl-2-{[methyl(5,6,7,8-tetrahydro-8-quinolinyl)amino]methyl}-1H-benzimidazole-4-carboxamide;
N-Methyl-N-{[4-(1-piperazinylcarbonyl)-1H-benzimidazol-2-yl]methyl}-5,6,7,8-tetrahydro-8-quinolinamine;
N-[2-(1H-Imidazol-4-yl)ethyl]-2-{[methyl(5,6,7,8-tetrahydro-8-quinolinyl)amino]methyl}-1H-benzimidazole-4-carboxamide;
N-[2-(1-Methyl-1H-imidazol-4-yl)ethyl]-2-{[methyl(5,6,7,8-tetrahydro-8-quinolinyl)amino]methyl}-1H-benzimidazole-4-carboxamide;
N-(2-Aminoethyl)-2-{[methyl(5,6,7,8-tetrahydro-8-quinolinyl)amino]methyl}-1H-benzimidazole-4-carboxamide;
2-{[Methyl(5,6,7,8-tetrahydro-8-quinolinyl)amino]methyl}-N-[2-(1-piperidinyl)propyl]-1H-benzimidazole-4-carboxamide;
2-{[Methyl(5,6,7,8-tetrahydro-8-quinolinyl)amino]methyl}-N-[3-(1-pyrrolidinyl)propyl]-1H-benzimidazole-4-carboxamide;
N-[3-(Dimethylamino)propyl]-2-{[methyl(5,6,7,8-tetrahydro-8-quinolinyl)amino]methyl}-1H-benzimidazole-4-carboxamide;
N-({4-[(4-Amino-1-piperidinyl)carbonyl]-1H-benzimidazol-2-yl}methyl)-N-methyl-5,6,7,8-tetrahydro-8-quinolinamine;
N-({4-[(3-Amino-1-pyrrolidinyl)carbonyl]-1H-benzimidazol-2-yl}methyl)-N-methyl-5,6,7,8-tetrahydro-8-quinolinamine;
N-{[4-({[2-(1H-Imidazol-4-yl)ethyl]amino}methyl)-1H-benzimidazol-2-yl]methyl}-N-methyl-5,6,7,8-tetrahydro-8-quinolinamine;
N-Methyl-N-{[4-(1-piperazinylmethyl)-1H-benzimidazol-2-yl]methyl}-5,6,7,8-tetrahydro-8-quinolinamine;
N-methyl-N-{[4-(1-piperazinyl)-1H-benzimidazol-2-yl]methyl}-5,6,7,8-tetrahydro-8-quinolinamine;
N-methyl-N-{[4-(4-methyl-1-piperazinyl)-1H-benzimidazol-2-yl]methyl}-5,6,7,8-tetrahydro-8-quinolinamine; and
N-methyl-N-({4-[4-(2-methylpropyl)-1-piperazinyl]-1H-benzimidazol-2-yl}methyl)-5,6,7,8-tetrahydro-8-quinolinamine; and pharmaceutically acceptable salts, and esters thereof.

Further, more particularly preferred compounds of the present invention include:
N-[2-(1H-Imidazol-4-yl)ethyl]-1-methyl-2-{[methyl(5,6,7,8-tetrahydro-8-quinolinyl)amino]methyl}-1H-benzimidazole-7-carboxamide;
N-[2-(1H-Imidazol-4-yl)ethyl]-2-{[methyl(5,6,7,8-tetrahydro-8-quinolinyl)amino]methyl}-1H-benzimidazole-4-carboxamide;
N-[2-(1-Methyl-1H-imidazol-4-yl)ethyl]-2-{[methyl(5,6,7,8-tetrahydro-8-quinolinyl)amino]methyl}-1H-benzimidazole-4-carboxamide;
N-(2-Aminoethyl)-2-{[methyl(5,6,7,8-tetrahydro-8-quinolinyl)amino]methyl}-1H-benzimidazole-4-carboxamide;
N-({4-[(3-Amino-1-pyrrolidinyl)carbonyl]-1H-benzimidazol-2-yl}methyl)-N-methyl-5,6,7,8-tetrahydro-8-quinolinamine;
N-Methyl-N-{[4-(1-piperazinylmethyl)-1H-benzimidazol-2-yl]methyl}-5,6,7,8-tetrahydro-8-quinolinamine;
N-methyl-N-{[4-(1-piperazinyl)-1H-benzimidazol-2-yl]methyl}-5,6,7,8-tetrahydro-8-quinolinamine; and
N-methyl-N-{[4-(4-methyl-1-piperazinyl)-1H-benzimidazol-2-yl]methyl}-5,6,7,8-tetrahydro-8-quinolinamine; and pharmaceutically acceptable salts or esters thereof.

One aspect of the present invention includes compounds selected from the group consisting of:
N-[2-(1H-Imidazol-4-yl)ethyl]-1-methyl-2-{[methyl(5,6,7,8-tetrahydro-8-quinolinyl)amino]methyl}-1H-benzimidazole-7-carboxamide;
N-[2-(1H-Imidazol-4-yl)ethyl]-1-methyl-2-{[methyl(5,6,7,8-tetrahydro-8-quinolinyl)amino]methyl}-1H-benzimidazole-4-carboxamide;
N-[2-(1H-Imidazol-4-yl)ethyl]-1-methyl-2-{[methyl(5,6,7,8-tetrahydro-8-quinolinyl)amino]methyl}-1H-benzimidazole-4-carboxamide;
N-[2-(1-Methyl-1H-imidazol-4-yl)ethyl]-2-{[methyl(5,6,7,8-tetrahydro-8-quinolinyl)amino]methyl}-1H-benzimidazole-4-carboxamide;
N-(2-Aminoethyl)-2-{[methyl(5,6,7,8-tetrahydro-8-quinolinyl)amino]methyl}-1H-benzimidazole-4-carboxamide;
2-{[Methyl(5,6,7,8-tetrahydro-8-quinolinyl)amino]methyl}-N-[2-(1-piperidinyl)propyl]-1H-benzimidazole-4-carboxamide;
2-{[Methyl(5,6,7,8-tetrahydro-8-quinolinyl)amino]methyl}-N-[3-(1-pyrrolidinyl)propyl]-1H-benzimidazole-4-carboxamide;
N-[3-(Dimethylamino)propyl]-2-{[methyl(5,6,7,8-tetrahydro-8-quinolinyl)amino]methyl}-1H-benzimidazole-4-carboxamide;
N-({4-[(4-Amino-1-piperidinyl)carbonyl]-1H-benzimidazol-2-yl}methyl)-N-methyl-5,6,7,8-tetrahydro-8-quinolinamine;
N-({4-[(3-Amino-1-pyrrolidinyl)carbonyl]-1H-benzimidazol-2-yl}methyl)-N-methyl-5,6,7,8-tetrahydro-8-quinolinamine;
N-{[4-({[2-(1H-Imidazol-4-yl)ethyl]amino}methyl)-1H-benzimidazol-2-yl]methyl}-N-methyl-5,6,7,8-tetrahydro-8-quinolinamine;
N-Methyl-N-{[4-(1-piperazinylmethyl)-1H-benzimidazol-2-yl]methyl}-5,6,7,8-tetrahydro-8-quinolinamine;
N-methyl-N-({4-[4-(2-methylpropyl)-1-piperazinyl]-1H-benzimidazol-2-yl}methyl)-5,6,7,8-tetrahydro-8-quinolinamine;
2-{[Methyl(5,6,7,8-tetrahydroquinolin-8-yl)amino]methyl}-1H-benzimidazole-5-carboxamide;
N-Methyl-N-[2-(methylamino)ethyl]-2-{[methyl(5,6,7,8-tetrahydro-8-quinolinyl)amino]methyl}-1H-benzimidazole-5-carboxamide;
N-[2-(Dimethylamino)ethyl]-2-{[methyl(5,6,7,8-tetrahydro-8-quinolinyl)amino]methyl}-1H-benzimidazole-5-carboxamide;
N-[2-(Methylamino)ethyl]-2-{[methyl(5,6,7,8-tetrahydro-8-quinolinyl)amino]methyl}-1H-benzimidazole-5-carboxamide;
N-[2-(Dimethylamino)ethyl]-N-methyl-2-{[methyl(5,6,7,8-tetrahydro-8-quinolinyl)amino]methyl}-1H-benzimidazole-5-carboxamide;
N-[2-(1H-imidazol-4-yl)ethyl]-N-methyl-2-{[methyl(5,6,7,8-tetrahydro-8-quinolinyl)amino]methyl}-1H-benzimidazole-4-carboxamide;
N-methyl-N-({4-[(2-methyl-1-piperazinyl)carbonyl]-1H-benzimidazol-2-yl}methyl)-5,6,7,8-tetrahydro-8-quinolinamine;
N-({4-[(1S,4S)-2,5-diazabicyclo[2.2.1]hept-2-ylcarbonyl]-1H-benzimidazol-2-yl}methyl)-N-methyl-5,6,7,8-tetrahydro-8-quinolinamine;
N-{[4-(hexahydro-1H-1,4-diazepin-1-ylcarbonyl)-1H-benzimidazol-2-yl]methyl}-N-methyl-5,6,7,8-tetrahydro-8-quinolinamine;
N-({4-[3-(Dimethylamino)propyl]-1H-benzimidazol-2-yl}methyl)-N-methyl-5,6,7,8-tetrahydro-8-quinolinamine;
N-Methyl-N-({4-[3-(1-pyrrolidinyl)propyl]-1H-benzimidazol-2-yl}methyl)-5,6,7,8-tetrahydro-8-quinolinamine;
N-methyl-N-({4-[3-(1-piperidinyl)propyl]-1H-benzimidazol-2-yl}methyl)-5,6,7,8-tetrahydro-8-quinolinamine;
N-{[4-(3-aminopropyl)-1H-benzimidazol-2-yl]methyl}-N-methyl-5,6,7,8-tetrahydro-8-quinolinamine;
2-{[Methyl(5,6,7,8-tetrahydro-8-quinolinyl)amino]methyl}-N-[3-(4-morpholinyl)propyl]-1H-benzimidazole-4-carboxamide;
N-(1H-Benzimidazol-2-ylmethyl)-2-{[methyl(5,6,7,8-tetrahydro-8-quinolinyl)amino]methyl}-1H-benzimidazole-4-carboxamide;
2-{[Methyl(5,6,7,8-tetrahydro-8-quinolinyl)amino]methyl}-N-3-pyrrolidinyl-1H-benzimidazole-4-carboxamide;
N-[3-(1H-Imidazol-1-yl)propyl]-2-{[methyl(5,6,7,8-tetrahydro-8-quinolinyl)amino]methyl}-1H-benzimidazole-4-carboxamide;
N-({4-[(4-Amino-1-piperidinyl)carbonyl]-1H-benzimidazol-2-yl}methyl)-N-methyl-5,6,7,8-tetrahydro-8-quinolinamine;
N-({4-[(3-amino-1-pyrrolidinyl)carbonyl]-1H-benzimidazol-2-yl}methyl)-N-methyl-5,6,7,8-tetrahydro-8-quinolinamine;
N-Methyl-N-({4-[(4-methylhexahydro-1H-1,4-diazepin-1-yl)carbonyl]-1H-benzimidazol-2-yl}methyl)-5,6,7,8-tetrahydro-8-quinolinamine;
[2-(Dimethylamino)ethyl](2-{[methyl(5,6,7,8-tetrahydro-8-quinolinyl)amino]methyl}-1H-benzimidazol-4-yl)amine;
Methyl[2-(methylamino)ethyl](2-{[methyl(5,6,7,8-tetrahydro-8-quinolinyl)amino]methyl}-1H-benzimidazol-4-yl)amine;
[2-(Dimethylamino)ethyl]methyl(2-{[methyl(5,6,7,8-tetrahydro-8-quinolinyl)amino]methyl}-1H-benzimidazol-4-yl)amine;
N-Methyl-N-({4-[4-(1-methylethyl)-1-piperazinyl]-1H-benzimidazol-2-yl}methyl)-5,6,7,8-tetrahydro-8-quinolinamine;
N-(1-Methylethyl)-N-{[4-(4-methyl-1-piperazinyl)-1H-benzimidazol-2-yl]methyl}-5,6,7,8-tetrahydro-8-quinolinamine;
N-(1-methylethyl)-N-({4-[4-(1-methylethyl)-1-piperazinyl]-1H-benzimidazol-2-yl}methyl)-5,6,7,8-tetrahydro-8-quinolinamine;
N-{1-methyl-1-[4-(4-methyl-1-piperazinyl)-1H-benzimidazol-2-yl]ethyl}-5,6,7,8-tetrahydro-8-quinolinamine;
N-Methyl-N-{1-methyl-1-[4-(4-methyl-1-piperazinyl)-1H-benzimidazol-2-yl]ethyl}-5,6,7,8-tetrahydro-8-quinolinamine;
N-({4-[4-(Aminoacetyl)-1-piperazinyl]-1H-benzimidazol-2-yl}methyl)-N-methyl-5,6,7,8-tetrahydro-8-quinolinamine;
(8R)—N-Methyl-N-{[4-(4-methyl-1-piperazinyl)-1H-benzimidazol-2-yl]methyl}-5,6,7,8-tetrahydro-8-quinolinamine;
(8S)—N-Methyl-N-{[4-(4-methyl-1-piperazinyl)-1H-benzimidazol-2-yl]methyl}-5,6,7,8-tetrahydro-8-quinolinamine;
(8R)—N-Ethyl-N-{[4-(4-methyl-1-piperazinyl)-1H-benzimidazol-2-yl]methyl}-5,6,7,8-tetrahydro-8-quinolinamine;
(8S)—N-Ethyl-N-{[4-(4-methyl-1-piperazinyl)-1H-benzimidazol-2-yl]methyl}-5,6,7,8-tetrahydro-8-quinolinamine;
(8R)—N-{[4-(4-methyl-1-piperazinyl)-1H-benzimidazol-2-yl]methyl}-N-propyl-5,6,7,8-tetrahydro-8-quinolinamine;
(8S)—N-{[4-(4-methyl-1-piperazinyl)-1H-benzimidazol-2-yl]methyl}-N-propyl-5,6,7,8-tetrahydro-8-quinolinamine;
(8S)—N—(1-Methylethyl)-N-{[4-(4-methyl-1-piperazinyl)-1H-benzimidazol-2-yl]methyl}-5,6,7,8-tetrahydro-8-quinolinamine;
(8R)—N-(Cyclopropylmethyl)-N-{[4-(4-methyl-1-piperazinyl)-1H-benzimidazol-2-yl]methyl}-5,6,7,8-tetrahydro-8-quinolinamine;
(8S)—N-(Cyclopropylmethyl)-N-{[4-(4-methyl-1-piperazinyl)-1H-benzimidazol-2-yl]methyl}-5,6,7,8-tetrahydro-8-quinolinamine;
(8S)—N-({4-[(8aR)-hexahydropyrrolo[1,2-a]pyrazin-2(1H)-yl]-1H-benzimidazol-2-yl}methyl)-N-methyl-5,6,7,8-tetrahydro-8-quinolinamine;
(8S)—N-Ethyl-N-({4-[(8aR)-hexahydropyrrolo[1,2-a]pyrazin-2(1H)-yl]-1H-benzimidazol-2-yl}methyl)-5,6,7,8-tetrahydro-8-quinolinamine;
(8S)—N-({4-[(8aR)-hexahydropyrrolo[1,2-a]pyrazin-2(1H)-yl]-1H-benzimidazol-2-yl}methyl)-N-propyl-5,6,7,8-tetrahydro-8-quinolinamine;
(8S)—N-methyl-N-({4-[(1R,5R)-7-methyl-3,7-diazabicyclo[3.3.1]non-3-yl]-1H-benzimidazol-2-yl}methyl)-5,6,7,8-tetrahydro-8-quinolinamine;
N-Cyclopropyl-N-{[4-(4-methyl-1-piperazinyl)-1H-benzimidazol-2-yl]methyl}-5,6,7,8-tetrahydro-8-quinolinamine;
(8S)—N-{(1S)-1-[4-(methyloxy)phenyl]ethyl}-N-{[4-(4-methyl-1-piperazinyl)-1H-benzimidazol-2-yl]methyl}-5,6,7,8-tetrahydro-8-quinolinamine;
(8S)—N-{[4-(4-Methyl-1-piperazinyl)-1H-benzimidazol-2-yl]methyl}-N-{2-[(phenylmethyl)oxy]ethyl}-5,6,7,8-tetrahydro-8-quinolinamine;
2-{{[4-(4-Methyl-1-piperazinyl)-1H-benzimidazol-2-yl]methyl}[(8S)-5,6,7,8-tetrahydro-8-quinolinyl]amino}ethanol;
3-{{[4-(4-Methyl-1-piperazinyl)-1H-benzimidazol-2-yl]methyl}[(8S)-5,6,7,8-tetrahydro-8-quinolinyl]amino}-1-propanol;
(8S)—N-{[4-(4-methyl-1-piperazinyl)-1H-benzimidazol-2-yl]methyl}-N-(phenylmethyl)-5,6,7,8-tetrahydro-8-quinolinamine;
N-Methyl-N-{[1-methyl-7-(4-methyl-1-piperazinyl)-1H-benzimidazol-2-yl]methyl}-5,6,7,8-tetrahydro-8-quinolinamine;
N-Ethyl-N-{[1-methyl-7-(4-methyl-1-piperazinyl)-1H-benzimidazol-2-yl]methyl}-5,6,7,8-tetrahydro-8-quinolinamine;
N-{[1-Methyl-7-(4-methyl-1-piperazinyl)-1H-benzimidazol-2-yl]methyl}-N-propyl-5,6,7,8-tetrahydro-8-quinolinamine;
N-(Cyclopropylmethyl)-N-{[1-methyl-7-(4-methyl-1-piperazinyl)-1H-benzimidazol-2-yl]methyl}-5,6,7,8-tetrahydro-8-quinolinamine;
(8S)—N-methyl-N-{[1-methyl-7-(1-piperazinyl)-1H-benzimidazol-2-yl]methyl}-5,6,7,8-tetrahydro-8-quinolinamine;
(8S)—N-Methyl-N-{[1-methyl-7-(4-methyl-1-piperazinyl)-1H-benzimidazol-2-yl]methyl}-5,6,7,8-tetrahydro-8-quinolinamine;
(8S)—N-{[1-ethyl-7-(4-methyl-1-piperazinyl)-1H-benzimidazol-2-yl]methyl}-N-methyl-5,6,7,8-tetrahydro-8-quinolinamine;
(8S)—N-{[1-Ethyl-7-(4-methyl-1-piperazinyl)-1H-benzimidazol-2-yl]methyl}-N-(phenylmethyl)-5,6,7,8-tetrahydro-8-quinolinamine;
(8S)—N-Ethyl-N-{[1-ethyl-7-(4-methyl-1-piperazinyl)-1H-benzimidazol-2-yl]methyl}-5,6,7,8-tetrahydro-8-quinolinamine;
N-{[5-Chloro-4-(4-methyl-1-piperazinyl)-1H-benzimidazol-2-yl]methyl}-N-methyl-5,6,7,8-tetrahydro-8-quinolinamine;
N-{[4-Chloro-7-(4-methyl-1-piperazinyl)-1H-benzimidazol-2-yl]methyl}-N-methyl-5,6,7,8-tetrahydro-8-quinolinamine;
N-Methyl-N-{(1R)-1-[4-(4-methyl-1-piperazinyl)-1H-benzimidazol-2-yl]ethyl}-5,6,7,8-tetrahydro-8-quinolinamine;
N-Methyl-N-{(1R)-1-[4-(4-methyl-1-piperazinyl)-1H-benzimidazol-2-yl]-2-[(phenylmethyl)oxy]ethyl}-5,6,7,8-tetrahydro-8-quinolinamine;
N-Methyl-N-{(1S)-1-[4-(4-methyl-1-piperazinyl)-1H-benzimidazol-2-yl]-2-[(phenylmethyl)oxy]ethyl}-5,6,7,8-tetrahydro-8-quinolinamine;
N-Methyl-N-{[4-(1-piperazinylcarbonyl)-1H-benzimidazol-2-yl]methyl}-5,6,7,8-tetrahydro-8-quinolinamine;
(8R)—N-Methyl-N-{[1-methyl-7-(4-methyl-1-piperazinyl)-1H-benzimidazol-2-yl]methyl}-5,6,7,8-tetrahydro-8-quinolinamine;
N-[2-(1H-Imidazol-4-yl)ethyl]-2-{[methyl(5,6,7,8-tetrahydro-8-quinolinyl)amino]methyl}-1H-benzimidazole-4-carboxamide;
2-{{[1-Methyl-7-(1-piperazinyl)-1H-benzimidazol-2-yl]methyl}[(8S)-5,6,7,8-tetrahydro-8-quinolinyl]amino}ethanol;
3-{{[1-Methyl-7-(1-piperazinyl)-1H-benzimidazol-2-yl]methyl}[(8S)-5,6,7,8-tetrahydro-8-quinolinyl]amino}-1-propanol;
2-{{[1-Methyl-7-(4-methyl-1-piperazinyl)-1H-benzimidazol-2-yl]methyl}[(8S)-5,6,7,8-tetrahydro-8-quinolinyl]amino}ethanol;
3-{{[1-Methyl-7-(4-methyl-1-piperazinyl)-1H-benzimidazol-2-yl]methyl}[(8S)-5,6,7,8-tetrahydro-8-quinolinyl]amino}-1-propanol;
N-Methyl-N-{[4-(4-methyl-1-piperazinyl)-1H-benzimidazol-2-yl]methyl}-6,7-dihydro-5H-cyclopenta[b]pyridin-7-amine;
N-{[4-(4-methyl-1-piperazinyl)-1H-benzimidazol-2-yl]methyl}-6,7,8,9-tetrahydro-5H-cyclohepta[b]pyridin-9-amine;
N-methyl-N-{[4-(4-methyl-1-piperazinyl)-1H-benzimidazol-2-yl]methyl}-6,7,8,9-tetrahydro-5H-cyclohepta[b]pyridin-9-amine; and pharmaceutically acceptable salts or esters thereof.

One aspect of the present invention includes compounds selected from the group consisting of:
N-methyl-N-{[4-(1-piperazinyl)-1H-benzimidazol-2-yl]methyl}-5,6,7,8-tetrahydro-8-quinolinamine;
N-methyl-N-{[4-(4-methyl-1-piperazinyl)-1H-benzimidazol-2-yl]methyl}-5,6,7,8-tetrahydro-8-quinolinamine;
N-methyl-N-({4-[4-(2-methylpropyl)-1-piperazinyl]-1H-benzimidazol-2-yl}methyl)-5,6,7,8-tetrahydro-8-quinolinamine;
N-Methyl-N-({4-[4-(1-methylethyl)-1-piperazinyl]-1H-benzimidazol-2-yl}methyl)-5,6,7,8-tetrahydro-8-quinolinamine;
N-(1-Methylethyl)-N-{[4-(4-methyl-1-piperazinyl)-1H-benzimidazol-2-yl]methyl}-5,6,7,8-tetrahydro-8-quinolinamine;
(8R)—N-Methyl-N-{[4-(4-methyl-1-piperazinyl)-1H-benzimidazol-2-yl]methyl}-5,6,7,8-tetrahydro-8-quinolinamine;
(8S)—N-Methyl-N-{[4-(4-methyl-1-piperazinyl)-1H-benzimidazol-2-yl]methyl}-5,6,7,8-tetrahydro-8-quinolinamine;
(8S)—N-Ethyl-N-{[4-(4-methyl-1-piperazinyl)-1H-benzimidazol-2-yl]methyl}-5,6,7,8-tetrahydro-8-quinolinamine;
(8S)—N-{[4-(4-methyl-1-piperazinyl)-1H-benzimidazol-2-yl]methyl}-N-propyl-5,6,7,8-tetrahydro-8-quinolinamine;
(8S)—N-(1-Methylethyl)-N-{[4-(4-methyl-1-piperazinyl)-1H-benzimidazol-2-yl]methyl}-5,6,7,8-tetrahydro-8-quinolinamine;
(8S)—N-(Cyclopropylmethyl)-N-{[4-(4-methyl-1-piperazinyl)-1H-benzimidazol-2-yl]methyl}-5,6,7,8-tetrahydro-8-quinolinamine;
(8S)—N-({4-[(8aR)-hexahydropyrrolo[1,2-a]pyrazin-2(1H)-yl]-1H-benzimidazol-2-yl}methyl)-N-methyl-5,6,7,8-tetrahydro-8-quinolinamine;
(8S)—N-Ethyl-N-({4-[(8aR)-hexahydropyrrolo[1,2-a]pyrazin-2(1H)-yl]-1H-benzimidazol-2-yl}methyl)-5,6,7,8-tetrahydro-8-quinolinamine;
(8S)—N-({4-[(8aR)-hexahydropyrrolo[1,2-a]pyrazin-2(1H)-yl]-1H-benzimidazol-2-yl}methyl)-N-propyl-5,6,7,8-tetrahydro-8-quinolinamine;
2-{{[4-(4-Methyl-1-piperazinyl)-1H-benzimidazol-2-yl]methyl}[(8S)-5,6,7,8-tetrahydro-8-quinolinyl]amino}ethanol;
3-{{[4-(4-Methyl-1-piperazinyl)-1H-benzimidazol-2-yl]methyl}[(8S)-5,6,7,8-tetrahydro-8-quinolinyl]amino}-1-propanol;
(8S)—N-{[4-(4-methyl-1-piperazinyl)-1H-benzimidazol-2-yl]methyl}-N-(phenylmethyl)-5,6,7,8-tetrahydro-8-quinolinamine;
N-Methyl-N-{[1-methyl-7-(4-methyl-1-piperazinyl)-1H-benzimidazol-2-yl]methyl}-5,6,7,8-tetrahydro-8-quinolinamine;
N-Ethyl-N-{[1-methyl-7-(4-methyl-1-piperazinyl)-1H-benzimidazol-2-yl]methyl}-5,6,7,8-tetrahydro-8-quinolinamine;
N-{[1-Methyl-7-(4-methyl-1-piperazinyl)-1H-benzimidazol-2-yl]methyl}-N-propyl-5,6,7,8-tetrahydro-8-quinolinamine;
N-(Cyclopropylmethyl)-N-{[1-methyl-7-(4-methyl-1-piperazinyl)-1H-benzimidazol-2-yl]methyl}-5,6,7,8-tetrahydro-8-quinolinamine;
(8S)—N-methyl-N-{[1-methyl-7-(1-piperazinyl)-1H-benzimidazol-2-yl]methyl}-5,6,7,8-tetrahydro-8-quinolinamine;
(8S)—N-Methyl-N-{[1-methyl-7-(4-methyl-1-piperazinyl)-1H-benzimidazol-2-yl]methyl}-5,6,7,8-tetrahydro-8-quinolinamine;
(8S)—N-{[1-ethyl-7-(4-methyl-1-piperazinyl)-1H-benzimidazol-2-yl]methyl}-N-methyl-5,6,7,8-tetrahydro-8-quinolinamine;
(8S)—N-{[1-Ethyl-7-(4-methyl-1-piperazinyl)-1H-benzimidazol-2-yl]methyl}-N-(phenylmethyl)-5,6,7,8-tetrahydro-8-quinolinamine;
(8S)—N-Ethyl-N-{[1-ethyl-7-(4-methyl-1-piperazinyl)-1H-benzimidazol-2-yl]methyl}-5,6,7,8-tetrahydro-8-quinolinamine;
(8S)—N-Ethyl-N-{[1-methyl-7-(4-methyl-1-piperazinyl)-1H-benzimidazol-2-yl]methyl}-5,6,7,8-tetrahydro-8-quinolinamine;
(8R)—N-Methyl-N-{[1-methyl-7-(4-methyl-1-piperazinyl)-1H-benzimidazol-2-yl]methyl}-5,6,7,8-tetrahydro-8-quinolinamine;
(8S)—N-{[1-Methyl-7-(4-methyl-1-piperazinyl)-1H-benzimidazol-2-yl]methyl}-N-propyl-5,6,7,8-tetrahydro-8-quinolinamine;
(8S)—N-{[1-Methyl-7-(4-methyl-1-piperazinyl)-1H-benzimidazol-2-yl]methyl}-N-(phenylmethyl)-5,6,7,8-tetrahydro-8-quinolinamine;
2-{{[1-Methyl-7-(4-methyl-1-piperazinyl)-1H-benzimidazol-2-yl]methyl}[(8S)-5,6,7,8-tetrahydro-8-quinolinyl]amino}ethanol;
3-{{[1-Methyl-7-(4-methyl-1-piperazinyl)-1H-benzimidazol-2-yl]methyl}[(8S)-5,6,7,8-tetrahydro-8-quinolinyl]amino}-1-propanol;
N-Methyl-N-{[4-(4-methyl-1-piperazinyl)-1H-benzimidazol-2-yl]methyl}-6,7-dihydro-5H-cyclopenta[b]pyridin-7-amine;
N-methyl-N-{[4-(4-methyl-1-piperazinyl)-1H-benzimidazol-2-yl]methyl}-6,7,8,9-tetrahydro-5H-cyclohepta[b]pyridin-9-amine; and pharmaceutically acceptable salts or esters thereof.

One aspect of the present invention includes compounds selected from the group consisting of:
N-Methyl-N-{[1-methyl-7-(4-methyl-1-piperazinyl)-1H-benzimidazol-2-yl]methyl}-5,6,7,8-tetrahydro-8-quinolinamine;
N-Ethyl-N-{[1-methyl-7-(4-methyl-1-piperazinyl)-1H-benzimidazol-2-yl]methyl}-5,6,7,8-tetrahydro-8-quinolinamine;
N-{[1-Methyl-7-(4-methyl-1-piperazinyl)-1H-benzimidazol-2-yl]methyl}-N-propyl-5,6,7,8-tetrahydro-8-quinolinamine;
N-(Cyclopropylmethyl)-N-{[1-methyl-7-(4-methyl-1-piperazinyl)-1H-benzimidazol-2-yl]methyl}-5,6,7,8-tetrahydro-8-quinolinamine;
(8S)—N-methyl-N-{[1-methyl-7-(1-piperazinyl)-1H-benzimidazol-2-yl]methyl}-5,6,7,8-tetrahydro-8-quinolinamine;
(8S)—N-Methyl-N-{[1-methyl-7-(4-methyl-1-piperazinyl)-1H-benzimidazol-2-yl]methyl}-5,6,7,8-tetrahydro-8-quinolinamine;
(8S)—N-{[1-ethyl-7-(4-methyl-1-piperazinyl)-1H-benzimidazol-2-yl]methyl}-N-methyl-5,6,7,8-tetrahydro-8-quinolinamine;
(8S)—N-{[1-Ethyl-7-(4-methyl-1-piperazinyl)-1H-benzimidazol-2-yl]methyl}-N-(phenylmethyl)-5,6,7,8-tetrahydro-8-quinolinamine;
(8S)—N-Ethyl-N-{[1-ethyl-7-(4-methyl-1-piperazinyl)-1H-benzimidazol-2-yl]methyl}-5,6,7,8-tetrahydro-8-quinolinamine;
(8S)—N-Ethyl-N-{[1-methyl-7-(4-methyl-1-piperazinyl)-1H-benzimidazol-2-yl]methyl}-5,6,7,8-tetrahydro-8-quinolinamine;
(8R)—N-Methyl-N-{[1-methyl-7-(4-methyl-1-piperazinyl)-1H-benzimidazol-2-yl]methyl}-5,6,7,8-tetrahydro-8-quinolinamine;
(8S)—N-{[1-Methyl-7-(4-methyl-1-piperazinyl)-1H-benzimidazol-2-yl]methyl}-N-propyl-5,6,7,8-tetrahydro-8-quinolinamine;
(8S)—N-{[1-Methyl-7-(4-methyl-1-piperazinyl)-1H-benzimidazol-2-yl]methyl}-N-(phenylmethyl)-5,6,7,8-tetrahydro-8-quinolinamine;
2-{{[1-Methyl-7-(4-methyl-1-piperazinyl)-1H-benzimidazol-2-yl]methyl}[(8S)-5,6,7,8-tetrahydro-8-quinolinyl]amino}ethanol;
3-{{[1-Methyl-7-(4-methyl-1-piperazinyl)-1H-benzimidazol-2-yl]methyl}[(8S)-5,6,7,8-tetrahydro-8-quinolinyl]amino}-1-propanol;
   and pharmaceutically acceptable salts or esters thereof.

One aspect of the present invention includes the compounds substantially as hereinbefore defined with reference to any one of the Examples.

One aspect of the present invention includes a pharmaceutical composition comprising one or more compounds of the present invention and a pharmaceutically acceptable carrier.

One aspect of the present invention includes one or more compounds of the present invention for use as an active therapeutic substance.

One aspect of the present invention includes one or more compounds of the present invention for use in the treatment or prophylaxis of diseases and conditions caused by inappropriate activity of CXCR4.

One aspect of the present invention includes one or more compounds of the present invention for use in the treatment or prophylaxis of diseases and conditions caused by inappropriate activity of CCR5.

One aspect of the present invention includes one or more compounds of the present invention for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. Preferably the condition or disease is HIV infection, rheumatoid arthritis, inflammation, or cancer.

One aspect of the present invention includes the use of one or more compounds of the present invention in the manufacture of a medicament for use in the treatment or prophylaxis of a condition or disease modulated by a chemokine receptor. Preferably the chemokine receptor is CXCR4 or CCR5.

One aspect of the present invention includes use of one or more compounds of the present invention in the manufacture of a medicament for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. Preferably the use relates to a medicament wherein the condition or disorder is HIV infection, rheumatoid arthritis, inflammation, or cancer.

One aspect of the present invention includes a method for the treatment or prophylaxis of a condition or disease modulated by a chemokine receptor comprising the administration of one or more compounds of the present invention. Preferably the chemokine receptor is CXCR4 or CCR5.

One aspect of the present invention includes a method for the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers comprising the administration of one or more compounds of the present invention.

One aspect of the present invention includes a method for the treatment or prophylaxis of HIV infection, rheumatoid arthritis, inflammation, or cancer comprising the administration of one or more compounds of the present invention.

### DETAILED DESCRIPTION OF THE INVENTION

Terms are used within their accepted meanings. The following definitions are meant to clarify, but not limit, the terms defined.

As used herein the term “alkyl” refers to a straight or branched chain hydrocarbon, preferably having from one to twelve carbon atoms. Examples of “alkyl” as used herein include, but are not limited to, methyl, ethyl, propyl, isopropyl, isobutyl, n-butyl, tert-butyl, isopentyl, n-pentyl. In one aspect, alkyl may be optionally substituted with at least one of cycloalkyl, hydroxyl, or oxo.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds. Examples include, but are not limited to, vinyl, allyl, and the like.

As used herein the term “alkynyl” refers to a straight or branched chain aliphatic hydrocarbon containing one or more carbon-to-carbon triple bonds. Examples include, but are not limited to, ethynyl and the like.

As used herein, the term “alkylene” refers to an optionally substituted straight or branched chain divalent hydrocarbon radical, preferably having from one to ten carbon atoms. Examples of “alkylene” as used herein include, but are not limited to, methylene, ethylene, n-propylene, n-butylene, and the like. Preferred substituent groups include alkyl, hydroxyl or oxo.

As used herein, the term “alkenylene” refers to a straight or branched chain divalent hydrocarbon radical, preferably having from one to ten carbon atoms, containing one or more carbon-to-carbon double bonds. Examples include, but are not limited to, vinylene, allylene or 2-propenylene, and the like.

As used herein, the term “alkynylene” refers to a straight or branched chain divalent hydrocarbon radical, preferably having from one to ten carbon atoms, containing one or more carbon-to-carbon triple bonds. Examples include, but are not limited to, ethynylene and the like.

As used herein, the term “cycloalkyl” refers to an optionally substituted non-aromatic cyclic hydrocarbon ring. Exemplary “cycloalkyl” groups include, but are not limited to, cyclopropyl, cyclobutyl, cyclopentyl, cyclohexyl, and cycloheptyl. As used herein, the term “cycloalkyl” includes an optionally substituted fused polycyclic hydrocarbon saturated ring and aromatic ring system, namely polycyclic hydrocarbons with less than maximum number of non-cumulative double bonds, for example where a saturated hydrocarbon ring (such as a cyclopentyl ring) is fused with an aromatic ring (herein “aryl,” such as a benzene ring) to form, for example, groups such as indane. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “cycloalkenyl” refers to an optionally substituted non-aromatic cyclic hydrocarbon ring containing one or more carbon-to-carbon double bonds which optionally includes an alkylene linker through which the cycloalkenyl may be attached. Exemplary “cycloalkenyl” groups include, but are not limited to, cyclopropenyl, cyclobutenyl, cyclopentenyl, cyclohexenyl, and cycloheptenyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “cycloalkylene” refers to a divalent, optionally substituted non-aromatic cyclic hydrocarbon ring. Exemplary “cycloalkylene” groups include, but are not limited to, cyclopropylene, cyclobutylene, cyclopentylene, cyclohexylene, and cycloheptylene. Preferred substituent groups include alkyl, hydroxyl or oxo.

As used herein, the term “cycloalkenylene” refers to a divalent optionally substituted non-aromatic cyclic hydrocarbon ring containing one or more carbon-to-carbon double bonds. Exemplary “cycloalkenylene” groups include, but are not limited to, cyclopropenylene, cyclobutenylene, cyclopentenylene, cyclohexenylene, and cycloheptenylene.

As used herein, the term “heterocycle” or “heterocyclyl” refers to an optionally substituted mono- or polycyclic ring system containing one or more degrees of unsaturation and also containing one or more heteroatoms. Preferred heteroatoms include N, O, and/or S, including N-oxides, sulfur oxides, and dioxides. More preferably, the heteroatom is N.

Preferably the heterocyclyl ring is three to twelve-membered and is either fully saturated or has one or more degrees of unsaturation. Such rings may be optionally fused to one or more of another “heterocyclic” ring(s) or cycloalkyl ring(s). Examples of “heterocyclic” groups include, but are not limited to, tetrahydrofuran, pyran, 1,4-dioxane, 1,3-dioxane, piperidine, piperazine, pyrrolidine, morpholine, tetrahydrothiopyran, and tetrahydrothiophene. Preferred substituent groups include alkyl, —(C═O)alkyl, —SO₂alkyl, alkenyl, alkynyl, alkoxy, hydroxyl, halogen, haloalkyl, cycloalkyl, cycloalkoxy, cyano, amide, amino, and alkylamino. The substituent on the heterocycle can be linked by a carbon atom or a heteroatom.

As used herein, the term “aryl” refers to an optionally substituted benzene ring or to an optionally substituted fused benzene ring system, for example anthracene, phenanthrene, or naphthalene ring systems. Examples of “aryl” groups include, but are not limited to, phenyl, 2-naphthyl, and 1-naphthyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “heteroaryl” refers to an optionally substituted monocyclic five to seven membered aromatic ring, or to an optionally substituted fused bicyclic aromatic ring system comprising two of such aromatic rings. These heteroaryl rings contain one or more nitrogen, sulfur, and/or oxygen atoms, where N-oxides, sulfur oxides, and dioxides are permissible heteroatom substitutions. Preferably, the heteroatom is N.

Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, benzimidizolyl, imidazopyridinyl, pyrazolopyridinyl, and pyrazolopyrimidinyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents as perfluoroalkyl groups and the like.

As used herein the term “alkoxy” refers to a group —OR′, where R′ is alkyl as defined.

As used herein the term “cycloalkoxy” refers to a group —OR′, where R′ is cycloalkyl as defined.

As used herein the term “alkoxycarbonyl” refers to groups such as: 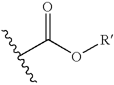
where the R′ represents an alkyl group as herein defined.

As used herein the term “aryloxycarbonyl” refers to groups such as: 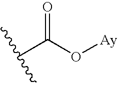
where the Ay represents an aryl group as herein defined.

As used herein the term “nitro” refers to a group —NO₂.

As used herein the term “cyano” refers to a group —CN.

As used herein the term “azido” refers to a group —N₃.

As used herein the term amino refers to a group —NR′R″, where R′ and R″ independently represent H, alkyl, alkenyl, alkynyl, cycloalkyl, heterocyclyl, aryl, or heteroaryl. Similarly, the term “alkylamino” includes an alkylene linker through which the amino group is attached. Examples of “alkylamino” as used herein include groups such as —(CH₂)ₓNH₂, where x is preferably 1 to 6.

As used herein the term “amide” refers to a group —C(O)NR′R″, where R′ and R″ independently represent H, alkyl, alkenyl, alkynyl, cycloalkyl, heterocyclyl, aryl, or heteroaryl. Examples of “amide” as used herein include groups such as —C(O)NH₂, —C(O)NH(CH₃), —C(O)N(CH₃)₂, and the like.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted specifically. Rather, those of ordinary skill in the art will appreciate that the phrase is included to provide for obvious modifications, which are encompassed within the scope of the appended claims.

The compounds of formulas (I) may crystallize in more than one form, a characteristic known as polymorphism, and such polymorphic forms (“polymorphs”) are within the scope of formula (I). Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically and/or diastereomerically enriched mixtures. Also included within the scope of the invention are the individual isomers of the compounds represented by formula (I), as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of Formula I, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “modulators” as used herein is intended to encompass antagonist, agonist, inverse agonist, partial agonist or partial antagonist, inhibitors and activators. In one preferred embodiment of the present invention, the compounds demonstrate protective effects against HIV infection by inhibiting binding of HIV to a chemokine receptor such as CXCR4 and/or CCR5 of a target cell. The invention includes a method that comprises contacting the target cell with an amount of the compound that is effective at inhibiting the binding of the virus to the chemokine receptor.

In addition to the role chemokine receptors play in HIV infection this receptor class has also been implicated in a wide variety of diseases. Thus CXCR4 modulators may also have a therapeutic role in the treatment of diseases associated with hematopoiesis, including but not limited to, controlling the side effects of chemotherapy, enhancing the success of bone marrow transplantation, enhancing wound healing and burn treatment, as well as combating bacterial infections in leukemia. In addition, compounds may also have a therapeutic role in diseases associated with inflammation, including but not limited to inflammatory or allergic diseases such as asthma, allergic rhinitis, hypersensitivity lung diseases, hypersensitivity pneumonitis, eosinophilic pneumonitis, delayed-type hypersensitivity, interstitial lung disease (ILD) (e.g. idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies, insect sting allergies; autoimmune diseases such as rheumatoid arthritis, psoriatic arthritis, systemic lupus erythematosus, myastenia gravis, juvenile onset diabetes; glomerulonephritis, autoimmune throiditis, graft rejection, including allograft rejection or graft-versus-host disease; inflammatory bowel diseases, such as Crohn's disease and ulcerative colitus; spondyloarthropathies; scleroderma; psoriasis (including T-cell-mediated psoriasis) and inflammatory dermatoses such as dermatitis, eczema, atopic dermatitis, allergic contact dermatitis, urticaria, vasculitis (e.g. necrotizing, cutaneous, and hypersensitivity vasculitis); eoosinophilic myotis, eosinophilic fasciitis; and cancers.

For use in therapy, therapeutically effective amounts of a compound of formula (I), as well as salts, solvates, and physiological functional derivatives thereof, may be administered as the raw chemical. Additionally, the active ingredient may be presented as a pharmaceutical composition.

Accordingly, the invention further provides pharmaceutical compositions that include effective amounts of compounds of the formula (I) and salts, solvates, and physiological functional derivatives thereof, and one or more pharmaceutically acceptable carriers, diluents, or excipients. The compounds of formula (I) and salts, solvates, and physiologically functional derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the formula (I) or salts, solvates, and physiological functional derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of formula (I) for the treatment of humans suffering from frailty, generally, should be in the range of 0.1 to 100 mg/kg body weight of recipient (mammal) per day. More usually the effective amount should be in the range of 0.1 to 10 mg/kg body weight per day. Thus, for a 70 kg adult mammal one example of an actual amount per day would usually be from 7 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of formula (I) per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the invention, with regard to certain conditions and disorders for which the compounds of the present invention are believed useful certain routes will be preferable to others.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) and salts, solvates, and physiological functional derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of formula (I) and salts, solvates, and physiologically functional derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, and physiologically functional derivatives thereof, may be employed alone or in combination with other therapeutic agents. The compound(s) of formula (I) and the other pharmaceutically active agent(s) may be administered together or separately and, when administered separately, administration may occur simultaneously or sequentially, in any order. The amounts of the compound(s) of formula (I) and the other pharmaceutically active agent(s) and the relative timings of administration will be selected in order to achieve the desired combined therapeutic effect. The administration in combination of a compound of formula (I) salts, solvates, or physiologically functional derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds of the present invention may be used in the treatment of a variety of disorders and conditions and, as such, the compounds of the present invention may be used in combination with a variety of other suitable therapeutic agents useful in the treatment or prophylaxis of those disorders or conditions. The compounds may be used in combination with any other pharmaceutical composition where such combined therapy may be useful to modulate chemokine receptor activity and thereby prevent and treat inflammatory and/or immunoregulatory diseases.

The present invention may be used in combination with one or more agents useful in the prevention or treatment of HIV. Examples of such agents include:

Nucleotide reverse transcriptase inhibitors such as zidovudine, didanosine, lamivudine, zalcitabine, abacavir, stavidine, adefovir, adefovir dipivoxil, fozivudine, todoxil, and similar agents;

Non-nucleotide reverse transcriptase inhibitors (including an agent having anti-oxidation activity such as immunocal, oltipraz, etc.) such as nevirapine, delavirdine, efavirenz, loviride, immunocal, oltipraz, and similar agents;

Protease inhibitors such as saquinavir, ritonavir, indinavir, nelfinavir, aprenavir, palinavir, lasinavir, and similar agents;

Entry inhibitors such as T-20, T-1249, PRO-542, PRO-140, TNX-355, BMS-806, 5-Helix and similar agents;

Integrase inhibitors such as L-870, 180 and similar agents;

Budding inhibitors such as PA-344 and PA-457, and similar agents; and

Other CXCR4 and/or CCR5 inhibitors such as Sch-C, Sch-D, TAK779, UK 427, 857, TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

The scope of combinations of compounds of this invention with HIV agents is not limited to those mentioned above, but includes in principle any combination with any pharmaceutical composition useful for the treatment of HIV. As noted, in such combinations the compounds of the present invention and other HIV agents may be administered separately or in conjunction. In addition, one agent may be prior to, concurrent to, or subsequent to the administration of other agent(s).

The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of formula (I).

Those skilled in the art will recognize if a stereocenter exists in compounds of formula (I). Accordingly, the scope of the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### EXPERIMENTAL SECTION

Abbreviations:

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

g (grams); mg (milligrams);

L (liters); mL (milliliters);

μL (microliters); psi (pounds per square inch);

M (molar); mM (millimolar);

Hz (Hertz); MHz (megahertz);

mol (moles); mmol (millimoles);

RT (room temperature); h (hours);

min (minutes); TLC (thin layer chromatography);

mp (melting point); RP (reverse phase);

Tᵣ (retention time); TFA (trifluoroacetic acid);

TEA (triethylamine); THF (tetrahydrofuran);

TFAA (trifluoroacetic anhydride); CD₃OD (deuterated methanol);

CDCl₃ (deuterated chloroform); DMSO (dimethylsulfoxide);

SiO₂ (silica); atm (atmosphere);

EtOAc (ethyl acetate); CHCl₃ (chloroform);

HCl (hydrochloric acid); Ac (acetyl);

DMF (N,N-dimethylformamide); Me (methyl);

Cs₂CO₃ (cesium carbonate); EtOH (ethanol);

Et (ethyl); tBu (tert-butyl);

MeOH (methanol) p-TsOH (p-toluenesulfonic acid);

MP-TsOH (polystyrene resin bound equivalent of p-TsOH from Argonaut Technologies).

EDC: 1-(3-dimethylaminopropyl)-3-ethylcarbodiimide hydrochloride.

HOBT: 1-Hydroxybenzotriazole

HBTU: O-Benzotriazol-1-yl-N,N,N′,N′-tetramethyluronium hexafluorophosphate

BOPCl: Bis(2-oxo-3-oxazolidinyl)phosphinic chloride

MP-carbonate: Macroporous triethylammonium methylpolystyrene carbonate.

Unless otherwise indicated, all temperatures are expressed in ° C. (degrees Centigrade). All reactions conducted at room temperature unless otherwise noted.

¹H-NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on Micromass Platform or ZMD mass spectrometers from Micromass Ltd., Altricham, UK, using either Atmospheric Chemical Ionization (APCI) or Electrospray Ionization (ESI).

Analytical thin layer chromatography was used to verify the purity of intermediate(s) which could not be isolated or which were too unstable for full characterization as well as to follow the progress of reaction(s).

The absolute configuration of compounds can be assigned by Ab Initio Vibrational Circular Dichroism (VCD) Spectroscopy. The experimental VCD spectra were acquired in CDCl₃ using a Bomem Chiral® VCD spectrometer operating between 2000 and 800 cm⁻¹. The Gaussian 98 Suite of computational programs was used to calculate model VCD spectrums. The stereochemical assignments were made by comparing this experimental spectrum to the VCD spectrum calculated for a model structure with (R)- or (S)-configuration. Incorporated by reference with regard to such spectroscopy are: J. R. Chesseman, M. J. Frisch, F. J. Devlin and P. J. Stephens, Chem. Phys. Lett. 252 (1996) 211; P. J. Stephens and F. J. Devlin, Chirality 12 (2000) 172; and Gaussian 98, Revision A.11.4, M. J. Frisch et al., Gaussian, Inc., Pittsburgh Pa., 2002.

Compounds of formula (I) where all variables are as defined herein, and specifically wherein R is H and t is 1, can be prepared according to Scheme 1. Compounds of formula (I) wherein t is 0 or 2 can be made in a similar fashion as would be evident to one of skill in the art.

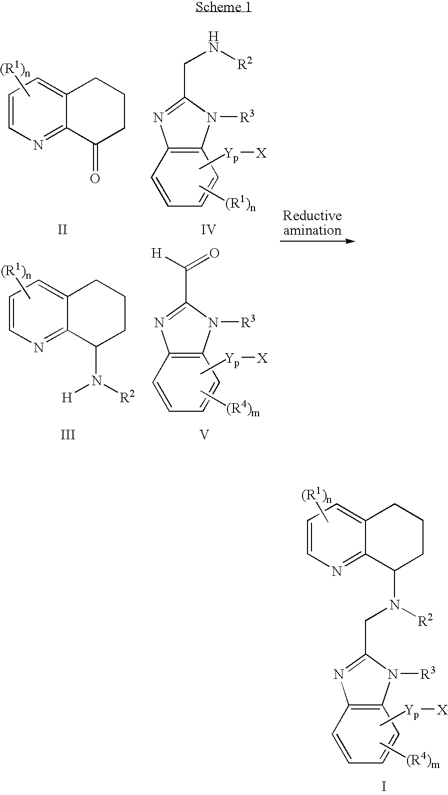

More specifically, compounds of formula (I) can be prepared by reacting a compound of formula (II) with a compound (IV) or alternatively reacting a compound of formula (III) with a compound of formula (V) under reductive conditions. The reductive amination can be carried out by treating the compound of formula (II) or (III) with a compound of formula (IV) or (V), respectively, in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally, the reaction can be run in presence of acid, such as acetic acid and the like.

Compounds of formula (II) can be prepared as described in the literature (J. Org. Chem., 2002, 67, 2197-2205, herein incorporated by reference with regard to such synthesis). Compounds of formula (III) can be prepared by reductive amination of compound of formula (II) using processes well known to those skilled in the art of organic synthesis. Compounds of formula (IV) and (V) can be prepared by methods similar to those described in the literature (Tet. Lett. 1998, 39, 7467-7470; WO02/092575; WO03/053344; WO03/106430; Science of Synthesis 2002, 12, 529-612; each incorporated by reference with regard to such synthesis).

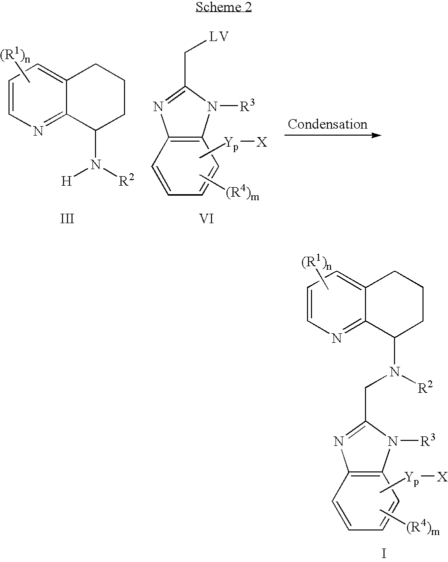

Compound of formula (I) wherein t is 1, each R is H and all other variables are as defined above, can be prepared by reaction of a compound of formula (III) with a compound of formula (VI) where LV is a leaving group (e.g., halogen, mesylate, tosylate) as outlined in Scheme 2. This condensation is typically carried out in a suitable solvent optionally in the presence of a base, optionally with heating. Suitable solvents include tetrahydrofuran, dioxane, acetonitrile, nitromethane, N,N-dimethylformamide, and the like. Suitable bases include triethylamine, pyridine, dimethylaminopyridine, N,N-diisopropylethylamine, potassium carbonate, sodium carbonate, and the like. The reaction can be carried out at room temperature or optionally heated to 30-200° C. A catalyst, such as potassium iodide, tertbutylammonium iodide, or the like, can optionally be added to the reaction mixture.

Compound of formula (VI) can be prepared in a similar fashion as described in the literature (Bioorg Med. Chem. Lett. 2003, 13, 3177; Bioorg. Med. Chem. 2004, 12, 5181, herein incorporated by reference with regard to such synthesis).

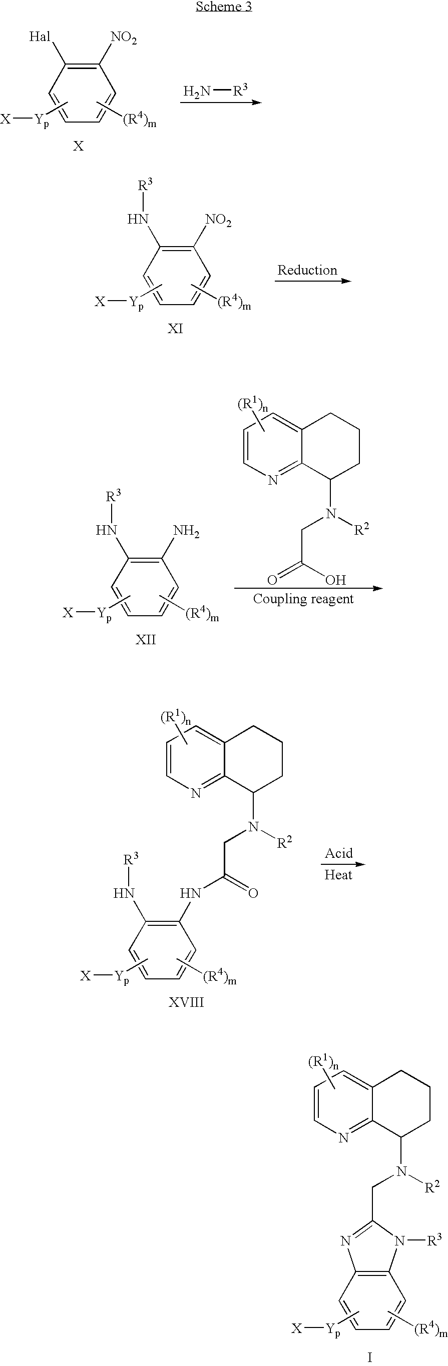

More specifically, compounds of formula (I) wherein t is 1, each R is H and all other variables are as defined above, can be prepared by treatment of compound of formula (XVIII) under acidic conditions optionally with heating. The reaction can be carried out by treating the compound of formula (XVIII) with a suitable acid optionally in the presence of an inert solvent. The reaction may be heated to 50-200° C. or performed at ambient temperature. Suitable acids include acetic acid, trifluoroacetic acid, hydrochloric acid, and the like. The reaction can be carried out using the acid as a solvent. Other suitable solvents include tetrahydrofuran, acetonitrile, toluene, and the like.

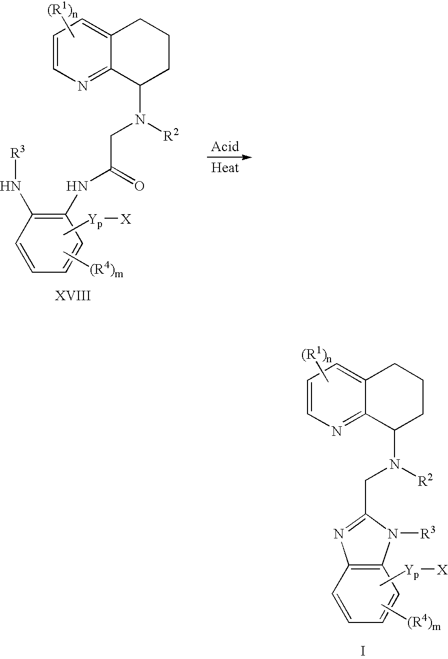

More specifically, as illustrated below, compounds of formula (XVIII) can be prepared by coupling of a compound of formula (XII) with a compound of formula (XVII). This coupling can be carried out using a variety of coupling reagent well know to those skilled in the art of organic synthesis (e.g., EDC, HOBt/HBTu; BOPCl). The reaction can be carried out with heating or at ambient temperature. Suitable solvents for this reaction include acetonitrile, tetrahydrofuran, and the like. Compounds of formula (XII) are commercially available or can be prepared by methods known in the literature. Compounds of formula (XVII) can be prepared from tetrahydroquinoline-8-one and a protected glycine derivative by reductive amination, followed by deprotection.

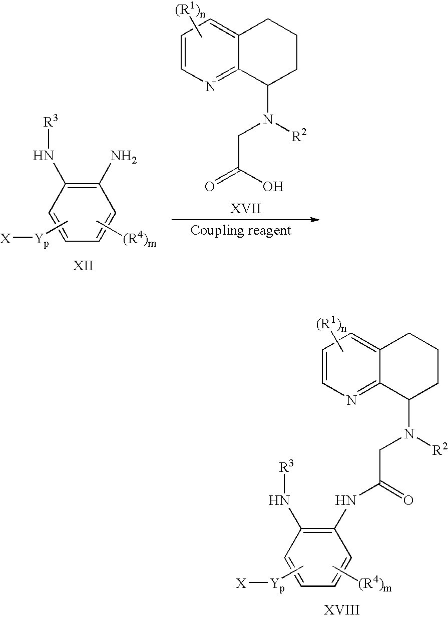

Compounds of formula (I) can be prepared as outlined in Scheme 4, where Z is a suitable protecting group, t is 1, R is H and all other variables are as defined in connection with compound of formula (I). Compounds of formula (I) where t is 0 or 2 can be made in a similar fashion as would be evident to one skilled in the art.

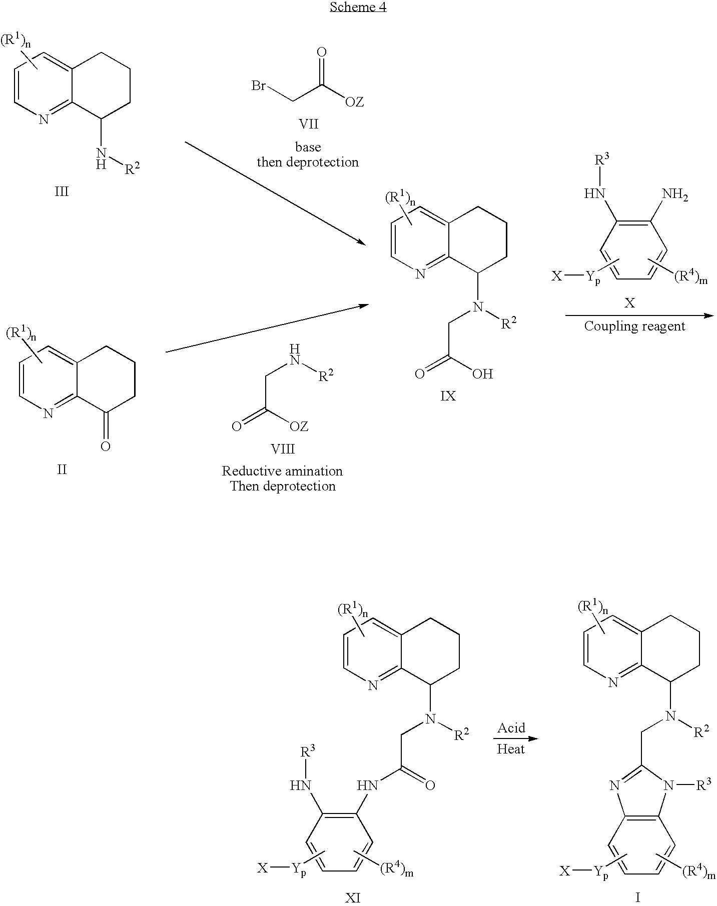

Compounds of formula (I) wherein t is 1 and each R is H and all other variables are as hereinbefore defined can be prepared by treatment of a compound of formula (XI) under acidic conditions optionally with heating. The reaction can be carried out by treating the compound of formula (XI) with a suitable acid optionally in the presence of an inert solvent, such as but not limited to tetrahydrofuran, acetonitrile, toluene, and the like. The reaction may be heated to 50-200° C. or performed at ambient temperature. Suitable acids include acetic acid, trifluoroacetic acid, hydrochloric acid, and the like. The reaction can be carried out using the acid as a solvent.

More specifically, as illustrated below, compounds of formula (XI) can be prepared by coupling of a compound of formula (X) with a compound of formula (IX). This coupling can be carried out using a variety of coupling reagents well known to those skilled in the art of organic synthesis (e.g., EDC, HOBt/HBTu; BOPCl). The reaction can be carried out with heating or at ambient temperature. Suitable solvents for this reaction include acetonitrile, tetrahydrofuran, and the like. Compounds of formula (X) can be prepared by methods known in the literature (e.g. from 3-chloro-2-nitroaniline). Compounds of formula (IX) can be prepared from a compound of formula (II) and a protected glycine derivative (VIII) by reductive amination, followed by deprotection. Alternatively compound of formula (IX) can be prepared from compound of formula (III) and compound of formula (VII) via methods well known to those skilled in the art of organic synthesis.

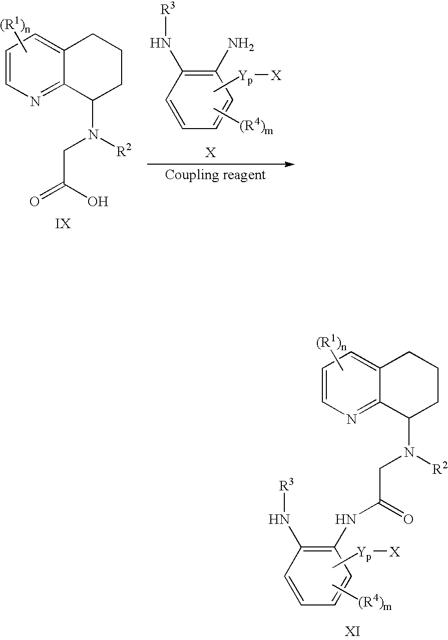

A compound of formula (I) where either one or both R are not H and all other variables are as defined for formula (I) can be made according to Scheme 4 by using an amino acid other than glycine as is evident to one skilled in the art.

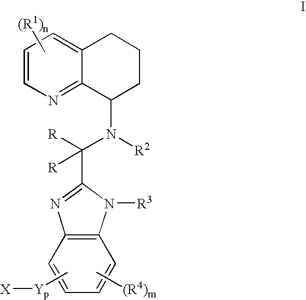

A compound of formula I-B wherein R³ is H; t is 1; each R is H; W is alkyl or another suitable protecting group and all other variables are as defined herein, can be prepared according to Scheme 5.

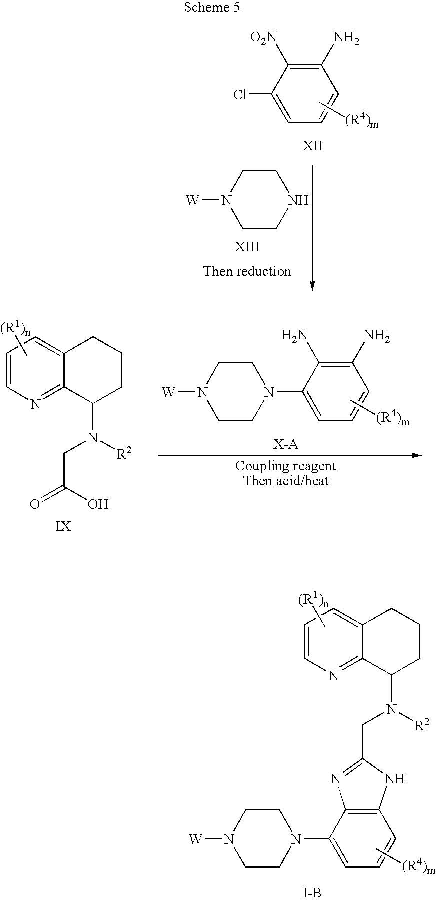

Generally the process of preparing compound of formula (I-B) where R³ is H and all variables are as defined herein above includes the steps of:
a) Reacting a compound of formula (XII) with a compound of formula (XIII), followed by reduction to form a compound of formula (X-A); and
b) Coupling a compound of formula (X-A) with a compound of formula (IX) and treating the coupled product with acid and heat to form a compound of formula I-B.

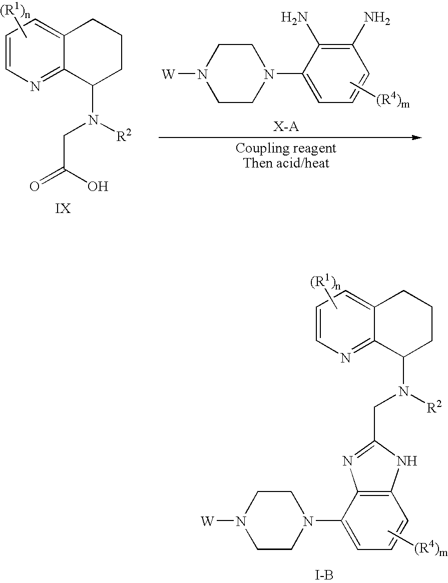

More specifically compounds of formula (I-B) can be prepared by coupling of compound of formula (X-A) and compound of formula (IX) followed by treatment with acid. Typical coupling reagents include EDC, HOBt/HBTu and BOPCl. Compounds of formula (I-B) can be prepared by treatment of the intermediate amide under acidic conditions optionally with heating. The reaction can also be carried out by treatment with a suitable acid optionally in the presence of an inert solvent. Suitable acids include acetic acid, trifluoroacetic acid, hydrochloric acid, and the like. Suitable solvents for this reaction include acetonitrile, tetrahydrofuran, and the like. The reaction may be heated to 50-200° C. or performed at ambient temperature. The reaction can be carried out using the acid as a solvent. Other suitable solvents include toluene, and the like. Compounds of formula (IX) can be prepared as described previously.

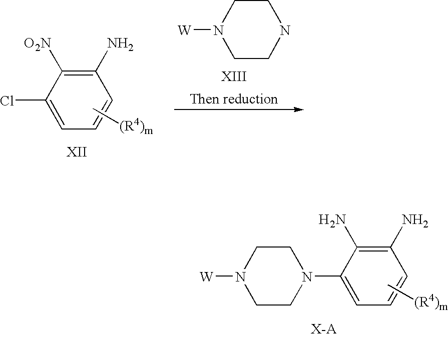

Compound of formula (X-A) can be prepared from a compound of formula (XII) and a compound of formula (XIII) by condensation optionally in the presence of solvent and optionally with heating or in a microwave, followed by reduction. Compounds of formula (XII) and (XIII) are readily commercially available or can be prepared by conditions well known to those skilled in the art of organic chemistry.

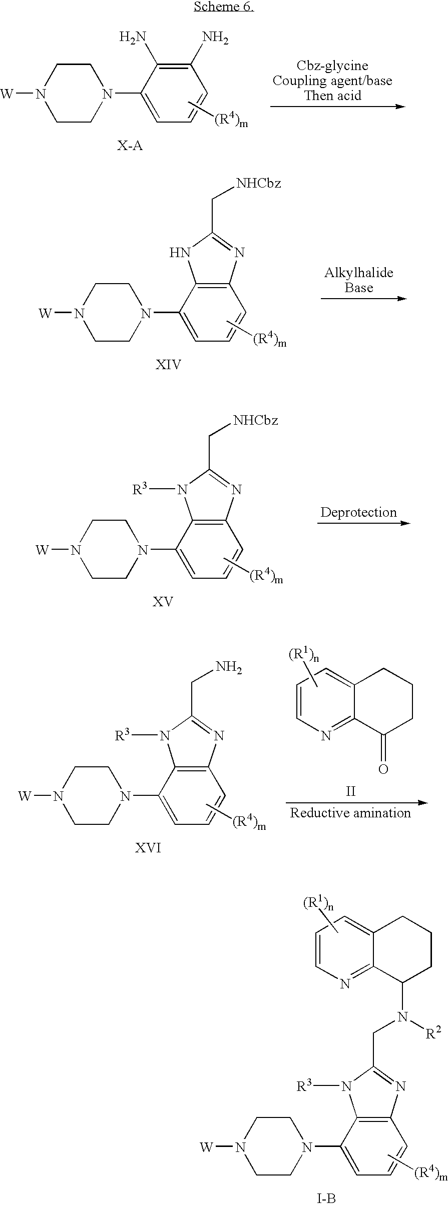

Generally the process of preparing compound of formula I-B where t is 1, R³ is alkyl, W is alkyl or a suitable protecting group; and all other variables are as defined hereinabove include the steps of:
a) Preparing a compound of formula (XIV) from a compound of formula (X-A) and protected glycine;
b) Preparing a compound of formula (XV) from a compound of formula (XIV);
c) Preparing a compound for formula (XVI) from a compound of formula (XV); and
d) Reacting a compound of formula (II) with compound of formula (XVI) to form compound of formula (I-B).

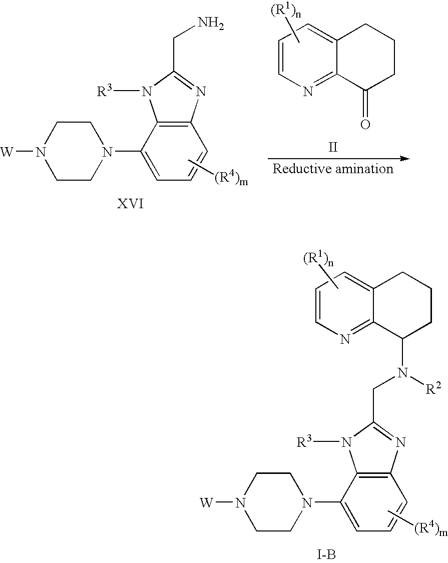

More specifically a compound of formula (I-B) can be prepared from a compound of formula (XVI) and a compound of formula (II) via reductive amination. The reductive amination can be carried out by treating the compound of formula (II) with a compound of formula (XVI) in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like. For compounds of formula (I-B) where R² is H a second reductive amination step can be used to convert one compound of formula (I-B) to a different compound of formula (I-B) wherein R² is alkyl.

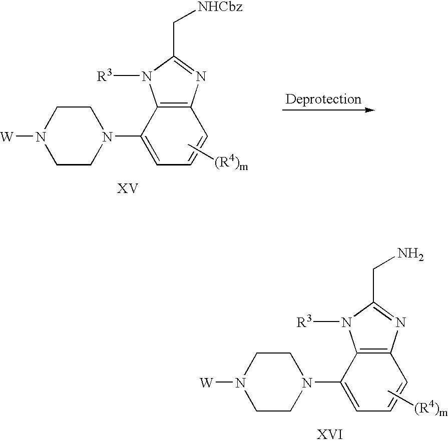

More specifically a compound of formula (XVI) can be prepared from a compound of formula (XV) by deprotection. For deprotection of Cbz protecting groups catalytic reduction or treatment with acid are among suitable deprotection methods. For the catalytic reduction suitable catalysts include Pd/C and the like under hydrogen atmosphere. Suitable solvents include alcohols and the like. For acidic reductions suitable acids include trifluoroacetic acid, hydrochloric acid and the like.

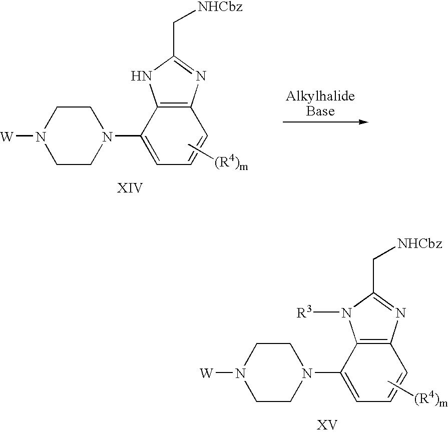

More specifically a compound of formula (XV) can be prepared from a compound of formula (XIV). Treatment of a compound of formula (XIV) with a suitable alkylhalide in a solvent, optionally with heating and optionally in the presence of base gives compound of formula (XV) as one of the obtained isomers. Suitable alkylhalides include methyliodide, ethyliodide and the like. Suitable solvents include dimethylformamide, dimethylsulfoxide, N-methylpyrrolidinone, nitromethane, acetonitrile and the like. Suitable bases include potassium carbonate, cesium carbonate, sodium hydride and the like. Reaction can optionally be heated between 20-200° C. or carried out in a microwave.

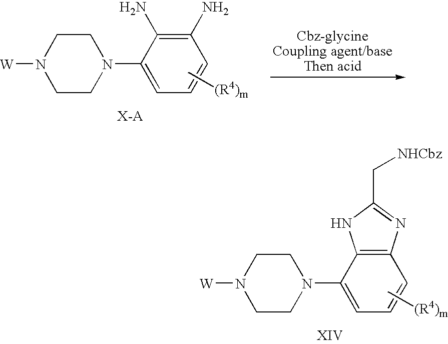

More specifically a compound of formula (XIV) can be prepared from a compound of formula (X-A). Treatment of a compound of formula (X-A) with Cbz-glycine and a suitable coupling agent (EDC, HOBt/HBTu and BOPCl) followed by treatment of the resulting amide under acidic conditions optionally with heating. The reaction can be carried out by treatment with a suitable acid optionally in the presence of an inert solvent. The reaction may be heated to 50-200° C. or performed at ambient temperature. Suitable acids include acetic acid, trifluoroacetic acid, hydrochloric acid, and the like. The reaction can be carried out using the acid as a solvent. Other suitable solvents include tetrahydrofuran, acetonitrile, toluene, and the like.

As is evident to one skilled in the art of organic synthesis an alternative in of preparing a compound of formula (XV) would be to use a compound of formula (XVII) as a starting material and upon ring closure obtain a benzimidazole isomer, as shown below. Treatment of a compound of formula (X-A) with Cbz-glycine and a suitable coupling agent (EDC, HOBt/HBTu and BOPCl) followed by treatment of the resulting amide under acidic conditions optionally with heating. The reaction can be carried out by treatment with a suitable acid optionally in the presence of an inert solvent. The reaction may be heated to 50-200° C. or performed at ambient temperature. Suitable acids include acetic acid, trifluoroacetic acid, hydrochloric acid, and the like. The reaction can be carried out using the acid as a solvent. Other suitable solvents include tetrahydrofuran, acetonitrile, toluene, and the like.

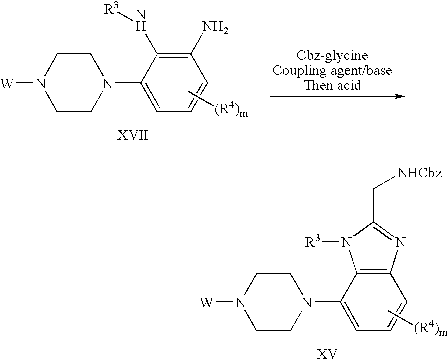

A compound of formula (I) where t is 1, either one or both R are not H and all other variables are as defined for formula (I) can be made according to Scheme 6 by using an aminoacid other than glycine as is evident to one skilled in the art.

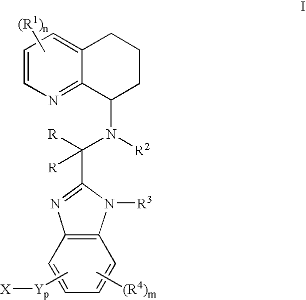

A compound of formula (I-B) where R³ is alkyl, t is 1 and all other variables are as defined above and W is alkyl or a suitable protecting group can be prepared as outlined in Scheme 7. Compounds of formula (I-B) where t is 0 or 2 can be made using similar methods as is evident to one skilled in the art.

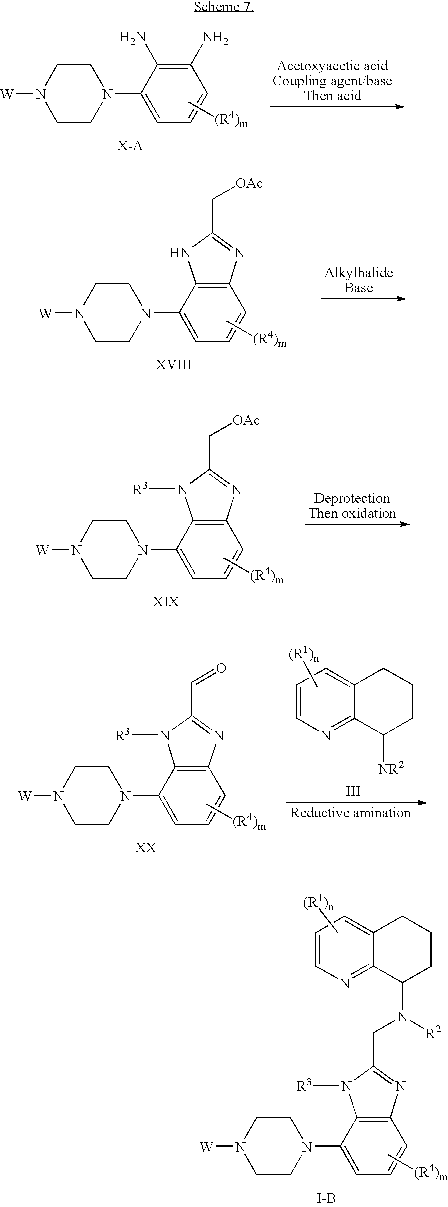

Generally the process of preparing compound of formula (I-B) wherein R³ is alkyl, t is 1, W is alkyl or a suitable protecting group, and all other variables are as defined hereinabove include the steps of:
a) Preparing a compound of formula (XVIII) from a compound of formula (X-A) and acetoxyacetic acid or a related acetic acid derivative;
b) Preparing a compound of formula (XIX) from a compound of formula (XVIII);
c) Preparing a compound for formula (XX) from a compound of formula (XIX); and
d) Reacting a compound of formula (III) with compound of formula (XX) to form compound of formula (I-B).

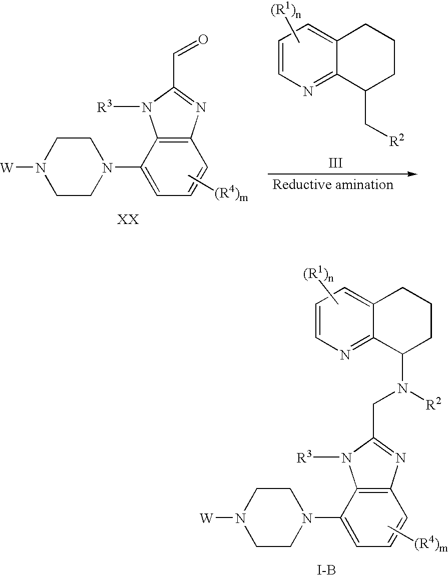

More specifically a compound of formula (I-B) can be prepared from a compound of formula (XX) and a compound of formula (III) via reductive amination. The reductive amination can be carried out by treating the compound of formula (III) with a compound of formula (XX) in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like.

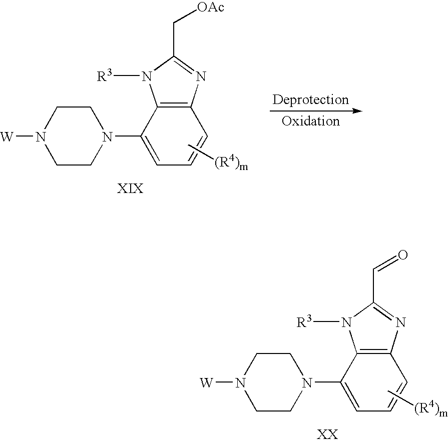

More specifically a compound of formula (XX) can be prepared from a compound of formula (XIX) by deprotection, followed by oxidation of the alcohol to aldehyde. For deprotection of OAc protecting groups treatment with aq base is among suitable deprotection methods. Suitable methods for oxidations of the alcohol include treatment with MnO₂ and related oxidatants in a suitable solvent, such as acetonitrile, dichloromethane, chloroform and the like.

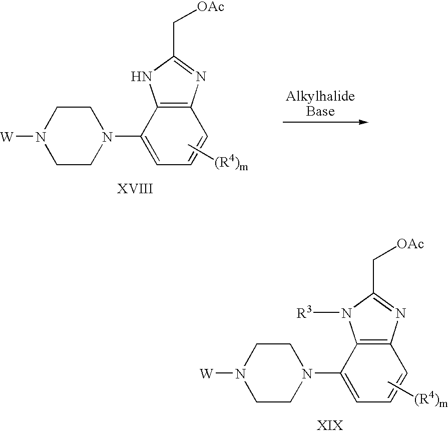

More specifically a compound of formula (XIX) can be prepared from a compound of formula (XVIII). Treatment of a compound of formula (XVIII) with a suitable alkylhalide in a solvent, optionally with heating and optionally in the presence of base gives compound of formula (XIX) as one of the obtained isomers. Suitable alkylhalides include methyliodide, ethyliodide and the like. Suitable solvents include dimethylformamide, dimethylsulfoxide, N-methylpyrrolidinone, nitromethane, acetonitrile and the like. Suitable bases include potassium carbonate, cesium carbonate, sodium hydride and the like. Reaction can optionally be heated between 20-200° C. or carried out in a microwave.

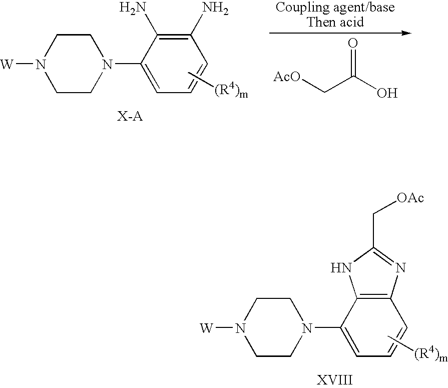

More specifically a compound of formula (XVIII) can be prepared from a compound of formula (X-A). Treatment of a compound of formula (X-A) with acetoxyacetic acid (or related acetic acid derivatives, such as hydroxyacetic acid) and a suitable coupling agent (HATU, EDC, HOBt/HBTu and BOPCl) followed by treatment of the resulting amide under acidic conditions optionally with heating gives compound of formula (XVIII). The reaction can be carried out by treatment with a suitable acid optionally in the presence of an inert solvent. The reaction may be heated to 50-200° C. or performed at ambient temperature. Suitable acids include acetic acid, trifluoroacetic acid, hydrochloric acid, and the like. The reaction can be carried out using the acid as a solvent. Other suitable solvents include acetonitrile, toluene, and the like.

An alternative would be to use a compound of formula (XXI) as a starting material and upon ring closure obtain a benzimidazole isomer, as shown below. Treatment of a compound of formula (XVII) with an acetic acid derivative and a suitable coupling agent (EDC, HOBt/HBTu and BOPCl) followed by treatment of the resulting amide under acidic conditions optionally with heating. The reaction can be carried out by treatment with a suitable acid optionally in the presence of an inert solvent. The reaction may be heated to 50-200° C. or performed at ambient temperature. Suitable acids include acetic acid, trifluoroacetic acid, hydrochloric acid, and the like. The reaction can be carried out using the acid as a solvent. Other suitable solvents include tetrahydrofuran, acetonitrile, toluene, and the like.

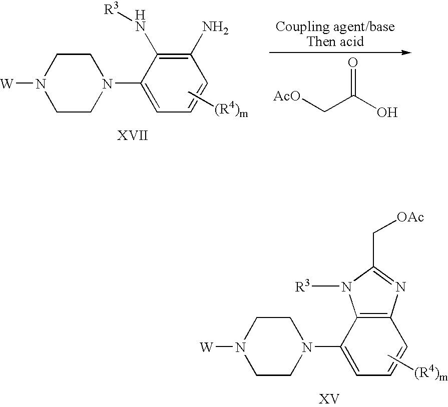

### EXAMPLES

### Example 1

### N-Ethyl-5,6,7,8-tetrahydro-8-quinolinamine

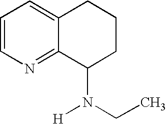

To 6,7-dihydro-8(5H)-quinolinone (1.0 g, 6.8 mmol, J. Org. Chem., 2002, 67, 2197-2205) dissolved in dichloroethane (75 mL) was added a 2M solution of ethyl amine in tetrahydrofuran (5.1 mL, 10.2 mmol) and acetic acid (0.4 mL, 10.2 mmol). Sodium triacetoxyborohydride (2.1 g, 10.2 mmol) was added in 4 portions over 3 h. The mixture was stirred at room temperature for 2 h, sat. sodium bicarbonate was added (25 mL) and the bi-phasic mixture stirred vigorously for 5 min. The layers were separated and the aq. portion extracted with methylene chloride containing 0.1% methanol (2×50 mL). The organic layers were combined, dried over sodium sulfate, filtered and evaporated under reduced pressure to give a brown oil. Purification by silica gel chromatography with methylene chloride and 2N ammonia in methanol afforded N-ethyl-5,6,7,8-tetrahydro-8-quinolinamine as a clear oil (0.6 g, 50% yield). ¹H-NMR (DMSO-d₆): δ 8.34 (d, 1H), 7.47 (d, 1H), 7.16 (dd, 1H), 3.65 (t, 1H), 2.74-2.58 (m, 4H), 2.48-1.97 (m, 1H), 1.91-1.84 (m, 1H), 1.66-1.57 (m, 2H), 1.06 (t, 3H). MS m/z 177.1 (M+1).

### Example 2

### N-Methyl-5,6,7,8-tetrahydro-8-quinolinamine

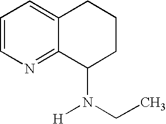

N-Methyl-5,6,7,8-tetrahydro-8-quinolinamine was prepared from 6,7-dihydro-8(5H)-quinolinone and methyl amine in a similar manner as described above to give a clear oil (0.55 g, 50% yield). ¹H-NMR (DMSO-d₆): δ 8.33 (d, 1H), 7.47 (d, 1H), 7.16 (dd, 1H), 3.52 (t, 1H), 2.71 (t, 2H), 2.37 (s, 3H), 2.00-1.95 (m, 1H), 1.91-1.84 (m, 1H), 1.68-1.60 (m, 2H). MS m/z 163 (M+1).

### Example 3

### 2-{[Methyl(5,6,7,8-tetrahydro-8-quinolinyl)amino]methyl}-1H-benzimidazole-4-carboxylic acid

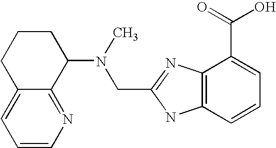

### A) Methyl 2,3-diaminobenzoate

Methyl 2-amino-3-nitrobenzoate (10 g, 51 mmol) was dissolved in ethanol (500 mL) under nitrogen. Palladium on carbon (10% w/w, 2.7 g, 2.6 mmol) was added under nitrogen. The reaction was placed under a hydrogen atmosphere (1 atm) and stirred for 16 h at room temperature. The reaction was flushed with nitrogen, filtered through diatomaceous earth and concentrated to provide the product (8.39 g, 99%) as a white solid. ¹H-NMR (DMSO-d₆) δ 7.07 (d, J=8.1 Hz, 1H), 6.68 (d, J=8.1 Hz, 1H), 6.37 (t, J=8.1 Hz, 1H), 6.18 (br s, 2H), 4.75 (br s, 2H), 3.74 (s, 3H).

### B) Methyl 2-[({[(phenylmethyl)oxy]carbonyl}amino)methyl]-1H-benzimidazole-4-carboxylate

To a solution of methyl 2,3-diaminobenzoate (8.39, 50.5 mmol) in acetonitrile (100 mL) was added bis(2-oxo-3-oxazolidinyl)phosphinic chloride (14.3 g, 56.1 mmol), carbobenzyloxyglycine (16.0 g, 76.5 mmol), and N,N-diisopropylethylamine (13.2 g, 17.7 mL, 102 mmol). The solution was stirred for 16 h at room temperature. The reaction was concentrated, diluted with ethyl acetate (200 mL) and water (200 mL), separated, dried over sodium sulfate, filtered and concentrated. The crude amide was dissolved in acetic acid (100 mL) and heated at 70° C. for 150 min. The reaction mixture was cooled, concentrated, diluted with ethyl acetate (200 mL) and saturated aqueous sodium bicarbonate (200 mL), separated, dried over sodium sulfate, filtered and concentrated to red oil. The crude material was purified on silica (5% methanol/dichloromethane) to provide the product (15.1 g, 87%) as a tan solid. ¹H-NMR (DMSO-d₆) δ 12.19 (s, 1H), 7.86-7.82 (m, 2H), 7.77 (d, 1H), 7.35-7.23 (m, 6H), 5.04 (s, 2H), 4.49 (d, J=6.1 Hz, 2H), 3.92 (s, 3H).

### C) Methyl 2-{[methyl(5,6,7,8-tetrahydro-8-quinolinyl)amino]methyl}-1H-benzimidazole-4-carboxylate

Methyl 2-[({[(phenylmethyl)oxy]carbonyl}amino)methyl]-1H-benzimidazole-4-carboxylate (7.0 g, 21 mmol), was dissolved in ethanol (200 mL) under nitrogen atmosphere. Palladium on carbon (10% w/w, 2.2 g, 2.1 mmol) was added. The reaction was placed under a hydrogen atmosphere (1 atm) and stirred for 16 h at room temperature. The reaction was flushed with nitrogen, filtered through diatomaceous earth and concentrated to give the amine as a white solid. This amine, 6,7-dihydro-8(5H)-quinolinone (3.0 g, 21 mmol) and acetic acid (1.9 g, 31 mmol) were dissolved in 1,2-dichloroethane (200 mL), sodium triacetoxyborohydride (6.6 g, 31 mmol) was added portionwise over 30 min and the reaction was stirred for 2 h at room temperature. The reaction mixture was diluted with saturated aqueous sodium bicarbonate (100 mL), phases separated, the organic phase dried over sodium sulfate, filtered and concentrated. The crude secondary amine, formaldehyde (37% aqueous solution, 3.1 mL, 41 mmol) and acetic acid (1.9 g, 31 mmol) were dissolved in 1,2-dichloroethane (200 mL), sodium triacetoxyborohydride (6.6 g, 31 mmol) was added portionwise over 30 min and the reaction was stirred for 1 h at room temperature. The reaction mixture was diluted with saturated aqueous sodium bicarbonate (100 mL), separated, dried over sodium sulfate, filtered and concentrated. The crude tertiary amine was purified on silica (2% methanol/dichloromethane) to provide the product (4.9 g, 68%) as a yellow foam. ¹H-NMR (DMSO-d₆) δ 12.85 (s, 1H), 8.61 (d, 1H), 7.85-7.78 (m, 2H), 7.56-7.54 (m, 1H), 7.27-7.23 (m, 2H), 3.98 (s, 3H), 3.98-.94 (m, 1H), 3.87-3.86 (m, 2H), 2.88-2.66 (m, 2H), 2.33 (s, 3H), 2.08-1.95 (m, 2H), 1.91-1.85 (m, 1H), 1.73-1.64 (m, 1H).

### D) 2-{[Methyl(5,6,7,8-tetrahydro-8-quinolinyl)amino]methyl}-1H-benzimidazole-4-carboxylic acid

Methyl 2-{[methyl(5,6,7,8-tetrahydro-8-quinolinyl)amino]methyl}-1H-benzimidazole-4-carboxylate (1.5 g, 4.3 mmol) was dissolved in methanol (5 mL) and tetrahydrofuran (5 mL) and aqueous lithium hydroxide (206 mg in 10 mL water, 8.6 mmol) was added. The reaction mixture was stirred for 72 h at 70° C. The reaction was concentrated and purified using reverse phase chromatography (10% to 40% acetonitrile/water/0.1% trifluoroacetic acid gradient) to provide the product (1.2 g, 84%) as a tan solid: ¹H-NMR (DMSO-d₆) δ 11.99 (s, 1H), 8.59-8.58 (m, 1H), 7.55-7.41 (m, 3H), 7.17-7.14 (m, 1H), 7.01-6.97 (m, 1H), 4.10 (br s, 1H), 3.97-3.82 (m, 3H), 2.86-2.64 (m, 2H), 2.27 (s, 3H), 2.11-2.01 (m, 1H), 1.98-1.80 (m, 2H), 1.70-1.60 (m, 1H).

### Example 4

### Methyl 1-methyl-2-[({[(phenylmethyl)oxy]carbonyl}amino)methyl]-1H-benzimidazole-7-carboxylate; and

### Methyl 1-methyl-2-[({[(phenylmethyl)oxy]carbonyl}amino)methyl]-1H-benzimidazole-4-carboxylate

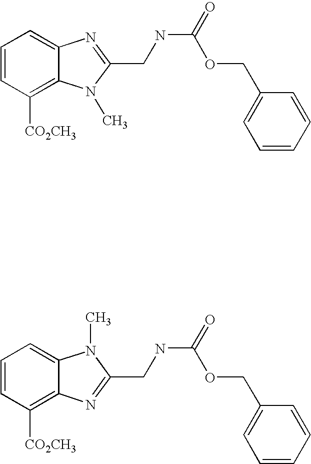

A slurry of methyl 2-[({[(phenylmethyl)oxy]carbonyl}amino)methyl]-1H-benzimidazole-4-carboxylate (170 mg, 0.50 mmol), cesium carbonate (244 mg, 0.75 mmol) and iodomethane (170 mg, 1.2 mmol) in N,N-dimethylformamide (10 mL) was stirred at room temperature for 16 h. The reaction mixture was filtered through diatomaceous earth, concentrated and purified on silica (30% to 100% ethyl acetate/hexanes gradient) to provide the products as white solids:

Methyl 1-methyl-2-[({[(phenylmethyl)oxy]carbonyl}amino)methyl]-1H-benzimidazole-7-carboxylate (44 mg, 25%). ¹H-NMR (CDCl₃): δ 7.96 (d, 1H), 7.54 (d, 1H), 7.36-7.32 (m, 6H), 5.14 (s, 2H), 4.76 (d, 2H), 4.01 (s, 3H), 3.84 (s, 3H).

Methyl 1-methyl-2-[({[(phenylmethyl)oxy]carbonyl}amino)methyl]-1H-benzimidazole-4-carboxylate (94 mg, 53%). ¹H-NMR (CDCl₃) δ 7.87 (d, 1H), 7.78 (d, 1H), 7.37-7.24 (m, 6H), 5.16 (s, 2H), 4.68 (s, 2H), 3.97 (s, 3H), 3.92 (s, 3H).

### Example 5

### Methyl 1-methyl-2-{[methyl(5,6,7,8-tetrahydro-8-quinolinyl)amino]methyl}-1H-benzimidazole-7-carboxylate

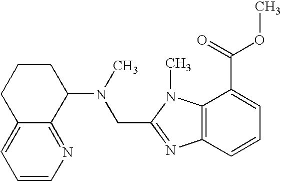

Methyl 1-methyl-2-[({[(phenylmethyl)oxy]carbonyl}amino)methyl]-1H-benzimidazole-7-carboxylate (500 mg, 1.4 mmol) was dissolved in ethanol (200 mL) under nitrogen. Palladium on carbon (10% w/w, 150 mg, 0.14 mmol) was added. The reaction was placed under a hydrogen atmosphere (1 atm) and stirred for 16 h at room temperature. The reaction was flushed with nitrogen, filtered through diatomaceous earth and concentrated to give the amine as a clear oil (220 mg). This amine (220 mg, 1.07 mmol), 6,7-dihydro-8(5H)-quinolinone (160 mg, 1.1 mmol), acetic acid (96 mg, 1.6 mmol) and sodium triacetoxyborohydride (340 mg, 1.6 mmol) were dissolved in 1,2-dichloroethane (20 mL) and stirred for 16 h at room temperature. The reaction mixture was diluted with dichloromethane (100 mL) and saturated aqueous sodium bicarbonate (100 mL), the phases separated, the organic phase dried over sodium sulfate, filtered, and concentrated to a red oil. The crude secondary amine, formaldehyde (37% aqueous solution, 0.16 mL, 2.1 mmol), acetic acid (96 mg, 1.6 mmol) and sodium triacetoxyborohydride (340 mg, 1.6 mmol) were dissolved in 1,2-dichloroethane (20 mL) and stirred for 2 h at room temperature. The reaction mixture was diluted with dichloromethane (100 mL) and saturated aqueous sodium bicarbonate (100 mL), the phases separated, the organic phase dried over sodium sulfate, filtered and concentrated. The crude tertiary amine was purified on silica (2% 2M ammonia in methanol/dichloromethane) to provide the product as a red oil (220 mg, 43%). ¹H-NMR (CDCl₃) δ 8.48 (d, 1H), 7.85 (d, 1H), 7.70 (d, 1H), 7.34 (d, 1H), 7.20 (t, 1H), 7.06-7.03 (m, 1H), 4.12 (d, J=13.5 Hz, 1H), 3.99-3.96 (m, 8H), 2.88-2.80 (m, 1H), 2.74-2.67 (m, 1H), 2.34 (s, 3H), 2.11-2.01 (m, 2H), 2.00-1.80 (m, 1H), 1.75-1.67 (m, 1H).

### Example 6

### Methyl 1-methyl-2-{[methyl(5,6,7,8-tetrahydro-8-quinolinyl)amino]methyl}-1H-benzimidazole-4-carboxylate

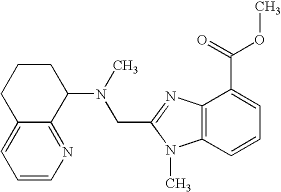

Methyl 1-methyl-2-[({[(phenylmethyl)oxy]carbonyl}amino)methyl]-1H-benzimidazole-4-carboxylate (1.0 g, 2.8 mmol) was dissolved in ethanol (200 mL) under nitrogen. Palladium on carbon (10% w/w, 300 mg, 0.28 mmol) was added and the solution was flushed with nitrogen. The reaction was placed under a hydrogen atmosphere (1 atm) and stirred for 16 h at room temperature. The reaction was flushed with nitrogen, filtered through diatomaceous earth and concentrated to give the amine as a clear oil (490 mg). The amine (490 mg, 2.4 mmol), 6,7-dihydro-8(5H)-quinolinone (350 mg, 2.4 mmol), acetic acid (220 mg, 3.6 mmol) and sodium triacetoxyborohydride (760 mg, 3.6 mmol) were dissolved in 1,2-dichloroethane (20 mL) and stirred for 16 h at room temperature. The reaction mixture was diluted with dichloromethane (100 mL) and saturated aqueous sodium bicarbonate (100 mL), the phases were separated, the organic phase was dried over sodium sulfate, filtered and concentrated to an oil. The crude secondary amine, formaldehyde (37% aqueous solution, 0.36 mL, 4.8 mmol), acetic acid (220 mg, 3.6 mmol) and sodium triacetoxyborohydride (760 mg, 3.6 mmol) were dissolved in 1,2-dichloroethane (20 mL) and stirred for 2 h at room temperature. The reaction mixture was diluted with dichloromethane (100 mL) and saturated aqueous sodium bicarbonate (100 mL), the phases separated, the organic phase dried over sodium sulfate, filtered, and concentrated. The crude tertiary amine was purified on silica (2% 2M ammonia in methanol/dichloromethane) to provide the product (480 mg, 47%) as a red oil. ¹H-NMR (CDCl₃) δ 8.46 (d, 1H), 7.88 (d, 1H), 7.48 (d, 1H), 7.33 (d, 1H), 7.26 (t, 1H), 7.04-7.01 (m, 1H), 4.18 (d, 1H), 4.05-3.95 (m, 8H), 2.88-2.80 (m, 1H), 2.72-2.66 (m, 1H), 2.35 (s, 3H), 2.13-2.01 (m, 3H), 1.77-1.66 (m, 1H).

### Example 7

### N-[2-(1H-Imidazol-4-yl)ethyl]-1-methyl-2-{[methyl(5,6,7,8-tetrahydro-8-quinolinyl)amino]methyl}-1H-benzimidazole-7-carboxamide

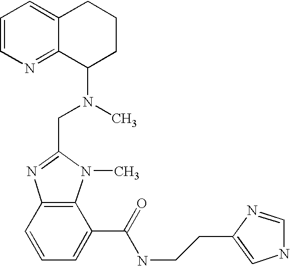

Methyl 1-methyl-2-{[methyl(5,6,7,8-tetrahydro-8-quinolinyl)amino]methyl}-1H-benzimidazole-7-carboxylate (220 mg, 0.6 mmol) was dissolved methanol (5 mL), tetrahydrofuran (5 mL) and aqueous lithium hydroxide (1N, 2 mL, 2 mmol) and the reaction was stirred for 16 h at 70° C. An additional portion of aqueous lithium hydroxide (1N, 2 mL, 2 mmol) was added and the reaction was stirred for 72 h at 70° C. The reaction was cooled to room temperature and concentrated and the acid was carried on crude. The crude acid (105 mg, 0.3 mmol), bis(2-oxo-3-oxazolidinyl)phosphinic chloride (109 mg, 0.43 mmol), histamine (48 mg, 0.43 mmol), and N,N-diisopropylethylamine (56 mg, 0.43 mmol) were dissolved in acetonitrile (5 mL) and N,N-dimethylformamide (2 mL) and the reaction was stirred for 16 h at room temperature. The reaction mixture was concentrated and purified using reverse phase HPLC (0% to 70% acetonitrile/water/0.1% trifluoroacetic acid gradient) to provide the product (42.3 mg, 18%) as a tan solid. ¹H-NMR (DMSO-d₆) δ 9.01 (s, 1H), 8.82-8.79 (m, 1H), 8.51 (d, 1H), 7.78-7.71 (m, 2H), 7.51 (s, 1H), 7.40-7.37 (m, 1H), 7.29-723 (m, 2H), 4.96-4.91 (m, 1H), 4.76 (d, 1H), 4.55 (d, 1H), 3.65 (s, 3H), 3.62-3.57 (m, 2H), 2.96-2.90 (m, 3H), 2.86-2.81 (m, 5H), 2.14-2.04 (m, 2H), 1.85-1.71 (m, 1H). MS m/z 444 (M+1).

### Example 8

### N-Methyl-N-{[1-methyl-7-(1-piperazinylcarbonyl)-1H-benzimidazol-2-yl]methyl}-5,6,7,8-tetrahydro-8-quinolinamine

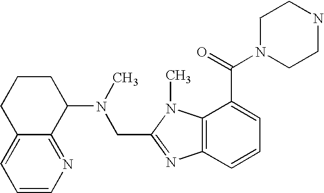

1-methyl-2-{[methyl(5,6,7,8-tetrahydro-8-quinolinyl)amino]methyl}-1H-benzimidazole-7-carboxylic acid (105 mg, 0.3 mmol), bis(2-oxo-3-oxazolidinyl)phosphinic chloride (109 mg, 0.43 mmol), N-butoxycarbonylpiperizine (80, 0.43 mmol), and N,N-diisopropylethylamine (56 mg, 0.43 mmol) were dissolved in acetonitrile (5 mL) and N,N-dimethylformamide (2 mL) and the reaction was stirred for 16 h at room temperature. The reaction mixture was concentrated and purified using reverse phase HPLC (0% to 70% acetonitrile/water/0.1% trifluoroacetic acid gradient) to provide the protected amine. The amine was dissolved in dichloromethane (2 mL) and trifluoroacetic acid (2 mL) and stirred for 3 h. The reaction was concentrated and dried to provide the product (30.0 mg, 13%) as a sticky yellow solid. ¹H-NMR (DMSO-d₆) δ 9.01 (br s, 1H), 8.51 (d, 1H), 7.78-7.69 (m, 2H), 7.40-7.27 (m, 3H), 5.01-4.90 (m, 1H), 4.81-4.71 (m, 1H), 4.59-4.52 (m, 1H), 4.00-3.84 (m, 2H), 3.62 (s, 3H), 3.54-3.40 (m, 2H), 3.29-2.97 (m, 4H), 2.88-2.82 (m, 5H), 2.53-2.49 (m, 1H), 2.16-2.06 (m, 2H), 1.82-1.69 (m, 1H). MS m/z 419 (M+1).

### Example 9

### N-[2-(1H-Imidazol-4-yl)ethyl]-1-methyl-2-{[methyl(5,6,7,8-tetrahydro-8-quinolinyl)amino]methyl}-1H-benzimidazole-4-carboxamide

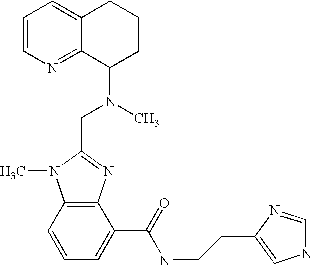

Methyl 1-methyl-2-{[methyl(5,6,7,8-tetrahydro-8-quinolinyl)amino]methyl}-1H-benzimidazole-4-carboxylate (480 mg, 1.3 mmol), was dissolved methanol (5 mL), tetrahydrofuran (5 mL) and aqueous lithium hydroxide (1N, 2 mL, 2 mmol) and the reaction was stirred for 16 h at 70° C. The reaction was cooled to room temperature and concentrated and the resulting acid, 1-methyl-2-{[methyl(5,6,7,8-tetrahydro-8-quinolinyl)amino]methyl}-1H-benzimidazole-4-carboxylic acid, was used for the next step without purification. This crude acid (100 mg, 0.29 mmol), bis(2-oxo-3-oxazolidinyl)phosphinic chloride (109 mg, 0.43 mmol), histamine (48 mg, 0.43 mmol), and N,N-diisopropylethylamine (56 mg, 0.43 mmol) were dissolved in N,N-dimethylformamide (5 mL) and the reaction was stirred for 16 h at room temperature. The reaction mixture was concentrated and purified using reverse phase HPLC (0% to 70% acetonitrile/water/0.1% trifluoroacetic acid gradient) to provide the product (40.7 mg, 18%) as a tan solid. ¹H-NMR (DMSO-d₆) δ 9.52-9.50 (m, 1H), 8.95 (s, 1H), 8.49 (d, 1H), 7.87-7.80 (m, 2H), 7.81 (d, 1H), 7.43-7.33 (m, 3H), 5.00-4.97 (m, 1H), 4.82 (d, 1H), 4.60 (d, 1H), 3.83 (s, 3H), 3.78-3.58 (m, 2H), 2.86-2.81 (m, 7H), 2.43-2.35 (m, 1H), 2.14-2.05 (m, 2H), 1.83-1.71 (m, 1H). MS m/z 444 (M+1).

### Example 10

### N-Methyl-N-{[1-methyl-4-(1-piperazinylcarbonyl)-1H-benzimidazol-2-yl]methyl}-5,6,7,8-tetrahydro-8-quinolinamine

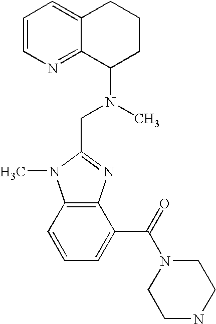

1-Methyl-2-{[methyl(5,6,7,8-tetrahydro-8-quinolinyl)amino]methyl}-1H-benzimidazole-4-carboxylic acid (100 mg, 0.29 mmol), bis(2-oxo-3-oxazolidinyl)phosphinic chloride (109 mg, 0.43 mmol), N-butoxycarbonylpiperizine (80, 0.43 mmol), and N,N-diisopropyl-ethylamine (56 mg, 0.43 mmol) were dissolved in N,N-dimethylformamide (5 mL) and the reaction was stirred for 16 h at room temperature. The reaction mixture was concentrated and purified using reverse phase HPLC (0% to 70% acetonitrile/water/0.1% trifluoroacetic acid gradient) to provide the protected amine. The amine was dissolved in dichloromethane (2 mL) and trifluoroacetic acid (2 mL) and stirred for 3 h. The reaction was concentrated and purified using reverse phase HPLC (0% to 70% acetonitrile/water/0.1% trifluoroacetic acid gradient) to provide the product (25.3 mg, 11%) as an off-white solid. ¹H-NMR (DMSO-d₆) δ 8.59 (d, 1H), 8.03 (d, 1H), 7.73 (d, 1H), 7.61-7.58 (m, 1H), 7.47 (t, 1H), 7.41 (d, 1H), 4.69-4.64 (m, 1H), 4.56-4.39 (m, 2H), 3.98-3.91 (m, 4H), 3.77-3.69 (m, 1H), 3.64-3.56 (m, 2H), 3.51-3.36 (m, 2H), 3.28-3.20 (m, 2H), 3.02-2.96 (m, 2H), 2.70 (s, 3H), 2.49-2.42 (m, 1H), 2.28-2.16 (m, 2H), 1.99-1.89 (m, 1H). MS m/z 419 (M+1).

### Example 11

### N-(4-Aminobutyl)-2-{[methyl(5,6,7,8-tetrahydro-8-quinolinyl)amino]methyl}-1H-benzimidazole-4-carboxamide

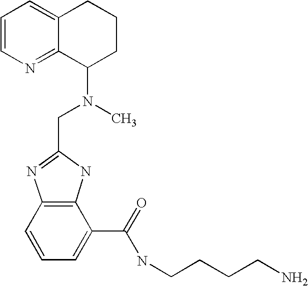

### A) 2-(Chloromethyl)-1H-benzimidazole-4-carboxylic acid hydrochloride

2,3-Diaminobenzoic acid (950 mg, 6.2 mmol), and chloroacetic acid (650 mg, 6.9 mmol) were dissolved in hydrochloric acid (5N, 25 mL) and the solution was heated at reflux for 24 h. The reaction mixture was concentrated to an oily brown solid and triturated with dichloromethane, ether and acetone. The resulting solid was filtered, dissolved in methanol and concentrated to provide a red solid (900 mg, 69%). ¹H-NMR (DMSO-d₆) δ 13.30 (br s), 12.65 (s, 1H), 7.88 (d, 1H), 7.82 (d, 1H), 7.30 (t, 1H), 4.94 (s, 2H).

### B) N-(4-Aminobutyl)-2-{[methyl(5,6,7,8-tetrahydro-8-quinolinyl)amino]methyl}-1H-benzimidazole-4-carboxamide

2-(Chloromethyl)-1H-benzimidazole-4-carboxylic acid hydrochloride (150 mg, 0.76 mmol), N-methyl-5,6,7,8-tetrahydro-8-quinolinamine hydrochloride (293 mg, 0.76 mmol), N,N-diisopropylethylamine (300 mg, 2.3 mmol) and potassium iodide (190 mg, 1.1 mmol) were dissolved in acetonitrile (10 mL) and the solution was heated at 60° C. for 16 h. The reaction mixture was concentrated to a brown solid and the resulting acid was carried on crude. The crude acid (85 mg, 0.25 mmol), bis(2-oxo-3-oxazolidinyl)phosphinic chloride (130 mg, 0.50 mmol), N-(4-aminobutyl)carbamic acid tert-butyl ester (70 mg, 0.37 mmol), and N,N-diisopropylethylamine (65 mg, 0.50 mmol) were dissolved in acetonitrile (5 mL) and the reaction was stirred for 72 h at 60° C. An additional equivalent of bis(2-oxo-3-oxazolidinyl)phosphinic chloride (100 mg, 0.40 mmol) and N-(3-aminopropyl)carbamic acid tert-butyl ester (50 mg, 0.30 mmol) were added and the reaction was stirred for 16 h at 65° C. The crude reaction mixture was run through MP-TSOH, the resin was rinsed with dichloromethane several times and the free amine was released from the resin with 2 M ammonia in methanol. The methanol solution was concentrated and purified using reverse phase HPLC (0% to 70% acetonitrile/water/0.1% trifluoroacetic acid gradient), concentrated, diluted with ethyl acetate and saturated aqueous sodium bicarbonate, the phases separated, the organic phase dried over sodium sulfate, filtered, and concentrated to provide the product (7.1 mg, 7%) as a white solid. ¹H-NMR (DMSO-d₆) δ 8.52-8.49 (m, 1H), 7.77 (d, 1H), 7.68 (d, 1H), 7.50 (d, 1H), 7.26-7.18 (m, 2H), 4.14-4.09 (m, 1H), 4.00-3.93 (m, 2H), 3.42-3.38 (m, 2H), 2.84-2.76 (m, 1H), 2.72-2.59 (m, 2H), 2.31 (s, 3H), 2.11-2.00 (m, 3H), 1.71-1.45 (m, 4H), 1.24-1.15 (m, 2H). MS m/z 407 (M+1).

### Example 12

### 2-{[Ethyl(5,6,7,8-tetrahydro-8-quinolinyl)amino]methyl}-N-[2-(1H-imidazol-4-yl)ethyl]-1H-benzimidazole-5-carboxamide

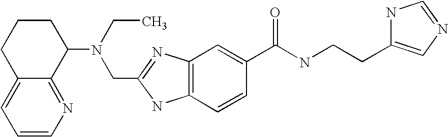

### A) 1-(1,1-Dimethylethyl) 5-methyl 2-{[ethyl(5,6,7,78-tetrahydro-8-quinolinyl)amino]methyl}-1H-benzimidazole-1,5-dicarboxylate

To a solution of N-ethyl-5,6,7,8-tetrahydro-8-quinolinamine (0.725 g, 4.0 mmol) in acetonitrile (50 mL) was added N,N-diisopropylethyl amine (1.5 mL, 8.0 mmol), 1-(1,1-dimethylethyl) 6-methyl 2-(chloromethyl)-1H-benzimidazole-1,5-dicarboxylate (WO 02/092575A1, incorporated by reference with regard to synthesis) (1.62 g, 4.5 mmol) and potassium iodide (0.35 g, 2.0 mmol). The reaction mixture was placed in an oil bath at 65° C. and stirred under nitrogen for 15 h. The solvent was evaporated under reduced pressure, dissolved in ethyl acetate (50 mL) and washed with sat. aqueous sodium bicarbonate (25 mL). The layers were separated, aq. layer extracted with ethyl acetate (3×25 mL), organic layers combined, dried over sodium sulfate, filtered and evaporated under reduced pressure to give a brown oil. Purification by silica gel chromatography with methylene chloride and 2N ammonia in methanol afforded 1-(1,1-dimethylethyl) 5-methyl 2-{[ethyl(5,6,7,8-tetrahydro-8-quinolinyl)amino]methyl}-1H-benzimidazole-1,5-dicarboxylate (0.6 g, 31% yield). ¹H-NMR (DMSO-d6): δ 12.65 (br s, 1H), 11.78 (br s, 1H), 8.50-8.43 (m, 2H), 8.00 (s, 2H), 7.65-7.61 (m, 1H), 7.50-7.47 (m, 2H), 7.19-7.16 (m, 2H), 6.79 (s, 1H), 4.07-4.01 (m, 2H), 3.95-3.91 (d, 1H), 3.44-3.41 (m, 2H), 2.78-2.63 (m, 4H), 2.12-2.06 (m, 1H), 1.93-1.79 (m, 2H), 1.67-1.59 (m, 1H), 0.90 (t, 3H); MS m/z 465.2 (M+1).

### B): 2-{[Ethyl(5,6,7,8-tetrahydro-8-quinolinyl)amino]methyl}-N-[2-(1H-imidazol-4-yl)ethyl]-1H-benzimidazole-5-carboxamide

1-(1,1-Dimethylethyl) 5-methyl 2-{[ethyl(5,6,7,8-tetrahydro-8-quinolinyl)amino]methyl}-1H-benzimidazole-1,5-dicarboxylate (2.5 g, 5.4 mmol) was dissolved in 30 ml of a 1:1:1 mixture of tetrahydrofuran, methanol, water. Lithium hydroxide (0.39 g, 16.1 mmol) freshly pulverized with a mortar and pestle was added, a reflux condensor attached and the system placed in an oil bath at 70° C. The reaction was stirred for 16 h then cooled to room temperature. Cold hydrochloric acid (3.2 mL) was added dropwise and the solvent was evaporated under reduced pressure. The resulting oil was azeotroped with toluene (1×15 ml) and then ether (3×50 mL) and placed under high vacuum for 16 hours to give a yellow solid. The crude acid was dissolved in anhydrous N,N-dimethylformamide (50 mL), resin-bound tetrafluorophenol 1.44 mmol/g (Argonaut Technologies, 1.44 mmol/g) (5.6 g, 8.0 mmol), DMAP (0.5 g, 4.0 mmol) and O-(7-azabenzotriazol-1-yl)-N,N,N′,N′-trimethyluronium hexafluorophosphate (HATU) (3.0 g, 8.0 mmol) were added and the reaction mixture was gently stirred for 15 h. The loaded resin was washed with DMF (3×25 mL), methylene chloride (3×25 mL) and dried under high vacuum to give 6.5 g of orange beads. To a portion of the loaded resin (0.625 g, 0.9 mmol) in DMF (5 mL) was added histamine (0.1 g, 0.9 mmol) and the mixture stirred for 14 h under nitrogen. The resin was filtered, washed with N,N-dimethylformamide (3×10 mL) and the combined filtrate evaporated under reduced pressure to yield. Further purification by reverse phase HPLC (0% to 70% acetonitrile/water/0.1% trifluoroacetic acid gradient), neutralization of the desired fractions and extraction from the aqueous layer (sat. with NaCl) with 1% methanol in ethyl acetate (3×25 ml) afforded 2-{[ethyl(5,6,7,8-tetrahydro-8-quinolinyl)amino]methyl}-N-[2-(1H-imidazol-4-yl)ethyl]-1H-benzimidazole-5-carboxamide as an off white solid (0.03 g, 7% yield). ¹H-NMR (CDCl₃): δ12.6 (br, s), 11.8 (br, s), 8.47 (d, 2H), 8.00 (s, 1H), 7.44 (m, 2H), 7.17, (t, 1H), 6.80 (s, 1H), 4.02 (t, 1H). 3.91 (Abq, 2H), 3.46 (q, 2H), 2.81-2.63 (m, 6H), 2.11-2.08 (m, 1H), 1.92-1.78 (m, 2H), 1.69-1.66 (m, 1H), 0.90 (t, 3H); MS m/z 444.4 (M+1).

### Example 13

### N-(3-Aminopropyl)-2-{[ethyl(5,6,7,8-tetrahydro-8-quinolinyl)amino]methyl}-1H-benzimidazole-5-carboxamide

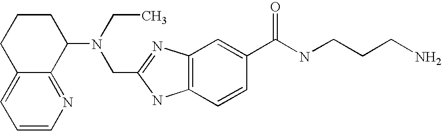

N-(3-Aminopropyl)-2-{[ethyl(5,6,7,8-tetrahydro-8-quinolinyl)amino]methyl}-1H-benzimidazole-5-carboxamide was prepared from 1-(1,1-dimethylethyl) 5-methyl 2-{[ethyl(5,6,7,8-tetrahydro-8-quinolinyl)amino]methyl}-1H-benzimidazole-1,5-dicarboxylate and diaminopropane in a similar manner as described above to give a white solid (0.05 g, 14% yield). ¹H-NMR (DMSO-d₆): δ 8.56 (d, 1H), 8.52 (br s, 1H), 8.08 (s, 1H), 7.69 (d, 1H), 7.56 (t, 1H), 7.56 (dd, 1H), 4.08 (t, 1H), 4.05 (Abq, 2H), 3.36 (q, 2H), 2.87-2.53 (m, 6H), 2.14 (m, 1H), 1.94-1.86 (m, 2H), 1.72-1.66 (m, 3H), 0.96 (t, 3H). MS m/z 407.2 (M+1).

### Example 14

### N-(2-Aminoethyl)-2-{[ethyl(5,6,7,8-tetrahydro-8-quinolinyl)amino]methyl}-1H-benzimidazole-5-carboxamide

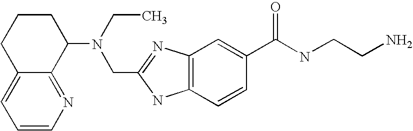

N-(2-Aminoethyl)-2-{[ethyl(5,6,7,8-tetrahydro-8-quinolinyl)amino]methyl}-1H-benzimidazole-5-carboxamide was prepared from 1-(1,1-dimethylethyl) 5-methyl 2-{[ethyl(5,6,7,8-tetrahydro-8-quinolinyl)amino]methyl}-1H-benzimidazole-1,5-dicarboxylate and ethylene diamine in a similar manner as described above to give a yellow solid (0.03 g, 8% yield). ¹H-NMR (DMSO-d₆): δ 8.50 (d, 1H), 8.44 (br s, 1H), 8.06 (s, 1H), 7.66 (d, 1H), 7.51 (d, 1H), 7.47 (d, 1H), 7.17 (dd, 1H), 4.02 (t, 1H), 3.98 (Abq, 2H), 3.36 (q, 2H), 2.80-2.62 (m, 6H), 2.09 (m, 1H), 1.91-1.80 (m, 2H), 1.63-1.60 (m, 1H), 0.89 (t, 3H). MS m/z 393.2 (M+1).

### Example 15

### N-(3-Aminopropyl)-2-{[ethyl(5,6,7,8-tetrahydro-8-quinolinyl)amino]methyl}-1H-benzimidazole-4-carboxamide

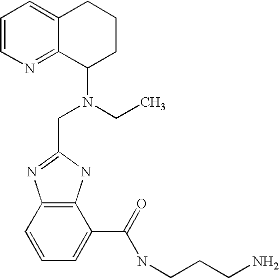

2-(Chloromethyl)-1H-benzimidazole-4-carboxylic acid hydrochloride (Bioorg. Med. Chem. 2004, 12, 5181; Bioorg. Med. Chem. Lett. 2003, 13, 3177 herein incorporated by reference with regard to such synthesis; 290 mg, 1.2 mmol), N-ethyl-5,6,7,8-tetrahydro-8-quinolinamine (210 mg, 1.2 mmol), N,N-diisopropylethylamine (470 mg, 3.6 mmol) and potassium iodide (300 mg, 1.8 mmol) were dissolved in acetonitrile (10 mL) and the solution was heated at 60° C. for 16 h. The reaction mixture was concentrated to give 2-{[ethyl(5,6,7,8-tetrahydro-8-quinolinyl)amino]methyl}-1H-benzimidazole-4-carboxylic acid as a brown solid and this acid was carried on crude. This crude acid (140 mg, 0.40 mmol), bis(2-oxo-3-oxazolidinyl)phosphinic chloride (200 mg, 0.80 mmol), N-(3-aminopropyl)carbamic acid tert-butyl ester (105 mg, 0.60 mmol), and N,N-diisopropylethylamine (103 mg, 0.80 mmol) were dissolved in acetonitrile (5 mL) and the reaction was stirred for 72 h at 60° C. An additional equivalent of bis(2-oxo-3-oxazolidinyl)phosphinic chloride (100 mg, 0.40 mmol) and N-(3-aminopropyl)carbamic acid tert-butyl ester (50 mg, 0.30 mmol) were added and the reaction was stirred for 16 h at 65° C. The crude reaction mixture was treated with trifluoroacetic acid for deprotection, then run through MP-TSOH, the resin was rinsed with dichloromethane several times and the free amine was released from the resin with 2 M ammonia in methanol. The methanol solution was concentrated and purified using reverse phase HPLC (0% to 70% acetonitrile/water/0.1% trifluoroacetic acid gradient), concentrated, diluted with ethyl acetate and saturated aqueous sodium bicarbonate, the phases separated, the organic phase dried over sodium sulfate, filtered, and concentrated to provide the product (16.2 mg, 10%) as a yellow solid. ¹H-NMR (DMSO-d₆) δ 8.56 (br s, 1H), 7.77-7.72 (m, 2H), 7.47 (d, 1H), 7.26-7.19 (m, 2H), 4.10-3.93 (m, 3H), 3.50-3.41 (m, 2H), 2.82-2.65 (m, 6H), 2.16-2.13 (m, 1H), 1.97-1.63 (m, 5H), 0.93 (t, 3H). MS m/z 407 (M+1).

### Example 16

### N-(4-Aminobutyl)-2-{[ethyl(5,6,7,8-tetrahydro-8-quinolinyl)amino]methyl}-1H-benzimidazole-4-carboxamide

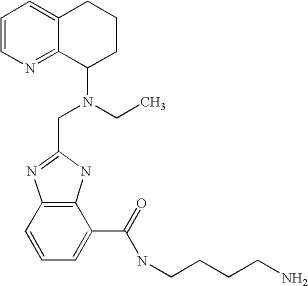

2-{[Ethyl(5,6,7,8-tetrahydro-8-quinolinyl)amino]methyl}-1H-benzimidazole-4-carboxylic acid (140 mg, 0.40 mmol), bis(2-oxo-3-oxazolidinyl)phosphinic chloride (200 mg, 0.80 mmol), N-(4-aminobutyl)carbamic acid tert-butyl ester (113 mg, 0.60 mmol), and N,N-diisopropylethylamine (103 mg, 0.80 mmol) were dissolved in acetonitrile (5 mL) and the reaction was stirred for 72 h at 60° C. An additional equivalent of bis(2-oxo-3-oxazolidinyl)phosphinic chloride (100 mg, 0.40 mmol) and N-(4-aminobutyl)carbamic acid tert-butyl ester (55 mg, 0.30 mmol) were added and the reaction was stirred for 16 h at 65° C. Following deprotection as described above, the crude reaction mixture was run through MP-TSOH, the resin was rinsed with dichloromethane several times and the free amine was released from the resin with 2 M ammonia in methanol. The methanol solution was concentrated and purified using reverse phase HPLC (0% to 70% acetonitrile/water/0.1% trifluoroacetic acid gradient), concentrated, diluted with ethyl acetate and saturated aqueous sodium bicarbonate, separated, dried over sodium sulfate, filtered and concentrated to provide the product (9.7 mg, 6%) as a yellow solid. ¹H-NMR (DMSO-d₆) δ 8.56 (br s, 1H), 7.75-7.50 (m, 2H), 7.49 (d, 1H), 7.25-7.19 (m, 2H), 4.13-3.99 (m, 3H), 3.42-3.40 (m, 2H), 2.78-2.65 (m), 2.12-2.09 (m, 1H), 1.96-1.85 (m, 2H), 1.68-1.52 (m, 5H), 0.93 (t, 3H). MS m/z 421 (M+1).

### Example 17

### N-{[5-(Aminomethyl)-1H-benzimidazol-2-yl]methyl}-N-methyl-5,6,7,8-tetrahydro-8-quinolinamine

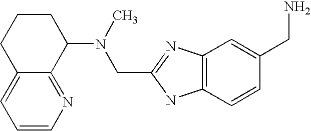

### A) 2-(Aminomethyl)-1H-benzimidazole-5-carbonitrile

A solution of 3,4-diaminobenzonitrile (5.0 g, 38 mmol), bis(2-oxo-3-oxazolidinyl)phosphinic chloride (14.3 g, 56 mmol), carbobenzyloxyglycine (7.9 g, 38 mmol), and N,N-diisopropylethylamine (7.3 g, 9.8 mL, 56 mmol) in acetonitrile (100 mL) was stirred for 16 h at room temperature. Additional bis(2-oxo-3-oxazolidinyl)phosphinic chloride (4.8 g, 19 mmol) and carbobenzyloxyglycine (4.0 g, 19 mmol) were added and the reaction mixture was stirred for 3 h. The reaction mixture was diluted with ethyl acetate (200 mL) and water (200 mL), phases separated, organic phase dried over sodium sulfate and concentrated to an oil. The crude amide was dissolved in acetic acid (200 mL) and stirred for 4 h at room temperature. The reaction was concentrated, diluted with dichloromethane (200 mL) and aqueous saturated sodium bicarbonate (200 mL), the phases separated, the organic phase dried over sodium sulfate, and concentrated to a red oil. The crude reaction was purified on silica (2% methanol/dichloromethane) to provide the benzimidazole (6.2 g) as a red solid. ¹H-NMR (DMSO-d₆) δ 12.85 (d, 1H), 8.08 (s, 1H), 7.98-7.95 (m, 1H), 7.70 (d, 1H), 7.69-7.50 (m, 2H), 7.37-7.29 (m, 4H), 5.06 (s, 2H), 4.46 (d, 2H). The protected benzimidazole (6.2 g, 20 mmol) was dissolved in ethanol (300 mL) and the solution was flushed with nitrogen. Palladium on carbon (10% w/w, 1.1 g, 1 mmol) was added and the solution was again flushed with nitrogen. The reaction was placed under a hydrogen atmosphere (1 atm) and stirred for 16 h at room temperature. The reaction was filtered through diatomaceous earth, concentrated and purified on silica (2% 2M ammonia in methanol/dichloromethane) to provide the product (3.8 g, 59% over 3 steps) as a red foam. ¹H-NMR (DMSO-d₆) δ 7.99 (s, 1H), 7.62 (d, 1H), 7.50 (d, 1H), 3.94 (s, 2H).

### B) 2-{[Methyl(5,6,7,8-tetrahydro-8-quinolinyl)amino]methyl}-1H-benzimidazole-5-carbonitrile

2-(Aminomethyl)-1H-benzimidazole-5-carbonitrile (3.6 g, 20 mmol), 6,7-dihydro-8(5H)-quinolinone (3.0 mg, 20 mmol), acetic acid (1.8 g, 30 mmol) and sodium triacetoxyborohydride (6.4 g, 30 mmol) were dissolved in 1,2-dichloroethane (150 mL) and stirred for 16 h at room temperature. The reaction mixture was diluted with water (200 mL), phases separated, organic phase dried over sodium sulfate, filtered and concentrated to a red oil. The crude secondary amine, formaldehyde (37% aqueous solution, 4.5 mL, 60 mmol), acetic acid (1.8 g, 30 mmol) and sodium triacetoxyborohydride (6.4 g, 30 mmol) were dissolved in 1,2-dichloroethane (150 mL) and stirred for 16 h at room temperature. The reaction mixture was diluted with dichloromethane (100 mL) and water (200 mL), separated, dried over sodium sulfate, filtered and concentrated. The crude tertiary amine was purified on silica (2% 2M ammonia in methanol/dichloromethane) to provide the product as a red solid (3.8 g, 60% over two steps). ¹H-NMR (DMSO-d₆): δ 9.59 (s, 1H), 8.49 (d, 1H), 7.97 (s, 1H), 7.66 (d, 1H), 7.51-7.46 (m, 2H), 7.19-7.16 (m, 1H), 4.11 (s, 2H), 4.01-3.97 (m, 2H), 3.05-2.96 (m, 1H), 2.85-2.78 (m, 1H), 2.73-2.66 (m, 1H), 2.33 (s, 3H), 2.10-1.93 (m, 2H), 1.74-1.64 (m, 1H). MS m/z 318 (M+1).

### C) N-{[5-(Aminomethyl)-1H-benzimidazol-2-yl]methyl}-N-methyl-5,6,7,8-tetrahydro-8-quinolinamine

2-{[methyl(5,6,7,8-tetrahydro-8-quinolinyl)amino]methyl}-1H-benzimidazole-5-carbonitrile (880 mg, 2.8 mmol) was dissolved in methanolic ammonia (7M, 35 mL), Raney nickel (catalytic) was added and the solution was flushed with nitrogen. The reaction was placed under a hydrogen atmosphere (60 psi) and stirred for 72 h. The reaction was filtered through diatomaceous earth and concentrated to provide the product (420 mg, 47%) as a brown oil. A small portion was purified using reverse phase HPLC (0% to 70% acetonitrile/water/0.1% trifluoroacetic acid gradient) to provide the product as a brown solid trifluoroacetate salt: ¹H-NMR (DMSO-d₆) δ 8.61 (d, 1H), 8.26 (br s, 3H), 7.90 (d, 1H), 7.78 (s, 1H), 7.71 (d, 1H), 7.56-7.53 (m, 1H), 7.40 (d, 1H), 4.47-4.72 (m, 1H), 4.60 (d, 1H), 4.44 (d, 1H), 4.18-4.14 (m, 2H), 2.88-2.78 (m, 2H), 2.65 (s, 3H), 2.36-2.27 (m, 1H), 2.09-1.92 (m, 2H), 1.83-1.70 (m, 1H).

### Example 18

### N-[(2-{[Methyl(5,6,7,8-tetrahydro-8-quinolinyl)amino]methyl}-1H-benzimidazol-5-yl)methyl]-1,3-propanediamine

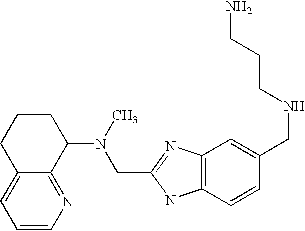

### A) Tert-butyl (3-oxopropyl)carbamate

Tert-butyl (3-hydroxypropyl)carbamate (250 mg, 1.4 mmol) was dissolved in dichloromethane (5 mL), cooled to 0° C. and Dess-Martin periodinane (600 mg, 1.4 mmol) was added and the reaction was warmed to room temperature and stirred overnight. The reaction was quenched with 10% aqueous sodium thiosulfate (20 mL) and saturated aqueous sodium bicarbonate (20 mL), stirred for 10 min, separated, concentrated to provide the product (240 mg, 99%) as a clear oil. ¹H-NMR (DMSO-d₆) δ 9.60 (s, 1H), 6.86 (br s, 1H), 3.21-3.16 (m, 2H), 2.51-2.47 (m, 2H), 1.34 (s, 9H).

### B) N-[(2-{[Methyl(5,6,7,8-tetrahydro-8-quinolinyl)amino]methyl}-1H-benzimidazol-5-yl)methyl]-1,3-propanediamine

N-{[5-(Aminomethyl)-1H-benzimidazol-2-yl]methyl}-N-methyl-5,6,7,8-tetrahydro-8-quinolinamine (100 mg, 0.30 mmol), tert-butyl-N-(3-oxopropyl)carbamate (52 mg, 0.30 mmol) and acetic acid (27 mg, 0.45 mmol) were dissolved in 1,2-dichloroethane (10 mL). Sodium triacetoxyborohydride (95 mg, 0.45 mmol) was added portionwise over 30 min. The reaction was stirred for 16 h at room temperature. The reaction mixture was concentrated and purified using reverse phase HPLC (0% to 70% acetonitrile/water/0.1% trifluoroacetic acid gradient) to provide the protected amine. The carbamate was dissolved in dichloromethane (5 mL) and trifluoroacetic acid (2 mL) and stirred for 3 h at room temperature. The reaction was concentrated, dissolved in water and lyophilized to provide the trifluoroacetate salt (2.5 mg, 1%) as a white crystalline solid: MS m/z 379 (M+1).

### Example 19

### N-Methyl-N-[(5-{[(4-piperidinylmethyl)amino]methyl}-1H-benzimidazol-2-yl)methyl]-5,6,7,8-tetrahydro-8-quinolinamine

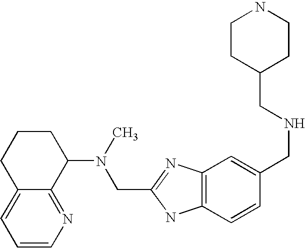

### A) Tert-butyl 4-formyl-1-piperidinecarboxylate

Tert-butyl 4-hydroxymethyl-1-piperidinecarboxylate (130 mg, 0.60 mmol) was dissolved in dichloromethane (5 mL), cooled to 0° C. and Dess-Martin periodinane (260 mg, 0.60 mmol) was added and the reaction was warmed to room temperature and stirred overnight. The reaction was quenched with 10% aqueous sodium thiosulfate (20 mL) and saturated aqueous sodium bicarbonate (20 mL), stirred for 10 min, separated, concentrated to provide the product (125 mg, 98%) as a clear oil. ¹H-NMR (DMSO-d₆) δ 9.56 (s, 1H), 5.74 (s, 1H), 3.79-3.76 (m, 2H), 2.89-2.84 (m, 2H), 1.82-1.77 (m, 2H), 1.37 (s, 9H), 1.37 (s, 9H), 1.34-1.27 (m, 2H).

### B) N-Methyl-N-[(5-{[(4-piperidinylmethyl)amino]methyl}-1H-benzimidazol-2-yl)methyl]-5,6,7,8-tetrahydro-8-quinolinamine

N-{[5-(aminomethyl)-1H-benzimidazol-2-yl]methyl}-N-methyl-5,6,7,8-tetrahydro-8-quinolinamine (100 mg, 0.30 mmol), tert-butyl-4-formyl-1-piperidinecarboxylate (64 mg, 0.30 mmol) and acetic acid (27 mg, 0.45 mmol) were dissolved in 1,2-dichloroethane (10 mL). Sodium triacetoxyborohydride (95 mg, 0.45 mmol) was added portionwise over 30 min. The reaction was stirred for 16 h at room temperature. The reaction mixture was concentrated and purified using reverse phase HPLC (0% to 70% acetonitrile/water/0.1% trifluoroacetic acid gradient) to provide the protected amine. The carbamate was dissolved in dichloromethane (5 mL) and trifluoroacetic acid (2 mL) and stirred for 3 h at room temperature. The reaction was concentrated, dissolved in water and lyophilized to provide the product (14.5 mg, 6% as a light brown solid: ¹H-NMR DMSO-d₆) δ 8.92-8.84 (m, 1H), 8.63-8.52 (m, 1H), 7.80-7.75 (m, 2H), 7.68 (d, 1H), 7.47-7.44 (m, 1H), 7.38-7.35 (m, 2H), 4.81-4.76 (m, 1H), 4.60 (d, 1H), 4.45 (d, 1H), 4.26-4.23 (m, 2H), 3.28-3.23 (m, 2H), 2.87-2.81 (m, 6H), 2.72 (s, 3H), 2.42-2.29 (m, 1H), 2.07-1.72 (m, 7H), 1.33-1.24 (m, 1H). MS m/z 419 (M+1).

### Example 20

### N-{[4-(Aminomethyl)phenyl]methyl}-2-{[methyl(5,6,7,8-tetrahydro-8-quinolinyl)amino]methyl}-1H-benzimidazole-5-carboxamide

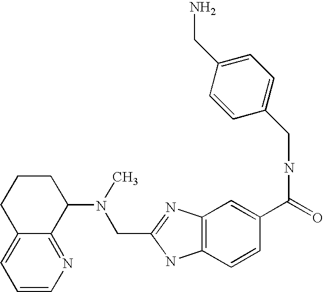

2-{[Methyl(5,6,7,8-tetrahydro-8-quinolinyl)amino]methyl}-1H-benzimidazole-5-carboxylic acid (190 mg, 0.56 mmol), bis(2-oxo-3-oxazolidinyl)phosphinic chloride (280 mg, 1.1 mmol), tert-butyl N-[4-(aminomethyl)benzyl]carbamate (260 mg, 1.1 mmol), and N,N-diisopropylethylamine (220 mg, 1.7 mmol) were dissolved in acetonitrile (3 mL) and N,N-dimethylformamide (2 mL) and the reaction was stirred at 35° C. for 16 h. The reaction was concentrated and purified using reverse phase HPLC (0% to 70% acetonitrile/water/0.1% trifluoroacetic acid gradient) to provide the protected amine. The carbamate was dissolved in dichloromethane (2 mL) and trifluoroacetic acid (2 mL), stirred for 2 h, concentrated, and lyophilized from water to provide the product (35 mg, 8%) as a tan trifluoroacetate salt: ¹H-NMR (CD₃OD) δ 8.31-8.30 (m, 2H), 7.88-7.86 (m, 2H), 7.48-7.37 (m, 6H), 4.60-4.33 (m, 1H), 4.30-4.25 (m, 2H), 4.23 (d, 1H), 4.10-4.09 (m, 2H), 3.95-3.83 (m, 2H), 3.28 (d, 1H), 3.03-3.00 (m, 1H), 2.38 (s, 3H), 2.35-1.92 (m, 4H). MS m/z 455 (M+1).

### Example 21

### N-Methyl-N-{[4-(1-piperazinylcarbonyl)-1H-benzimidazol-2-yl]methyl}-5,6,7,8-tetrahydro-8-quinolinamine

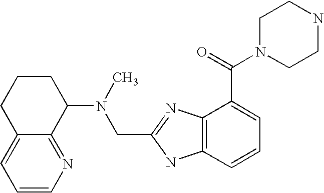

2-{[Methyl(5,6,7,8-tetrahydro-8-quinolinyl)amino]methyl}-1H-benzimidazole-4-carboxylic acid (190 mg, 0.56 mmol), bis(2-oxo-3-oxazolidinyl)phosphinic chloride (220 mg, 0.85 mmol), N-butoxycarbonylpiperizine (104 mg, 0.56 mmol), and N,N-diisopropylethylamine (110 mg, 0.85 mmol) were dissolved in acetonitrile (5 mL) and N,N-dimethylformamide (2 mL) and the reaction was stirred for 16 h at 35° C. The reaction mixture was concentrated and purified using reverse phase HPLC (0% to 70% acetonitrile/water/0.1% trifluoroacetic acid gradient) to provide the protected amine (56 mg). The amine was dissolved in dichloromethane (3 mL) and trifluoroacetic acid (2 mL) and stirred for 3 h. The reaction was concentrated, dissolved in water and lyophilized to provide the product (68 mg, 30%) as an off-white trifluoroacetate salt: ¹H-NMR (DMSO-d₆) δ 8.62 (d, 1H), 7.94 (d, 1H), 7.72 (d, 1H), 7.58-7.54 (m, 1H), 7.41-7.35 (m, 2H), 4.74-4.70 (m, 1H), 4.60 (d, 1H), 4.48 (d, 1H), 4.38 (s, 2H), 3.25 (br s, 4H), 3.13 (br s, 4H), 2.88-2.80 (m, 2H), 2.65 (s, 3H), 2.36-2.30 (m, 1H), 2.08-1.96 (m, 2H), 1.82-1.72 (m, 1H). MS m/z 405 (M+1).

### Example 22

### N-Methyl-N-({4-[(4-methyl-1-piperazinyl)carbonyl]-1H-benzimidazol-2-yl}methyl)-5,6,7,8-tetrahydro-8-quinolinamine

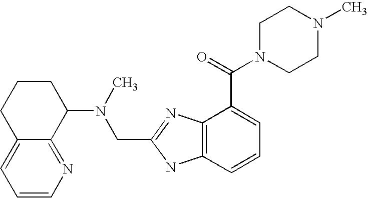

N-methyl-N-{[4-(1-piperazinylcarbonyl)-1H-benzimidazol-2-yl]methyl}-5,6,7,8-tetrahydro-8-quinolinamine tris(trifluoroacetate) (28 mg, 0.04 mmol) formaldehyde (37% aqueous solution, 0.2 mL), acetic acid (catalytic) and sodium triacetoxyborohydride (17 mg, 0.08 mmol) were dissolved in 1,2-dichloroethane (5 mL) and stirred for 3 h at room temperature. The reaction concentrated and purified using reverse phase HPLC (0% to 70% acetonitrile/water/0.1% trifluoroacetic acid gradient) to provide the amine. The reaction was concentrated, dissolved in water and lyophilized to provide the product (21 mg, 67%) as a yellow crystalline trifluoroacetate salt: ¹H-NMR (DMSO-d₆) δ 10.90 (br s, 1H), 8.60 (d, 1H), 7.86 (d, 1H), 7.76 (d, 1H), 7.52-7.49 (m, 1H), 7.35-7.33 (m, 2H), 4.77-4.73 (m, 1H), 4.63 (d, 1H), 4.49 (d, 1H), 3.47-3.31 (m, 4H), 3.17-3.05 (m, 4H), 2.87-2.80 (m, 2H), 2.79 (s, 3H), 2.67 (s, 3H), 2.38-2.31 (m, 1H), 2.09-1.98 (m, 2H), 1.82-1.62 (m, 1H). MS m/z 419 (M+1).

### Example 23

### N-[2-(1H-Imidazol-4-yl)ethyl]-2-{[methyl(5,6,7,8-tetrahydro-8-quinolinyl)amino]methyl}-1H-benzimidazole-4-carboxamide

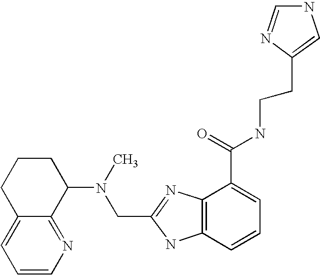

2-{[Methyl(5,6,7,8-tetrahydro-8-quinolinyl)amino]methyl}-1H-benzimidazole-4-carboxylic acid (190 mg, 0.56 mmol), bis(2-oxo-3-oxazolidinyl)phosphinic chloride (220 mg, 0.85 mmol), histamine (0.56 mmol), and N,N-diisopropylethylamine (110 mg, 0.85 mmol) were dissolved in acetonitrile (5 mL) and N,N-dimethylformamide (2 mL) and the reaction was stirred for 16 h at 35° C. The reaction mixture was concentrated and purified using reverse phase HPLC (0% to 70% acetonitrile/water/0.1% trifluoroacetic acid gradient) and lyophilized from water to provide the product (21 mg, 6%) as a light brown trifluoroacetate salt: ¹H-NMR (DMSO-d₆) δ 9.20-9.18 (m, 1H), 8.99 (s, 1H), 8.53 (d, 1H), 7.82-7.75 (m, 3H), 7.45-7.40 (m, 2H), 7.34-7.30 (m, 1H), 4.78-4.74 (m, 1H), 4.67 (d, 1H), 4.53 (d, 1H), 4.36-4.34 (m, 1H), 3.86-3.82 (m, 1H), 3.68-3.57 (m, 2H), 2.81-2.73 (m, 2H), 2.73 (s, 3H), 2.42-2.35 (m, 1H), 2.07-1.97 (m, 2H), 1.79-1.72 (m, 1H). MS m/z 430 (M+1).

### Example 24

### N-[2-(1-Methyl-1H-imidazol-4-yl)ethyl]-2-{[methyl(5,6,7,8-tetrahydro-8-quinolinyl)amino]methyl}-1H-benzimidazole-4-carboxamide

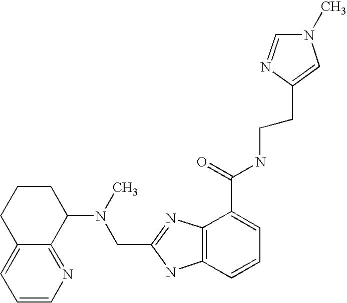

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (115 mg, 0.45 mmol), [2-(1-methyl-1H-imidazol-4-yl)ethyl]amine dihydrochloride (59 mg, 0.30 mmol), and N,N-diisopropylethylamine (155 mg, 1.20 mmol) were dissolved in acetonitrile (3 mL) and N,N-dimethylformamide (2 mL) and the reaction was stirred for 72 h at room temperature. The reaction mixture was concentrated and purified using reverse phase HPLC (0% to 70% acetonitrile/water/0.1% trifluoroacetic acid gradient) and lyophilized from water to provide the product (25 mg, 12%) as a yellow trifluoroacetate salt: ¹H-NMR (DMSO-d₆) δ 9.18 (t, 1H), 8.95 (s, 1H), 8.53 (d, 1H), 7.83-7.75 (m, 3H), 7.46 (s, 1H), 7.44-7.40 (m, 1H), 7.33 (t, 1H), 4.78-4.74 (m, 1H), 4.68 (d, 1H), 4.54 (d, 1H), 2.94-2.88 (m, 2H), 2.84-2.79 (m, 2H), 2.73 (s, 3H), 2.44-2.33 (m, 1H), 2.08-1.99 (m, 2H), 1.80-1.71 (m, 1H). MS m/z 444 (M+1).

### Example 25

### 2-{[Methyl(5,6,7,8-tetrahydro-8-quinolinyl)amino]methyl}-1H-benzimidazole-4-carboxamide

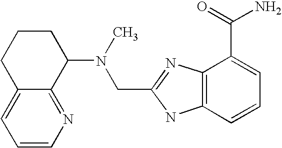

Methyl 2-{[methyl(5,6,7,8-tetrahydro-8-quinolinyl)amino]methyl}-1H-benzimidazole-4-carboxylate Example 6 was converted to the activated polymer supported tetrafluorophenolic ester as in Example 17. The loaded resin (550 mg, 0.40 mmol) was diluted with N,N-dimethylformamide (10 mL) and ammonia was bubbled through the slurry for 10 min. The reaction was stirred for 16 h at room temperature, filtered, concentrated and purified using reverse phase HPLC (0% to 70% acetonitrile/water/0.1% trifluoroacetic acid gradient) and lyophilized from water to provide the product (31 mg, 14%) as a white trifluoroacetate salt: ¹H-NMR (DMSO-d₆) δ 8.58-8.55 (m, 1H), 8.53-8.52 (m, 1H), 7.87-7.81 (m, 2H), 7.74-7.69 (m, 2H), 7.42-7.38 (m, 1H), 7.32 (t, 1H), 4.79-4.70 (m, 2H), 4.59 (d, 1H), 2.84-2.77 (m, 5H), 2.44-2.37 (m, 1H), 2.10-1.98 (m, 2H), 1.79-1.70 (m, 1H). MS m/z 336 (M+1).

### Example 26

### N-(2-Aminoethyl)-2-{[methyl(5,6,7,8-tetrahydro-8-quinolinyl)amino]methyl}-1H-benzimidazole-4-carboxamide

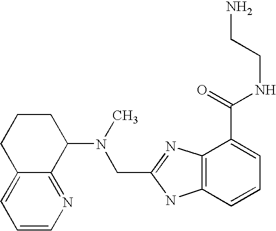

Methyl 2-{[methyl(5,6,7,8-tetrahydro-8-quinolinyl)amino]methyl}-1H-benzimidazole-4-carboxylate was converted to the activated polymer supported tetrafluorophenolic ester as described in a previous example. The loaded resin (550 mg, 0.40 mmol) was diluted with N,N-dimethylformamide (10 mL) and 1,2-ethanediamine (48 mg, 0.8 mmol) was added. The reaction was stirred for 16 h at room temperature, filtered, concentrated and purified using reverse phase HPLC (0% to 70% acetonitrile/water/0.1% trifluoroacetic acid gradient) and lyophilized from water to provide the product as a trifluoroacetate salt (38 mg, 13%) as a yellow solid: ¹H-NMR (DMSO-d₆) δ 9.18-9.12 (m, 1H), 8.55-8.54 (m, 1H), 7.87-7.77 (m, 4H), 7.45-7.34 (m, 2H), 4.80-4.72 (m, 1H), 4.70 (d, 1H), 4.57 (d, 1H), 3.64 (m, 2H), 3.05-2.99 (m, 2H), 2.87-2.78 (m, 1H), 2.75 (s, 3H), 2.45-2.33 (m, 2H), 2.10-1.97 (m, 2H), 1.82-1.74 (m, 1H). MS m/z 379 (M+1).

### Example 27

### 2-{[Methyl(5,6,7,8-tetrahydro-8-quinolinyl)amino]methyl}-N-[2-(1-piperidinyl)propyl]-1H-benzimidazole-4-carboxamide

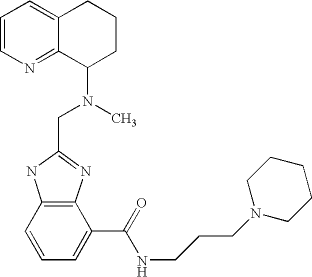

Methyl 2-{[methyl(5,6,7,8-tetrahydro-8-quinolinyl)amino]methyl}-1H-benzimidazole-4-carboxylate was converted to the activated polymer supported tetrafluorophenolic ester as described before. The loaded resin (400 mg, 0.30 mmol) was diluted with N,N-dimethylformamide (6 mL) and [2-(1-piperidinyl)propyl]amine (85 mg, 0.60 mmol) was added. The reaction was stirred for 16 h at room temperature, filtered, concentrated and purified using reverse phase HPLC (0% to 70% acetonitrile/water/0.1% trifluoroacetic acid gradient) and lyophilized from water to provide the product (13 mg, 5%) as a oily yellow solid: ¹H-NMR (DMSO-d₆): 8.56 (d, 1H), 7.86-7.78 (m, 4H), 7.47-7.44 (m, 1H), 7.37-7.33 (m, 2H), 4.80-4.76 (m, 1H), 4.71 (d, 1H), 4.56 (d, 1H), 3.44-3.39 (m, 4H), 3.10-3.04 (m, 2H), 2.88-2.77 (m, 4H), 2.77 (s, 3H), 2.43-2.31 (m, 1H), 2.09-1.91 (m, 4H), 1.81-1.59 (m, 6H), 1.40-1.29 (m, 1H).

### Example 28

### 2-{[Methyl(5,6,7,8-tetrahydro-8-quinolinyl)amino]methyl}-N-[3-(1-pyrrolidinyl)propyl]-1H-benzimidazole-4-carboxamide

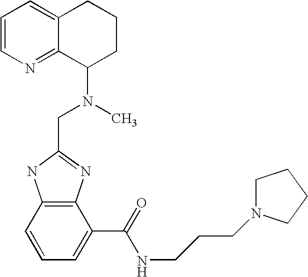

Methyl 2-{[methyl(5,6,7,8-tetrahydro-8-quinolinyl)amino]methyl}-1H-benzimidazole-4-carboxylate was converted to the activated polymer supported tetrafluorophenolic ester as described above. The loaded resin (400 mg, 0.30 mmol) was diluted with N,N-dimethylformamide (6 mL) and [2-(1-pyrrolidinyl)propyl]amine (77 mg, 0.60 mmol) was added. The reaction was stirred for 16 h at room temperature, filtered, concentrated and purified using reverse phase HPLC (0% to 70% acetonitrile/water/0.1% trifluoroacetic acid gradient) and lyophilized from water to provide the product (13 mg, 5%) as a yellow oil: ¹H-NMR (DMSO-d₆) 9.65-9.64 (m, 1H), 9.21-9.17 (m, 1H), 8.56 (d, 1H), 7.86-7.83 (m, 2H), 7.78 (d, 1H), 7.46-7.42 (m, 1H), 7.35 (t, 1H), 4.85-4.77 (m, 1H), 4.72 (d, 1H), 4.58 (d, 1H), 3.58-3.52 (m, 2H), 3.46-3.42 (m, 2H), 3.21-3.15 (m, 3H), 3.01-2.93 (m, 3H) 2.85-2.81 (m, 2H), 2.75 (s, 3H), 2.45-2.37 (m, 1H), 2.10-1.72 (m, 7H). MS m/z 447 (M+1).

### Example 29

### N-[3-(Dimethylamino)propyl]-2-{[methyl(5,6,7,8-tetrahydro-8-quinolinyl)amino]methyl}-1H-benzimidazole-4-carboxamide

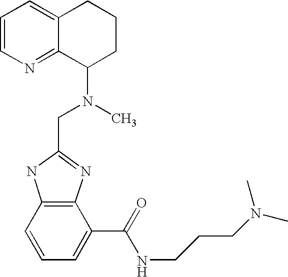

Methyl 2-{[methyl(5,6,7,8-tetrahydro-8-quinolinyl)amino]methyl}-1H-benzimidazole-4-carboxylate was converted to the activated polymer supported tetrafluorophenolic ester as described above. The loaded resin (400 mg, 0.30 mmol) was diluted with N,N-dimethylformamide (6 mL) and N,N-dimethylpropylenediamine (0.60 mmol) was added. The reaction was stirred for 16 h at room temperature, filtered, concentrated and purified using reverse phase HPLC (0% to 70% acetonitrile/water/0.1% trifluoroacetic acid gradient) and lyophilized from water to provide the product as a trifluoroacetate salt (13 mg, 5%) as a yellow oil: ¹H-NMR (DMSO-d₆) 9.51-9.43 (m, 1H), 9.22-9.17 (m, 1H), 8.55 (d, 1H), 7.88-7.83 (m, 2H), 7.77 (d, 1H), 7.47-7.41 (m, 1H), 7.35 (t, 1H), 4.84-4.74 (m, 1H), 4.71 (d, 1H), 4.58 (d, 1H), 3.44-3.39 (m, 2H), 3.12-3.07 (m, 2H), 2.86-2.76 (m, 10H), 2.44-2.35 (m, 1H), 2.09-2.00 (m, 2H), 1.93-1.85 (m, 2H), 1.78-1.68 (m, 2H). MS m/z 421 (M+1).

### Example 30

### N-({4-[(4-Amino-1-piperidinyl)carbonyl]-1H-benzimidazol-2-yl}methyl)-N-methyl-5,6,7,8-tetrahydro-8-quinolinamine

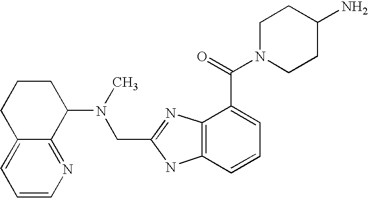

2-{[Methyl(5,6,7,8-tetrahydro-8-quinolinyl)amino]methyl}-1H-benzimidazole-4-carboxylic acid (80 mg, 0.24 mmol), bis(2-oxo-3-oxazolidinyl)phosphinic chloride (91 mg, 0.37 mmol), tert-butyl 4-piperidinylcarbamate (96 mg, 0.48 mmol), and N,N-diisopropylethylamine (62 mg, 0.48 mmol) were dissolved in N,N-dimethylformamide (5 mL) and the reaction was stirred for 72 h at room temperature. The reaction mixture was concentrated and purified using reverse phase HPLC (0% to 70% acetonitrile/water/0.1% trifluoroacetic acid gradient) to provide the protected amine. The carbamate was dissolved in dichloromethane (5 mL) and trifluoroacetic acid (2 mL), stirred for 2 h, concentrated, purified using reverse phase HPLC (0% to 70% acetonitrile/water/0.1% trifluoroacetic acid gradient) and lyophilized from water to provide the product (32 mg, 17%) as a white solid: ¹H-NMR (DMSO-d₆) δ 8.54 (d, 1H), 7.88 (br s, 2H), 7.77 (d, 1H), 7.72 (d, 1H), 7.45-7.42 (m, 1H), 7.30 (t, 1H), 7.21 (m, 1H), 4.79-4.75 (m, 1H), 4.60 (d, 1H), 4.47 (d, 1H), 3.33-3.22 (m, 1H), 3.05-2.89 (m, 2H), 2.85-2.79 (m, 2H), 2.73 (s, 3H), 2.42-2.32 (m, 2H), 2.07-1.71 (m, 6H), 1.52-1.37 (m, 2H). MS m/z 419 (M+1).

### Example 31

### N-({4-[(3-Amino-1-pyrrolidinyl)carbonyl]-1H-benzimidazol-2-yl}methyl)-N-methyl-5,6,7,8-tetrahydro-8-quinolinamine

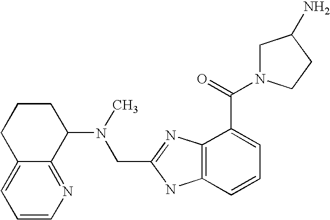

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (115 mg, 0.45 mmol), tert-butyl 3-pyrrolidinylcarbamate (89 mg, 0.48 mmol), and N, N-diisopropylethylamine (89 mg, 0.48 mmol) were dissolved in N,N-dimethylformamide (4 mL) and the reaction was stirred for 16 h at room temperature. The reaction mixture was concentrated and purified using reverse phase HPLC (0% to 70% acetonitrile/water/0.1% trifluoroacetic acid gradient) to provide the protected amine. The carbamate was dissolved in dichloromethane (5 mL) and trifluoroacetic acid (2 mL), stirred for 2 h, concentrated, purified using reverse phase HPLC (0% to 70% acetonitrile/water/0.1% trifluoroacetic acid gradient) and lyophilized from water to provide the product (17 mg, 18%) as a white solid: ¹H-NMR (DMSO-d₆) δ 8.55 (d, 1H), 8.10-7.93 (m, 2H), 7.77-7.73 (m, 2H), 7.46-7.39 (m, 2H), 7.31 (t, 1H), 4.77-4.73 (m, 1H), 4.65 (d, 1H), 4.52 (d, 1H), 3.89-3.76 (m, 2H), 3.67-3.56 (m, 2H), 2.84-2.73 (m, 4H), 2.44-2.30 (m, 1H), 2.20-2.13 (m, 1H), 2.07-1.90 (m, 4H), 1.79-1.66 (m, 2H). MS m/z 405 (M+1).

### Example 32

### N-{[4-({[2-(1H-Imidazol-4-yl)ethyl]amino}methyl)-1H-benzimidazol-2-yl]methyl}-N-methyl-5,6,7,8-tetrahydro-8-quinolinamine

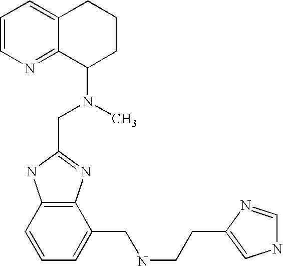

### A) 2-{[Methyl(5,6,7,8-tetrahydro-8-quinolinyl)amino]methyl}-1H-benzimidazole-4-carbaldehyde

Methyl 2-{[methyl(5,6,7,8-tetrahydro-8-quinolinyl)amino]methyl}-1H-benzimidazole-4-carboxylate (1.0 g, 2.9 mmol) was dissolved in anhydrous tetrahydrofuran (20 mL) and cooled to 0° C. Lithium aluminum hydride (1 M in THF, 2.9 mL) was added dropwise and the reaction was warmed to room temperature. An additional portion of lithium aluminum hydride (1 M in THF, 2.9 mL) was added dropwise and the reaction was stirred for 30 min. The reaction was quenched by the addition of aqueous potassium sodium tartrate (5%, 50 mL) and ethyl acetate (50 mL). The biphasic solution was separated, dried over sodium sulfate, filtered and concentrated to a yellow foam. The alcohol was dissolved in dichloromethane (20 mL), Dess-Martin periodinane (1.3 g, 0.31 mmol) was added portion-wise over two minutes and the reaction was stirred overnight at room temperature. The reaction was quenched with 5% aqueous sodium thiosulfate (50 mL) and saturated aqueous sodium bicarbonate (50 mL), stirred for 1 h, extracted with dichloromethane (2×100 mL), concentrated and purified on silica (2% to 5% 2M ammonia in methanol/dichloromethane gradient) to provide the product (700 mg, 71%) as a brown foam. ¹H-NMR (DMSO-d₆) δ 13.38 (br s, 1H), 10.18 (s, 1H), 8.77-8.64 (m, 1H), 7.92-7.77 (m, 2H), 7.55-7.53 (m, 1H), 7.37-7.33 (m, 1H), 7.26-7.23 (m, 1H), 4.04-3.92 (m, 3H), 2.87-2.66 (m, 2H), 2.32 (s, 3H), 2.11-2.03 (m, 1H), 1.99-1.85 (m, 2H), 1.73-1.64 (m, 1H).

### B) N-{[4-({[2-(1H-Imidazol-4-yl)ethyl]amino}methyl)-1H-benzimidazol-2-yl]methyl}-N-methyl-5,6,7,8-tetrahydro-8-quinolinamine

2-{[Methyl(5,6,7,8-tetrahydro-8-quinolinyl)amino]methyl}-1H-benzimidazole-4-carbaldehyde (100 mg, 0.30 mmol), histamine (69 mg, 9.60 mmol), acetic acid (28 mg, 0.47 mmol) and sodium triacetoxyborohydride (100 mg, 0.47 mmol) were dissolved in 1,2-dichloroethane (10 mL) and stirred for 16 h at room temperature. The reaction concentrated and purified using reverse phase HPLC (0% to 70% acetonitrile/water/0.1% trifluoroacetic acid gradient) to provide the product (84.5 mg, 36%) as an off-white solid. ¹H-NMR (DMSO-d₆) δ 9.00 (s, 1H), 8.56 (d, 1H), 7.86 (d, 1H), 7.69 (d, 1H), 7.49-7.48 (m, 2H), 7.40-7.32 (m, 2H), 4.81-4.76 (m, 1H), 4.60 (d, 1H), 4.55 (s, 2H), 4.46 (d, 1H), 3.32 (t, 2H), 3.09 (t, 2H), 2.87-2.79 (m, 2H), 2.69 (s, 3H), 2.39-2.31 (m, 1H), 2.09-1.98 (m, 2H), 1.82-1.70 (m, 1H). MS m/z 416 (M+1).

### Example 33

### N-Methyl-N-{[4-(1-piperazinylmethyl)-1H-benzimidazol-2-yl]methyl}-5,6,7,8-tetrahydro-8-quinolinamine

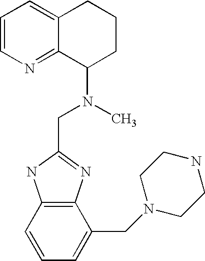

2-{[Methyl(5,6,7,8-tetrahydro-8-quinolinyl)amino]methyl}-1H-benzimidazole-4-carbaldehyde (100 mg, 0.30 mmol), N-butoxycarbonylpiperizine (115 mg, 0.60 mmol), acetic acid (28 mg, 0.47 mmol) and sodium triacetoxyborohydride (100 mg, 0.47 mmol) were dissolved in 1,2-dichloroethane (10 mL) and stirred for 16 h at room temperature. The reaction concentrated and purified using reverse phase HPLC (0% to 70% acetonitrile/water/0.1% trifluoroacetic acid gradient) to provide the protected amine. The carbamate was dissolved in dichloromethane (2 mL) and trifluoroacetic acid (2 mL) and stirred for 3 h at room temperature. The reaction was concentrated, dissolved in water and lyophilized to provide the product (68 mg, 30%) as an off-white solid: ¹H-NMR (DMSO-d₆) δ 8.62 (d, 1H), 7.94 (d, 1H), 7.72 (d, 1H), 7.58-7.54 (m, 1H), 7.41-7.35 (m, 2H), 4.74-4.70 (m, 1H), 4.60 (d, 1H), 4.48 (d, 1H), 4.38 (s, 2H), 3.25 (br s, 4H), 3.13 (br s, 4H), 2.88-2.80 (m, 2H), 2.65 (s, 3H), 2.36-2.30 (m, 1H), 2.08-1.96 (m, 2H), 1.82-1.72 (m, 1H). MS m/z 391 (M+1).

### Example 34

### N-methyl-N-{[4-(1-piperazinyl)-1H-benzimidazol-2-yl]-methyl}-5,6,7,8-tetrahydro-8-quinolinamine

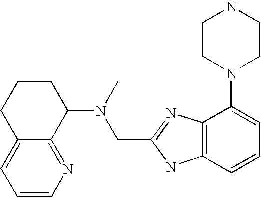

### A) 1,1-Dimethylethyl 4-(3-amino-2-nitrophenyl)-1-piperazinecarboxylate

3-Chloro-2-nitroaniline (3.0 g, 17.4 mmol) and Boc-piperazine (3.6 g, 19.1 mmol) were dissolved in DMF (50 mL), potassium carbonate (4.8 g, 34.7 mmol) was added and the reaction placed in an oil bath at 130° C. under nitrogen atmosphere. The mixture was stirred for 72 h, cooled to room temperature and the solvent evaporated. The residue was dissolved in ethyl acetate (100 mL), washed with 10% lithium bromide, dried over sodium sulfate, filtered and evaporated. Purification by silica gel chromatography and gradient elution from 0 to 2% methanol in methylene chloride afforded an orange solid (2.25 g, 40%). ¹H-NMR (DMSO-d₆): δ 7.16 (t, 1H), 6.61 (d, 1H), 6.43 (d, 1H), 5.92 (s, 2H), 3.41-3.35 (m, 4H), 2.86-2.82 (m, 4H), 1.44 (s, 9H). MS m/z 345.1 (M+23).

### B) 1,1-Dimethylethyl 4-{2-[({[(phenylmethyl)oxy]carbonyl}amino)methyl]-1H-benzimidazol-4-yl}-1-piperazinecarboxylate

1,1-Dimethylethyl 4-(3-amino-2-nitrophenyl)-1-piperazinecarboxylate (2.2 g, 6.8 mmol) was dissolved in ethanol (75 mL) and purged with nitrogen for 15 min. 10% Pd/C (0.25 g) was added, the system purged with H₂ and stirred under an atmosphere of hydrogen for 3 h. The reaction was filtered through a pad of celite and evaporated to leave an oily residue that was azeotroped with ether (2×25 mL) and place under high vacuum for 14 h. The aniline was dissolved in acetonitrile, Cbz-glycine (1.6 g, 7.5 mmol), DIPEA (1.3 mL, 7.5 mmol) and BOP-Cl (1.9 g, 7.5 mmol) were added and the reaction stirred for 14 h. The solvent was evaporated and the resulting oil dissolved in ethyl acetate (50 mL), washed with sat. NaHCO₃ (2×25 mL), dried over sodium sulfate, filtered and evaporated. The crude amide was cyclized to the benzimidazole by heating (65° C.) the residue in acetic acid (100 mL) and stirring for 3 h. The crude product was purified by column chromatography (CC) with 1 to 5% 2N NH₃/methanol in methylene chloride to afford 1,1-dimethylethyl 4-{2-[({[(phenylmethyl)oxy]carbonyl}amino)methyl]-1H-benzimidazol-4-yl}-1-piperazinecarboxylate as a brown solid (2.2 g, 69%). ¹H-NMR (DMSO-d₆): δ 12.2 (br s, 1H), 7.91 (t, 1H), 7.41-7.34 (m, 5H), 7.04 (m, 2H), 6.55 (m, 1H), 5.10 (s, 2H), 4.45 (d, 2H), 3.55 (m, 4H), 3.50-3.36 (m, 4H), 1.44 (s, 9H). MS m/z 466.1 (M+1).

### C) 1,1-Dimethylethyl 4-(2-{[methyl(5,6,7,8-tetrahydro-8-quinolinyl)amino]methyl}-1H-benzimidazol-4-yl)-1-piperazinecarboxylate

1,1-Dimethylethyl 4-(2-{[methyl(5,6,7,8-tetrahydro-8-quinolinyl)amino]methyl}-1H-benzimidazol-4-yl)-1-piperazinecarboxylate was prepared from 1,1-dimethylethyl 4-{2-[({[(phenylmethyl)oxy]carbonyl}amino)methyl]-1H-benzimidazol-4-yl}-1-piperazinecarboxylate (2.2 g, 4.7 mmol) via deprotection and reductive amination first with 6,7-dihydro-8(5H)quinolinone and then formaldehyde in a similar manner as described before to give a tan solid (0.8 g, 35% yield). ¹H-NMR (DMSO-d₆): δ 12.2 (br s, 1H), 8.51 (d, 1H), 7.55 (d, 1H), 7.22 (d, 1H), 7.08-7.00 (m, 2H), 6.50 (m, 1H), 4.08 (Abq, 2H), 4.00 (t, 1H), 3.54 (m, 4H), 3.54-3.38 (m, 4H), 2.85-2.68 (m, 2H), 2.27 (s, 3H), 2.16-1.94 (m, 3H), 1.76-1.64 (m, 1H), 1.45 (s, 9H). MS m/z 477.2 (M+1).

### D) N-Methyl-N-{[4-(1-piperazinyl)-1H-benzimidazol-2-yl]methyl}-5,6,7,8-tetrahydro-8-quinolinamine

1,1-Dimethylethyl 4-(2-{[methyl(5,6,7,8-tetrahydro-8-quinolinyl)amino]methyl}-1H-benzimidazol-4-yl)-1-piperazinecarboxylate (0.5 g, 0.1 mmol) was dissolved in anhydrous methanol (2 mL). 4N hydrochloric acid in dioxane (2 mL) was added and the reaction stirred for 2.5 h at which time the solvent was evaporated. The resulting oil was neutralized with sat. aqueous sodium bicarbonate and extracted with 1% methanol in ethyl acetate (3×25 mL). The organic layers were combined dried over sodium sulfate filtered and evaporated. Purification by reverse phase HPLC (0% to 70% acetonitrile/water/0.1% trifluoroacetic acid gradient), neutralization of the desired fractions and extraction from the aqueous layer (sat. with NaCl) with 1% methanol in ethyl acetate (3×25 ml) afforded N-methyl-N-{[4-(1-piperazinyl)-1H-benzimidazol-2-yl]methyl}-5,6,7,8-tetrahydro-8-quinolinamine as a white solid (0.3 g, 76% yield). ¹H-NMR (DMSO-d₆): δ 12.3 (br s, 1H), 8.51 (d, 1H), 7.55 (d, 1H), 7.22 (d, 1H), 7.08-7.00 (m, 2H), 6.50 (m, 1H), 4.07 (Abq, 2H), 3.95 (t, 1H), 3.55 (m, 4H), 3.14 (m, 4H), 3.14 (m, 4H), 2.88-2.68 (m, 2H), 2.27 (s, 3H), 2.16-1.94 (m, 3H), 1.76-1.64 (m, 1H), 1.45. MS m/z 377.2 (M+1).

### Example 35

### N-methyl-N-{[4-(4-methyl-1-piperazinyl)-1H-benzimidazol-2-yl]methyl}-5,6,7,8-tetrahydro-8-quinolinamine

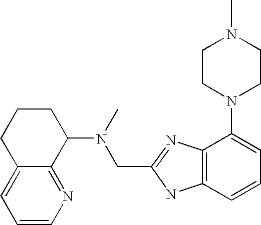

N-methyl-N-{[4-(4-methyl-1-piperazinyl)-1H-benzimidazol-2-yl]methyl}-5,6,7,8-tetrahydro-8-quinolinamine was prepared in a similar fashion as described above from N-methyl-N-{[4-(1-piperazinyl)-1H-benzimidazol-2-yl]methyl}-5,6,7,8-tetrahydro-8-quinolinamine (0.5 g, 0.1 mmol) and formaldehyde (15 mL, 0.2 mmol) to afford the product as a white solid (0.3 g, 60% yield). ¹H-NMR (DMSO-d₆): δ 12.2 (br s, 1H), 8.44 (d, 1H), 7.48 (d, 1H), 7.16 (dd, 1H), 7.00-6.93 (m, 2H), 6.43 (m, 1H), 4.00 (Abq, 2H), 3.91 (t, 1H), 3.44 (m, 4H), 2.84-2.74 (m, 1H), 2.70-2.56 (m, 5H), 2.31 (s, 3H), 2.21 (s, 3H), 2.16-1.88 (m, 3H), 1.68-1.56 (m, 1H). MS m/z 391.1 (M+1). HRMS (M+1) Calc'd for C23H31N6 (391.2610). Found 391.2595.

The R and S isomers can be separated using chiral chromatography or by supercritical fluid chromatography, SFC conditions: Chiralpcel OJ-H (3 cm), 1500 psi, 27 deg C., 2 ml/min, 5% methanol (0.5% DIPEA), 30% CH₂Cl₂.

### Example 36

### N-methyl-N-({4-[4-(2-methylpropyl)-1-piperazinyl]-1H-benzimidazol-2-yl}methyl)-5,6,7,8-tetrahydro-8-quinolinamine

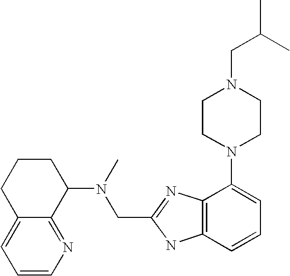

N-Methyl-N-({4-[4-(2-methylpropyl)-1-piperazinyl]-1H-benzimidazol-2-yl}methyl)-5,6,7,8-tetrahydro-8-quinolinamine was prepared in a similar fashion as described above from N-methyl-N-{[4-(1-piperazinyl)-1H-benzimidazol-2-yl]methyl}-5,6,7,8-tetrahydro-8-quinolinamine (0.5 g, 0.1 mmol) and isobutyraldehyde (18 mL, 0.2 mmol) to afford the product as a white solid (0.4 g, 72% yield). ¹H-NMR (DMSO-d₆): δ 12.2 (br s, 1H), 8.46 (d, 1H), 7.49 (d, 1H), 7.17 (dd, 1H), 7.00-6.93 (m, 2H), 6.41 (d, 1H), 4.01 (Abq, 2H), 3.92 (t, 1H), 3.43 (m, 4H), 2.85-2.75 (m, 1H), 2.66 (m, 1H), 2.58-2.46 (m, 4H), 2.22 (s, 3H), 2.10-2.00 (m, 3H), 1.95-1.88 (m, 2H), 1.80 (sept, 1H), 1.70-1.57 (m, 1H), 0.86 (d, 6H). MS m/z 433.1 (M+1). HRMS (M+1) calc'd for C26H37N6 (433.3080). Found 433.3076.

### Example 37

### 2-{[Methyl(5,6,7,8-tetrahydroquinolin-8-yl)amino]methyl}-1H-benzimidazole-5-carboxamide

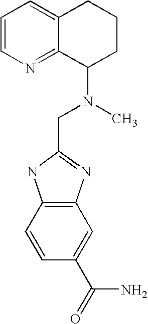

2-{[Methyl(5,6,7,8-tetrahydro-8-quinolinyl)amino]methyl}-1H-benzimidazole-5-carboxylic acid (190 mg, 0.56 mmol) was dissolved in N,N-dimethylformamide (2 mL). HATU (430 mg, 1.1 mmol), and N,N-diisopropylethylamine (220 mg, 1.7 mmol) were added to the reaction, ammonia was bubbled through the suspension for 10 minutes and the reaction was stirred for 16 h. The reaction was diluted with ethyl acetate (20 mL) and water (20 mL), separated and the organic phase concentrated. The crude material was purified using reverse phase HPLC (0% to 70% acetonitrile/water/0.1% trifluoroacetic acid gradient) to provide the product as a brown solid (6.4 mg, 2%, tan solid) as the trifluoroacetate salt: MS m/z 336 (M+1).

### Example 38

### 2-{[Methyl(5,6,7,8-tetrahydroquinolin-8-yl)amino]methyl}-N-(piperidin-4-ylmethyl)-1H-benzimidazole-5-carboxamide

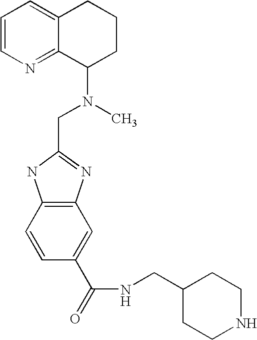

2-{[Methyl(5,6,7,8-tetrahydro-8-quinolinyl)amino]methyl}-1H-benzimidazole-5-carboxylic acid (100 mg, 0.30 mmol), bis(2-oxo-3-oxazolidinyl)phosphinic chloride (153 mg, 0.6 mmol), 1,1-dimethylethyl 4-(aminomethyl)-1-piperidinecarboxylate (130 mg, 0.60 mmol), and N,N-diisopropylethylamine (116 mg, 0.90 mmol) were dissolved in N,N-dimethylformamide (3 mL) and the reaction was stirred at 40° C. for 16 h. The reaction was concentrated, dissolved in methanol (2 mL) and 4 N HCl in dioxane (2 mL), stirred for 2 h and concentrated. The reaction mixture was purified using reverse phase HPLC (0% to 70% acetonitrile/water/0.1% trifluoroacetic acid gradient) and the fractions concentrated to provide the product (8.4 mg, 4%, light brown crystalline solid) as the trifluoroacetate salt: ¹H-NMR (DMSO-d₆) δ 8.58-8.52 (m, 3H), 8.26-8.16 (m, 1H), 8.14 (s, 1H), 7.81-7.76 (m, 2H), 7.65 (d, 1H, J=8.4 Hz), 7.47-7.44 (m, 1H), 4.85-4.80 (m, 1H), 4.62 (d, 1H, J=15 Hz), 4.46 (d, 1H, J=15 Hz), 3.27-3.23 (m, 2H), 3.19-3.16 (m, 2H), 2.85-2.78 (m, 2H), 2.73 (s, 3H), 2.39-2.33 (m, 1H), 2.08-1.96 (m, 2H), 1.85-1.72 (m, 4H), 1.37-1.26 (m, 2H). MS m/z 433.16 (M+1).

### Example 39

### 2-{[Methyl(5,6,7,8-tetrahydro-8-quinolinyl)amino]methyl}-N-(3-pyridinylmethyl)-1H-benzimidazole-5-carboxamide

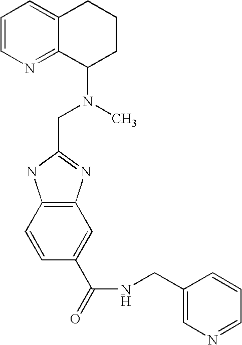

2-{[Methyl(5,6,7,8-tetrahydro-8-quinolinyl)amino]methyl}-N-(3-pyridinylmethyl)-1H-benzimidazole-5-carboxamide was prepared in a similar manner to 2-{[(methyl(5,6,7,8-tetrahydroquinolin-8-yl)amino]methyl}-N-(piperidin-4-ylmethyl)-1H-benzimidazole-5-carboxamide from 2-{[Methyl(5,6,7,8-tetrahydro-8-quinolinyl)amino]methyl}-1H-benzimidazole-5-carboxylic acid (190 mg, 0.56 mmol), bis(2-oxo-3-oxazolidinyl)phosphinic chloride (280 mg, 1.1 mmol), 2-(methylamino)pyridine (120 mg, 1.1 mmol), and N,N-diisopropylethylamine (220 mg, 1.7 mmol) to afford the trifluoroacetate salt as yellow crystalline solid (46 mg, 11%): ¹H-NMR (DMSO-d₆) δ 9.26-9.23 (m, 1H), 8.76 (s, 1H), 8.68-8.66 (m, 1H), 8.59-8.58 (m, 1H), 8.21 (s, 1H), 8.19-8.17 (m, 1H), 7.86-7.83 (m, 2H), 7.76-7.70 (m, 2H), 7.51-7.48 (m, 1H), 4.84-4.80 (m, 1H), 4.65-4.59 (m, 3H), 4.48 (d, 1H, J=15 Hz), 2.87-2.62 (m, 2H), 2.73 (s, 3H), 2.40-2.33 (m, 1H), 2.10-1.97 (m, 2H), 1.82-1.72 (m, 1H). MS m/z 427.1 (M+1).

### Example 40

### 2-{[Methyl(5,6,7,8-tetrahydro-8-quinolinyl)amino]methyl}-N-(4-pyridinylmethyl)-1H-benzimidazole-5-carboxamide

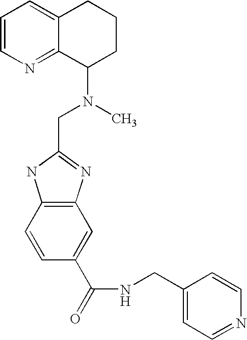

2-{[Methyl(5,6,7,8-tetrahydro-8-quinolinyl)amino]methyl}-N-(4-pyridinylmethyl)-1H-benzimidazole-5-carboxamide was prepared in a similar manner to 2-{[methyl(5,6,7,8-tetrahydroquinolin-8-yl)amino]methyl}-N-(piperidin-4-ylmethyl)-1H-benzimidazole-5-carboxamide from 2-{[methyl(5,6,7,8-tetrahydro-8-quinolinyl)amino]methyl}-1H-benzimidazole-5-carboxylic acid (190 mg, 0.56 mmol), bis(2-oxo-3-oxazolidinyl)phosphinic chloride (280 mg, 1.1 mmol), amine (120 mg, 1.1 mmol), and N,N-diisopropylethylamine (220 mg, 1.7 mmol) to afford the trifluoroacetate salt as a brown crystalline solid (66 mg, 19%): ¹H-NMR (DMSO-d₆) δ 9.36-9.34 (m, 1H), 8.78-8.76 (m, 2H), 8.59-8.58 (m, 1H), 8.25 (s, 1H), 7.88-7.80 (m, 4H), 7.43 (d, 1H, J=8.5 Hz), 7.51-7.48 (m, 1H), 4.85-4.81 (m, 1H), 4.70-4.69 (m, 2H), 4.65 (d, 1H, J=15 Hz), 4.49 (d, 1H, J=15 Hz), 2.88-2.77 (m, 2H), 2.73 (s, 3H), 2.40-2.35 (m, 1H), 2.09-1.98 (m, 2H), 1.82-1.73 (m, 1H). MS m/z 427.15 (M+1).

### Example 41

### N-(Cyclohexylmethyl)-2-{[methyl(5,6,7,8-tetrahydro-8-quinolinyl)amino]methyl}-1H-benzimidazole-5-carboxamide

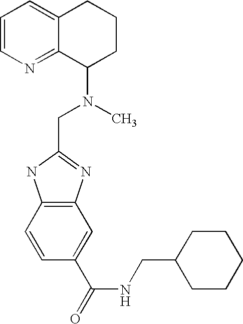

N-(Cyclohexyl methyl)-2-{[methyl (5,6,7,8-tetrahydro-8-quinolinyl)amino]methyl}-1H-benzimidazole-5-carboxamide was prepared in a similar manner to 2-{[methyl(5,6,7,8-tetrahydroquinolin-8-yl)amino]methyl}-N-(piperidin-4-ylmethyl)-1H-benzimidazole-5-carboxamide from 2-{[methyl(5,6,7,8-tetrahydro-8-quinolinyl)amino]methyl}-1H-benzimidazole-5-carboxylic acid (190 mg, 0.56 mmol), bis(2-oxo-3-oxazolidinyl)phosphinic chloride (280 mg, 1.1 mmol), 4-(aminomethyl)cyclohexane (130 mg, 1.1 mmol), and N,N-diisopropylethylamine (220 mg, 1.7 mmol) to afford the trifluoroacetate salt as a yellow solid (24.7 mg, 7%): ¹H-NMR (DMSO-d₆) δ 8.59-8.58 (m, 1H), 8.48-8.45 (m, 1H), 8.16 (s, 1H), 7.85-7.99 (m, 2H), 7.67 (d, 1H, J=8.6 Hz), 7.51-7.48 (m, 1H), 4.85-4.81 (m, 1H), 4.63 (d, 1H, J=15 Hz), 4.47 (d, 1H, J=15 Hz), 3.13-3.10 (m, 2H), 2.87-2.82 (m, 2H), 2.74 (s, 3H), 2.39-2.34 (m, 1H), 2.07-1.99 (m, 2H), 1.79-1.53 (m, 7H), 1.19-1.11 (m, 3H), 0.95-0.87 (m, 2H). MS m/z 432.2 (M+1).

### Example 42

### N-Methyl-N-{[5-({4-[2-(1-pyrrolidinyl)ethyl]-1-piperidinyl}carbonyl)-1H-benzimidazol-2-yl]methyl}-5,6,7,8-tetrahydro-8-quinolinamine

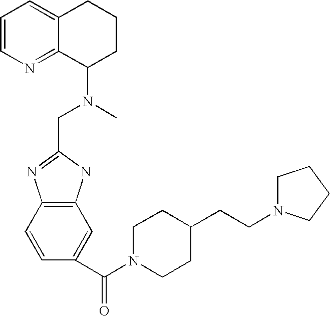

2-{[Methyl(5,6,7,8-tetrahydro-8-quinolinyl)amino]methyl}-1H-benzimidazole-5-carboxylic acid (190 mg, 0.56 mmol), bis(2-oxo-3-oxazolidinyl)phosphinic chloride (215 mg, 0.84 mmol), 4-[2-(1-pyrrolidinyl)ethyl]piperidine (155 mg, 0.84 mmol), and N,N-diisopropylethylamine (0.15 mL, 0.84 mmol) were dissolved in acetonitrile (5 mL) and the reaction was stirred at 40° C. for 16 h. The reaction was concentrated and the crude material purified using reverse phase HPLC (0% to 70% acetonitrile/water/0.1% trifluoroacetic acid gradient). Neutralization of the desired fractions with sat. NaHCO₃, extracted with EtOAc (4×10 mL), combined, dried over Na₂SO₄, filtered and evaporated to yield a yellow solid (17 mg, 6%): ¹H-NMR (DMSO-d₆) δ12.5 (br s, 1H), 8.45 (d, 1H), 7.48 (m, 3H), 7.17 (t, 1H), 7.11 (s, 1H), 4.04 (s, 2H), 3.92 (t, 1H), 2.83-2.75 (m, 2H), 2.68-2.50 (m, 6H), 2.24 (s, 3H), 2.07-2.01 (m, 1H), 1.95-1.87 (m, 2H), 1.70-1.53 (m, 7H), 1.45-1.41 (m, 2H), 1.21 (s, 1H), 1.13-1.02 (m, 2H). MS m/z 501 (M+1).

### Example 43

### 2-{[ethyl(5,6,7,8-tetrahydro-8-quinolinyl)amino]methyl}-N-[2-(1-methyl-1H-imidazol-5-yl)ethyl]-1H-benzimidazole-5-carboxamide

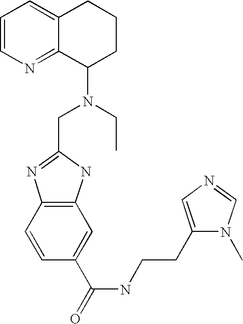

2-{[Ethyl(5,6,7,8-tetrahydro-8-quinolinyl)amino]methyl}-N-[2-(1-methyl-1H-imidazol-5-yl)ethyl]-1H-benzimidazole-5-carboxamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[ethyl(5,6,7,8-tetrahydro-8-quinolinyl)amino]methyl}-1H-benzimidazole-5-carboxylic acid pentafluorophenyl ester bound resin (0.625 g, 0.9 mmol) and [2-(1-methyl-1H-imidazol-5-yl)ethyl]amine dihydrochloride (0.165 g, 0.9 mmol) to give a yellow solid (0.035 g, 9% yield). ¹H-NMR (DMSO-d₆): δ 8.50 (d, 1H), 8.46 (t, 1H), 8.01 (s, 1H), 7.63 (d, 1H), 7.49 (m, 3H), 7.18 (m, 1H), 6.88 (s, 1H), 4.65 (dd, 1H), 4.06 (½ ABq, 1H), 4.04 (t, 1H), 3.93 (½ABq, 1H), 3.56 (s, 3H), 3.44 (dd, 2H), 2.80-2.64 (m, 4H), 2.14-2.05 (m, 1H), 1.96-1.7 (m, 2H), 1.68-1.55 (m, 1H), 0.91 (t, 3H). MS m/z 458 (M+1).

### Example 44

### N-(5-Aminopentyl)-2-{[ethyl(5,6,7,8-tetrahydro-8-quinolinyl)amino]methyl}-1H-benzimidazole-5-carboxamide

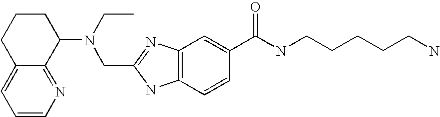

N-(5-Aminopentyl)-2-{[ethyl(5,6,7,8-tetrahydro-8-quinolinyl)amino]methyl}-1H-benzimidazole-5-carboxamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[ethyl(5,6,7,8-tetrahydro-8-quinolinyl)amino]methyl}-1H-benzimidazole-5-carboxylic acid pentafluorophenyl ester bound resin (0.625 g, 0.9 mmol) and diaminopentane (0.09 g, 0.9 mmol) to give a white solid (0.02 g, 5% yield). ¹H-NMR (DMSO-d₆): δ 8.56 (d, 1H), 8.48 (t, 1H), 8.08 (s, 1H), 7.69 (d, 1H), 7.56 (t, 2H), 7.24 (dd, 1H), 4.09 (t, 1H), 4.06 (q, 2H), 3.30 (d, 2H), 2.76-2.70 (m, 6H), 2.21-2.10 (m, 1H), 2.01-1.82 (m, 2H), 1.75-1.64 (m, 2H), 1.60-1.49 (m, 3H), 1.43-1.33 (m, 2H), 0.96 (t, 3H). MS m/z 435 (M+1).

### Example 45

### N-Ethyl-N-{[5-(4-morpholinylcarbonyl)-1H-benzimidazol-2-yl]methyl}-5,6,7,8-tetrahydro-8-quinolinamine

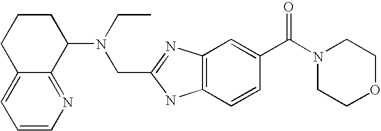

N-Ethyl-N-{[5-(4-morpholinylcarbonyl)-1H-benzimidazol-2-yl]methyl}-5,6,7,8-tetrahydro-8-quinolinamine was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[ethyl(5,6,7,8-tetrahydro-8-quinolinyl)amino]methyl}-1H-benzimidazole-5-carboxylic acid pentafluorophenyl ester bound resin (0.625 g, 0.9 mmol) and morpholine (0.08 mL, 0.9 mmol) to give a white solid (0.036 g, 10% yield). ¹H-NMR (DMSO-d₆): δ 12.6 (br s, 1H), 8.53 (d, 1H), 7.53 (m, 2H), 7.49 (d, 1H), 7.17 (m, 2H), 4.04 (½ ABq, 1H), 4.02 (t, 1H), 3.91 (½ABq, 1H), 3.57 (s, 4H), 3.49 (s, 4H), 2.82-2.64 (m, 4H), 2.15-2.06 (m, 1H), 1.96-1.76 (m, 2H), 1.69-1.55 (m, 1H), 0.91 (t, 3H). MS m/z 420 (M+1).

### Example 46

### N-{[5-({4-[2-(Diethylamino)ethyl]-1-piperazinyl}carbonyl)-1H-benzimidazol-2-yl]methyl}-N-ethyl-5,6,7,8-tetrahydro-8-quinolinamine

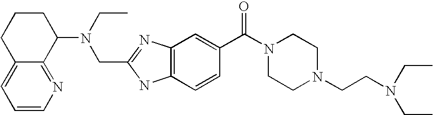

N-{[5-({4-[2-(Diethylamino)ethyl]-1-piperazinyl}carbonyl)-1H-benzimidazol-2-yl]methyl}-N-ethyl-5,6,7,8-tetrahydro-8-quinolinamine was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[ethyl(5,6,7,8-tetrahydro-8-quinolinyl)amino]methyl}-1H-benzimidazole-5-carboxylic acid pentafluorophenyl ester bound resin (0.625 g, 0.9 mmol) and N,N-diethyl-2-(1-piperazinyl)ethanamine (0.17 g, 0.9 mmol) to give a white solid (0.036 g, 10% yield). ¹H-NMR (DMSO-d₆): δ 12.6 (br s, 1H), 8.51 (d, 1H), 7.53 (m, 2H), 7.49 (d, 1H), 7.17 (dd, 1H), 7.12 (s, 1H), 4.04 (½ ABq, 1H), 4.02 (t, 1H), 3.91 (½ABq, 1H), 3.46 (s, 4H), 3.32 (s, 4H), 2.81-2.62 (m, 4H), 2.47-2.38 (m, 8H), 2.15-2.06 (m, 1H), 1.96-1.76 (m, 2H), 1.68-1.55 (m, 1H), 0.91 (t, 9H). MS m/z 518 (M+1).

### Example 47

### Methyl N-[(2-{[ethyl(5,6,7,8-tetrahydro-8-quinolinyl)amino]methyl}-1H-benzimidazol-5-yl)carbonyl]-L-histidinate

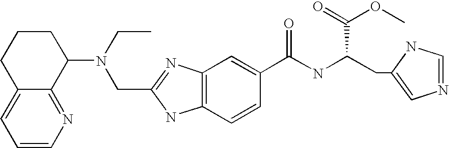

Methyl N-[(2-{[ethyl(5,6,7,8-tetrahydro-8-quinolinyl)amino]methyl}-1H-benzimidazol-5-yl)carbonyl]-L-histidinate was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[ethyl(5,6,7,8-tetrahydro-8-quinolinyl)amino]methyl}-1H-benzimidazole-5-carboxylic acid pentafluorophenyl ester bound resin (0.625 g, 0.9 mmol) and L-histidine methyl ester (0.15 g, 0.9 mmol) to give a white solid (0.05 g, 10% yield). ¹H-NMR (DMSO-d₆): δ 12.7 (br s,), 11.8 (br s, 1H), 8.77 (s, 1H), 8.51 (d, 1H), 8.02 (s, 1H), 7.63 (s, 1H), 7.56 (s, 1H), 7.47 (d, 1H), 7.18 (m, 1H), 6.89 (s, 1H), 4.65 (dd, 1H), 4.06 (½ ABq, 1H), 4.04 (t, 1H), 3.91 (½ABq, 1H), 3.57 (s, 4H), 3.59 (s, 3H), 3.00 (s, 2H), 2.80-2.64 (m, 4H), 2.14-2.05 (m, 1H), 1.96-1.7 (m, 2H), 1.68-1.57 (m, 1H), 0.91 (t, 3H). MS m/z 502 (M+1).

### Example 48

### N-Ethyl-N-({5-[(4-methyl-1-piperazinyl)carbonyl]-1H-benzimidazol-2-yl}methyl)-5,6,7,8-tetrahydro-8-quinolinamine

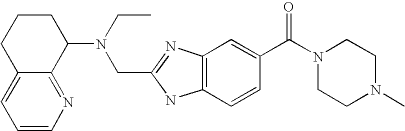

N-Ethyl-N-({5-[(4-methyl-1-piperazinyl)carbonyl]-1H-benzimidazol-2-yl}methyl)-5,6,7,8-tetrahydro-8-quinolinamine was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[ethyl(5,6,7,8-tetrahydro-8-quinolinyl)amino]methyl}-1H-benzimidazole-5-carboxylic acid pentafluorophenyl ester bound resin (0.150 g, 0.22 mmol) and methylpiperazine (0.25 g, 0.25 mmol) to give a yellow foam (0.015 g, 15% yield). ¹H-NMR (DMSO-d₆): δ 12.6 (br s, 1H), 8.57 (d, 1H), 7.64-7.56 (s, 1H), 7.54 (d, 2H), 7.23 dd, 1H), 7.20-7.13 (s, 1H), 4.10 (½ ABq, 1H), 4.07 (t, 1H), 3.97 (½ABq, 1H), 3.54 (s, 4H), 2.85-2.68 (m, 4H), 2.34 (s, 4H), 2.22 (s, 3H), 2.19-2.01 (m, 1H), 2.02-1.80 (m, 2H), 1.75-1.64 (m, 1H), 0.97 (t, 3H). MS m/z 433 (M+1).

### Example 49

### N-[2-(Dimethylamino)ethyl]-2-{[ethyl(5,6,7,8-tetrahydro-8-quinolinyl)amino]methyl}-N-methyl-1H-benzimidazole-5-carboxamide

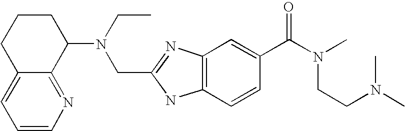

N-[2-(Dimethylamino)ethyl]-2-{[ethyl(5,6,7,8-tetrahydro-8-quinolinyl)amino]methyl}-N-methyl-1H-benzimidazole-5-carboxamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[ethyl(5,6,7,8-tetrahydro-8-quinolinyl)amino]methyl}-1H-benzimidazole-5-carboxylic acid pentafluorophenyl ester bound resin (0.150 g, 0.22 mmol) and N,N,N′-trimethyl-1,2-ethanediamine (0.25 g, 0.25 mmol) to give a yellow foam (0.009 g, 15% yield). ¹H-NMR (DMSO-d₆): δ 12.6 (br s, 1H), 8.56 (d, 1H), 7.64-7.55 (s, 1H), 7.54 (d, 2H), 7.23 dd, 1H), 7.20-7.13 (s, 1H), 4.65 (dd, 1H), 4.10 (½ ABq, 1H), 4.07 (t, 1H), 3.97 (½ABq, 1H), 3.36 (s, 6H), 2.99 (s, 3H), 2.30-2.10 (m, 3H), 2.1-1.80 (m, 4H), 1.79-1.61 (m, 1H), 0.97 (t, 3H). MS m/z 435 (M+1).

### Example 50

### N-[2-(Dimethylamino)ethyl]-2-{[ethyl(5,6,7,8-tetrahydro-8-quinolinyl)amino]methyl}-1H-benzimidazole-5-carboxamide

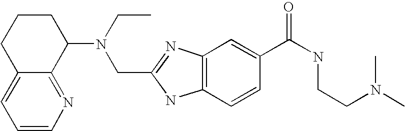

N-[2-(Dimethylamino)ethyl]-2-{[ethyl(5,6,7,8-tetrahydro-8-quinolinyl)amino]methyl}-1H-benzimidazole-5-carboxamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[ethyl(5,6,7,8-tetrahydro-8-quinolinyl)amino]methyl}-1H-benzimidazole-5-carboxylic acid pentafluorophenyl ester bound resin (0.150 g, 0.22 mmol) and N,N-dimethyl-1,2-ethanediamine (0.25 g, 0.25 mmol) to give a yellow foam (0.009 g, 15% yield). ¹H-NMR (DMSO-d₆): δ 12.6 (br s, 1H), 8.56 (d, 1H), 8.32 (t, 1H), 8.07 (s, 1H), 7.73-7.63 (s, 1H), 7.59 (s, 1H), 7.54 (d, 1H), 7.23 dd, 1H), 4.65 (dd, 1H), 4.12 (½ ABq, 1H), 4.09 (t, 1H), 4.02 (½ABq, 1H), 3.40 (dd, 2H), 3.36 (s, 6H), 2.99 (s, 3H), 2.85-2.68 (m, 4H), 2.48 (t, 2H), 2.24 (s, 3H), 2.21-2.09 (m, 1H), 2.00-1.83 (m, 2H), 1.79-1.61 (m, 1H), 0.97 (t, 3H). MS m/z 421 (M+1).

### Example 51

### N-Methyl-N-[2-(methylamino)ethyl]-2-{[methyl(5,6,7,8-tetrahydro-8-quinolinyl)amino]methyl}-1H-benzimidazole-5-carboxamide

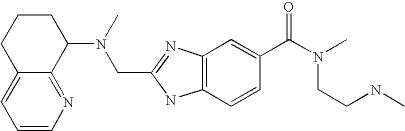

N-Methyl-N-[2-(methylamino)ethyl]-2-{[methyl(5,6,7,8-tetrahydro-8-quinolinyl)amino]methyl}-1H-benzimidazole-5-carboxamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[methyl(5,6,7,8-tetrahydro-8-quinolinyl)amino]methyl}-1H-benzimidazole-5-carboxylic acid pentafluorophenyl ester bound resin (0.450 g, 0.37 mmol) and N,N′-dimethyl-1,2-ethanediamine (0.35 g, 0.4 mmol) to give a yellow foam (0.025 g, 15% yield). ¹H-NMR (DMSO-d₆): δ 8.56 (s, 1H), 7.64 (m, 2H), 7.59 (m, 3H), 4.20-4.01 (m, 4H), 3.81 (m, 1H), 3.11 (m, 2H), 2.80 (s, 3H), 2.65 (s, 3H), 2.1 (m, 4H), 2.07-1.90 (m, 2H), 1.78-1.62 (m, 1H). MS m/z 407 (M+1).

### Example 52

### N-[2-(Dimethylamino)ethyl]-2-{[methyl(5,6,7,8-tetrahydro-8-quinolinyl)amino]methyl}-1H-benzimidazole-5-carboxamide

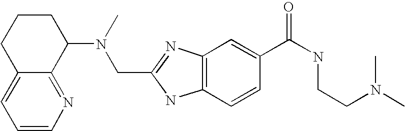

N-[2-(Dimethylamino)ethyl]-2-{[ethyl(5,6,7,8-tetrahydro-8-quinolinyl)amino]methyl}-1H-benzimidazole-5-carboxamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[methyl(5,6,7,8-tetrahydro-8-quinolinyl)amino]methyl}-1H-benzimidazole-5-carboxylic acid pentafluorophenyl ester bound resin (0.450 g, 0.37 mmol) and N,N-dimethyl-1,2-ethanediamine (0.35 g, 0.4 mmol) to give a yellow foam (0.015 g, 10% yield). ¹H-NMR (DMSO-d₆): δ 8.57 (s, 1H), 7.66 (d, 2H), 7.34-7.30 (m, 3H), 4.16 (dd, 2H), 4.06 (t, 1H), 3.82 (m, 1H), 3.52-3.41 (m, 1H), 3.15-3.01 (m, 1H), 2.87-2.75 (m, 4H), 2.69 (s, 3H), 2.16 (m, 4H), 2.07-1.88 (m, 2H), 1.77-1.63 (m, 1H). MS m/z 407 (M+1).

### Example 53

### N-[2-(Methylamino)ethyl]-2-{[methyl(5,6,7,8-tetrahydro-8-quinolinyl)amino]methyl}-1H-benzimidazole-5-carboxamide

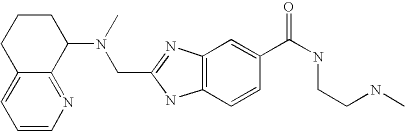

N-[2-(Methylamino)ethyl]-2-{[methyl(5,6,7,8-tetrahydro-8-quinolinyl)amino]methyl}-1H-benzimidazole-5-carboxamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[methyl(5,6,7,8-tetrahydro-8-quinolinyl)amino]methyl}-1H-benzimidazole-5-carboxylic acid pentafluorophenyl ester bound resin (0.450 g, 0.37 mmol) and 1,1-dimethylethyl (2-aminoethyl)methylcarbamate (0.35 g, 0.4 mmol) to give a yellow foam (0.012 g, 8% yield). ¹H-NMR (DMSO-d₆): δ 8.55 (s, 1H), 7.82 (d, 1H), 7.73 (d, 1H), 7.55 (d, 1H), 7.29 (t, 1H), 7.23 (dd, 1H), 4.16 (s, 2H), 4.03 (t, 1H), 3.58 (dd, 2H), 2.91-2.68 (m, 2H), 2.86 (t, 2H), 2.44 (s, 3H), 2.35 (s, 3H), 2.19-2.08 (m, 1H), 2.06-1.98 (m, 2H), 1.73-1.68 (m, 1H). MS m/z 393 (M+1).

### Example 54

### N-(3-Amino-2,2-dimethylpropyl)-2-{[methyl(5,6,7,8-tetrahydro-8-quinolinyl)amino]methyl}-1H-benzimidazole-5-carboxamide

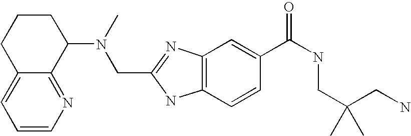

N-(3-Amino-2,2-dimethylpropyl)-2-{[methyl(5,6,7,8-tetrahydro-8-quinolinyl)amino]methyl}-1H-benzimidazole-5-carboxamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[methyl(5,6,7,8-tetrahydro-8-quinolinyl)amino]methyl}-1H-benzimidazole-5-carboxylic acid pentafluorophenyl ester bound resin (0.450 g, 0.37 mmol) and 2,2-dimethyl-1,3-propanediamine (0.40 g, 0.4 mmol) to give a yellow foam (0.022 g, 13% yield). ¹H-NMR (DMSO-d₆): δ 8.53 (d, 1H), 7.84 (d, 1H), 7.75 (d, 1H), 7.55 (d, 1H), 7.32 (t, 1H), 7.23 (dd, 1H), 4.17 (s, 2H), 4.03 (t, 1H), 3.37-3.35 (m, 2H), 2.93-2.73 (m, 2H), 2.35 (s, 3H), 2.13-1.93 (m, 4H), 1.76-1.65 (m, 2H). MS m/z 421 (M+1).

### Example 55

### N-[2-(Dimethylamino)ethyl]-N-methyl-2-{[methyl(5,6,7,8-tetrahydro-8-quinolinyl)amino]methyl}-1H-benzimidazole-5-carboxamide

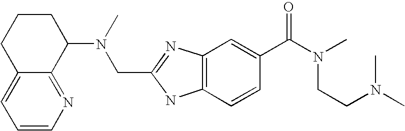

N-[2-(Dimethylamino)ethyl]-N-methyl-2-{[methyl(5,6,7,8-tetrahydro-8-quinolinyl)amino]methyl}-1H-benzimidazole-5-carboxamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from 2-{[methyl(5,6,7,8-tetrahydro-8-quinolinyl)amino]methyl}-1H-benzimidazole-5-carboxylic acid pentafluorophenyl ester bound resin (0.450 g, 0.37 mmol) and 1 N,N,N′-trimethyl-1,2-ethanediamine (0.35 g, 0.4 mmol) to give a yellow foam (0.012 g, 8% yield). ¹H-NMR (DMSO-d₆): δ 8.54 (d, 1H), 7.82 (d, 1H), 7.58 (t, 1H), 7.56 (d, 1H), 7.24 (dd, 1H), 7.20 (t, 1H), 7.12 (s, 1H), 4.10 (s, 2H), 3.97 (t, 1H), 3.67 (s, 1H), 3.07 (s, 2H), 2.92-2.66 (m, 4H), 2.29 (s, 9H), 2.13-1.91 (m, 4H), 1.82 (s, 3H), 1.75-1.62 (m, 1H). MS m/z 421 (M+1).

### Example 56

### Phenylmethyl 4-[(2-{[methyl(5,6,7,8-tetrahydro-8-quinolinyl)amino]methyl}-1H-benzimidazol-5-yl)carbonyl]-1-piperazinecarboxylate (Intermediate)

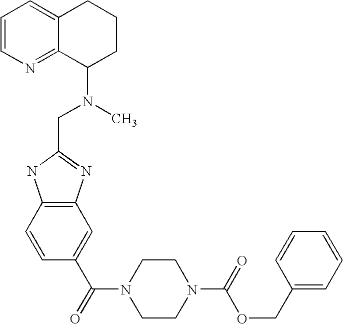

Phenylmethyl 4-[(2-{[methyl(5,6,7,8-tetrahydro-8-quinolinyl)amino]methyl}-1H-benzimidazol-5-yl)carbonyl]-1-piperazinecarboxylate was prepared from 2-{[methyl(5,6,7,8-tetrahydro-8-quinolinyl)amino]methyl}-1H-benzimidazole-5-carboxylic acid (190 mg, 0.56 mmol), bis(2-oxo-3-oxazolidinyl)phosphinic chloride (280 mg, 1.1 mmol), amine (150 mg, 1.1 mmol), and N,N-diisopropylethylamine (220 mg, 1.7 mmol) to afford the trifluoroacetate salt as a yellow solid (20 mg, 5%): ¹H-NMR (DMSO-d₆) 8.8.59-8.58 (m, 1H), 7.87-7.85 (m, 1H), 7.70-7.68 (m, 2H), 7.52-7.49 (m, 1H), 7.34-7.27 (m, 6H), 5.07 (s, 2H), 4.78-4.74 (m, 1H), 4.59 (d, 1H, J=15.2 Hz), 4.43 (d, 1H, J=15.2 Hz), 3.58-3.34 (br m, 8H), 2.86-2.78 (m, 2H), 2.68 (s, 3H), 2.38-2.32 (m, 1H), 2.09-1.95 (m, 2H), 1.81-1.71 (m, 1H). MS m/z 539.24 (M+1).

### Example 57

### N-Methyl-N-{[5-(1-piperazinylcarbonyl)-1H-benzimidazol-2-yl]methyl}-5,6,7,8-tetrahydro-8-quinolinamine

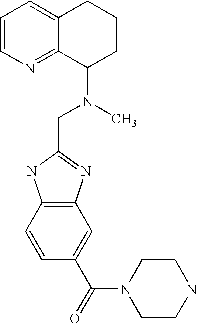

Phenylmethyl 4-[(2-{[methyl(5,6,7,8-tetrahydro-8-quinolinyl)amino]methyl}-1H-benzimidazol-5-yl)carbonyl]-1-piperazinecarboxylate (10 mg, 0.13 mmol) and palladium (10% w/w on carbon) was dissolved in ethanol (10 mL) and placed under a hydrogen atmosphere. After stirring for 4 h, the reaction was filtered through diatomaceous earth and concentrated to provide the product (4.1 mg, 42%, white solid) as the trifluoroacetate salt: MS m/z 405 (M+1).

### Example 58

### N-Methyl-N-({5-[(4-methyl-1-piperazinyl)carbonyl]-1H-benzimidazol-2-yl}methyl)-5,6,7,8-tetrahydro-8-quinolinamine

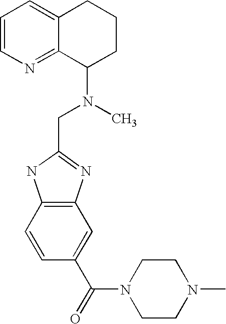

N-Methyl-N-({5-[(4-methyl-1-piperazinyl)carbonyl]-1H-benzimidazol-2-yl}methyl)-5,6,7,8-tetrahydro-8-quinolinamine was prepared from 2-{[methyl(5,6,7,8-tetrahydro-8-quinolinyl)amino]methyl}-1H-benzimidazole-5-carboxylic acid (190 mg, 0.56 mmol), bis(2-oxo-3-oxazolidinyl)phosphinic chloride (280 mg, 1.1 mmol), 1-methylpiperazine (110 mg, 1.1 mmol), and N,N-diisopropylethylamine (220 mg, 1.7 mmol) to afford the trifluoroacetate salt as a white solid (75.4 mg, 17%): ¹H-NMR (DMSO-d₆) δ 10.61 (br s, 1H), 8.62-8.61 (m, 1H), 7.94-7.92 (m, 1H), 7.78 (s, 1H), 7.73 (d, 1H, J=8.4 Hz), 7.58-7.54 (m, 1H), 7.40 (d, 1H, J=8.4 Hz), 4.75-4.72 (m, 1H), 4.59 (d, 1H, J=15.2 Hz), 4.43 (d, 1H, J=15.2 Hz), 3.62-3.08 (br m, 8H), 2.86-2.84 (m, 2H), 2.80 (s, 3H), 2.61 (s, 3H), 2.33-2.30 (m, 1H), 2.06-1.95 (m, 2H), 1.81-1.70 (m, 1H). MS m/z 419.20 (M+1).

### Example 59

### N-Methyl-N-[(5-{[4-(phenylmethyl)-1-piperazinyl]carbonyl}-1H-benzimidazol-2-yl)methyl]-5,6,7,8-tetrahydro-8-quinolinamine

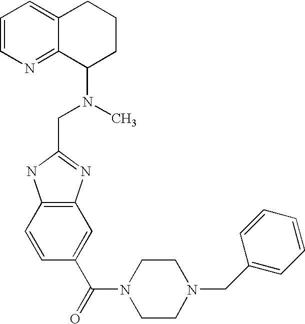

N-Methyl-N-[(5-{[4-(phenylmethyl)-1-piperazinyl]carbonyl}-1H-benzimidazol-2-yl)methyl]-5,6,7,8-tetrahydro-8-quinolinamine was prepared from 2-{[methyl(5,6,7,8-tetrahydro-8-quinolinyl)amino]methyl}-1H-benzimidazole-5-carboxylic acid (190 mg, 0.56 mmol), bis(2-oxo-3-oxazolidinyl)phosphinic chloride (280 mg, 1.1 mmol), 1-benzylpiperazine (200 mg, 1.1 mmol), and N,N-diisopropylethylamine (220 mg, 1.7 mmol) to afford the trifluoroacetate salt as a white solid (96 mg, 20%): ¹H-NMR (CD₃OD) δ 8.76-8.74 (m, 1H), 8.30-8.28 (m, 1H), 7.97-7.94 (m, 1H), 7.87-7.82 (m, 2H), 7062-7.59 (m, 1H), 7.53-7.47 (m, 5H), 4.58-4.54 (m, 2H), 4.50-4.44 (m, 3H), 4.39-4.32 (m, 4H), 3.94-3.91 (m, 3H), 3.30-3.26 (m, 2H), 3.06-3.00 (m, 2H), 2.39 (s, 3H), 2.39-2.31 (m, 1H), 2.23-2.17 (m, 1H), 2.10-2.01 (m, 1H), 1.97-1.87 (m, 1H). MS m/z 495.38 (M+1).

### Example 60

### N-[(5-{[4-(4-Chlorophenyl)-1-piperazinyl]carbonyl}-1H-benzimidazol-2-yl)methyl]-N-methyl-5,6,7,8-tetrahydro-8-quinolinamine

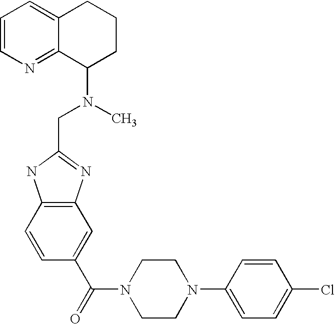

N-[(5-{[4-(4-Chlorophenyl)-1-piperazinyl]carbonyl}-1H-benzimidazol-2-yl)methyl]-N-methyl-5,6,7,8-tetrahydro-8-quinolinamine was prepared from 2-{[methyl(5,6,7,8-tetrahydro-8-quinolinyl)amino]methyl}-1H-benzimidazole-5-carboxylic acid (190 mg, 0.56 mmol), bis(2-oxo-3-oxazolidinyl)phosphinic chloride (280 mg, 1.1 mmol), (4-chlorophenyl)piperazine dihydrochloride (300 mg, 1.1 mmol), and N,N-diisopropylethylamine (380 mg, 2.8 mmol) to afford the trifluoroacetate salt as an off white solid (60 mg, 13%): ¹H-NMR (DMSO-d₆) δ 8.75-8.74 (m, 1H), 8.27-8.25 (m, 1H), 7.87-7.79 (m, 3H), 7.58-7.56 (m, 1H), 7.22-7.18 (m, 2H), 6.97-6.93 (m, 2H), 4.58-4.54 (m, 1H), 4.52 (d, 1H, J=15.8 Hz), 4.33 (d, 1H, J=15.8 Hz), 3.98-3.55 (br m, 4H), 3.27-3.08 (br m, 4H), 3.02-2.99 (m, 2H), 2.41 (s, 3H), 2.37-2.31 (m, 1H), 2.23-2.18 (m, 1H), 2.11-2.01 (m, 1H), 1.96-1.85 (m, 1H). MS m/z 515.15 (M+1).

### Example 61

### N-(5-Aminopentyl)-2-{[methyl(5,6,7,8-tetrahydro-8-quinolinyl)amino]methyl}-1H-benzimidazole-4-carboxamide

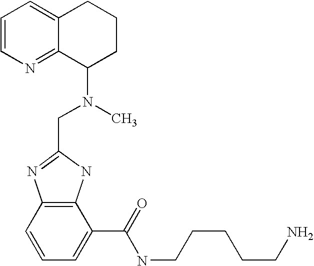

2-{[Methyl(5,6,7,8-tetrahydro-8-quinolinyl)amino]methyl}-1H-benzimidazole-4-carboxylic acid (180 mg, 0.54 mmol), bis(2-oxo-3-oxazolidinyl)phosphinic chloride (275 mg, 1.1 mmol), Boc protected amine (164 mg, 0.81 mmol), and N,N-diisopropylethylamine (140 mg, 1.1 mmol) were dissolved in acetonitrile (5 mL) and the reaction was stirred at 60° C. for 16 h. The reaction diluted with ethyl acetate (20 mL) and water (20 mL), separated and the organic phase concentrated. The crude carbamate was dissolved in dichloromethane (5 mL) and trifluoroacetic acid (2 mL) and stirred for 4 h. The reaction was concentrated, diluted with ethyl acetate (20 mL) and saturated aqueous sodium bicarbonate (20 mL), separated and the organic phase concentrated. The reaction mixture was purified using reverse phase HPLC (0% to 70% acetonitrile/water/0.1% trifluoroacetic acid gradient), the fractions concentrated, diluted with ethyl acetate (20 mL) and saturated aqueous sodium bicarbonate (20 mL), separated and the organic phase concentrated to provide the product (50 mg, 23%) as a tan solid. MS m/z 336 (M+1).

### Example 62

### N-Methyl-N-[(4-{[4-(phenylmethyl)-1-piperazinyl]carbonyl}-1H-benzimidazol-2-yl)methyl]-5,6,7,8-tetrahydro-8-quinolinamine

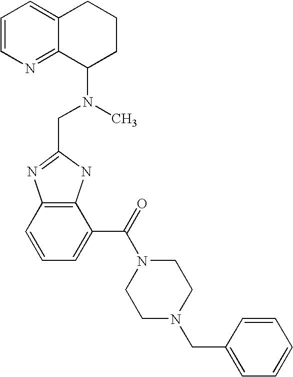

N-Methyl-N-[(4-{[4-(phenylmethyl)-1-piperazinyl]carbonyl}-1H-benzimidazol-2-yl)methyl]-5,6,7,8-tetrahydro-8-quinolinamine was prepared in a similar manner to 2-{[methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (115 mg, 0.45 mmol), N-benzylpiperazine (53 mg, 0.30 mmol), and N,N-diisopropylethylamine (58 mg, 0.45 mmol) to afford the trifluoroacetate salt as a yellow solid (95 mg, 38%): ¹H-NMR (DMSO-d₆) δ 8.58-8.56 (m, 1H), 7.85-7.83 (m, 1H), 7.78-7.75 (m, 1H), 7.50-7.45 (m, 6H), 7.36-7.34 (m, 2H), 4.76-4.72 (m, 1H), 4.60 (d, 1H, J=14.8 Hz), 4.48 (d, 1H, J=14.8 Hz), 4.42-4.37 (m, 1H), 3.84-3.80 (m, 1H), 3.49-2.98 (br m, 8H), 2.91-2.78 (m, 2H), 2.69 (s, 3H), 2.37-2.31 (m, 1H), 2.08-1.97 (m, 2H), 1.80-1.69 (m, 1H). MS m/z 495.19 (M+1).

### Example 63

### N-{[4-(Aminomethyl)phenyl]methyl}-2-{[methyl(5,6,7,8-tetrahydro-8-quinolinyl)amino]methyl}-1H-benzimidazole-4-carboxamide

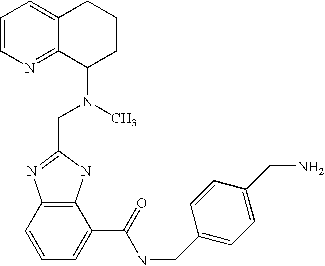

2-{[Methyl(5,6,7,8-tetrahydro-8-quinolinyl)amino]methyl}-1H-benzimidazole-4-carboxylic acid (190 mg, 0.56 mmol), bis(2-oxo-3-oxazolidinyl)phosphinic chloride (220 mg, 0.85 mmol), 1,1-dimethylethyl {[4-(aminomethyl)phenyl]methyl}carbamate (104 mg, 0.56 mmol), and N,N-diisopropylethylamine (110 mg, 0.85 mmol) were dissolved in acetonitrile (5 mL) and N,N-dimethylformamide (2 mL) and the reaction was stirred at 35° C. for 16 h. The reaction was concentrated and purified using reverse phase HPLC (0% to 70% acetonitrile/water/0.1% trifluoroacetic acid gradient) to provide the protected amine. The carbamate was dissolved in dichloromethane (3 mL) and trifluoroacetic acid (2 mL) and stirred for 3 h. The reaction was concentrated and purified using reverse phase HPLC (0% to 70% acetonitrile/water/0.1% trifluoroacetic acid gradient) to provide the product (56 mg, 13%, light brown solid) as the trifluoroacetate salt: ¹H-NMR (DMSO-d₆) δ 8.47-8.46 (m, 1H), 8.11 (br s, 2H), 7.90-7.88 (m, 1H), 7.83-7.81 (m, 1H), 7.73-7.64 (m, 4H), 7.39-7.32 (m, 5H), 4.58-4.55 (m, 2H), 4.13-4.08 (m, 3H), 4.00-3.95 (m, 2H), 2.82-2.75 (m, 2H), 2.72 (s, 3H), 2.31-2.26 (m, 1H), 2.05-1.94 (m, 2H), 1.63-1.57 (m, 1H). MS m/z 455.15 (M+1).

### Example 64

### N-[2-(Methyloxy)ethyl]-2-{[methyl(5,6,7,8-tetrahydro-8-quinolinyl)amino]methyl}-1H-benzimidazole-4-carboxamide

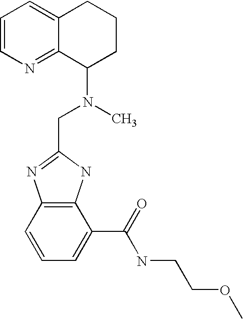

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (115 mg, 0.45 mmol), (methyloxy)ethanamine (34 mg, 0.45 mmol), and N,N-diisopropylethylamine (58 mg, 0.45 mmol) were dissolved in acetonitrile (3 mL) and N,N-dimethylformamide (2 mL) and the reaction was stirred at 35° C. for 64 h. The reaction was concentrated and purified using reverse phase HPLC (0% to 70% acetonitrile/water/0.1% trifluoroacetic acid gradient) to provide the product (9 mg, 5%, yellow solid) as the trifluoroacetate salt: ¹H-NMR (DMSO-d₆) δ 9.26 (br s, 1H), 8.55-8.54 (m, 1H), 7.88-7.86 (m, 1H), 7.82-7.76 (m, 2H), 7.45-7.42 (m, 1H), 7.37-7.33 (m, 1H), 4.84-4.80 (m, 1H), 4.70 (d, 1H, J=15 Hz), 4.55 (d, 1H, J=15 Hz), 3.53-3.51 (m, 2H), 3.48-3.47 (m, 2H), 3.24 (s, 3H), 2.90-2.80 (m, 2H), 2.79 (s, 3H), 2.46-2.34 (m, 1H), 2.10-2.00 (m, 2H), 1.84-1.70 (m, 1H). MS m/z 394.16 (M+1).

### Example 65

### N-[(4-{[4-(Aminoacetyl)-1-piperazinyl]carbonyl}-1H-benzimidazol-2-yl)methyl]-N-methyl-5,6,7,8-tetrahydro-8-quinolinamine

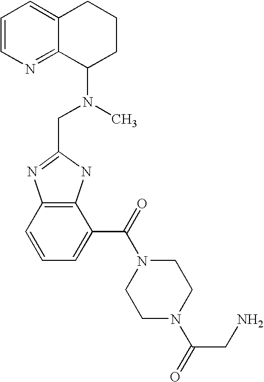

### A) Tert-butyl-[2-oxo-2-(1-piperazinyl)ethyl]carbamate

N-Benzyloxypiperazine (624 mg, 2.8 mmol), bis(2-oxo-3-oxazolidinyl)phosphinic chloride (1.07 g, 4.2 mmol), N-(tert-butylcarboxy)glycine (500 mg, 2.8 mmol), and N,N-diisopropylethylamine (540 mg, 4.2 mmol) were dissolved in acetonitrile (20 mL) and the reaction was stirred for 64 h. The reaction was diluted with ethyl acetate (30 mL) and water (50 mL), separated and the organic phase dried over sodium sulfate, filtered and concentrated to provide the product as an oil: ¹H-NMR (DMSO-d₆) δ 7.37-7.28 (m, 5H), 6.73-6.70 (m, 1H), 5.06 (s, 2H), 3.76-3.73 (m, 2H), 3.39-3.34 (m, 8H), 1.34 (s, 9H). The protected amine and Pd/C (10% w/w, catalytic) were dissolved in ethanol (300 mL) and placed under a hydrogen atmosphere for 16 h. The reaction was filtered through celite and concentrated to provide the product (670 mg, 98% over 2 steps) as a white solid: ¹H-NMR (DMSO-d₆) δ 6.68-6.66 (m, 1H), 3.73-3.71 (m, 2H), 3.34-3.29 (m, 4H), 2.66-2.62 (m, 4H), 1.35 (s, 9H).

### B) N-[(4-{[4-(Aminoacetyl)-1-piperazinyl]carbonyl}-1H-benzimidazol-2-yl)methyl]-N-methyl-5,6,7,8-tetrahydro-8-quinolinamine

2-{[Methyl(5,6,7,8-tetrahydro-8-quinolinyl)amino]methyl}-1H-benzimidazole-4-carboxylic acid (150 mg, 0.44 mmol), bis(2-oxo-3-oxazolidinyl)phosphinic chloride (167 mg, 0.66 mmol), tert-butyl-[2-oxo-2-(1-piperazinyl)ethyl]carbamate (160 mg, 0.66 mmol), and N,N-diisopropylethylamine (85 mg, 0.66 mmol) were dissolved in acetonitrile (3 mL) and N,N-dimethylformamide (2 mL) and the reaction was stirred for 16 h. The reaction was concentrated and purified using reverse phase HPLC (0% to 70% acetonitrile/water/0.1% trifluoroacetic acid gradient) to provide the protected amine. The carbamate was dissolved in dichloromethane (3 mL) and trifluoroacetic acid (2 mL), stirred for 3 h, concentrated and lyophilized from water to provide the product (113 mg, 32%, yellow solid) as the trifluoroacetate salt: ¹H-NMR (DMSO-d₆) δ 8.55-8.54 (m, 1H), 8.05 (br s, 3H), 7.81-7.79 (m, 1H), 7.75-7.74 (m, 1H), 7.48-7.44 (m, 1H), 7.36-7.30 (m, 2H), 4.84-4.79 (m, 1H), 4.64 (d, 1H, J=15 Hz), 4.51 (d, 1H, J=15 Hz), 3.91-3.86 (m, 2H), 3.57-3.32 (m, 8H), 2.87-2.80 (m, 2H), 2.74 (s, 3H), 2.41-2.31 (m, 1H), 2.09-2.00 (m, 2H), 1.81-1.71 (m, 1H). MS m/z 462.20 (M+1).

### Example 66

### 2-{4-[(2-{[Methyl(5,6,7,8-tetrahydro-8-quinolinyl)amino]methyl}-1H-benzimidazol-4-yl)carbonyl]-1-piperazinyl}acetamide

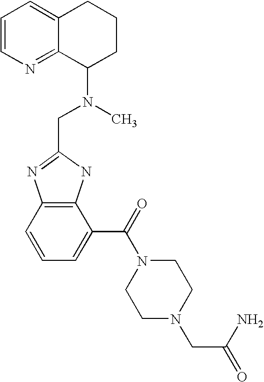

### A) 2-(1-piperazinyl)acetamide

N-Benzyloxypiperazine (575 mg, 2.6 mmol), 2-bromoacetamide (360 mg, 2.6 mmol) and N,N-diisopropylethylamine (85 mg, 0.66 mmol) were dissolved in acetonitrile (15 mL) and the reaction was stirred for 64 h. The reaction was diluted with ethyl acetate (30 mL) and water (50 mL), separated and the organic phase dried over sodium sulfate, filtered and concentrated to provide the protected amine as an oil: ¹H-NMR (DMSO-d₆) δ 7.35-7.28 (m, 5H), 7.19 (br s, 1H), 7.09 (br s, 1H), 5.04 (s, 2H), 3.41-3.37 (m, 4H), 2.84 (s, 2H), 2.38-2.36 (m, 4H). The protected amine and Pd/C (10% w/w, catalytic) were dissolved in ethanol (200 mL) and placed under a hydrogen atmosphere for 16 h. The reaction was filtered through celite and concentrated to provide the product (290 mg, 78% over 2 steps, white solid) as the trifluoroacetate salt: ¹H-NMR (DMSO-d₆) δ 7.08 (br s, 2H), 4.33-4.30 (m, 1H), 2.75 (s, 2H), 2.68-2.66 (m, 4H), 2.30-2.28 (m, 4H).

### B) 2-{4-[(2-{[Methyl(5,6,7,8-tetrahydro-8-quinolinyl)amino]methyl}-1H-benzimidazol-4-yl)carbonyl]-1-piperazinyl}acetamide

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (115 mg, 0.45 mmol), 2-(1-piperazinyl)acetamide (64 mg, 0.45 mmol), and N,N-diisopropylethylamine (58 mg, 0.45 mmol) were dissolved in acetonitrile (3 mL) and N,N-dimethylformamide (2 mL) and the reaction was stirred for 16 h. The reaction was concentrated and purified using reverse phase HPLC (0% to 70% acetonitrile/water/0.1% trifluoroacetic acid gradient) and lyophilized from water to provide the product (35 mg, 15%, yellow solid) as the trifluoroacetate salt: ¹H-NMR (DMSO-d₆) δ 8.56-8.55 (m, 1H), 7.97 (br s, 1H), 7.82-7.80 (m, 1H), 7.76-7.74 (m, 1H), 7.69 (br s, 1H), 7.48-7.45 (m, 1H), 7.33-7.32 (m, 2H), 4.76-4.72 (m, 1H), 4.61 (d, 1H, J=14.8 Hz), 4.498 (d, 1H, J=14.8 Hz), 3.92 (s, 2H), 3.70-3.09 (br m, 8H), 2.89-2.76 (m, 2H), 2.72 (s, 3H), 2.38-2.32 (m, 1H), 2.08-1.97 (m, 2H), 1.79-1.70 (m, 1H). MS m/z 462.30 (M+1).

### Example 67

### N-[2-(1-methyl-1H-imidazol-5-yl)ethyl]-2-{[methyl(5,6,7,8-tetrahydro-8-quinolinyl)amino]methyl}-1H-benzimidazole-4-carboxamide

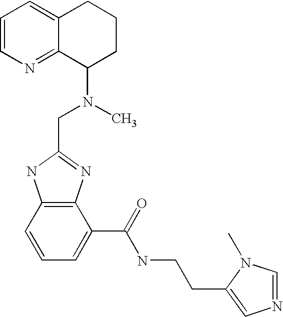

2-{[Methyl(5,6,7,8-tetrahydro-8-quinolinyl)amino]methyl}-1H-benzimidazole-4-carboxylic acid (50 mg, 0.15 mmol), bis(2-oxo-3-oxazolidinyl)phosphinic chloride (56 mg, 0.25 mmol), [2-(1-methyl-1H-imidazol-5-yl)ethyl]amine dihydrochloride (59 mg, 0.30 mmol), and N,N-diisopropylethylamine (78 mg, 0.60 mmol) were dissolved in N,N-dimethylformamide (5 mL) and the reaction was stirred for 16 h at room temperature. The reaction mixture was concentrated and purified using reverse phase HPLC (0% to 70% acetonitrile/water/0.1% trifluoroacetic acid gradient) and lyophilized from water to provide the product (39 mg, 33%, yellow solid) as the trifluoroacetate salt: ¹H-NMR (DMSO-d₆) δ 9.26-9.24 (m, 1H), 8.99 (s, 1H), 8.54-8.53 (m, 1H), 7.83-7.76 (m, 2H), 7.49 (s, 1H), 7.44-7.41 (m, 1H), 7.35-7.31 (m, 1H), 4.79-4.75 (m, 1H), 4.68 (d, 1H, J=15 Hz), 4.54 (d, 1H, J=15 Hz), 3.79 (s, 3H), 3.67-3.62 (m, 2H), 2.96-2.91 (m, 2H), 2.84-2.80 (m, 2H), 2.72 (s, 3H), 2.42-2.34 (m, 1H), 2.09-1.98 (m, 2H), 1.80-1.71 (m, 1H). MS m/z 444.24 (M+1).

### Example 68

### N-Methyl-N-[(4-{[4-(2-pyridinylmethyl)-1-piperazinyl]-carbonyl}-1H-benzimidazol-2-yl)methyl]-5,6,7,8-tetrahydro-8-quinolinamine

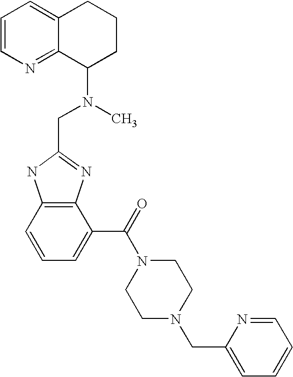

2-{[Methyl(5,6,7,8-tetrahydro-8-quinolinyl)amino]methyl}-1H-benzimidazole-4-carboxylic acid (75 mg, 0.22 mmol), bis(2-oxo-3-oxazolidinyl)phosphinic chloride (84 mg, 0.33 mmol), 1-(2-pyridinylmethyl)piperazine (58 mg, 0.33 mmol), and N,N-diisopropylethylamine (43 mg, 0.33 mmol) were dissolved in N,N-dimethylformamide (5 mL) and the reaction was stirred for 16 h at room temperature. The reaction mixture was concentrated and purified using reverse phase HPLC (0% to 70% acetonitrile/water/0.1% trifluoroacetic acid gradient) and lyophilized from water to provide the product (90 mg, 49%, yellow solid) as the trifluoroacetate salt: ¹H-NMR (DMSO-d₆) δ 8.67-8.66 (m, 1H), 8.56-8.55 (m, 1H), 7.95-7.90 (m, 1H), 7.82-7.80 (m, 1H), 7.77-7.73 (m, 1H), 7.53-7.45 (m, 3H), 7.34-7.32 (m, 2H), 4.77-4.72 (m, 1H), 4.62-4.58 (d, 1H, J=14.9 Hz), 4.49-4.46 (m, 3H), 3.93-3.62 (br m, 4H), 3.36-3.22 (br m, 4H), 2.88-2.75 (m, 2H), 2.71 (s, 3H), 2.38-2.29 (m, 1H), 2.06-1.96 (m, 2H), 1.79-1.68 (m, 1H). MS m/z 496.22 (M+1).

### Example 69

### 2-{[Methyl(5,6,7,8-tetrahydro-8-quinolinyl)amino]methyl}-N-[2-(3-pyridinyl)ethyl]-1H-benzimidazole-4-carboxamide

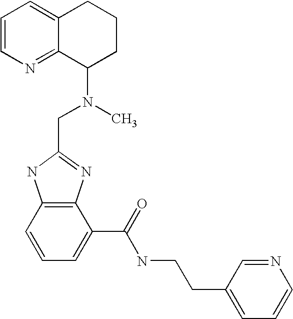

2-{[Methyl(5,6,7,8-tetrahydro-8-quinolinyl)amino]methyl}-1H-benzimidazole-4-carboxylic acid pentafluorophenyl ester bound resin (400 mg, 0.30 mmol) was diluted with N,N-dimethylformamide (10 mL) and 2-(3-pyridinyl)ethyl]amine (73 mg, 0.6 mmol) was added. The reaction was stirred for 16 h at room temperature, filtered, concentrated and purified using reverse phase HPLC (0% to 70% acetonitrile/water/0.1% trifluoroacetic acid gradient) and lyophilized from water to provide the product (11 mg, 5%, brown solid) as the trifluoroacetate salt: ¹H-NMR (DMSO-d₆) δ 9.16-9.14 (m, 1H), 8.62 (s, 1H), 8.57-8.55 (m, 1H), 8.52-8.50 (m, 1H), 8.02 (d, 1H, J=8.5 Hz), 7.81-7.73 (m, 3H), 7.61-7.57 (m, 1H), 7.42-7.39 (m, 1H), 7.33-7.29 (m, 1H), 4.78-4.74 (m, 1H), 4.67 (d, 1H, J=14.7 Hz), 4.53 (d, 1H, J=14.7 Hz), 3.68-3.61 (m, 2H), 2.98-2.95 (m, 2H), 2.84-2.78 (m, 2H), 2.72 (s, 3H), 2.41-2.35 (m, 1H), 2.08-1.99 (m, 2H), 1.79-1.69 (m, 1H). MS m/z 441.08 (M+1).

### Example 70

### N-[2-(3,5-dimethyl-1H-pyrazol-4-yl)ethyl]-2-{[methyl(5,6,7,8-tetrahydro-8-quinolinyl)amino]methyl}-1H-benzimidazole-4-carboxamide

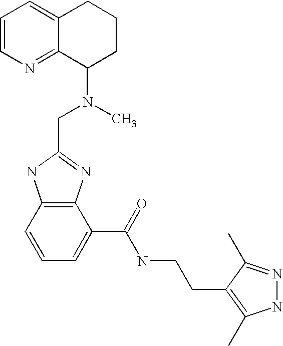

2-{[Methyl(5,6,7,8-tetrahydro-8-quinolinyl)amino]methyl}-1H-benzimidazole-4-carboxylic acid pentafluorophenyl ester bound resin (400 mg, 0.30 mmol) was diluted with N,N-dimethylformamide (10 mL) and [2-(3,5-dimethyl-1H-pyrazol-4-yl)ethyl]methylamine (84 mg, 0.6 mmol) was added. The reaction was stirred for 16 h at room temperature, filtered, concentrated, purified using reverse phase HPLC (10% to 70% acetonitrile/water/0.1% trifluoroacetic acid gradient) and lyophilized from water to provide the product (1.5 mg, 2%, yellow solid) as the trifluoroacetate salt: MS m/z 458.09 (M+1).

### Example 71

### 2-{[Methyl(5,6,7,8-tetrahydro-8-quinolinyl)amino]methyl}-N-[2-(4-pyridinyl)ethyl]-1H-benzimidazole-4-carboxamide

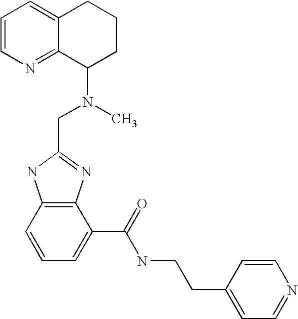

2-{[Methyl(5,6,7,8-tetrahydro-8-quinolinyl)amino]methyl}-1H-benzimidazole-4-carboxylic acid pentafluorophenyl ester bound resin (400 mg, 0.30 mmol) was diluted with N,N-dimethylformamide (10 mL) and 2-(4-pyridinyl)ethyl]amine (0.50 mL) was added. The reaction was stirred for 16 h at 50° C., filtered, concentrated and purified using reverse phase HPLC (0% to 70% acetonitrile/water/0.1% trifluoroacetic acid gradient) and lyophilized from water to provide the product (10 mg, 4%, red solid) as the trifluoroacetate salt: ¹H-NMR (DMSO-d₆) δ 9.24-9.21 (m, 1H), 8.71-8.70 (m, 2H), 8.55-8.54 (m, 1H), 7.83-7.74 (m, 5H), 7.46-7.43 (m, 1H), 7.35-7.31 (m, 1H), 4.79-4.75 (m, 1H), 4.68 (d, 1H, J=14.9 Hz), 4.53 (d, 1H, J=14.9 Hz), 3.75-3.69 (m, 2H), 3.11-3.08 (m, 2H), 2.85-2.79 (m, 2H), 2.71 (s, 3H), 2.42-2.33 (m, 1H), 2.10-1.99 (m, 2H), 1.83-1.72 (m, 1H). MS m/z 441 (M+1).

### Example 72

### 2-{[Methyl(5,6,7,8-tetrahydro-8-quinolinyl)amino]methyl}-N-[3-(2-oxo-1-pyrrolidinyl)propyl]-1H-benzimidazole-4-carboxamide

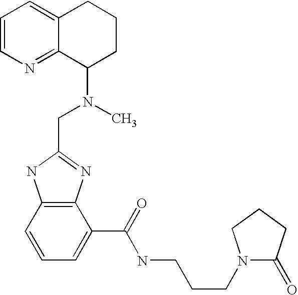

2-{[Methyl(5,6,7,8-tetrahydro-8-quinolinyl)amino]methyl}-1H-benzimidazole-4-carboxylic acid pentafluorophenyl ester bound resin (400 mg, 0.30 mmol) was diluted with N,N-dimethylformamide (6 mL) and 1-(3-aminopropyl)-2-pyrrolidinone (85 mg, 0.60 mmol) was added. The reaction was stirred for 72 h at room temperature, filtered, concentrated and purified using reverse phase HPLC (0% to 70% acetonitrile/water/0.1% trifluoroacetic acid gradient) and lyophilized from water to provide the product (6.5 mg, 3%, yellow solid) as the trifluoroacetate salt: ¹H-NMR (DMSO-d₆) δ 9.27-9.23 (m, 1H), 8.52-8.51 (m, 1H), 7.85-7.80 (m, 2H), 7.75-7.73 (m, 1H), 7.43-7.40 (m, 1H), 7.37-7.33 (m, 1H), 4.83-4.79 (m, 1H), 4.73 (d, 1H, J=14.8 Hz), 4.58 (d, 1H, J=14.8 Hz), 3.35-3.20 (m, 7H), 2.87-2.81 (m, 5H), 2.44-2.38 (m, 1H), 2.22-2.17 (m, 2H), 2.09-2.02 (m, 2H), 1.93-1.88 (m, 2H), 1.82-1.68 (m, 2H). MS m/z 461.22 (M+1).

### Example 73

### N-[3-(4-Methyl-1-piperazinyl)propyl]-2-{[methyl(5,6,7,8-tetrahydro-8-quinolinyl)amino]methyl}-1H-benzimidazole-4-carboxamide

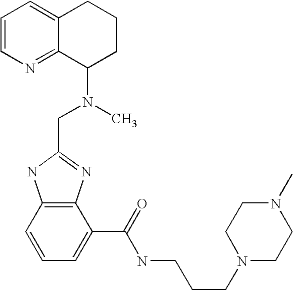

2-{[Methyl(5,6,7,8-tetrahydro-8-quinolinyl)amino]methyl}-1H-benzimidazole-4-carboxylic acid pentafluorophenyl ester bound resin (400 mg, 0.30 mmol) was diluted with N,N-dimethylformamide (6 mL) and [3-(4-methyl-1-piperazinyl)propyl]amine (94 mg, 0.60 mmol) was added. The reaction was stirred for 72 h at room temperature, filtered, concentrated and purified using reverse phase HPLC (0% to 70% acetonitrile/water/0.1% trifluoroacetic acid gradient) and lyophilized from water to provide the product (14.8 mg, 6%) as yellow solid: ¹H-NMR (DMSO-d₆) δ 9.20-9.16 (m, 1H), 8.56-8.55 (m, 1H), 7.86-7.78 (m, 3H), 7.47-7.43 (m, 1H), 7.37-7.33 (m, 1H), 4.80-4.76 (m, 1H), 4.70 (d, 1H, J=14.7 Hz), 4.56 (d, 1H, J=14.7 Hz), 3.65-3.35 (m, 6H), 3.02-2.93 (m, 3H), 2.85-2.79 (m, 5H), 2.74 (s, 3H), 2.43-2.36 (m, 1H), 2.11-1.99 (m, 2H), 1.91-1.70 (m, 3H). MS m/z 476.32 (M+1).

### Example 74

### 2-{[Methyl(5,6,7,8-tetrahydro-8-quinolinyl)amino]methyl}-N-[3-(4-morpholinyl)propyl]-1H-benzimidazole-4-carboxamide

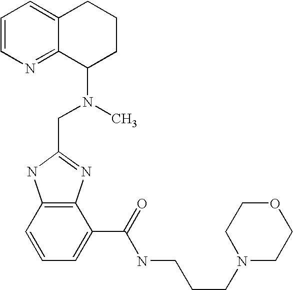

2-{[Methyl(5,6,7,8-tetrahydro-8-quinolinyl)amino]methyl}-1H-benzimidazole-4-carboxylic acid pentafluorophenyl ester bound resin (400 mg, 0.30 mmol) was diluted with N,N-dimethylformamide (6 mL) and [[3-(4-morpholinyl)propyl]amine (86 mg, 0.60 mmol) was added. The reaction was stirred for 72 h at room temperature, filtered, concentrated and purified using reverse phase HPLC (0% to 70% acetonitrile/water/0.1% trifluoroacetic acid gradient) and lyophilized from water to provide the product (11.7 mg, 5%, yellow solid) as the trifluoroacetate salt: ¹H-NMR (DMSO-d₆) δ 9.83 (br s, 1H), 9.22-9.18 (m, 1H), 8.56-8.55 (m, 1H), 7.86-7.83 (m, 2H), 7.79-7.77 (m, 1H), 7.46-7.43 (m, 1H), 7.37-7.33 (m, 1H), 4.81-4.75 (m, 1H), 4.71 (d, 1H, J=14.8 Hz), 4.57 (d, 1H, J=14.8 Hz), 4.00-3.93 (m, 2H), 3.67-3.58 (m, 2H), 3.43-3.39 (m, 4H), 3.18-3.13 (m, 2H), 3.10-2.98 (m, 2H), 2.87-2.79 (m, 2H), 2.75 (s, 3H), 2.43-2.35 (m, 1H), 2.11-2.00 (m, 2H), 1.97-1.87 (m, 2H), 1.83-1.70 (m, 1H). MS m/z 463.23 (M+1).

### Example 75

### N-(1H-Benzimidazol-2-ylmethyl)-2-{[methyl(5,6,7,8-tetrahydro-8-quinolinyl)amino]methyl}-1H-benzimidazole-4-carboxamide

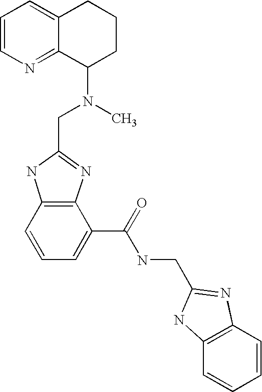

2-{[Methyl(5,6,7,8-tetrahydro-8-quinolinyl)amino]methyl}-1H-benzimidazole-4-carboxylic acid pentafluorophenyl ester bound resin (400 mg, 0.30 mmol) was diluted with N,N-dimethylformamide (5 mL) and 2-(aminomethyl)benzimidazole dihydrochloride (132 mg, 0.60 mmol) and N,N-diisopropylethylamine (155 mg, 1.20 mmol) were added. The reaction was stirred for 16 h at room temperature, filtered, concentrated and purified using reverse phase HPLC (0% to 70% acetonitrile/water/0.1% trifluoroacetic acid gradient) and lyophilized from water to provide the product (6.7 mg, 3%, yellow solid) as the trifluoroacetate salt: ¹H-NMR (DMSO-d₆) δ 9.95-9.92 (m, 1H), 8.44-8.43 (m, 1H), 7.95 (d, 1H, J=8.4 Hz), 7.88 (d, 1H, J=8.4 Hz), 7.67-7.63 (m, 3H), 7.41-7.37 (m, 2H), 7.35-7.31 (m, 1H), 4.99-4.94 (m, 2H), 4.78-4.73 (m, 1H), 4.67 (d, 1H, J=15.6 Hz), 4.53 (d, 1H, J=15.6 Hz), 2.72 (s, 3H), 2.68-2.62 (m, 2H), 2.42 (m, 1H), 2.04-1.94 (m, 2H), 1.78-1.64 (m, 1H). MS m/z 466.27 (M+1).

### Example 76

### 2-{[Methyl(5,6,7,8-tetrahydro-8-quinolinyl)amino]methyl}-N-[2-(2-pyridinyl)ethyl]-1H-benzimidazole-4-carboxamide

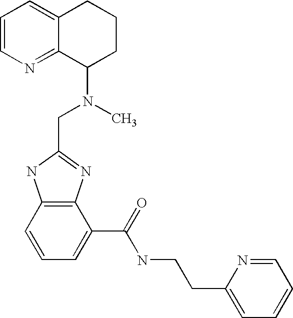

2-{[Methyl(5,6,7,8-tetrahydro-8-quinolinyl)amino]methyl}-1H-benzimidazole-4-carboxylic acid pentafluorophenyl ester bound resin (400 mg, 0.30 mmol) was diluted with N,N-dimethylformamide (5 mL) and [2-(2-pyridinyl)ethyl]amine (2 mL) was added. The reaction was stirred for 40 h at room temperature, filtered, concentrated and purified using reverse phase HPLC (0% to 70% acetonitrile/water/0.1% trifluoroacetic acid gradient) and lyophilized from water to provide the product (14 mg, 6%, yellow solid) as the trifluoroacetate salt: ¹H-NMR (DMSO-d₆) δ 9.22-9.19 (m, 1H), 8.64 (d, 1H, J=6.1 Hz), 8.53 (d, 1H, J=6.1 Hz), 8.04-7.99 (m, 1H), 7.81-7.74 (m, 3H), 7.56-7.48 (m, 2H), 7.43-7.39 (m, 1H), 7.33-7.29 (m, 1H), 4.78-4.73 (m, 1H), 4.66 (d, 1H, J=14.7 Hz), 4.51 (d, 1H, J=14.7 Hz), 3.77-3.72 (m, 2H), 3.15-3.11 (m, 2H), 2.84-2.76 (m, 2H), 2.72 (s, 3H), 2.41-2.33 (m, 1H), 2.08-1.97 (m, 2H), 1.79-1.68 (m, 1H). MS m/z 441.24 (M+1).

### Example 77

### 2-{[Methyl(5,6,7,8-tetrahydro-8-quinolinyl)amino]methyl}-N-4-piperidinyl-1H-benzimidazole-4-carboxamide

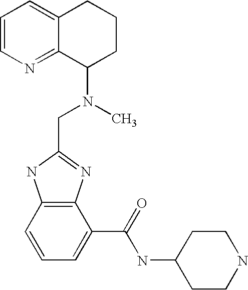

2-{[Methyl(5,6,7,8-tetrahydro-8-quinolinyl)amino]methyl}-1H-benzimidazole-4-carboxylic acid (80 mg, 0.24 mmol), bis(2-oxo-3-oxazolidinyl)phosphinic chloride (91 mg, 0.37 mmol), 1,1-dimethylethyl 4-amino-1-piperidinecarboxylate (96 mg, 0.48 mmol), and N,N-diisopropylethylamine (62 mg, 0.48 mmol) were dissolved in N,N-dimethylformamide (5 mL) and the reaction was stirred for 72 h. The reaction mixture was filtered, concentrated and purified using reverse phase HPLC (0% to 70% acetonitrile/water/0.1% trifluoroacetic acid gradient) to provide the protected amine as a white solid. The carbamate was dissolved in dichloromethane (5 mL) and trifluoroacetic acid (2 mL) and stirred for 2 h. The reaction was concentrated and lyophilized from water to provide the product (80 mg, 44%, yellow solid) as the trifluoroacetate salt: ¹H-NMR (DMSO-d₆) δ 9.03-9.00 (m, 1H), 8.64-8.59 (m, 1H), 8.53-8.53 (m, 1H), 8.44-8.37 (m, 1H), 7.88-7.82 (m, 2H), 7.77-7.75 (m, 1H), 7.44-7.41 (m, 1H), 7.36-7.32 (m, 2H), 4.78-4.75 (m, 1H), 4.68 (d, 1H, J=14.7 Hz), 4.54 (d, 1H, J=14.7 Hz), 4.15-4.06 (m, 1H), 3.35-3.29 (m, 2H), 3.10-3.00 (m, 2H), 2.84-2.79 (m, 2H), 2.75 (s, 3H), 2.42-2.34 (m, 1H), 2.09-1.98 (m, 4H), 1.80-1.64 (m, 3H). MS m/z 419.11 (M+1).

### Example 78

### 2-{[Methyl(5,6,7,8-tetrahydro-8-quinolinyl)amino]methyl}-N-3-pyrrolidinyl-1H-benzimidazole-4-carboxamide

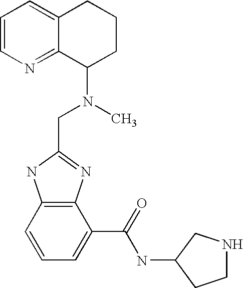

2-{[Methyl(5,6,7,8-tetrahydro-8-quinolinyl)amino]methyl}-1H-benzimidazole-4-carboxylic acid (80 mg, 0.24 mmol), bis(2-oxo-3-oxazolidinyl)phosphinic chloride (91 mg, 0.37 mmol) tert-butyl 3-amino-1-pyrrolidinecarboxylate (90 mg, 0.48 mmol), and N,N-diisopropylethylamine (62 mg, 0.48 mmol) were dissolved in N,N-dimethylformamide (5 mL) and the reaction was stirred for 72 h. The reaction mixture was filtered, concentrated and purified using reverse phase HPLC (0% to 70% acetonitrile/water/0.1% trifluoroacetic acid gradient) to provide the protected amine as a white solid. The carbamate was dissolved in dichloromethane (5 mL) and trifluoroacetic acid (2 mL) and stirred for 2 h. The reaction was concentrated and lyophilized from water to provide the product (54.7 mg, 31%, sticky brown solid) as the trifluoroacetate salt: ¹H-NMR (DMSO-d₆) δ 9.12 (br s, 1H), 8.96 (br s, 1H), 8.88 (br s, 1H), 8.53 (d, 1H), 7.87-7.85 (m, 2H), 7.76 (d, 1H), 7.44-7.34 (m, 2H), 4.78-4.67 (m, 2H), 4.60-4.54 (m, 2H), 4.35 (t, 1H), 3.83 (t, 1H), 3.54-3.45 (m, 1H), 3.39-3.14 (m, 3H), 2.76-2.74 (m, 1H), 2.73 (s, 3H), 2.35-2.21 (m, 2H), 2.09-1.98 (m, 2H), 1.82-1.69 (m, 1H). MS m/z 405.07 (M+1).

### Example 79

### N-[3-(1H-Imidazol-1-yl)propyl]-2-{[methyl(5,6,7,8-tetrahydro-8-quinolinyl)amino]methyl}-1H-benzimidazole-4-carboxamide

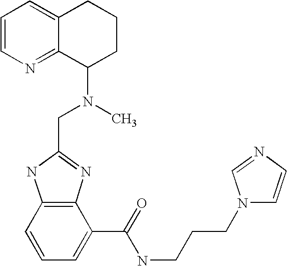

2-{[Methyl(5,6,7,8-tetrahydro-8-quinolinyl)amino]methyl}-1H-benzimidazole-4-carboxylic acid (80 mg, 0.24 mmol), bis(2-oxo-3-oxazolidinyl)phosphinic chloride (91 mg, 0.37 mmol), [3-(1H-imidazol-1-yl)propyl]amine (mg, 0.48 mmol), and N,N-diisopropylethylamine (62 mg, 0.48 mmol) were dissolved in N,N-dimethylformamide (5 mL) and the reaction was stirred for 72 h. The reaction mixture was filtered, concentrated and purified using reverse phase HPLC (0% to 70% acetonitrile/water/0.1% trifluoroacetic acid gradient) to provide the product (38 mg, 20%, brown solid) as the trifluoroacetate salt: ¹H-NMR (DMSO-d₆) δ 9.17-9.13 (m, 1H), 9.12 (s, 1H), 8.52-8.51 (m, 1H), 7.84-7.80 (m, 3H), 7.76-7.74 (m, 1H), 7.69-7.68 (m, 1H), 7.43-7.40 (m, 1H), 7.36-7.32 (m, 1H), 4.80-4.76 (m, 1H), 4.70 (d, 1H, J=15.0 Hz), 4.56 (d, 1H, J=15.0 Hz), 4.27-4.23 (m, 2H), 3.39-3.33 (m, 2H), 2.84-2.78 (m, 2H), 2.73 (s, 3H), 2.43-2.35 (m, 1H), 2.11-2.01 (m, 4H), 1.79-1.71 (m, 1H). MS m/z 444.04 (M+1).

### Example 80

### N-[3-(Dimethylamino)-2,2-dimethylpropyl]-2-{[methyl(5,6,7,8-tetrahydro-8-quinolinyl)amino]methyl}-1H-benzimidazole-4-carboxamide

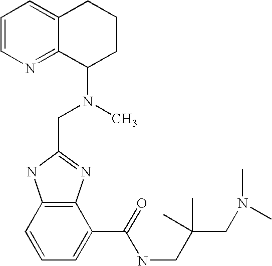

2-{[Methyl(5,6,7,8-tetrahydro-8-quinolinyl)amino]methyl}-1H-benzimidazole-4-carboxylic acid (80 mg, 0.24 mmol), bis(2-oxo-3-oxazolidinyl)phosphinic chloride (91 mg, 0.37 mmol), N,N,2,2-tetramethyl-1,3-propanediamine (63 mg, 0.48 mmol), and N,N-diisopropylethylamine (62 mg, 0.48 mmol) were dissolved in N,N-dimethylformamide (5 mL) and the reaction was stirred for 72 h. The reaction mixture was filtered, concentrated and purified using reverse phase HPLC (0% to 70% acetonitrile/water/0.1% trifluoroacetic acid gradient) to provide the product (43 mg, 23%, yellow solid) as the trifluoroacetate salt: ¹H-NMR (DMSO-d₆) δ 9.46-9.43 (m, 1H), 9.05-9.01 (m, 1H), 8.56-8.55 (m, 1H), 7.93-7.91 (m, 1H), 7.86-7.81 (m, 2H), 7.49-7.46 (m, 1H), 7.41-7.37 (m, 1H), 4.85-4.81 (m, 1H), 4.71-4.67 (m, 1H), 4.54-4.50 (m, 1H), 3.45-3.32 (m, 2H), 3.01-3.00 (m, 2H), 2.87 (d, 6H, J=4.6 Hz), 2.84-2.81 (m, 2H), 2.76 (s, 3H), 2.43-2.36 (m, 1H), 2.09-2.02 (m, 2H), 1.81-1.71 (m, 1H), 1.05 (d, 6H, J=11.7 Hz). MS m/z 449.14 (M+1).

### Example 81

### N-[2-(1H-Indol-3-yl)ethyl]-2-{[methyl(5,6,7,8-tetrahydro-8-quinolinyl)amino]methyl}-1H-benzimidazole-4-carboxamide

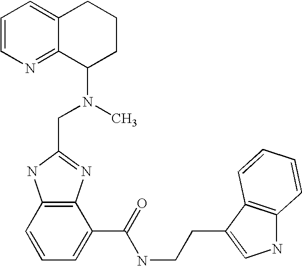

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (115 mg, 0.45 mmol), [2-(1H-indol-3-yl)ethyl]amine (77 mg, 0.48 mmol), and N,N-diisopropylethylamine (78 mg, 0.60 mmol) were dissolved in N,N-dimethylformamide (5 mL) and the reaction was stirred for 72 h. The reaction mixture was filtered, concentrated and purified using reverse phase HPLC (0% to 70% acetonitrile/water/0.1% trifluoroacetic acid gradient) to provide the product (20 mg, 8%, yellow solid) as the trifluoroacetate salt: ¹H-NMR (DMSO-d₆) δ 10.80 (s, 1H), 9.24-9.21 (m, 1H), 8.50-8.48 (m, 1H), 7.86-7.79 (m, 2H), 7.72-7.70 (m, 1H), 7.57-7.55 (m, 1H), 7.39-7.30 (m, 3H), 7.16 (s, 1H), 7.06-7.02 (m, 1H), 6.96-6.93 (m, 1H), 4.78-4.74 (m, 1H), 4.68 (d, 1H, J=14.9 Hz), 4.55 (d, 1H, J=14.9 Hz), 3.66-3.55 (m, 2H), 2.97-2.93 (m, 2H), 2.81-2.74 (m, 2H), 2.72 (s, 3H), 2.40-2.32 (m, 1H), 2.05-1.94 (m, 2H), 1.80-1.66 (m, 1H). MS m/z 479.08 (M+1).

### Example 82

### N-({4-[(4-Amino-1-piperidinyl)carbonyl]-1H-benzimidazol-2-yl}methyl)-N-methyl-5,6,7,8-tetrahydro-8-quinolinamine

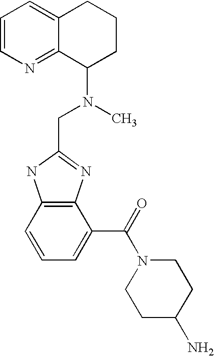

2-{[Methyl(5,6,7,8-tetrahydro-8-quinolinyl)amino]methyl}-1H-benzimidazole-4-carboxylic acid (80 mg, 0.24 mmol), bis(2-oxo-3-oxazolidinyl)phosphinic chloride (91 mg, 0.37 mmol), tert-butyl-4-piperidinylcarbamate (96 mg, 0.48 mmol), and N,N-diisopropylethylamine (62 mg, 0.48 mmol) were dissolved in N,N-dimethylformamide (5mL) and the reaction was stirred for 72-h. The reaction mixture was filtered, concentrated and purified using reverse phase HPLC (0% to 70% acetonitrile/water/0.1% trifluoroacetic acid gradient) to provide the protected amine as a white solid. The carbamate was dissolved in dichloromethane (5 mL) and trifluoroacetic acid (2 mL) and stirred for 2 h. The reaction was concentrated, purified using reverse phase HPLC (0% to 70% acetonitrile/water/0.1% trifluoroacetic acid gradient) and lyophilized from water to provide the product (32 mg, 17%, white solid) as the trifluoroacetate salt: ¹H-NMR (DMSO-d₆) δ 8.54-8.53 (m, 1H), 7.88 (br s, 2H), 7.78-7.76 (m, 1H), 7.72-7.70 (m, 1H), 7.45-7.42 (m, 1H), 7.32-7.28 (m, 1H), 7.22-7.20 (m, 1H), 4.79-4.75 (m, 1H), 4.60 (d, 1H, J=15.0 Hz), 4.47 (d, 1H, J=15.0 Hz), 3.70-3.56 (m, 1H), 3.31-3.20 (m, 2H), 3.10-2.89 (m, 2H), 2.85-2.79 (m, 2H), 2.74 (s, 3H), 2.40-2.29 (m, 1H), 2.08-1.67 (m, 5H), 1.49-1.35 (m, 2H). MS m/z 419.08 (M+1).

### Example 83

### N-({4-[(3-amino-1-pyrrolidinyl)carbonyl]-1H-benzimidazol-2-yl}methyl)-N-methyl-5,6,7,8-tetrahydro-8-quinolinamine

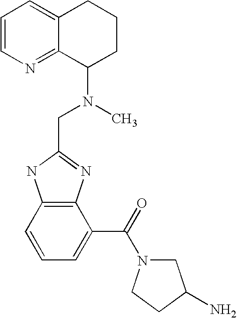

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (115 mg, 0.45 mmol), tert-butyl 3-pyrrolidinylcarbamate (77 mg, 0.48 mmol), and N, N-diisopropylethylamine (78 mg, 0.60 mmol) were dissolved in N,N-dimethylformamide (5 mL) and the reaction was stirred for 72 h. The reaction mixture was filtered, concentrated and purified using reverse phase HPLC (0% to 70% acetonitrile/water/0.1% trifluoroacetic acid gradient) to provide the protected amine as a white solid. The carbamate was dissolved in dichloromethane (5 mL) and trifluoroacetic acid (2 mL) and stirred for 2 h. The reaction was concentrated, purified using reverse phase HPLC (0% to 70% acetonitrile/water/0.1% trifluoroacetic acid gradient) and lyophilized from water to provide the product (17 mg, 3%, white solid) as the trifluoroacetate salt: ¹H-NMR 8.07 (br s, 1H), 7.94 (br s, 1H), 7.77-7.73 (m, 2H), 7.44-7.38 (m, 2H), 7.33-7.29 (m, 1H), 4.78-4.74 (m, 1H), 4.67-4.63 (m, 1H), 4.54-4.50 (m, 1H), 3.91-3.70 (m, 2H), 3.67-3.54 (m, 2H), 3.47-3.37 (m, 1H), 2.85-2.78 (m, 2H), 2.73 (s, 3H), 2.43-2.33 (m, 1H), 2.23-1.88 (m, 4H), 1.78-1.66 (m, 1H). MS m/z 405.07 (M+1).

### Example 84

### N-({4-[(4-Acetyl-1-piperazinyl)carbonyl]-1H-benzimidazol-2-yl}methyl)-N-methyl-5,6,7,8-tetrahydro-8-quinolinamine

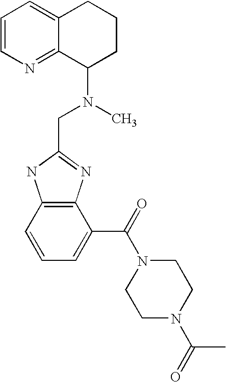

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (151 mg, 0.60 mmol), 1-acetylpiperazine (76 mg, 0.48 mmol), and N,N-diisopropylethylamine (78 mg, 0.60 mmol) were dissolved in N,N-dimethylformamide (5 mL) and the reaction was stirred for 72 h. The reaction mixture was filtered, concentrated and purified using reverse phase HPLC (0% to 70% acetonitrile/water/0.1% trifluoroacetic acid gradient) to provide the product (67 mg, 33%, white solid) as the trifluoroacetate salt: ¹H-NMR (DMSO-d₆) δ 8.53-8.51 (m, 1H), 7.77-7.71 (m, 2H), 7.44-7.41 (m, 1H), 7.33-7.26 (m, 2H), 4.89-4.84 (m, 1H), 4.66 (d, 1H, J=15.2 Hz), 4.52 (d, 1H, J=15.2 Hz), 3.72-3.19 (m, 8H), 2.89-2.79 (m, 2H), 2.77 (s, 3H), 2.40-2.30 (m, 1H), 2.09-1.89 (m, 5H), 1.80-1.71 (m, 1H). MS m/z 447.05 (M+1).

### Example 85

### N-[(4-{[4-(Ethylsulfonyl)-1-piperazinyl]carbonyl}-1H-benzimidazol-2-yl)methyl]-N-methyl-5,6,7,8-tetrahydro-8-quinolinamine

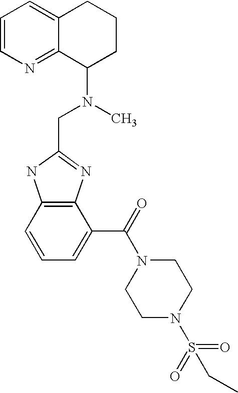

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (151 mg, 0.60 mmol), 1-(ethylsulfonyl)piperazine (105 mg, 0.48 mmol), and N,N-diisopropylethylamine-(78 mg, 0.60 mmol) were dissolved in N,N-dimethylformamide (5 mL) and the reaction was stirred for 72 h. The reaction mixture was filtered, concentrated and purified using reverse phase HPLC (0% to 70% acetonitrile/water/0.1% trifluoroacetic acid gradient) to provide the product (101 mg, 46%, white solid) as the trifluoroacetate salt: ¹H-NMR (DMSO-d₆) δ 8.55-8.53 (m, 1H), 7.79-7.72 (m, 2H), 7.46-7.43 (m, 1H), 7.34-7.28 (m, 2H), 4.84-4.80 (m, 1H), 4.66-4.62 (m, 1H), 4.53-4.49 (m, 1H), 3.76-3.11 (m, 8H), 3.08-3.03 (m, 2H), 2.90-2.81 (m, 2H), 2.77 (s, 3H), 2.42-2.30 (m, 1H), 2.09-1.96 (m, 2H), 1.82-1.71 (m, 1H), 1.21-1.17 (m, 3H). MS m/z 497.0 (M+1).

### Example 86

### N-Methyl-N-({4-[(4-methylhexahydro-1H-1,4-diazepin-1-yl)carbonyl]-1H-benzimidazol-2-yl}methyl)-5,6,7,8-tetrahydro-8-quinolinamine

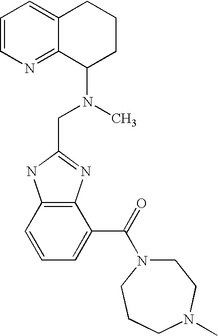

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (151 mg, 0.60 mmol), N-methyl homopiperazine (67 mg, 0.48 mmol), and N,N-diisopropylethylamine (78 mg, 0.60 mmol) were dissolved in N,N-dimethylformamide (5 mL) and the reaction was stirred for 72 h. The reaction mixture was filtered, concentrated and purified using reverse-phase HPLC (0% to 70% acetonitrile/water/0.1% trifluoroacetic acid gradient) to provide the product (82 mg, 35%, yellow solid) as the trifluoroacetate salt: ¹H-NMR (CDCl₃) δ 11.62 (br s, 1H), 9.05-8.96 (m, 1H), 8.17-8.15 (m, 1H), 7.93-7.91 (m, 1H), 7.79 (m, 1H), 7.54-7.47 (m, 2H), 4.58-4.31 (m, 3H), 4.17-3.88 (m, 1H), 3.80-3.55 (m, 3H), 3.49-3.20 (m, 2H), 3.03-3.00 (m, 2H), 2.93 (d, 3H, J=6.6 Hz), 2.63-2.52 (m, 1H), 2.45-2.23 (m, 4H), 2.09-1.80 (m, 3H). MS m/z 433.1 (M+1).

### Example 87

### N-Methyl-N-[(4-{[4-(1-methylethyl)-1-piperazinyl]carbonyl}-1H-benzimidazol-2-yl)methyl]-5,6,7,8-tetrahydro-8-quinolinamine

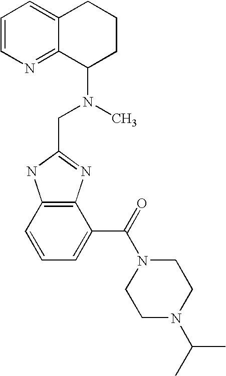

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (151 mg, 0.60 mmol), N-isopropyl piperazine (75 mg, 0.48 mmol), and N,N-diisopropylethylamine (78 mg, 0.60 mmol) were dissolved in N,N-dimethylformamide (5 mL) and the reaction was stirred for 72 h. The reaction mixture was filtered, concentrated and purified using reverse phase HPLC (0% to 70% acetonitrile/water/0.1% trifluoroacetic acid gradient) to provide the product (82 mg, 35%, yellow solid) as the trifluoroacetate salt: ¹H-NMR (DMSO-d₆) δ 9.87 (br s, 1H), 8.56-8.55 (m, 1H), 7.81-7.75 (m, 2H), 7.47-7.44 (m, 1H), 7.38-7.31 (m, 1H), 4.80-4.76 (m, 1H), 4.62 (d, 1H, J=14.8 Hz), 4.49 (d, 1H, J=14.8 Hz), 3.54-3.03 (m, 9H), 2.86-2.81 (m, 2H), 2.75 (s, 3H), 2.39-2.33 (m, 1H), 2.10-1.97 (m, 2H), 1.83-1.67 (m, 1H), 1.25 (d, 6H, J=6.6 Hz). MS m/z 447.09 (M+1).

### Example 88

### N-(1-methyl-4-piperidinyl)-2-{[methyl(5,6,7,8-tetrahydro-8-quinolinyl)amino]methyl}-1H-benzimidazole-4-carboxamide

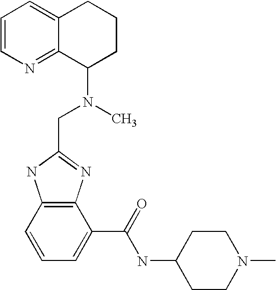

2-{[Methyl(5,6,7,8-tetrahydro-8-quinolinyl)amino]methyl}-1H-benzimidazole-4-carboxylic acid (80 mg, 0.24 mmol), bis(2-oxo-3-oxazolidinyl)phosphinic chloride (92 mg, 0.36 mmol), 1-methyl-4-piperidinamine (55 mg, 0.48 mmol), and N,N-diisopropylethylamine (47 mg, 0.36 mmol) were dissolved in N,N-dimethylformamide (5 mL) and the reaction was stirred for 16 h. The reaction mixture was filtered, concentrated and purified using reverse phase HPLC (0% to 70% acetonitrile/water/0.1% trifluoroacetic acid gradient) to provide the product (87 mg, 47%, yellow solid) as the trifluoroacetate salt: ¹H-NMR (DMSO-d₆) δ 9.72 (br s, 1H), 9.06-9.04 (m, 1H), 8.55-8.54 (m, 1H), 7.89-7.83 (m, 1H), 7.79-7.77 (m, 1H), 7.46-7.43 (m, 1H), 7.37-7.33 (m, 1H), 4.80-4.76 (m, 1H), 4.70 (d, 1H, J=15.0 Hz), 4.55 (d, 1H, J=15.0 Hz), 4.25-4.01 (m, 2H), 3.51-3.46 (m, 2H), 3.40-3.30 (m, 1H), 3.26-3.07 (m, 2H), 2.85-2.73 (m, 8H), 2.46-2.35 (m, 1H), 2.12-2.02 (m, 3H), 1.83-1.69 (m, 2H). MS m/z 433.12 (M+1).

### Example 89

### N-[(4-{[3-(Dimethylamino)-1-pyrrolidinyl]carbonyl}-1H-benzimidazol-2-yl)methyl]-N-methyl-5,6,7,8-tetrahydro-8-quinolinamine

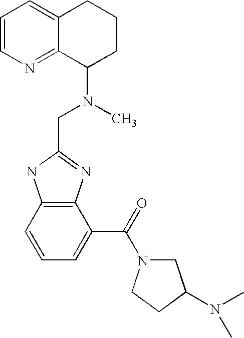

2-{[Methyl(5,6,7,8-tetrahydro-8-quinolinyl)amino]methyl}-1H-benzimidazole-4-carboxylic acid (90 mg, 0.24 mmol), bis(2-oxo-3-oxazolidinyl)phosphinic chloride (100 mg, 0.40 mmol), N,N-dimethyl-3-pyrrolidinamine (62 mg, 0.54 mmol), and N,N-diisopropylethylamine (52 mg, 0.40 mmol) were dissolved in N,N-dimethylformamide (5 mL) and the reaction was stirred for 16 h. The reaction mixture was filtered, concentrated and purified using reverse phase HPLC (0% to 60% acetonitrile/water/0.1% trifluoroacetic acid gradient) to provide the product (48 mg, 23%, red solid) as the trifluoroacetate salt: ¹H-NMR (DMSO-d₆) δ 10.31 (br s, 1H), 8.56-8.55 (m, 1H), 7.79-7.77 (m, 2H), 7.47-7.41 (m, 2H), 7.34-7.31 (m, 1H), 4.79-4.74 (m, 1H), 4.65 (d, 1H, J=14.7), 4.52 (d, 1H, J=14.7), 4.03-3.55 (m, 5H), 2.90-2.73 (m, 11H), 2.41-1.99 (m, 5H), 1.81-1.69 (m, 1H). MS m/z 433.32 (M+1).

### Example 90

### N-[2-(1H-imidazol-4-yl)ethyl]-N-methyl-2-{[methyl(5,6,7,8-tetrahydro-8-quinolinyl)amino]methyl}-1H-benzimidazole-4-carboxamide

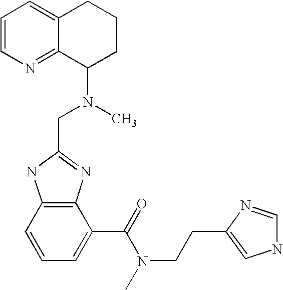

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (113 mg, 0.45 mmol), [2-(1H-imidazol-4-yl)ethyl]methylamine (75 mg, 0.60 mmol, prepared according to Tett. Lett. (1967), 23, 239-242), and N,N-diisopropylethylamine (58 mg, 0.45 mmol) were dissolved in N,N-dimethylformamide (5 mL) and the reaction was stirred for 16 h. The reaction mixture was filtered, concentrated and purified using reverse phase HPLC (0% to 60% acetonitrile/water/0.1% trifluoroacetic acid gradient) to provide the product (17 mg, 7%, yellow solid) as the trifluoroacetate salt: ¹H-NMR (DMSO-d₆) δ 8.99 (br s, 1H), 8.75 (br s, 1H), 8.53-8.51 (m, 1H), 7.78-7.76 (m, 1H), 7.69-7.66 (m, 1H), 7.57 (br s, 1H), 7.45-7.42 (m, 1H), 7.26 (br s, 1H), 7.14 (br s, 1H), 4.60 (d, 1H, J=14.9 Hz), 4.48 (d, 1H, J=14.9 Hz), 3.81-3.73 (m, 2H), 3.64-3.55 (m, 2H), 3.12-2.76 (m, 5H), 2.72 (s, 3H), 2.39-2.29 (m, 1H), 2.06-1.96 (m, 2H), 1.77-1.68 (m, 1H). MS m/z 444.06 (M+1).

### Example 91

### N-methyl-N-({4-[(2-methyl-1-piperazinyl)carbonyl]-1H-benzimidazol-2-yl}methyl)-5,6,7,8-tetrahydro-8-quinolinamine

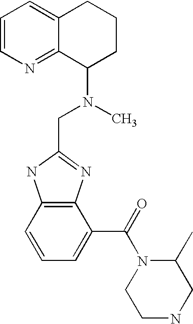

2-{[Methyl(5,6,7,8-tetrahydro-8-quinolinyl)amino]methyl}-1H-benzimidazole-4-carboxylic acid (50 mg, 0.15 mmol), bis(2-oxo-3-oxazolidinyl)phosphinic chloride (57 mg, 0.22 mmol), tert-butyl 3-methyl-1-piperazinecarboxylate (60 mg, 0.30 mmol) and N,N-diisopropylethylamine (28 mg, 0.22 mmol) were dissolved in N,N-dimethylformamide (5 mL) and the reaction was stirred for 16 h. The reaction mixture was filtered, concentrated and purified using reverse phase HPLC (0% to 60% acetonitrile/water/0.1% trifluoroacetic acid gradient) to provide the protected amine. The carbamate was dissolved in dichloromethane (5 mL) and trifluoroacetic acid (2 mL), stirred for one hour, concentrated and purified using reverse phase HPLC (0% to 60% acetonitrile/water/0.1% trifluoroacetic acid gradient) to provide the product (32 mg, 28%, yellow solid) as the trifluoroacetate salt: ¹H-NMR (DMSO-d₆) δ 9.40-9.32 (m, 1H), 8.80 (br s, 1H), 8.56-8.55 (m, 1H), 7.83-7.81 (m, 1H), 7.75-7.73 (m, 1H), 7.50-7.46 (m, 1H), 7.36-7.29 (m, 2H), 4.85-4.75 (m, 1H), 4.62 (d, 1H, J=15.8 Hz), 4.51-4.45 (m, 1H), 3.30-2.95 (m, 6H), 2.87-2.80 (m, 3H), 2.74 (s, 3H), 2.40-2.31 (m, 1H), 2.09-1.98 (m, 2H), 1.83-1.70 (m, 1H), 1.30-1.26 (m, 3H), MS m/z 419.07 (M+1).

### Example 92

### N-({4-[(1S,4S)-2,5-diazabicyclo[2.2.1]hept-2-ylcarbonyl]-1H-benzimidazol-2-yl}methyl)-N-methyl-5,6,7,8-tetrahydro-8-quinolinamine

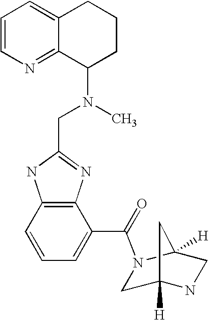

2-{[Methyl(5,6,7,8-tetrahydro-8-quinolinyl)amino]methyl}-1H-benzimidazole-4-carboxylic acid (100 mg, 0.30 mmol), bis(2-oxo-3-oxazolidinyl)phosphinic chloride (113 mg, 0.45 mmol), tert-butyl (1S,4S)-2,5-diazabicyclo[2.2.1]heptane-2-carboxylate (71 mg, 0.36 mmol) and N,N-diisopropylethylamine (58 mg, 0.45 mmol) were dissolved in N,N-dimethylformamide (5 mL) and the reaction was stirred for 16 h. The reaction mixture was filtered, concentrated and purified using reverse phase HPLC (0% to 70% acetonitrile/water/0.1% trifluoroacetic acid gradient) to provide the protected amine. The carbamate was dissolved in dichloromethane (5 mL) and trifluoroacetic acid (2 mL), stirred for 2 h, concentrated and purified using reverse phase HPLC (0% to 70% acetonitrile/water/0.1% trifluoroacetic acid gradient) to provide the product (9.2 mg, 4%, yellow solid) as the trifluoroacetate salt: ¹H-NMR (DMSO-d₆) δ 8.97 (br s, 1H), 8.81 (br s, 1H), 8.53-8.51 (m, 1H), 7.82-7.73 (m, 2H), 7.47-7.28 (m, 3H), 4.91-4.38 (m, 4H), 3.82-3.46 (m, 2H), 3.39-3.14 (m, 2H), 2.88-2.72 (m, 5H), 2.40-2.32 (m, 1H), 2.16-1.96 (m, 2H), 1.81-1.63 (m, 1H). MS m/z 417.10 (M+1).

### Example 93

### N-{[4-(hexahydro-1H-1,4-diazepin-1-ylcarbonyl)-1H-benzimidazol-2-yl]methyl}-N-methyl-5,6,7,8-tetrahydro-8-quinolinamine

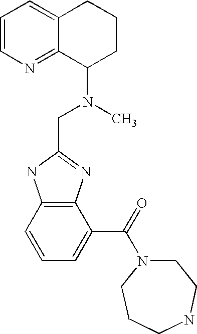

2-{[Methyl(5,6,7,8-tetrahydro-8-quinolinyl)amino]methyl}-1H-benzimidazole-4-carboxylic acid (90 mg, 0.27 mmol), bis(2-oxo-3-oxazolidinyl)phosphinic chloride (100 mg, 0.40 mmol), N-tert-butoxy homopiperazine (108 mg, 0.54 mmol) and N,N-diisopropylethylamine (52 mg, 0.4 mmol) were dissolved in N,N-dimethylformamide (5 mL) and the reaction was stirred for 16 h. The reaction mixture was filtered, concentrated and purified using reverse phase HPLC (0% to 60% acetonitrile/water/0.1% trifluoroacetic acid gradient) to provide the protected amine. The carbamate was dissolved in dichloromethane (5 mL) and trifluoroacetic acid (2 mL), stirred for 3 h, concentrated and purified using reverse phase HPLC (0% to 60% acetonitrile/water/0.1% trifluoroacetic acid gradient) to provide the product (52 mg, 25%, white solid) as the trifluoroacetate salt: ¹H-NMR (DMSO-d₆) δ 8.81 (br s, 1H), 8.56-8.55 (m, 1H), 7.82-7.79 (m, 1H), 7.73-7.71 (m, 1H), 7.48-4.75 (m, 1H), 7.33-7.31 (m, 2H), 4.82-4.74 (m, 1H), 4.63-4.59 (m, 1H), 4.50-4.46 (m, 1H), 3.90-3.82 (m, 1H), 3.80-3.68 (m, 1H), 3.61-3.49 (m, 1H), 3.39-3.12 (m, 5H), 2.87-2.80 (m, 2H), 2.75 (s, 3H), 2.41-2.30 (m, 1H), 2.09-1.95 (m, 3H), 1.90-1.71 (m, 2H). MS m/z 433.12 (M+1).

### Example 94

### N-({4-[3-(Dimethylamino)propyl]-1H-benzimidazol-2-yl}methyl)-N-methyl-5,6,7,8-tetrahydro-8-quinolinamine

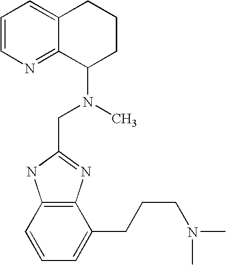

### A) 2-{[Methyl(5,6,7,8-tetrahydro-8-quinolinyl)amino]methyl}-1H-benzimidazole-4-carbaldehyde

A solution of methyl 2-{[methyl(5,6,7,8-tetrahydro-8-quinolinyl)amino]methyl}-1H-benzimidazole-4-carboxylate (1.0 g, 2.9 mmol) in anhydrous tetrahydrofuran (20 mL) was cooled in an ice bath and a solution of lithium aluminum hydride (1.0 M in THF, 2.9 mL, 2.9 mmol) was added dropwise. The reaction mixture was warmed to room temperature and stirred for 30 min. An additional portion of lithium aluminum hydride (2.9 mL, 2.9 mmol) was added and stirred for 30 min. The reaction was quenched with aqueous Rochelle's salt (5% w/v, 20 mL). The mixture was extracted with ethyl acetate (20 mL), dried over sodium sulfate, filtered and concentrated to provide the crude alcohol (900 mg, 98%) as a yellow foam. The alcohol was dissolved in dichlormethane (20 mL) and Dess-Martin periodane (1.3 g, 3.1 mmol) was added portionwise. The reaction was stirred at room temperature for one hour, quenched with aqueous sodium bisulfite (5% w/v, 50 mL) and saturated aqueous sodium bicarbonate (50 mL) and stirred for 30 min. The quenched reaction was extracted with dichloromethane (2×100 mL), concentrated and purified by column chromatography on silica (2% to 5% 2 M ammonia in methanol/dichloromethane gradient) to provide the product (700 mg, 71%) as a brown foam: ¹H-NMR (DMSO-d₆) δ 13.40 (s, 1H), 10.18 (s, 1H), 8.73 (s, 1H), 7.91-7.89 (m, 1H), 7.83-7.79 (m, 1H), 7.54 (d, 1H, J=7.7 Hz), 7.37-7.33 (m, 1H), 7.26-7.22 (m, 1H), 4.02-3.95 (m, 3H), 2.86-2.78 (m, 1H), 2.73-2.65 (m, 1H), 2.32 (s, 3H), 2.10-2.04 (m, 1H), 1.99-1.85 (m, 2H), 1.73-1.64 (m, 1H).

### B) 3-(2-{[Methyl(5,6,7,8-tetrahydro-8-quinolinyl)amino]methyl}-1H-benzimidazol-4-yl)-1-propanol

To a solution of 2-{[methyl(5,6,7,8-tetrahydro-8-quinolinyl)amino]methyl}-1H-benzimidazole-4-carbaldehyde (700 mg, 2.2 mmol) in toluene (20 mL) was added methyl(triphenylphosphoranylidene)acetate (880 mg, 2.6 mmol) in one portion. The reaction was stirred overnight at room temperature, concentrated and purified by column chromatography on silica (2% to 5% 2 M ammonia in methanol/dichloromethane gradient) to provide the ester (900 mg) as a brown oil. The ester was dissolved in anhydrous THF (10 mL) and lithium aluminum hydride (1.0 M in THF, 6.6 mL, 6.6 mmol) was added dropwise. The reaction was stirred for 2 h, quenched with water, washed with aqueous Rochelle's salt (5% w/v) and extracted with ethyl acetate (2×50 mL). The organic extracts were combined, dried over sodium sulfate, filtered and concentrated to give a mixture of the saturated alcohol and allylic alcohol. The mixture and palladium (10% w/w on carbon, catalytic) were dissolved in methanol (100 mL) and the mixture was placed under a hydrogen atmosphere (50 psi) for 48 h. The reaction mixture was filtered through diatomaceous earth, concentrated and purified using reverse phase HPLC (0% to 50% acetonitrile/water/0.1% trifluoroacetic acid gradient) to provide the product (295 mg, 37%, yellow solid) as the trifluoroacetate salt: ¹H-NMR (CDCl₃) δ 9.06-9.04 (m, 1H), 8.17-8.15 (m, 1H), 7.78-7.74 (m, 1H), 7.66-7.61 (m, 1H), 7.44-7.40 (m, 1H), 7.31-7.29 (m, 1H), 4.56-4.42 (m, 2H), 4.36-4.32 (m, 1H), 3.74-3.72 (m, 2H), 3.12-3.08 (m, 2H), 2.46-2.39 (m, 1H), 2.33 (s, 3H), 2.27-2.15 (m, 2H), 2.03-1.84 (m, 5H).

### C) 3-(2-{[Methyl(5,6,7,8-tetrahydro-8-quinolinyl)amino]methyl}-1H-benzimidazol-4-yl)propanal

To a solution of 3-(2-{[methyl(5,6,7,8-tetrahydro-8-quinolinyl)amino]methyl}-1H-benzimidazol-4-yl)-1-propanol (295 mg, 0.84 mmol) in dichloromethane (100 mL) was added IBX polystyrene (2.2 g, 2.40 mmol, NovaBiochem, 1.4 mmol/g) was added in one portion. The slurry was stirred for 16 h, filtered and concentrated to provide the product as a clear oil. The aldehyde was used crude in subsequent steps.

### D) N-({4-[3-(Dimethylamino)propyl]-1H-benzimidazol-2-yl}methyl)-N-methyl-5,6,7,8-tetrahydro-8-quinolinamine

To a solution of 3-(2-{([methyl(5,6,7,8-tetrahydro-8-quinolinyl)amino]methyl}-1H-benzimidazol-4-yl)propanal (60 mg, 0.17 mmol) in dichloroethane (5 mL) was added acetic acid (15 μL, 0.26 mmol), sodium triacetoxyborohydride (66 mg, 0.26 mmol) and dimethylamine (2 M in THF, 0.17 mL, 0.34 mmol). The reaction was stirred for 16 h, purified using reverse phase HPLC (0% to 50% acetonitrile/water/0.1% trifluoroacetic acid gradient) and lyophilized from water to provide the product (34 mg, 28%, clear oil) as the trifluoroacetate salt: ¹H-NMR (DMSO-d₆) δ 9.52 (br s, 1H), 8.57 (d, 1H), 7.82 (d, 1H), 7.53-7.46 (m, 2H), 7.24 (t, 1H), 7.14 (d, 1H), 4.77-4.72 (m, 1H), 4.58 (d, 1H), 4.45 (d, 1H), 3.09-3.05 (m, 2H), 2.96-2.92 (m, 2H), 2.86-2.80 (m, 2H), 2.86-2.74 (m, 7H), 2.71 (s, 3H), 2.37-2.31 (m, 1H), 2.07-1.98 (m, 3H), 1.81-1.70 (m, 1H). MS m/z 378.28 (M+1).

### Example 95

### N-Methyl-N-({4-[3-(1-pyrrolidinyl)propyl]-1H-benzimidazol-2-yl}methyl)-5,6,7,8-tetrahydro-8-quinolinamine

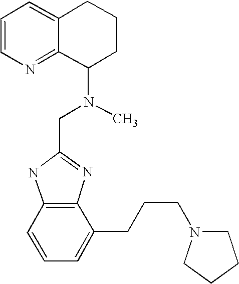

To a solution of 3-(2-{[methyl(5,6,7,8-tetrahydro-8-quinolinyl)amino]methyl}-1H-benzimidazol-4-yl)propanal (60 mg, 0.17 mmol) in dichloroethane (5 mL) was added acetic acid (15 μL, 0.26 mmol), sodium triacetoxyborohydride (66 mg, 0.26 mmol) and pyrrolidine (24 mg, 0.34 mmol). The reaction was stirred for 16 h, purified using reverse phase HPLC (0% to 50% acetonitrile/water/0.1% trifluoroacetic acid gradient) and lyophilized from water to provide the product (25 mg, 20%, clear oil) as the trifluoroacetate salt: ¹H-NMR (CDCl₃) δ 11.65 (br s, 1H), 9.16 (d, 1H), 8.16 (d, 1H), 7.77-7.74 (m, 1H), 7.66 (d, 1H), 7.45-7.42 (m, 1H), 7.31-7.29 (m, 1H), 4.60-4.48 (m, 2H), 4.35-4.31 (m, 1H), 3.79-3.76 (m, 2H), 3.33-3.28 (m, 2H), 3.03-2.95 (m, 5H), 2.49-2.44 (m, 1H), 2.35-2.30 (m, 2H), 2.26-1.84 (m, 8H). MS m/z 404.16 (M+1).

### Example 96

### N-methyl-N-({4-[3-(1-piperidinyl)propyl]-1H-benzimidazol-2-yl}methyl)-5,6,7,8-tetrahydro-8-quinolinamine

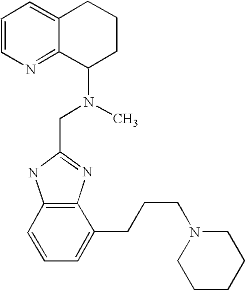

To a solution of 3-(2-{[methyl(5,6,7,8-tetrahydro-8-quinolinyl)amino]methyl}-1H-benzimidazol-4-yl)propanal (60 mg, 0.17 mmol) in dichloroethane (5 mL) was added acetic acid (15 μL, 0.26 mmol), sodium triacetoxyborohydride (66 mg, 0.26 mmol) and piperidine (29 mg, 0.34 mmol). The reaction was stirred for 16 h, purified using reverse phase HPLC (0% to 50% acetonitrile/water/0.1% trifluoroacetic acid gradient) and lyophilized from water to provide the product (24 mg, 19%, clear oil) as the trifluoroacetate salt: ¹H-NMR (CDCl₃) δ11.19 (br s, 1H), 9.14 (d, 1H), 8.15 (d, 1H), 7.77-7.76 (m, 1H), 7.66 (d, 1H), 7.43 (t, 1H), 7.30 (d, 1H), 4.60-4.47 (m, 2H), 4.35-4.31 (m, 1H), 3.62-3.59 (m, 2H), 3.23-3.17 (m, 2H), 3.02-2.98 (m, 4H), 2.73-2.64 (m, 3H), 2.50-2.44 (m, 1H), 2.35 (s, 3H), 2.35-2.33 (m, 1H), 2.25-2.21 (m, 2H), 2.06-1.83 (m, 6H), 1.46-1.37 (m, 1H). MS m/z 418.15 (M+1).

### Example 97

### N-{[4-(3-aminopropyl)-1H-benzimidazol-2-yl]methyl}-N-methyl-5,6,7,8-tetrahydro-8-quinolinamine

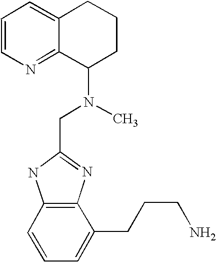

To a solution of 3-(2-{[methyl(5,6,7,8-tetrahydro-8-quinolinyl)amino]methyl}-1H-benzimidazol-4-yl)propanal (120 mg, 0.34 mmol) in methanol (7 mL) was added ammonium acetate (260 mg, 3.40 mmol) and sodium cyanoborohydride (110 mg, 1.7 mmol). The reaction was stirred for 16 h at 50° C. and cooled. 5 N HCl was added dropwise and the reaction was stirred for 2 h, concentrated, purified using reverse phase HPLC (0% to 50% acetonitrile/water/0.1% trifluoroacetic acid gradient) and lyophilized from water to provide the product (31 mg, 13%, tan solid) as the trifluoroacetate salt: ¹H-NMR (DMSO-d₆) δ 8.62 (d, 1H), 7.98 (d, 1H), 7.84 (br s, 2H), 7.61-7.54 (m, 2H), 7.31 (t, 1H), 7.19 (d, 1H), 4.68-4.64 (m, 1H), 4.55 (d, 1H), 4.40 (d, 1H), 3.01-2.97 (m, 2H), 2.87-2.79 (m, 4H), 2.54 (s, 3H), 2.30-2.23 (m, 1H), 2.07-2.01 (m, 1H), 1.99-1.89 (m, 3H), 1.81-1.70 (m, 1H). MS m/z 350.14 (M+1).

### Example 98

### N-[(4-amino-1H-benzimidazol-2-yl)methyl]-N-methyl-5,6,7,8-tetrahydro-8-quinolinamine

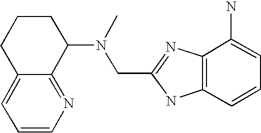

### A) 1,1-dimethylethyl (2-{[methyl(5,6,7,8-tetrahydro-8-quinolinyl)amino]methyl}-1H-benzimidazol-4-yl)carbamate

Methyl 2-{[methyl(5,6,7,8-tetrahydro-8-quinolinyl)amino]methyl}-1H-benzimidazole-4-carboxylate (1.3 g, 3.7 mmol) was dissolved in methanol (5 mL) and tetrahydrofuran (5 mL) and water (5 mL). Lithium hydroxide (265 mg, 11.1 mmol) was added. The reaction mixture was stirred for 72 h at 70° C. Evaporated to dryness, added benzene (10 mL), evaporated, added Et₂O (10 mL) and evaporated. Dried under vacuum. A portion (50%) was dissolved in tBuOH (20 mL), triethylamine (0.27 mL, 1.9 mmol) and diphenylphosphoryl azide (0.53 mL, 1.9 mmol) were added and the mixture heated to reflux under N₂ for 24 h. The solvent was evaporated and the residue dissolved in EtOAc and washed successively with 10 mL of each to the following; 10% citric acid, water, sat. NaHCOₛ, and sat. NaCl. The organic layer was dried over Na₂SO₄, filtered and evaporated. Column chromatography (1-5% 2N NH₃/MeOH in CH₂Cl₂ afforded a yellow solid (0.25 g, 30%): ¹H-NMR (DMSO-d₆) δ 8.46 (m, 1H), 7.48 (d, 2H), 7.16 (dd, 2H), 7.02 (m, 1H), 4.10 (br s, 1H), 4.03-3.99 (m, 2H), 3.91 (t, 1H), 2.83-2.74 (m, 1H), 2.70-2.62 (m, 1H), 2.27 (s, 3H), 2.10-2.00 (m, 1H), 1.98-1.88 (m, 2H), 1.68-1.59 (m, 1H) 1.47 (s, 9H). MS m/z 408 (M+1).

### B) N-[(4-amino-1H-benzimidazol-2-yl)methyl]-N-methyl-5,6,7,8-tetrahydro-8-quinolinamine

1,1-Dimethylethyl (2-{[methyl(5,6,7,8-tetrahydro-8-quinolinyl)amino]methyl}-1H-benzimidazol-4-yl)carbamate (0.25 g, 0.6 mmol) was dissolved in methanol (2 mL) and 4N HCl in dioxane and stirred at rt for 2 h. The solvent was evaporated and the residue dissolved in EtOAc and washed successively with 5 mL of sat. NaHCOₛ, and sat. NaCl. The organic layer was dried over Na₂SO₄, filtered and evaporated. Column chromatography (1-5% 2N NH₃/MeOH in CH₂Cl₂ afforded a yellow solid (0.175 g, 92%).

Alternatively N-[(4-amino-1H-benzimidazol-2-yl)methyl]-N-methyl-5,6,7,8-tetrahydro-8-quinolinamine could be synthesized by the following method:

### A) 2-(Chloromethyl)-4-nitro-1H-benzimidazole

(2-Amino-3-nitrophenyl)amine (2 g, 13 mmol) and chloroacetic acid (1.47 g, 15.6 mmol) were dissolved in 5N HCl (10 mL) and stirred at 120° C. for 12 h. The reaction mixture was cooled to rt and the solid filtered, washed successively with 3×5 mL of ice water, acetone and ether and then dried under vacuum to yield a brown solid (2.75 g, 85%): ¹H-NMR (D₂O) δ 8.33 (d, 1H), 8.09 (d, 1H), 7.62 (t, 1H), 5.07 (s, 2H). MS m/z 308 (M+1).

### B) N-methyl-N-[(4-nitro-1H-benzimidazol-2-yl)methyl]-5,6,7,8-tetrahydro-8-quinolinamine

N-Methyl-5,6,7,8-tetrahydro-8-quinolinamine (1.94 g, 7.7 mmol), KI (1.93 g, 11.6 mmol), DIPEA (3.4 mL, 19.3 mmol) and 2-(chloromethyl)-4-nitro-1H-benzimidazole hydrochloride salt (1.25 g, 7.7 mmol) were dissolved in acetonitrile (50 mL) and stirred at 70° C. for 12 h. The solvent was evaporated and water. The organic layer was dried over Na₂SO₄, filtered and evaporated. Column chromatography (0-2% 2N NH₃/MeOH in CH₂Cl₂ afforded a brown oil (1.4 g, 54%): ¹H-NMR (DMSO-d₆) δ 13.7 (br s, 1H), 8.61 (d, 1H), 8.10 (d, 1H), 8.04 (d, 1H), 7.56 (d, 1H), 7.36 (t, 1H), 7.26 (dd, 1H), 4.04-3.97 (m, 3H), 2.87-2.78 (m, 1H), 2.74-2.67 (m, 1H), 2.23 (s, 3H), 2.10-2.03 (m, 1H), 1.99-1.86 (m, 2H), 1.67-1.56 (m, 1H). MS m/z 338 (M+1).

### C) N-[(4-amino-1H-benzimidazol-2-yl)methyl]-N-methyl-5,6,7,8-tetrahydro-8-quinolinamine

N-methyl-N-[(4-nitro-1H-benzimidazol-2-yl)methyl]-5,6,7,8-tetrahydro-8-quinolinamine (0.4 g, 1.1 mmol) was dissolved in EtOH (10 mL) and purged with N₂. 10% palladium on carbon was added and the heterogeneous mixture purged with H₂. The mixture was stirred under atmospheric H₂ until disappearance of starting material by TLC. The solution was filtered through celite and evaporated to yield an oil (0.36 g, quantitative yield): ¹H-NMR (DMSO-d₆) δ 8.44 (d, 1H), 7.50 (d, 1H), 7.18 (dd, 2H), 6.79 (t, 1H), 6.65 (d, 1H), 6.27 (d, 1H), 5.15 (br s, 2H), 3.97 (s, 2H), 3.93 (t, 1H), 2.83-2.74 (m, 1H), 2.69-2.60 (m, 1H), 2.21 (s, 3H), 2.10-2.00 (m, 1H), 1.97-1.85 (m, 2H), 1.67-1.56 (m, 1H) 1.47 (s, 9H). MS m/z 308 (M+1).

### Example 99

### N¹-(2-{[methyl(5,6,7,8-tetrahydro-8-quinolinyl)amino]methyl}-1H-benzimidazol-4-yl)glycinamide

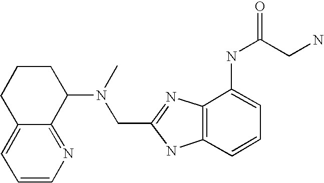

### N-[(4-amino-1H-benzimidazol-2-yl)methyl]-N-methyl-5,6,7,8-tetrahydro-8-quinolinamine

(010 g, 0.32 mmol), N-carbonylbenzyloxy glycine (0.075 g, 0.39 mmol), bis(2-oxo-3-oxazolidinyl)phosphinic chloride (0.09 g, 0.35 mmol), and N,N-diisopropylethylamine (0.07 mL, 0.39 mmol) were dissolved in acetonitrile (5 mL) and the reaction was stirred for 12 h at room temperature. The reaction was concentrated, diluted with ethyl acetate (25 mL), washed with sat. NaHCO₃ (5 mL), dried over Na₂SO₄, filtered and concentrated. The amide was purified using reverse phase HPLC (0% to 50% acetonitrile/water/0.1% trifluoroacetic acid gradient). The desired fractions were combined, NaCl added until the solution was saturated, neutralized and extracted with EtOAc (5×20 mL). The organic layers combined, dried over Na₂SO₄, filtered and concentrated to provide an oil. The compound was dissolved in EtOH (5 mL), purged with N₂, 10% palladium on carbon (0.01 g) added, the system flushed with H₂, and stirred for 2 h under an atmosphere of H₂. The product was purified by reverse phase HPLC according to the above protocol to yield a white foam (10 mg, 25%): ¹H-NMR (CD₃OD) δ 8.68 (d, 1H), 8.14 (d, 1H), 7.71 (dd, 1H), 7.65 (d, 1H), 7.56 (d, 1H), 7.42 (t, 1H), 4.56 (dd, 1H), 4.49 (½ ABq, 1H), 4.36 (½ABq, 1H), 4.02 (s, 2H), 2.97 (m, 2H), 2.51 (s, 3H), 2.40-2.32 (m, 1H), 2.22-2.16 (m, 1H), 2.11-2.02 (m, 1H), 1.95-1.84 (m, 1H). MS m/z 405 (M+1).

### Example 100

### N-(2-{[methyl(5,6,7,8-tetrahydro-8-quinolinyl)amino]methyl}-1H-benzimidazol-4-yl)-3-(1-piperidinyl)propanamide

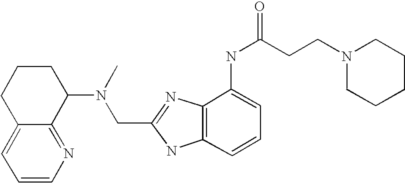

### N-[(4-Amino-1H-benzimidazol-2-yl)methyl]-N-methyl-5,6,7,8-tetrahydro-8-quinolinamine

(0.1 g, 0.32 mmol), 3-(1-piperidinyl)propanoic acid (0.055 g, 0.35 mmol), bis(2-oxo-3-oxazolidinyl)phosphinic chloride (0.09 g, 0.35 mmol), and N,N-diisopropylethylamine (0.07 mL, 0.39 mmol) were dissolved in acetonitrile (5 mL) and the reaction was stirred for 12 h at room temperature. The reaction was concentrated, diluted with ethyl acetate (25 mL), washed with sat. NaHCO₃ (5 mL), dried over Na₂SO₄, filtered and concentrated. The amide was purified using reverse phase HPLC (0% to 50% acetonitrile/water/0.1% trifluoroacetic acid gradient). The desired fractions were combined, NaCl added until the solution was saturated, neutralized and extracted with EtOAc (5×20 mL). The organic layers combined, dried over Na₂SO₄, filtered and concentrated to provide an foam (12 mg, 9%): ¹H-NMR (CD₃OD) δ 8.47 (d, 1H), 7.57 (d, 2H), 7.33 (d, 1H), 7.23 (dd, 1H), 7.16 (t, 1H), 4.11 (t, 1H), 4.09 (½ ABq, 1H), 3.92 (½ABq, 1H), 3.52 (t, 1H), 3.46 (t, 1H), 3.03 (m, 4H), 2.89 (t, 2H), 2.81 (t, 2H), 2.73 (t, 2H), 2.19-2.02 (m, 2H), 2.12 (s, 3H), 2.01-1.92 (m, 2H), 1.73 (m, 3H), 1.66 (m, 1H), 1.58 (m, 4H). MS m/z 447 (M+1).

### Example 101

### N-{[4-(1H-Imidazol-1-yl)-1H-benzimidazol-2-yl]methyl}-N-methyl-5,6,7,8-tetrahydro-8-quinolinamine

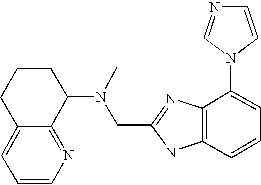

N-{[4-(1H-Imidazol-1-yl)-1H-benzimidazol-2-yl]methyl}-N-methyl-5,6,7,8-tetrahydro-8-quinolinamine was prepared according to the protocol of Liu, J. et al. Synthesis 2003, 17, 2661-2666. N-[(4-amino-1H-benzimidazol-2-yl)methyl]-N-methyl-5,6,7,8-tetrahydro-8-quinolinamine (0.2 g, 0.65 mmol) and 40 aq. glyoxal (0.080 mL, 0.68 mmol) were dissolved in methanol (5 mL) and the reaction was stirred for 14 h at room temperature. Solid NH₄Cl (0.07 g, 1.3 mmol), 37% aq. Formaldehyde (0.1 mL), 1.3 mmol) and methanol (8 mL) were added and the mixture for 1 hour. H₃PO₄ (0.2 mL) was added and heated to 80° C. for 5 hours. Cooled to rt and stirred for an additional 12 hour. The reaction was concentrated, diluted with ethyl acetate (25 mL), washed with sat. NaHCO₃ (5 mL), dried over Na₂SO₄, filtered and concentrated. The product was purified using reverse phase HPLC (0% to 50% acetonitrile/water/0.1% trifluoroacetic acid gradient). The desired fractions were combined, NaCl added until the solution was saturated, neutralized and extracted with EtOAc (5×20 mL). The organic layers combined, dried over Na₂SO₄, filtered and concentrated to provide a yellow solid (0.010 g, 4%): ¹H-NMR (DMSO-d₆) δ 8.63 (s, 1H), 8.45 (d, 1H), 8.00 (s, 1H), 7.48 (t, 2H), 7.37 (d, 1H), 7.22 (t, 1H), 7.17 (dd, 1H), 7.09 (s, 1H), 4.10 (dd, 2H), 3.96 (t, 1H), 2.82-2.74 (m, 1H), 2.69-2.62 (m, 1H), 2.26 (s, 3H), 2.09-2.02 (m, 1H), 1.97-1.88 (m, 2H), 1.68-1.58 (m, 1H). MS m/z 359 (M+1).

### Example 102

### N-(2-{[Methyl(5,6,7,8-tetrahydro-8-quinolinyl)amino]methyl}-1H-benzimidazol-4-yl)-3-pyridinecarboxamide

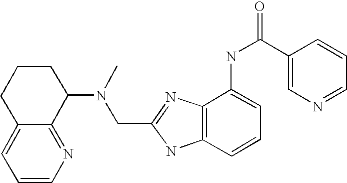

N-(2-{[Methyl(5,6,7,8-tetrahydro-8-quinolinyl)amino]methyl}-1H-benzimidazol-4-yl)-3-pyridinecarboxamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from N-[(4-amino-1H-benzimidazol-2-yl)methyl]-N-methyl-5,6,7,8-tetrahydro-8-quinolinamine (0.1 g, 0.32 mmol), nicotinic acid (0.045 g, 0.35 mmol), bis(2-oxo-3-oxazolidinyl)phosphinic chloride (0.09 g, 0.35 mmol), and N,N-diisopropylethylamine (0.07 mL, 0.39 mmol) in a similar manner as described herein to provide a yellow solid (0.02 g, 15%): ¹H-NMR (CD₃OD) δ 9.31 (d, 1H), 8.81 (d, 1H), 8.57 (d, 1H), 8.50 (d, 1H), 7.68 (dd, 1H), 7.63 (d, 1H), 7.48 (d, 1H), 7.30 (t, 1H), 7.27 (d, 1H), 4.18 (½ ABq, 1H), 4.17 (t, 1H), 4.00 (½ABq, 1H), 2.98-2.87 (m, 2H), 2.25-1.97 (m, 3H), 2.18 (s, 3H), 1.86-1.73 (m, 1H). MS m/z 413 (M+1).

### Example 103

### N-(2-{[Methyl(5,6,7,8-tetrahydro-8-quinolinyl)amino]methyl}-1H-benzimidazol-4-yl)-4-pyridinecarboxamide

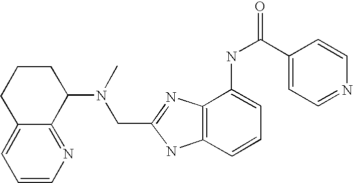

N-(2-{[Methyl(5,6,7,8-tetrahydro-8-quinolinyl)amino]methyl}-1H-benzimidazol-4-yl)-4-pyridinecarboxamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from N-[(4-amino-1H-benzimidazol-2-yl)methyl]-N-methyl-5,6,7,8-tetrahydro-8-quinolinamine (0.1 g, 0.32 mmol), isonicotinic acid (0.045 g, 0.35 mmol), bis(2-oxo-3-oxazolidinyl)phosphinic chloride (0.09 g, 0.35 mmol), and N,N-diisopropylethylamine (0.07 mL, 0.39 mmol) in a similar manner as described herein to provide a yellow solid (0.02 g, 15%): ¹H-NMR (CD₃OD) δ 8.82 (d, 2H), 8.49 (d, 1H), 8.10 (d, 2H), 7.74 (s, 1H), 7.62 (d, 1H), 7.48 (d, 1H), 7.30 (t, 1H), 7.26 (d, 1H), 4.17 (½ ABq, 1H), 4.15 (t, 1H), 4.00 (½ABq, 1H), 2.96-2.78 (m, 2H), 2.25-1.97 (m, 3H), 2.17 (s, 3H), 1.86-1.73 (m, 1H). MS m/z 413 (M+1).

### Example 104

### 1-Methyl-N-(2-{[methyl(5,6,7,8-tetrahydro-8-quinolinyl)amino]methyl}-1H-benzimidazol-4-yl)-L-prolinamide

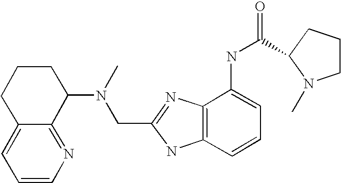

1-Methyl-N-(2-{[methyl(5,6,7,8-tetrahydro-8-quinolinyl)amino]methyl}-1H-benzimidazol-4-yl)-L-prolinamide was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from N-[(4-amino-1H-benzimidazol-2-yl)methyl]-N-methyl-5,6,7,8-tetrahydro-8-quinolinamine (0.1 g, 0.32 mmol), N-methyl-L-proline (0.047 g, 0.35 mmol), bis(2-oxo-3-oxazolidinyl)phosphinic chloride (0.09 g, 0.35 mmol), and N,N-diisopropylethylamine (0.07 mL, 0.39 mmol) in a similar manner as described herein to provide a tan solid (0.02 g, 15%): ¹H-NMR (CD₃OD) δ 8.54 (d, 1H), 7.98 (d, 1H), 7.64 (d, 1H), 7.35 (d, 1H), 7.30 (dd, 1H), 7.23 (t, 1H), 4.21 (t, 1H), 4.18 (½ ABq, 1H), 4.01 (½ABq, 1H), 3.15 (dd, 1H), 2.99-2.75 (m, 3H), 2.60-2.50 (m, 1H), 2.57 (s, 3H), 2.46-2.34 (m, 2H), 2.27-1.90 (m, 6H), 2.18 (s, 3H), 1.86-1.76 (m, 1H). MS m/z 413 (M+1).

### Example 105

### 2-Methyl-N-1-(2-{[methyl(5,6,7,8-tetrahydro-8-quinolinyl)amino]methyl}-1H-benzimidazol-4-yl)alaninamide

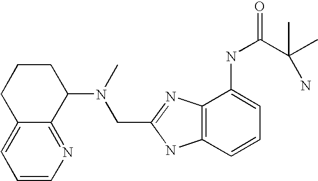

### A) 1 Phenylmethyl {1,1-dimethyl-2-[(2-{[methyl(5,6,7,8-tetrahydro-8-quinolinyl)amino]methyl}-1H-benzimidazol-4-yl)amino]-2-oxoethyl}carbamate

Phenylmethyl {1,1-dimethyl-2-[(2-{[methyl(5,6,7,8-tetrahydro-8-quinolinyl)amino]methyl}-1H-benzimidazol-4-yl)amino]-2-oxoethyl}carbamate was prepared in a similar manner to 2-{[ethyl(5,6,7,8-tetrahydro-8-quinolinyl)amino]methyl}-N-[2-(1H-imidazol-4-yl)ethyl]-1H-benzimidazole-5-carboxamide from N-[(4-amino-1H-benzimidazol-2-yl)methyl]-N-methyl-5,6,7,8-tetrahydro-8-quinolinamine (0.1 g, 0.32 mmol), 2-methyl-N-{[(phenylmethyl)oxy]carbonyl}alanine (0.1 g, 0.35 mmol), bis(2-oxo-3-oxazolidinyl)phosphinic chloride (0.09 g, 0.35 mmol), and N,N-diisopropylethylamine (0.07 mL, 0.39 mmol) in a similar manner to example x to provide an oil (0.02 g, 12%): ¹H-NMR (CD₃OD) δ 8.54 (d, 1H), 7.80 (s, 1H), 7.63 (d, 1H), 7.40-7.15 (m, 8H), 5.11 (s, 2H), 4.17 (t, 1H), 4.15 (½ ABq, 1H), 3.98 (½ABq, 1H), 3.15 (dd, 2H), 2.99-2.76 (m, 2H), 2.23-2.10 (m, 2H), 2.12 (s, 3H), 2.10-1.98 (m, 1H), 1.87-1.71 (m, 1H), 1.66 (s, 6H). MS m/z 527 (M+1).

### B) 2-Methyl-N-1-(2-{[methyl(5,6,7,8-tetrahydro-8-quinolinyl)amino]methyl}-1H-benzimidazol-4-yl)alaninamide

1 Phenylmethyl {1,1-dimethyl-2-[(2-{[methyl(5,6,7,8-tetrahydro-8-quinolinyl)amino]methyl}-1H-benzimidazol-4-yl)amino]-2-oxoethyl}carbamate (0.015 g, 0. mmol) was dissolved in EtOH (5 mL), purged with N₂, 10% palladium on carbon (0.01 g) added, the system flushed with H₂, and stirred for 2 h under an atmosphere of H₂. The product was purified using reverse phase HPLC (0% to 50% acetonitrile/water/0.1% trifluoroacetic acid gradient). The desired fractions were combined, NaCl added until the solution was saturated, neutralized and extracted with EtOAc (5×20 mL). The organic layers combined, dried over Na₂SO₄, filtered and concentrated to provide an oil (5 mg, 45%): ¹H-NMR (CD₃OD) δ 8.47 (d, 1H), 7.79 (s, 1H), 7.57 (d, 1H), 7.29 (d, 1H), 7.24 (dd, 1H), 7.1t7 (t, 1H), 4.14 (t, 1H), 4.12 (½ ABq, 1H), 3.94 (½ABq, 1H), 2.90-2.72 (m, 2H), 2.20-2.02 (m, 2H), 2.11 (s, 3H), 2.02-1.93 (m, 1H), 1.79-1.68 (m, 1H), 1.67 (s, 6H). MS m/z 393 (M+1).

### Example 106

### [3-(Dimethylamino)propyl](2-{[methyl(5,6,7,8-tetrahydro-8-quinolinyl)amino]methyl}-1H-benzimidazol-4-yl)amine

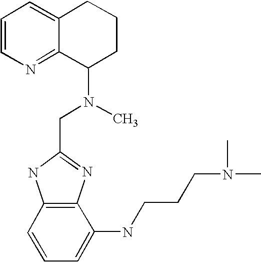

A) A solution of 3-chloro-2-nitroaniline (300 mg, 1.7 mmol) in N,N-dimethyl-1,3-propanediamine (1.5 mL) was heated in the microwave at 150° C. for 15 min. The solution was diluted with ethyl acetate (30 mL), washed with water (2×30 mL), dried over sodium sulfate, filtered and concentrated to a red oil. The crude material was purified on silica (0% to 10% 2M NH₃ in methanol/dichloromethane gradient) to provide N-[3-(dimethylamino)propyl]-2-nitro-1,3-benzenediamine (150 mg, 37%) as a red oil: ¹H-NMR (CDCl₃) δ 6.68 (t, 1H), 6.30 (d, 1H), 6.25 (d, 1H), 3.21-3.18 (m, 2H), 2.71-2.68 (m, 2H), 2.35 (s, 6H), 1.24-1.21 (m, 2H).

B) A solution of N-[3-(dimethylamino)propyl]-2-nitro-1,3-benzenediamine (150 mg, 0.63 mmol) and palladium on carbon (10% w/w, catalytic) in ethanol (100 mL) was stirred under a hydrogen atmosphere for 3 h. The reaction was filtered through celite, and concentrated to provide N¹-[3-(dimethylamino)propyl]-1,2,3-benzenetriamine (120 mg, 92%) as a brown oil: ¹H-NMR (CDCl₃) δ 6.70 (t, 1H), 6.30-6.26 (m, 2H), 3.19 (t, 2H), 2.50 (t, 2H), 2.31 (s, 6H), 1.89-1.83 (m, 2H).

C) A solution of N¹-[3-(dimethylamino)propyl]-1,2,3-benzenetriamine (120 mg, 0.58 mmol), N-methyl-N-(5,6,7,8-tetrahydro-8-quinolinyl)glycine (127 mg, 0.58 mmol), bis(2-oxo-3-oxazolidinyl)phosphinic chloride (221 mg, 0.87 mmol), and N,N-diisopropylethylamine (112 mg, 0.87 mmol) were dissolved in acetonitrile (10 mL) and the reaction was stirred for 16 h at room temperature. The reaction was diluted with ethyl acetate (50 mL), washed with water (50 mL), dried over sodium sulfate, filtered and concentrated. The crude amide was dissolved in acetic acid (10 mL) and heated at 70° C. for 3 h, concentrated, purified using reverse phase HPLC (0% to 50% acetonitrile/water/0.1% trifluoroacetic acid gradient) and repurified on silica (2% to 10% 2M NH₃ in methanol/dichloromethane gradient) to provide the product (37 mg, 16%, white solid) as the trifluoroacetate salt: ¹H-NMR (CDCl₃) δ 8.56 (d, 1H), 7.43 (d, 1H), 7.16-7.13 (m, 1H), 7.06 (t, 1H), 6.84 (d, 1H), 6.35 (d, 1H), 4.07 (d, 1H), 3.99-3.95 (m, 1H), 3.90 (d, 1H), 3.34 (t, 2H), 2.89-2.82 (m, 1H), 2.77-2.62 (m, 3H), 2.40 (s, 6H), 2.38 (s, 3H), 2.14-1.89 (m, 5H), 1.76-1.68 (m, 1H). MS m/z 393.27 (M+1).

### Example 107

### [2-(Dimethylamino)ethyl](2-{[methyl(5,6,7,8-tetrahydro-8-quinolinyl)amino]methyl}-1H-benzimidazol-4-yl)amine

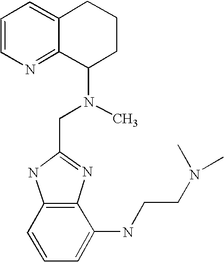

A) A solution of 3-chloro-2-nitroaniline (300 mg, 1.7 mmol) in N,N-dimethyl-1,3-ethanediamine (1.5 mL) was heated in the microwave at 150° C. for 15 min. The solution was diluted with dichloromethane and the crude material was purified on silica (0% to 10% 2M NH₃ in methanol/dichloromethane gradient) to provide N-[3-(dimethylamino)ethyl]-2-nitro-1,3-benzenediamine (126 mg, 33%) as a red solid: ¹H-NMR (DMSO-d₆) δ 8.32 (t, 1H), 7.30 (br s, 2H), 7.06 (t, 1H), 6.07 (d, 1H), 5.85 (d, 1H), 3.21-3.16 (m, 2H), 2.53-2.49 (m, 2H), 2.19 (s, 3H).

B) A solution of N-[3-(dimethylamino)ethyl]-2-nitro-1,3-benzenediamine (126 mg, 0.56 mmol) and palladium on carbon (10% w/w, catalytic) in ethanol (100 mL) was stirred under a hydrogen atmosphere for 16 h. The reaction was filtered through celite, and concentrated to provide N¹-[3-(dimethylamino)ethyl]-1,2,3-benzenetriamine (114 mg, >99%) as a red-brown: ¹H-NMR (DMSO-d₆) δ 6.40 (t, 1H), 6.06 (d, 1H), 5.96 (d, 1H), 3.07 (t, 2H), 2.56-2.49 (m, 2H), 2.22 (s, 6H).

C) A solution of N¹-[3-(dimethylamino)ethyl]-1,2,3-benzenetriamine (114 mg, 0.59 mmol), N-methyl-N-(5,6,7,8-tetrahydro-8-quinolinyl)glycine (129 mg, 0.59 mmol), bis(2-oxo-3-oxazolidinyl)phosphinic chloride (227 mg, 0.89 mmol), and N,N-diisopropylethylamine (115 mg, 0.89 mmol) were dissolved in acetonitrile (10 mL) and the reaction was stirred 16 h at room temperature. The reaction was concentrated, and the crude amide was dissolved in acetic acid (10 mL) and heated at 70° C. for 2 h and concentrated. The crude material was purified on silica (2% to 15% 2M NH₃ in methanol/dichloromethane gradient), repurified using reverse phase HPLC (0% to 35% acetonitrile/water/0.1% trifluoroacetic acid gradient) and lyophilized from water to provide the product (76 mg, 18%, sticky brown solid) as the trifluoroacetate salt: ¹H-NMR (CDCl₃) δ 9.06 (d, 1H), 8.13 (d, 1H), 7.73 (t, 1H), 7.32 (t, 1H), 7.07 (d, 1H), 6.57 (d, 1H), 4.48 (d, 1H), 4.31-4.24 (m, 2H), 3.75-3.72 (m, 2H), 3.50-3.46 (m, 2H), 3.00-2.98 (m, 1H), 2.94 (s, 6H), 2.51-2.42 (m, 1H), 2.31-2.23 (m, 4H), 2.03-1.84 (m, 3H). MS m/z 379.15 (M+1).

### Example 108

### Methyl[2-(methylamino)ethyl](2-{[methyl(5,6,7,8-tetrahydro-8-quinolinyl)amino]methyl}-1H-benzimidazol-4-yl)amine

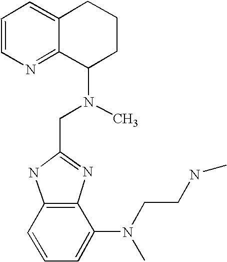

A) A solution of N,N′-dimethyl-1,2-ethanediamine (2.0 g, 22.7 mmol) and di-tert butyl dicarbonate (4.95 g, 22.7 mmol) in dichloromethane (50 mL) was stirred for 2 h, filtered and the filtrate concentrated to provide a mixture of the mono- and di-protected amines. The mixture (1 g) and 3-chloro-2-nitroaniline (500 mg, 2.9 mmol) were heated in the microwave at 150° C. for 45 min, and purified on silica (0% to 100% ethyl acetate/hexanes gradient) to provide tert-butyl {2-[(3-amino-2-nitrophenyl)(methyl)amino]ethyl}methylcarbamate (360 mg, 38%) as a red oil: ¹H-NMR (CDCl₃) δ 7.08 (t, 1H), 6.46-6.37 (m, 1H), 6.28-6.26 (m, 1H), 3.44-3.19 (m, 4H), 2.86-2.78 (m, 6H), 1.45 (s, 9H).

B) A solution of tert-butyl {2-[(3-amino-2-nitrophenyl)(methyl)amino]ethyl}methylcarbamate (360 mg, 1.11 mmol) and palladium on carbon (10% w/w, catalytic) in ethanol (50 mL) was stirred under a hydrogen atmosphere for 16 h. The reaction was filtered through celite and concentrated to provide tert-butyl {2-[(2,3-diaminophenyl)(methyl)amino]ethyl}methylcarbamate (306 mg, 94%) as a brown oil: ¹H-NMR (CDCl₃) δ 6.67-6.64 (m, 2H), 6.54-6.52 (m, 1H), 3.41-3.20 (m, 6H), 3.07-2.96 (m, 2H), 2.87-2.78 (m, 3H), 2.70-2.64 (m, 3H), 1.45 (s, 9H).

C) A solution of tert-butyl {2-[(2,3-diaminophenyl)(methyl)amino]ethyl}methylcarbamate (153 mg, 0.52 mmol), N-methyl-N-(5,6,7,8-tetrahydro-8-quinolinyl)glycine (114 mg, 0.52 mmol), bis(2-oxo-3-oxazolidinyl)phosphinic chloride (199 mg, 0.78 mmol), and N,N-diisopropylethylamine (101 mg, 0.78 mmol) were dissolved in acetonitrile (5 mL) and stirred at room temperature for 2 h. The reaction was concentrated, dissolved in acetic acid (50 mL) and heated at 70° C. for 2 h. The reaction was concentrated, dissolved in dichloromethane (10 mL) and trifluoroacetic acid (5 mL), stirred for 2 h and concentrated. The crude product was purified using reverse phase HPLC (0% to 40% acetonitrile/water/0.1% trifluoroacetic acid gradient) and lyophilized from water to provide the product (71 mg, 19%, sticky brown solid) as the trifluoroacetate salt: ¹H-NMR (CD₃OD) δ 8.71 (d, 1H), 8.19 (d, 1H), 7.77-7.74 (m, 1H), 7.42-7.35 (m, 2H), 7.05 (d, 1H), 4.60-4.56 (m, 1H), 4.54 (d, 1H), 4.42 (d, 1H), 3.67-3.63 (m, 2H), 3.36-3.33 (m, 2H), 3.03-2.96 (m, 2H), 2.95 (s, 3H), 2.79 (s, 3H), 2.51 (s, 3H), 2.40-2.33 (m, 1H), 2.25-2.18 (m, 1H), 2.13-2.02 (m, 1H), 1.96-1.85 (m, 1H). MS m/z 379.22 (M+1).

### Example 109

### [2-(Dimethylamino)ethyl]methyl(2-{[methyl(5,6,7,8-tetrahydro-8-quinolinyl)amino]methyl}-1H-benzimidazol-4-yl)amine

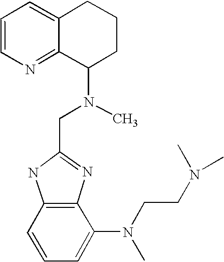

### A) Phenylmethyl ({4-[[2-(dimethylamino)ethyl](methyl)amino]-1H-benzimidazol-2-yl}methyl)carbamate

A) A solution of 3-chloro-2-nitroaniline (550 mg, 3.2 mmol) in N,N,N-trimethyl-1,2-ethanediamine (2 mL) was heated in the microwave at 150° C. for 15 min. The crude material was purified on silica (0% to 100% ethyl acetate/hexanes gradient) to provide N-[2-(dimethylamino)ethyl]-N-methyl-2-nitro-1,3-benzenediamine (380 mg, 50%) as a red solid: ¹H-NMR (CD₃OD) δ 7.09 (t, 1H), 6.45-6.40 (m, 2H), 3.15-3.11 (m, 2H), 2.73 (s, 3H), 2.51-2.47 (m, 2H), 2.2-3 (s, 6H). A solution of N-[2-(dimethylamino)ethyl]-N-methyl-2-nitro-1,3-benzenediamine (380 mg, 1.6 mmol) and palladium on carbon (10% w/w, catalytic) in ethanol (100 mL) was stirred under a hydrogen atmosphere for 60 h. The reaction was filtered through celite, and concentrated. The crude triamine, N-{[(phenylmethyl)oxy]carbonyl}glycine (368 mg, 1.76 mmol), bis(2-oxo-3-oxazolidinyl)phosphinic chloride (611 mg, 2.4 mmol), and N,N-diisopropylethylamine (310 mg, 2.4 mmol) were dissolved in acetonitrile (20 mL) and the reaction was stirred 16 h at room temperature. The reaction was concentrated, and the crude amide was dissolved in acetic acid (30 mL) and heated at 70° C. for 2 h and concentrated. The crude material was purified on silica (0% to 10% 2M NH₃ in methanol/dichloromethane gradient) to provide the product (255 mg, 42%) as a brown solid: ¹H-NMR (CD₃OD) δ 7.40-7.29 (m, 7H), 7.07 (d, 1H), 5.12 (s, 2H), 4.73 (s, 2H), 3.64-3.60 (m, 2H), 3.41-3.38 (m, 2H), 2.91 (s, 9H).

### B) [2-(Dimethylamino)ethyl]methyl(2-{[methyl(5,6,7,8-tetrahydro-8-quinolinyl)amino]methyl}-1H-benzimidazol-4-yl)amine

A solution of phenylmethyl ({4-[[2-(dimethylamino)ethyl](methyl)amino]-1H-benzimidazol-2-yl}methyl)carbamate (255 mg, 0.67 mmol) and palladium on carbon (10% w/w, catalytic) in ethanol (40 mL) was stirred under a hydrogen atmosphere for 16 h. The reaction was filtered through celite, and concentrated to provide the N-[2-(aminomethyl)-1H-benzimidazol-4-yl]-N,N′,N′-trimethyl-1,2-ethanediamine (160 mg, 96%): ¹H-NMR (CD₃OD) δ 7.21 (t, 1H), 7.13 (d, 1H), 6.76 (d, 1H), 4.46 (s, 2H), 3.95-3.92 (m, 2H), 3.52-3.49 (m, 2H), 3.06 (s, 6H), 2.97 (s, 3H).

C) N-[2-(aminomethyl)-1H-benzimidazol-4-yl]-N,N′,N′-trimethyl-1,2-ethanediamine (100 mg, 0.40 mmol), 6,7-dihydro-8(5H)-quinolinone (59 mg, 0.40 mmol), acetic acid (36 mg, 0.60 mmol) and sodium triacetoxyborohydride (127 mg, 0.60 mmol) were dissolved in 1,2-dichloroethane (5 mL) and stirred for 2 h at room temperature. The reaction mixture was concentrated and the crude secondary amine, formaldehyde (37% aqueous solution, 0.09 mL, 1.2 mmol), acetic acid (36 mg, 0.60 mmol) and sodium triacetoxyborohydride (127 mg, 0.60 mmol) were dissolved in 1,2-dichloroethane (5 mL) and stirred for 90 min at room temperature. The reaction mixture was concentrated and the crude tertiary amine was purified on silica (0% to 7% 2M ammonia in methanol/dichloromethane) to provide the product as a light tan solid (25 mg, 16%). ¹H-NMR (CD₃OD) δ 8.49 (d, 1H), 7.58 (d, 1H) 7.26-7.23 (m, 1H), 7.13-7.07 (m, 2H), 6.69-6.64 (m, 1H), 4.14-4.08 (m, 2H), 3.93 (d, 1H), 3.00 (s, 3H), 2.92-2.63 (m, 4H), 2.58-2.55 (m, 2H), 2.27 (s, 6H), 2.25-2.13 (m, 1H), 2.10 (s, 3H), 2.08-1.94 (m, 2H), 1.78-1.70 (m, 1H). MS m/z 393.29 (M+1).

### Example 110

### N-{[4-(4-Acetyl-1-piperazinyl)-1H-benzimidazol-2-yl]methyl}-N-methyl-5,6,7,8-tetrahydro-8-quinolinamine

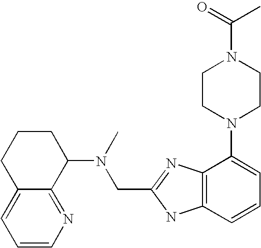

N-Methyl-N-{[4-(1-piperazinyl)-1H-benzimidazol-2-yl]methyl}-5,6,7,8-tetrahydro-8-quinolinamine (0.05 g, 0.1 mmol), DIPEA, (0.028 mL, 0.16 mmol) and acetic anhydride (0.015 mL, 0.15 mmol) were added to CH₂Cl₂ (3 mL) and stirred for 1 h. concentrated, diluted with ethyl acetate (25 mL), washed with sat. NaHCO₃ (5 mL), dried over Na₂SO₄, filtered and concentrated. The product was purified using reverse phase HPLC (0% to 50% acetonitrile/water/0.1% trifluoroacetic acid gradient). The desired fractions were combined, NaCl added until the solution was saturated, neutralized and extracted with EtOAc (5×20 mL). The organic layers combined, dried over Na₂SO₄, filtered and concentrated to provide a white solid (0.1 g, 18% yield). ¹H-NMR (DMSO-d₆): δ 12.2 (br s, 1H), 8.45 (d, 1H), 7.49 (d, 1H), 7.17 (dd, 1H), 7.02-6.93 (m, 2H), 6.45 (m, 1H), 4.02 (ABq, 2H), 3.93 (t, 1H), 3.59 (s, 4H), 3.45 (m, 4H), 2.83-2.75 (m, 1H), 2.66 (d, 1H), 2.23 (s, 3H), 2.01 (s, 3H), 2.08-2.00 (m, 1H), 1.93 (m, 1H), 1.68-1.56 (m, 1H). MS m/z 419 (M+1).

### Example 111

### N-Methyl-N-({4-[4-(1-methylethyl)-1piperazinyl]-1H-benzimidazol-2-yl}methyl)-5,6,7,8-tetrahydro-8-quinolinamine

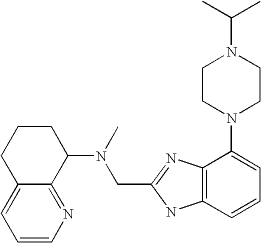

N-Methyl-N-({4-[4-(1-methylethyl)-1-piperazinyl]-1H-benzimidazol-2-yl}methyl)-5,6,7,8-tetrahydro-8-quinolinamine was prepared in a similar manner as described above from N-methyl-N-{[4-(1-piperazinyl)-1H-benzimidazol-2-yl]methyl}-5,6,7,8-tetrahydro-8-quinolinamine (0.15 g, 0.4 mmol) and acetone, (0.08 mL, 1.2 mmol) to provide a tan solid (0.05 g, 23% yield). ¹H-NMR (d6-DMSO): δ 8.46 (d, 1H), 7.50 (d, 1H), 7.18 (t, 1H), 6.98 (t, 2H), 6.42 (s, 1H), 4.02 (Abq, 2H), 3.92 (t, 1H), 3.41 (s, 4H), 2.77 (m, 1H), 2.75-2.55 (m, 5H), 2.22 (s, 3H), 2.10-1.99 (m, 1H), 1.98-1.88 (m, 2H), 1.70-1.59 (m, 1H), 1.02 (d, 6H). MS m/z 419 (M+1).

### Example 112

### N-(1-Methylethyl)-N-{[4-(4-methyl-1-piperazinyl)-1H-benzimidazol-2-yl]methyl}-5,6,7,8-tetrahydro-8-quinolinamine

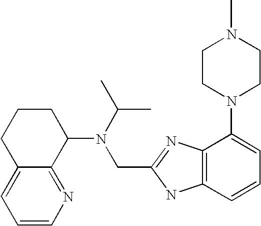

A) 6,7-Dihydro-8(5H)-quinolinone (4.0 g, 27 mmol), glycine benzyl ester (5.6 g, 34 mmol) and acetic acid (2.0 mL, 34 mmol) were dissolved in dichloroethane (50 mL). Sodium triacetoxyborohydride (7.2 g, 34 mmol) was added in three equal portions over 1 h and stirred for 14 h. A solution of sat. NaHCO₃ (25 mL) was added and the mixture stirred for 30 min., the layers separated and the aqueous layer extracted with CH₂Cl₂. The organic layers combined, dried over Na₂SO₄, filtered and concentrated to provide an oil. A portion of the intermediate (2.5 g, 8.4 mmol) was dissolved in dichloroethane (50 mL), acetone (1 mL, 12.6 mmol), acetic acid (0.75 mL, 12.6 mmol) and sodium triacetoxyborohydride (2.7 g, 12.6 mmol) were added and the reaction stirred 12 h. The reaction was worked up as above and purified by column chromatography (1% to 5% 2M NH₃ in methanol/dichloromethane gradient) to afford an oil (2.0 g, 86%): MS m/z 339 (M+1).

B) 1,1-Dimethylethyl 4-(3-amino-2-nitrophenyl)-1-piperazinecarboxylate (0.5 g, 1.55 mmol) and palladium on carbon (10% w/w, catalytic) in ethanol (25 mL) were stirred under a hydrogen atmosphere for 6 h. The reaction was filtered through celite, concentrated and placed under high vacuum to provide a redish oil. Phenylmethyl N-(1-methylethyl)-N-(5,6,7,8-tetrahydro-8-quinolinyl)glycinate (0.575 g, 1.7 mmol) and palladium on carbon (10% w/w, catalytic) in ethanol (50 mL) were stirred under a hydrogen atmosphere for 4 h. The reaction was filtered through celite, concentrated and placed under high vacuum to provide tan solid. The intermediates were dissolved in acetonitrile (25 mL) and combined with bis(2-oxo-3-oxazolidinyl)phosphinic chloride (0.435 g, 0.1.7 mmol), and N,N-diisopropylethylamine (0.3 mL, 1.7 mmol) and stirred at room temperature for 12 h. The reaction was concentrated, dissolved in acetic acid (50 mL) and heated at 70° C. for 2 h. The reaction was concentrated, dissolved in EtOAc (50 mL) and washed with sat. NaHCO₃ (3×10 mL), dried over Na₂SO₄, filtered, evaporated and purified by column chromatography (1% to 5% 2M NH₃ in methanol/dichloromethane gradient) to afford a yellow foam (0.25 g, 32%) The foam was dissolved in CH₂Cl₂ (2.5 mL) and trifluoroacetic acid (2.5 mL) and stirred for 2 h and concentrated. To the crude amine was added dichloroethane (5 mL), 37% formaldehyde (0.025 mL, 0.37 mmol), acetic acid (0.025 mL, 0.37 mmol) and sodium triacetoxyborohydride (0.78 g, 0.37 mmol). The reaction mixture was stirred 1 h and a solution of sat. NaHCO₃ (2.5 mL) was added and stirred for 15 min. The layers separated and the aqueous layer extracted with CH₂Cl₂ (3×5 mL), the organic layers combined, dried over Na₂SO₄, filtered, concentrated and purified using reverse phase HPLC (0% to 50% acetonitrile/water/0.1% trifluoroacetic acid gradient). The desired fractions were combined, NaCl added until the solution was saturated, neutralized and extracted with EtOAc (5×20 mL). The organic layers combined, dried over Na₂SO₄, filtered and concentrated to provide a white solid (57 mg, 55%): ¹H-NMR (DMSO-d₆) δ 13.1 (br s, 1H), 8.59 (d, 1H), 7.48 (d, 1H), 7.20 (dd, 1H), 7.09 (d, 1H), 6.94 (t, 1H), 6.44 (s, 1H) 4.02 (½ ABq, 1H), 4.00 (t, 1H), 3.92 (½ABq, 1H), 3.48 (s, 4H), 2.93 (sep, 1H), 2.80-2.61 (m, 6H), 2.37 (s, 3H), 2.08 (m, 1H), 1.84 (m, 2H), 1.60 (m, 1H), 1.05 (d, 3H), 0.91 (d, 3H). MS m/z 419 (M+1).

### Example 113

### N-(1-methylethyl)-N-({4-[4-(1-methylethyl)-piperazinyl]-1H-benzimidazol-2-yl}methyl)-5,6,7,8-tetrahydro-8-quinolinamine

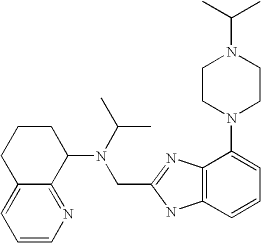

N-(1-methylethyl)-N-({4-[4-(1-methylethyl)-1-piperazinyl]-1H-benzimidazol-2-yl}methyl)-5,6,7,8-tetrahydro-8-quinolinamine was prepared in a similar manner as N-(1-methylethyl)-N-{[4-(4-methyl-1-piperazinyl)-1H-benzimidazol-2-yl]methyl}-5,6,7,8-tetrahydro-8-quinolinamine from 1,1-dimethylethyl 4-(2-{[(1-methylethyl)(5,6,7,8-tetrahydro-8-quinolinyl)amino]methyl}-1H-benzimidazol-4-yl)-1-piperazinecarboxylate (0.125 g, 0.25 mmol) by deprotection and reductive amination with acetone, (0.025 mL, 0.37 mmol) to provide a white solid (0.035 g, 31% yield): ¹H-NMR (CD₃OD) δ 13.1 (br s, 1H), 8.60 (s, 1H), 7.49 (d, 1H), 7.21 (t, 1H), 7.07 (d, 1H), 6.93 (t, 1H), 6.40 (s, 1H), 4.02 (½Abq, 1H), 4.00 (t, 1H), 3.91 (½Abq, 1H), 3.44-3.25 (m, 4H) 2.94 (m, 1H), 2.79-2.62 (m, 7H), 2.21-2.04 (m, 1H), 1.93-1.80 (m, 2H), 1.66-1.56 (m, 1H), 1.04 (d, 9H), 0.91 (d, 3H). MS m/z 447 (M+1).

### Example 114

### N-{1-methyl-1-[4-(4-methyl-1-piperazinyl)-1H-benzimidazol-2-yl]ethyl}-5,6,7,8-tetrahydro-8-quinolinamine

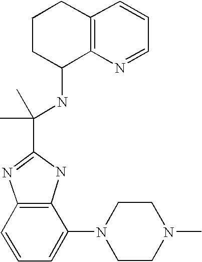

A) 3-(4-Methyl-1-piperazinyl)-2-nitroaniline (0.7 g, 3.0 mmol) and palladium on carbon (10% w/w, 70 mg) in ethanol (50 mL) were stirred under a hydrogen atmosphere for 3 h. The reaction was filtered through celite, concentrated and placed under high vacuum to provide a redish oil. The intermediate was dissolved in acetonitrile (10 mL), acetic acid (0.68 mL, 4.5 mmol), 2-methyl-N-{[(phenylmethyl)oxy]carbonyl}alanine (1.9 g, 4.5 mmol) and ), bis(2-oxo-3-oxazolidinyl)phosphinic chloride (1.15 g, 4.5 mmol) were added, the reaction stirred for 12 h. The reaction mixture was concentrated, diluted with acetic acid (10 mL), and stirred a 70° C. for 2 h. sat. The reaction mixture was concentrated and purified by column chromatography (1% to 5% 2M NH₃ in methanol/dichloromethane gradient) to afford a yellow foam (0.74 g, 60%): ¹H-NMR (DMSO-d₆) δ 12.0 (br s, 1H), 7. (s, 1H), 7.34 (m, 5H), 6.95 (dd, 1H), 6.91 (d, 1H), 6.4 (d, 1H), 4.94 (s, 2H), 3.45 (s, 4H), 2.58 (s, 4H), 2.21 (s, 3H), 1.62 (s, 6H). MS m/z 408 (M+1).

B) N-{1-methyl-1-[4-(4-methyl-1-piperazinyl)-1H-benzimidazol-2-yl]ethyl}-5,6,7,8-tetrahydro-8-quinolinamine was prepared in similar manner as described herein from phenylmethyl {1-methyl-1-[4-(4-methyl-1-piperazinyl)-1H-benzimidazol-2-yl]ethyl}carbamate (0.73 g, 1.79 mmol) by deprotection followed by reductive amination with 6,7-dihydro-8(5H)-quinolinone (263 mg, 1.79 mmol) to afford a white solid foam (0.18 g, 24%): ¹H-NMR (DMSO-d₆) δ 12.9 (br s, 1H), 8.50 (d, 1H), 7.50 (d, 1H), 7.22 (dd, 1H), 6.95 (dd, 1H), 6.91 (d, 1H), 6.40 (d, 1H), 3.79 (s, 1H), 3.49 (s, 4H), 2.58 (s, 4H), 2.75-2.60 (m, 2H), 2.22 (s, 3H), 1.76 (m, 2H), 1.67-1.60 (m, 1H), 1.59 (s, 3H), 1.56 (s, 3H), 1.51-1.42 (m, 1H). MS m/z 405 (M+1).

### Example 115

### N-Methyl-N-{1-methyl-1-[4-(4-methyl-1-piperazinyl)-1H-benzimidazol-2-yl]ethyl}-5,6,7,8-tetrahydro-8-quinolinamine

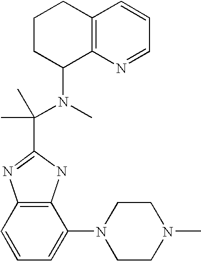

N-methyl-N-{1-methyl-1-[4-(4-methyl-1-piperazinyl)-1H-benzimidazol-2-yl]ethyl}-5,6,7,8-tetrahydro-8-quinolinamine was prepared in similar manner to [2-(dimethylamino)ethyl]methyl(2-{[methyl(5,6,7,8-tetrahydro-8-quinolinyl)amino]methyl}-1H-benzimidazol-4-yl)amine from N-{1-methyl-1-[4-(4-methyl-1-piperazinyl)-1H-benzimidazol-2-yl]ethyl}-5,6,7,8-tetrahydro-8-quinolinamine (0.17 g, 0.4 mmol) and formaldehyde (37% aqueous solution, 0.05 mL, 0.6 mmol) to afford a white solid (0.45 g, 26%): ¹H-NMR (CD₃OD) δ 8.58 (d, 1H), 7.46 (d, 1H), 7.21 (dd, 1H), 7.15 (d, 1H), 7.05 (t, 1H), 6.66 (d, 1H), 4.12 (t, 1H), 3.37 (s, 4H), 2.87 (s, 4H), 2.86-2.71 (m, 1H), 2.62 (m, 1H), 2.48 (s, 3H), 2.00 (m, 3H), 1.91 (m, 1H), 1.67 (s, 3H), 1.62 (s, 3H). MS m/z 419 (M+1).

### Example 116

### N-({4-[4-(Aminoacetyl)-1-piperazinyl]-1H-benzimidazol-2-yl}methyl)-N-methyl-5,6,7,8-tetrahydro-8-quinolinamine

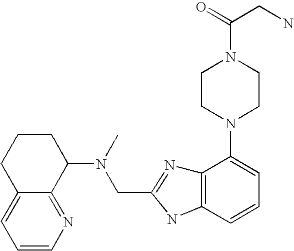

N-Methyl-N-{[4-(1-piperazinyl)-1H-benzimidazol-2-yl]methyl}-5,6,7,8-tetrahydro-8-quinolinamine (0.05 g, 0.1 mmol), bis(2-oxo-3-oxazolidinyl)phosphinic chloride (0.037 g, 0.14 mmol), N,N-diisopropylethylamine (28 mL, 0.16 mmol) and N—Boc-glycine (0.025 g, 0.14 mmol) were dissolved in acetonitrile (3 mL) and stirred for 12 h. The reaction was concentrated and purified using reverse phase HPLC (0% to 50% acetonitrile/water/0.1% trifluoroacetic acid gradient). The desired fractions were combined, NaCl added until the solution was saturated, neutralized and extracted with EtOAc (5×20 mL). The organic layers combined, dried over Na₂SO₄, filtered and concentrated to provide to afford an oil. The oil was dissolved in 5N HCl in dioxane (1 mL) and methanol (1 mL) and stirred for 1 hour. The solution was concentrated and purified by reverse phase HPLC according to the above protocol to afford a white solid (0.025 g, 45% yield). ¹H-NMR (DMSO-d₆): δ 8.44 (d, 1H), 7.49 (d, 1H), 7.17 (dd, 1H), 7.03 (s, 1H), 6.97 (t, 1H), 6.45 (s, 1H), 4.01 (ABq, 2H), 3.91 (t, 1H), 3.65 (s, 4H), 3.53 (m, 4H), 2.43 (s, 2H), 2.81-2.74 (m, 1H), 2.66 (d, 1H), 2.21 (s, 3H), 2.02 (m, 1H), 1.93 (m, 2H), 1.64 (m, 1H). MS m/z 434 (M+1).

### Example 117

### N-Methyl-N-({4-[4-(2-pyridinylcarbonyl)-1-piperazinyl]-1H-benzimidazol-2-yl}methyl)-5,6,7,8-tetrahydro-8-quinolinamine

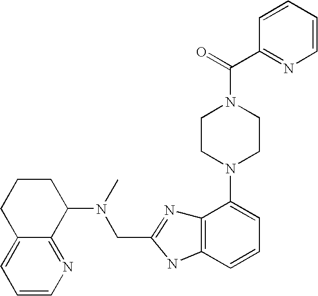

N-Methyl-N-({4-[4-(2-pyridinylcarbonyl)-1-piperazinyl]-1H-benzimidazol-2-yl}methyl)-5,6,7,8-tetrahydro-8-quinolinamine was prepared from N-methyl-N-{[4-(1-piperazinyl)-1H-benzimidazol-2-yl]methyl}-5,6,7,8-tetrahydro-8-quinolinamine (0.05 g, 0.1 mmol), bis(2-oxo-3-oxazolidinyl)phosphinic chloride (0.037 g, 0.14 mmol), N,N-diisopropylethylamine (28 mL, 0.16 mmol) and picolinic acid (0.025 g, 0.14 mmol). Reagents were dissolved in acetonitrile (3 mL) and stirred for 12 h. The reaction was concentrated and purified using reverse phase HPLC (0% to 50% acetonitrile/Water/0.1% trifluoroacetic acid gradient). The desired-fractions were combined, NaCl added until the solution was saturated, neutralized and extracted with EtOAc (5×20 mL). The organic layers combined, dried over Na₂SO₄, filtered and concentrated to provide to afford a white solid. The oil was dissolved in 5N HCl in dioxane (1 mL) and methanol (1 mL) and stirred for 1 hour. The solution was concentrated and purified by reverse phase HPLC according to the above protocol to afford a white solid (0.040 g, 62% yield). ¹H-NMR (DMSO-d₆): δ 12.2 (br, s), 8.60 (D, 1H) 8.47 (d, 1H), 7.93 (t, 1H), 7.61 (d, 1H), 7.48 (dd, 2H), 7.18 (d, 1H), 7.05 (d, 1H), 6.98 (t, 1H), 6.46 (d, 1H), 4.03 (ABq, 2H), 3.93 (t, 1H), 3.84 (s, 2H), 3.58 (m, 4H), 3.38 (s, 2H), 2.43 (s, 2H), 2.84-2.75 (m, 1H), 2.67 (d, 1H), 2.23 (s, 3H), 2.03 (m, 1H), 1.94 (m, 2H), 1.64 (m, 1H). MS m/z 482 (M+1).

### Example 118

### Ethyl N-[(8R)-5,6,7,8-tetrahydro-8-quinolinyl]glycinate

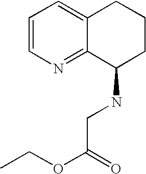

To a solution of (8R)—N-{(1R)-1-[4-(methyloxy)phenyl]ethyl}-5,6,7,8-tetrahydro-8-quinolinamine (4.4 g, 15.6 mmol) in dichloroethane (0.2 L) was added acetic acid (1.8 mL, 31.2 mmol), ethyloxoacetate (50% in toluene, 6.36 g, 31.2 mmol) and sodium triacetoxyborohydride (6.6 g, 31.2 mmol). The reaction was stirred for 14 h at room temperature, diluted with water (0.1 L) and dichloromethane (0.1 L), the layers separated, dried over sodium sulfate, filtered and concentrated. Purification by column chromatography (0% to 100% EtOAc/hexanes gradient) afforded an oil (4.2 g 73%). ¹H-NMR (DMSO-d₆) δ 8.37 (d, 1H), 7.41 (d, 3H), 7.12 (dd, 1H), 6.85 (d, 2H), 4.70 (q, 1H), 3.85 (q, 2H), 3.80 (t, 1H), 3.71 (s, 3H), 3.12 (ABq, 2H), 2.77-2.69 (m, 1H), 2.61-2.52 (m, 1H), 1.96 (m, 1H), 1.78 (m, 2H), 1.44 (m, 1H), 1.20 (d, 3H), 1.06 (t, 3H). The crude tertiary amine (2.2 g, 6.0 mmol) was dissolved in dichloro-methane (5 mL) and trifluoroacetic acid (5 mL) and stirred for 2 h at room temperature. The reaction was concentrated, diluted with EtOAc (100 mL), washed with saturated aqueous sodium bicarbonate (150 mL), dried over sodium sulfate, filtered and concentrated to afford a clear oil (149 mg, 59%): ¹H-NMR (DMSO-d₆) δ 8.35 (d, 1H), 7.48 (d, 1H), 7.17 (t, 1H), 4.10 (q, 2H), 3.66 (d, 1H), 3.48 (ABq, 2H), 2.73 (m, 2H), 2.00 (m, 1H), 1.88 (m, 1H), 1.67-1.53 (m, 2H), 1.19 (t, 3H).

### Example 119

### Ethyl N-methyl-N-[(8R)-5,6,7,8-tetrahydro-8-quinolinyl]glycinate

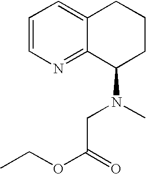

To a solution of ethyl N-[(8R)-5,6,7,8-tetrahydro-8-quinolinyl]glycinate (0.1 g, 0.42 mmol) in dichloroethane (0.2 L) was added acetic acid (0.035 mL, 0.63 mmol), formaldehyde (37%, 0.01 mL, 0.82 mmol) and sodium triacetoxyborohydride (134 mg, 6.3 mmol). The reaction was stirred for 2 h at room temperature, diluted with a sat. NaHCO₃ solution (0.1 L) and dichloromethane (0.1 L), the layers separated, dried over sodium sulfate, filtered and concentrated to give a yellow oil (0.27 g 52%). ¹H-NMR (CD₃OD) δ 8.35 (d, 1H), 7.50 (d, 1H), 7.17 (dd, 1H), 4.11 (q, 2H), 3.96 (dd, 1H), 3.41 (s, 2H), 2.90-2.82 (m, 1H), 2.73 (m, 1H), 2.37 (s, 3H), 2.07-1.97 (m, 3H), 1.69 (m, 1H), 1.23 (t, 3H). MS m/z 249 (M+1).

### Example 120

### Ethyl N-ethyl-N-[(8R)-5,6,7,8-tetrahydro-8-quinolinyl]glycinate

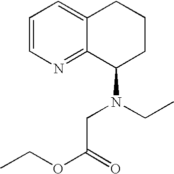

Ethyl N-ethyl-N-[(8R)-5,6,7,8-tetrahydro-8-quinolinyl]glycinate was prepared in a similar manner to ethyl N-methyl-N-[(8R)-5,6,7,8-tetrahydro-8-quinolinyl]glycinate from ethyl N-[(8R)-5,6,7,8-tetrahydro-8-quinolinyl]glycinate (0.47 g, 2.0 mmol), acetic acid (0.23 mL, 4.0 mmol), acetaldehyde (0.22 mL, 4.0 mmol) and sodium triacetoxyborohydride (848 mg, 4.0 mmol) to afford a yellow oil (0.27 g 52%). ¹H-NMR (CDCl₃) δ 8.38 (d, 1H), 7.45 (d, 1H), 7.00 (t, 1H), 4.11 (q, 2H), 3.54 (½ABq, 1H), 3.48 (½ABq, 1H), 2.85-2.75 (m, 2H), 2.70-2.63 (m, 1H), 2.14-2.05 (m, 1H), 2.05-1.97 (m, 1H), 1.83 (m, 1H), 1.68 (m, 1H), 1.22 (t, 3H), 1.07 (t, 3H). MS m/z 263 (M+1).

### Example 121

### Ethyl N-propyl-N[(8R)-5,6,7,8-tetrahydro-8-quinolinyl]glycinate

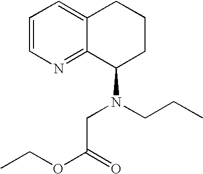

Ethyl N-propyl-N-[(8R)-5,6,7,8-tetrahydro-8-quinolinyl]glycinate was prepared in a similar manner to ethyl N-methyl-N-[(8R)-5,6,7,8-tetrahydro-8-quinolinyl]glycinate from propyl N-[(8R)-5,6,7,8-tetrahydro-8-quinolinyl]glycinate (0.47 g, 2.0 mmol), acetic acid (0.23 mL, 4.0 mmol), proprionaldhyde (0.29 mL, 4.0 mmol) and sodium triacetoxyborohydride (848 mg, 4.0 mmol) to afford a clear oil (0.52 g 95%). ¹H-NMR (CDCl₃) δ 8.42 (d, 1H), 7.34 (d, 1H), 7.05 (t, 1H), 4.12 (q, 2H), 3.74 (m, 1H), 3.60 (½ABq, 1H), 3.40 (½ABq, 1H), 2.82-2.77 (m, 1H), 2.72-2.65 (m, 3H), 2.16-2.07 (m, 1H), 2.06-1.97 (m, 1H), 1.86 (m, 1H), 1.48 (quin, 1H), 1.25 (t, 3H), 0.85 (t, 3H). MS m/z 277 (M+1).

### Example 122

### Ethyl N-(cyclopropylmethyl)-N-[(8R)-5,6,7,8-tetrahydro-8-quinolinyl]glycinate

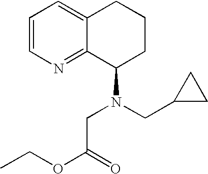

Ethyl N-(cyclopropylmethyl)-N-[(8R)-5,6,7,8-tetrahydro-8-quinolinyl]glycinate was prepared in a similar manner to ethyl N-methyl-N-[(8R)-5,6,7,8-tetrahydro-8-quinolinyl]glycinate from ethyl N-[(8R)-5,6,7,8-tetrahydro-8-quinolinyl]glycinate (0.47 g, 2.0 mmol), acetic acid (0.23 mL, 4.0 mmol), cyclopropanecarbaldehyde (0.3 mL, 4.0 mmol) and sodium triacetoxyborohydride (848 mg, 4.0 mmol) to afford a clear oil (0.44 g 76%). ¹H-NMR (CDCl₃) δ 8.42 (d, 1H), 7.34 (d, 1H), 7.03 (t, 1H), 4.22 (dd, 1H), 4.13 (q, 2H), 3.58 (ABq, 2H), 2.84-2.76 (m, 1H), 2.70 (m, 1H), 2.62 (dd, 1H), 2.17-2.08 (m, 1H), 2.06-1.97 (m, 1H), 1.92-1.38 (m, 1H), 1.75-1.65 (m, 1H), 1.25 (t, 3H), 0.97-0.86 (m, 1H), 0.44 (ddd, 2H), 0.08 (ddd, 2H). MS m/z 289 (M+1).

### Example 123

### N-Cyclopropyl-5,6,7,8-tetrahydro-8-quinolinamine

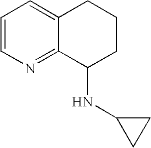

To a solution of 6,7-dihydro-8(5H)-quinolinone (200 mg, 1.36 mmol) in dichloroethane (7 mL) was added cyclopropylamine (95 μL, 1.37 mmol), acetic acid (117 μL, 2.04 mmol), and sodium triacetoxyborohydride (432 mg, 2.04 mmol). The mixture was stirred at room temperature for 2 hours and an additional 95 μL cyclopropylamine was added. The mixture was then stirred at room temperature 15 hours, diluted with dichloromethane, and extracted with aqueous sodium bicarbonate. The organic layer was separated and the aqueous extracted with additional dichloromethane. The organic layers were combined, dried over magnesium sulfate, concentrated, and purified by column chromatography (0-7.5% ammonium hydroxide-acetonitrile) to give a quantitative yield of N-cyclopropyl-5,6,7,8-tetrahydro-8-quinolinamine as a yellow oil. ¹H NMR (400 MHz, CDCl₃) δ 8.36 (d, J=4.5 Hz, 1H), 7.35 (d, J=7.8 Hz, 1H), 7.04 (dd, J=7.5, 4.7 Hz, 1H), 3.92 (m, 1H), 3.08 (br, 1H), 2.83-2.69 (m, 2H), 2.26 (m, 1H), 2.17 (m, 1H), 2.00-1.84 (m, 2H), 1.73 (m, 1H), 0.54 (m, 1H), 0.46-0.37 (m, 3H); MS m/z 189 (M+1).

### Example 124

### (8S)—N-Methyl-5,6,7,8-tetrahydro-8-quinolinamine

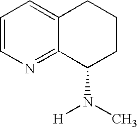

To a solution of (8S)—N-{(1S)-1-[4-(methyloxy)phenyl]ethyl}-5,6,7,8-tetrahydro-8-quinolinamine (2.0 g, 7.1 mmol) in dichloroethane (70 mL) was added acetic acid (637 mg, 10.6 mmol) formaldehyde (37% aqueous solution, 1.06 mL, 14.2 mmol) and sodium triacetoxyborohydride (2.25 g, 10.6 mmol). The reaction was stirred for 1 h at room temperature, an additional portion of formaldehyde (37% aqueous solution, 1.06 mL, 14.2 mmol) was added and the reaction was stirred for 2 h at room temperature. The reaction mixture was diluted with saturated aqueous sodium bicarbonate (100 mL) and extracted with chloroform/isopropanol (3:1, 2×100 mL). The combined organic extracts were dried over sodium sulfate, filtered and concentrated. The crude tertiary amine was dissolved in dichloromethane (10 mL) and trifluoroacetic acid (5 mL) and stirred for 16 h at room temperature. The reaction was concentrated, diluted with saturated aqueous sodium bicarbonate (200 mL) and extracted with chloroform/isopropanol (3:1, 2×200 mL), dried over sodium sulfate, filtered and concentrated. The crude amine was purified on silica (0% to 10% 2M NH₃ in methanol/dichloromethane gradient) to provide the product (1.04 g, 90%) as a brown oil: ¹H-NMR (CD₃OD) δ 8.37 (d, 1H), 7.54 (d, 1H), 7.21-7.18 (m, 1H), 3.73-3.70 (m, 1H), 3.30 (s, 3H), 2.89-2.76 (m, 2H), 2.22-2.16 (m, 1H), 2.06-1.96 (m, 1H), 1.82-1.71 (m, 1H). MS m/z 163 (M+1).

### Example 125

### (8R)—N-Methyl-5,6,7,8-tetrahydro-8-quinolinamine

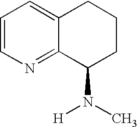

(8R)—N-Methyl-5,6,7,8-tetrahydro-8-quinolinamine was prepared in a similar manner to (8S)—N-Methyl-5,6,7,8-tetrahydro-8-quinolinamine from (8R)—N-{(1S)-1-[4-(methyloxy)phenyl]ethyl}-5,6,7,8-tetrahydro-8-quinolinamine (0.89 g, 3.2 mmol) to provide an oil (0.35 g, 53%) with ¹H-NMR and MS data matching that of (8S)—N-Methyl-5,6,7,8-tetrahydro-8-quinolinamine.

### Example 126

### (8S)—N-Ethyl-5,6,7,8-tetrahydro-8-quinolinamine

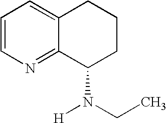

To a solution of (8S)—N-{(1S)-1-[4-(methyloxy)phenyl]ethyl}-5,6,7,8-tetrahydro-8-quinolinamine (300 mg, 1.06 mmol) prepared from (S)-(−)-1-(4methoxyphenyl)ethylamine and 6,7-dihydro-8(5H)quinolinone (J. Org. Chem., 2002, 67, 2197-2205, incorporated by reference with regard to such synthesis) in dichloroethane (3 mL) was added acetic acid (96 mg, 1.60 mmol), acetaldehyde (2 mL) and sodium triacetoxyborohydride (339 mg, 1.60 mmol). The reaction was stirred for 16 h at room temperature, diluted with saturated aqueous sodium bicarbonate (50 mL), extracted with dichloromethane (50 mL), dried over sodium sulfate, filtered and concentrated. The crude tertiary amine was dissolved in dichloromethane (3 mL) and trifluoroacetic acid (3 mL) and stirred for 16 h at room temperature. The reaction was concentrated, diluted with saturated aqueous sodium bicarbonate (50 mL) and extracted with chloroform/isopropanol (3:1, 2×50 mL), dried over sodium sulfate, filtered and concentrated. The crude amine was purified on silica (0% to 10% 2M NH₃ in methanol/dichloromethane gradient) to provide the product (145 mg, 78%) as a brown oil: ¹H-NMR (CDCl₃) δ 8.40 (d, 1H), 7.38 (d, 1H), 7.09-7.06 (m, 1H), 3.86-3.83 (m, 1H), 2.90-2.71 (m, 4H), 2.23-2.17 (m, 1H), 2.07-1.97 (m, 1H), 1.86-1.69 (m, 2H), 1.23 (t, 3H). MS m/z 177.17 (M+1).

### Example 127

### (8S)—N-Propyl-5,6,7,8-tetrahydro-8-quinolinamine

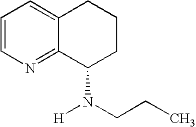

To a solution of (8S)—N-{(1S)-1-[4-(methyloxy)phenyl]ethyl}-5,6,7,8-tetrahydro-8-quinolinamine (300 mg, 1.06 mmol) in dichloroethane (3 mL) was added acetic acid (96 mg, 1.60 mmol), propionaldehyde (123 mg, 2.12 mmol) and sodium triacetoxyborohydride (339 mg, 1.60 mmol). The reaction was stirred for 16 h at room temperature, diluted with saturated aqueous sodium bicarbonate (50 mL), extracted with dichloromethane (50 mL), dried over sodium sulfate, filtered and concentrated. The crude tertiary amine was dissolved in dichloromethane (3 mL) and trifluoroacetic acid (3 mL) and stirred for 16 h at room temperature. The reaction was concentrated, diluted with saturated aqueous sodium bicarbonate (50 mL) and extracted with chloroform/isopropanol (3:1, 3×50 mL), dried over sodium sulfate, filtered and concentrated. The crude amine was purified on silica (0% to 10% 2M NH₃ in methanol/dichloromethane gradient) to provide the product (121 mg, 60%) as a brown oil: ¹H-NMR (CDCl₃) δ 8.39 (d, 1H), 7.36 (d, 1H), 7.07-7.04 (m, 1H), 3.80-3.77 (m, 1H), 2.87-2.64 (m, 4H), 2.19-2.13 (m, 1H), 2.04-1.96 (m, 1H), 1.82-1.68 (m, 2H), 1.67-1.56 (m, 2H), 0.97 (t, 3H). MS m/z 191.17 (M+1).

### Example 128

### (8S)—N-(1-methylethyl)-5,6,7,8-tetrahydro-8-quinolinamine

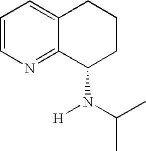

To a solution of (8S)—N-{(1S)-1-[4-(methyloxy)phenyl]ethyl}-5,6,7,8-tetrahydro-8-quinolinamine (25 mg, 0.16 mmol) in dichloroethane (1 mL) was added trifluoroacetic acid (1 mL) and stirred 4 h. The solution was evaporated and purified by column chromatography (0% to 10% 2M NH₃ in methanol/dichloromethane gradient) to provide the product as an oil ¹H-NMR (CDCl₃) δ 8.39 (d, 1H), 7.38 (d, 1H), 7.08 (dd, 1H), 4.07 (t, 1H), 3.50 (br s, 2H), 2.86-2.71 (m, 2H), 2.27-2.16 (m, 1H), 2.19-2.13 (m, 1H), 2.01-1.93 (m, 1H), 1.83-1.69 (m, 2H). MS m/z 149 (M+1). To (8S)-5,6,7,8-tetrahydro-8-quinolinamine was added methanol (1 mL), acetone (25 mL, 0.32 mmol) and trimethyl orthoformate (0.55 mL, 0.5 mmol). The reaction mixture was stirred for 1 h, then sodium borohydride (20 mg, 0.5 mmol) was added slowly and stirred for 1 h. A sat. solution of NaHCO₃ (2 mL) and EtOAc (5 mL) were added and the layers separated. The aqueous layer was extracted with EtOAc (3×5 mL), the layers combined dried over sodium sulfate, filtered, concentrated and purified by column chromatography (0% to 10% 2M NH₃ in methanol/dichloromethane gradient) to provide the product (10 mg, %) as a clear oil: ¹H-NMR (CDCl₃) δ 8.37 (d, 1H), 7.34 (d, 1H), 7.03 (dd, 1H), 4.07 (t, 1H), 3.04 (sept, 1H), 2.85-2.69 (m, 2H), 2.24 (br s, 1H), 2.15-2.09 (m, 1H), 2.01-1.94 (m, 1H), 1.77-1.68 (m, 2H) 1.17 (d, 6H). MS m/z 191.17 (M+1).

### Example 129

### (8S)—N-(Phenylmethyl)-5,6,7,8-tetrahydro-8-quinolinamine

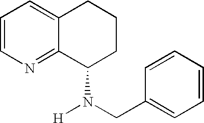

To a solution of (8S)—N-{(1S)-1-[4-(methyloxy)phenyl]ethyl}-5,6,7,8-tetrahydro-8-quinolinamine (300 mg, 1.06 mmol) in dichloroethane (3 mL) was added acetic acid (96 mg, 1.60 mmol), benzaldehyde (225 mg, 2.12 mmol) and sodium triacetoxyborohydride (339 mg, 1.60 mmol). The reaction was stirred for 16 h at room temperature, diluted with saturated aqueous sodium bicarbonate (50 mL), extracted with dichloromethane (50 mL), dried over sodium sulfate, filtered and concentrated. The crude tertiary amine was dissolved in dichloromethane (3 mL) and trifluoroacetic acid (3 mL) and stirred for 16 h at room temperature. The reaction was concentrated, diluted with saturated aqueous sodium bicarbonate (50 mL) and extracted with chloroform/isopropanol (3:1, 3×50 mL), dried over sodium sulfate, filtered and concentrated. The crude amine was purified on silica (0% to 10% 2M NH₃ in methanol/dichloromethane gradient) to provide the product (149 mg, 59%) as a clear oil: ¹H-NMR (CDCl₃) δ 8.39 (d, 1H), 7.42-7.40 (m, 2H), 7.37-7.30 (m, 3H), 7.25-7.21 (m, 1H), 7.07-7.04 (m, 1H), 4.00 (d, 1H), 3.90 (d, 1H), 3.86-3.84 (m, 1H), 2.87-2.71 (m, 2H), 2.23-2.16 (m, 1H), 2.07-1.99 (m, 1H), 1.87-1.70 (m, 2H). MS m/z 239.17 (M+1).

### Example 130

### N-Methyl-N-[(8S)-5,6,7,8-tetrahydro-8-quinolinyl]glycine

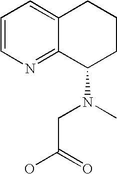

(8S)—N-Ethyl-5,6,7,8-tetrahydro-8-quinolinamine (0.55 g, 3.1 mmol), bromo benzylacetate (0.65 g, 2.8) and N,N-diisopropylethylamine (0.55 mL, 3.1 mmol) were dissolved in CH₂Cl₂ (25 mL). The reaction was stirred for 14 h at room temperature, diluted with sat. NaHCO₃ (10 mL), the layers separated, the aqueous layer extracted with CH₂Cl₂ (3×10 mL), the layers combined, dried over sodium sulfate, filtered and concentrated. Purification by column chromatography (1% to 10% 2M NH₃ in methanol/dichloromethane gradient) afforded an oil. The oil was dissolved in ethanol (25 mL), palladium on carbon (10% w/w, 5.0 mg) was added and the reaction was stirred under a hydrogen atmosphere for 4 h. The reaction was filtered through celite, concentrated, placed under high vacuum to afford a tan solid (0.6 g 82%). ¹H-NMR (DMSO-d₆) δ 8.41 (d, 1H), 7.60 (d, 1H), 7.28 (t, 1H), 4.35 (m, 1H), 3.41 (½ABq, 1H), 3.24 (½ABq, 1H), 2.81-2.74 (m, 2H), 2.44 (s, 3H), 2.12 (m, 1H), 1.95 (m, 1H), 1.82 (m, 1H), 1.70 (m, 1H). MS m/z 221 (M+1).

### Example 131

### N-Ethyl-N-[(8S)-5,6,7,8-tetrahydro-8-quinolinyl]glycine

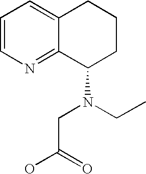

N-Ethyl-N-[(8S)-5,6,7,8-tetrahydro-8-quinolinyl]glycine was prepared in a similar manner to N-methyl-N-[(8S)-5,6,7,8-tetrahydro-8-quinolinyl]glycine from (8S)—N-ethyl-5,6,7,8-tetrahydro-8-quinolinamine (0.55 g, 2.8 mmol) and bromo benzylacetate (0.65 g, 2.8) to afford an oil (0.6 g, 70%). ¹H-NMR (CDCl₃) δ 8.42 (d, 1H), 7.61 (d, 1H), 7.29 (t, 1H), 4.39 (m, 1H), 3.40 (½ABq, 1H), 3.28 (½ABq, 1H), 2.94-2.87 (m, 1H), 2.85-2.66 (m, 3H), 2.54-2.47 (m, 2H), 2.19 (m, 1H), 1.94 (m, 1H), 1.83-1.68 (m, 2H), 1.05 (t, 3H). MS m/z 235 (M+1).

### Example 132

### N-Propyl-N-[(8S)-5,6,7,8-tetrahydro-8-quinolinyl]glycine

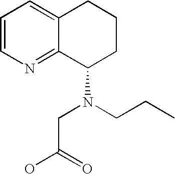

N-Propyl-N-[(8S)-5,6,7,8-tetrahydro-8-quinolinyl]glycine was prepared in a similar manner to N-methyl-N-[(8S)-5,6,7,8-tetrahydro-8-quinolinyl]glycine from (8S)—N-propyl-5,6,7,8-tetrahydro-8-quinolinamine (0.55 g, 2.7 mmol) and bromo benzylacetate (0.65 g, 2.8 mmol) to afford an oil (0.5 g, 53%). ¹H-NMR (CDCl₃) δ 8.40 (d, 1H), 7.60 (d, 1H), 7.28 (t, 1H), 4.29 (m, 1H), 3.53 (ABq, 2H), 2.80-2.70 (m, 2H), 2.54-2.47 (m, 2H), 2.14 (m, 1H), 1.95 (m, 1H), 1.81-1.67 (m, 2H), 1.51-1.40 (m, 2H), 0.79 (t, 3H). MS m/z 249 (M+1).

### Example 133A

### (8R)—N-Methyl-N-{[4-(4-methyl-1-piperazinyl)-1H-benzimidazol-2-yl]methyl}-5,6,7,8-tetrahydro-8-quinolinamine

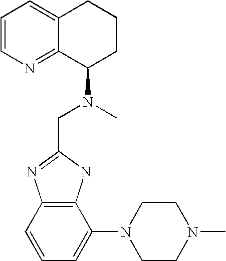

To a solution of ethyl N-methyl-N-[(8R)-5,6,7,8-tetrahydro-8-quinolinyl]glycinate (550 mg, 1.6 mmol) in THF/methanol/water (5 mL/5 mL/5 mL/) was added lithium hydroxide (192 mg, 8 mmol), stirred for 14 h, evaporated under reduced pressure to leave a white solid. (8R)—N-Methyl-N-{[4-(4-methyl-1-piperazinyl)-1H-benzimidazol-2-yl]methyl}-5,6,7,8-tetrahydro-8-quinolinamine was prepared in a similar manner to N-methyl-N-{[5-({4-[2-(1-pyrrolidinyl)ethyl]-1-piperidinyl}carbonyl)-1H-benzimidazol-2-yl]methyl}-5,6,7,8-tetrahydro-8-quinolinamine from the crude acid and 3-(4-methyl-1-piperazinyl)-1,2-benzenediamine (330 mg, 1.6 mmol), bis(2-oxo-3-oxazolidinyl)phosphinic chloride (814 mg, 3.2 mmol) and N,N-diisopropylethylamine (1.1 mL, 6.4 mmol) to yield a white solid (110 mg, 17%): ¹H-NMR (DMSO-d₆): δ 12.2 (br s, 1H), 8.44 (d, 1H), 7.48 (d, 1H), 7.16 (dd, 1H), 7.00-6.93 (m, 2H), 6.43 (m, 1H), 4.04 (ABq, 2H), 3.93 (t, 1H), 3.44 (m, 4H), 2.84-2.74 (m, 1H), 2.67-2.63 (m, 5H), 2.31 (s, 3H), 2.22 (s, 3H), 2.08-2.02 (m, 1H), 1.98-1.89 (m, 2H), 1.68-1.56 (m, 1H). MS m/z 391.1 (M+1).

### Example 133B

### (8S)—N-Methyl-N-{[4-(4-methyl-1-piperazinyl)-1H-benzimidazol-2-yl]methyl}-5,6,7,8-tetrahydro-8-quinolinamine

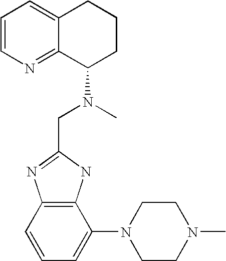

(8S)—N-Methyl-N-{[4-(4-methyl-1-piperazinyl)-1H-benzimidazol-2-yl]methyl}-5,6,7,8-tetrahydro-8-quinolinamine was prepared in a similar fashion as described herein. H-NMR and MS matched that of (8R)—N-Methyl-N-{[4-(4-methyl-1-piperazinyl)-1H-benzimidazol-2-yl]methyl}-5,6,7,8-tetrahydro-8-quinolinamine.

### Example 134

### (8R)—N-Ethyl-N-{[4-(4-methyl-1-piperazinyl)-1H-benzimidazol-2-yl]methyl}-5,6,7,8-tetrahydro-8-quinolinamine

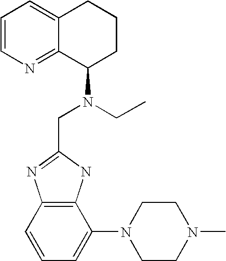

(8R)—N-Ethyl-N-{[4-(4-methyl-1-piperazinyl)-1H-benzimidazol-2-yl]methyl}-5,6,7,8-tetrahydro-8-quinolinamine was prepared in a similar manner to (8R)—N-methyl-N-{[4-(4-methyl-1-piperazinyl)-1H-benzimidazol-2-yl]methyl}-5,6,7,8-tetrahydro-8-quinolinamine from ethyl N-ethyl-N-[(8R)-5,6,7,8-tetrahydro-8-quinolinyl]glycinate (273 mg, 1.0 mmol) to afford a white solid (75 mg, 20%): ¹H-NMR (DMSO-d₆) δ 12.37 (s, 1H), 8.55 (d, 1H), 7.54 (d, 1H), 7.25-7.20 (m, 1H), 7.10 (d, 1H), 7.00 (t, 1H), 6.46 (d, 1H), 4.09-4.03 (m, 2H), 3.94 (d, 1H), 3.52-3.40 (m, 6H), 3.23-3.07 (m, 1H), 2.87-2.62 (m, 5H), 2.26 (s, 3H), 2.20-2.09 (m, 1H), 2.00-1.82 (m, 2H), 1.75-1.61 (m, 1H), 0.97 (t, 3H). MS m/z 405 (M+1).

### Example 135

### (8S)—N-Ethyl-N-{[4-(4-methyl-1-piperazinyl)-1H-benzimidazol-2-yl]methyl}-5,6,7,8-tetrahydro-8-quinolinamine

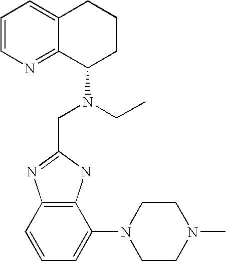

(8S)—N-Ethyl-N-{[4-(4-methyl-1-piperazinyl)-1H-benzimidazol-2-yl]methyl}-5,6,7,8-tetrahydro-8-quinolinamine was prepared in a similar manner as described herein via reductive amination from (8S)—N-ethyl-5,6,7,8-tetrahydro-8-quinolinamine and 4-(4-methyl-1-piperazinyl)-1H-benzimidazole-2-carbaldehyde to afford a white solid (40 mg, 38%). ¹H-NMR and MS data matched that of (8R)—N-ethyl-N-{[4-(4-methyl-1-piperazinyl)-1H-benzimidazol-2-yl]methyl}-5,6,7,8-tetrahydro-8-quinolinamine.

### Example 136

### (8R)—N-{[4-(4-methyl-1-piperazinyl)-1H-benzimidazol-2-yl]methyl}-N-propyl-5,6,7,8-tetrahydro-8-quinolinamine

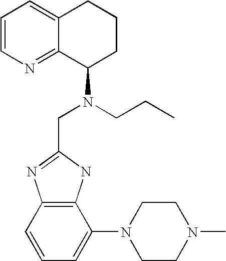

(8R)—N-{[4-(4-methyl-1-piperazinyl)-1H-benzimidazol-2-yl]methyl}-N-propyl-5,6,7,8-tetrahydro-8-quinolinamine was prepared in a similar manner to (8S)—N-(1-methylethyl)-N-{[4-(4-methyl-1-piperazinyl)-1H-benzimidazol-2-yl]methyl}-5,6,7,8-tetrahydro-8-quinolinamine from (8R)—N-propyl-5,6,7,8-tetrahydro-8-quinolinamine (0.05 mg, 0.26 mmol) and 4-(4-methyl-1-piperazinyl)-1H-benzimidazole-2-carbaldehyde (0.1 g, 0.39 mmol) to afford a white solid (25 mg, 23%): ¹H-NMR (DMSO-d6) δ 12.43 (s, 1H), 8.57-8.56 (m, 1H), 7.54 (d, 1H), 7.25-7.21 (m, 1H), 7.10 (d, 1H), 7.00 (t, 1H), 6.46 (d, 1H), 4.13-3.94 (m, 3H), 3.55-3.44 (m, 4H), 2.89-2.60 (m, 8H), 2.27 (s, 3H), 2.23-2.11 (m, 1H), 2.01-1.82 (m, 2H), 1.75-1.58 (m, 1H), 1.42-1.29 (m, 2H), 0.76 (t, 3H). MS m/z 419 (M+1).

### Example 137

### (8S)—N-{[4-(4-methyl-1-piperazinyl)-1H-benzimidazol-2-yl]methyl}-N-propyl-5,6,7,8-tetrahydro-8-quinolinamine

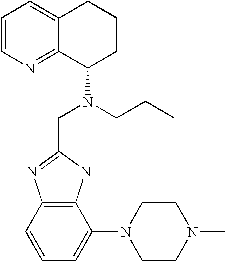

(8S)—N-{[4-(4-methyl-1-piperazinyl)-1H-benzimidazol-2-yl]methyl}-N-propyl-5,6,7,8-tetrahydro-8-quinolinamine was prepared in a similar manner to (8S)—N-(1-methylethyl)-N-{[4-(4-methyl-1-piperazinyl)-1H-benzimidazol-2-yl]methyl}-5,6,7,8-tetrahydro-8-quinolinamine from (8S)—N-propyl-5,6,7,8-tetrahydro-8-quinolinamine (0.025 mg, 0.13 mmol) and 4-(4-methyl-1-piperazinyl)-1H-benzimidazole-2-carbaldehyde (0.05 g, 0.2 mmol) to afford a white solid (16 mg, 29%): ¹H-NMR and MS data matched that of (8R)—N-{[4-(4-methyl-1-piperazinyl)-1H-benzimidazol-2-yl]methyl}-N-propyl-5,6,7,8-tetrahydro-8-quinolinamine.

### Example 138

### (8S)—N-(1-Methylethyl)-N-{[4-(4-methyl-1-piperazinyl)-1H-benzimidazol-2-yl]methyl}-5,6,7,8-tetrahydro-8-quinolinamine

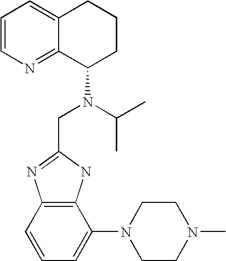

To a solution of (8S)-5,6,7,8-tetrahydro-8-quinolinamine (25 mg, 0.16 mmol) in methanol (3 mL) was added acetone (25 mL, 0.33 mmol) and trimethylorthoformate (55 mL, 0.55 mmol). The reaction was stirred for 1 h at room temperature and sodium borohydride (0.02 g, 0.55 mmol) added. The reaction was stirred 1 h at room temperature, aqueous sodium bicarbonate (2 mL) added and stirred for 0.5 h. The intermediate was evaporated and purified using reverse phase HPLC (0% to 50% acetonitrile/water/0.1% trifluoroacetic acid gradient). The desired fractions were combined, NaCl added until the solution was saturated, neutralized and extracted with EtOAc (5×20 mL). The organic layers combined, dried over Na₂SO₄, filtered and concentrated to provide an oil. The intermediate was dissolved in dichloroethane (5 mL), acetic acid (15 mL, 0.25 mmol) 4-(4-methyl-1-piperazinyl)-1H-benzimidazole-2-carbaldehyde (0.06 g, 0.25 mmol) and sodium triacetoxyborohydride (50 g, 0.25 mmol) were added, the reaction stirred for 12 h and sat. NaHCO₃ (5 mL) added and stirred for 15 min. The aqueous layer was separated and extracted with CH₂Cl₂ (3×5 mL), the organic layers combined, dried over Na₂SO₄, filtered and concentrated. The product was purified as above to afford an off-white solid (20 mg, 28%): ¹H-NMR (d6-DMSO) δ 13.1 (br s, 1H), 8.66 (d, 1H), 7.56 (d, 1H), 7.27 (t, 1H), 7.13 (d, 1H), 6.99 (t, 1H), 6.47 (s, 1H), 4.08 (½ABq, 1H), 4.06 (t, 1H), 3.98 (½ABq, 1H), 3.48 (s, 4H), 3.00 (sep, 1H), 2.85 (m, 1H), 2.70 (m, 1H), 2.58 (s, 4H), 2.30 (s, 3H), 2.13 (m, 1H), 1.94 (m, 2H), 1.68 (m, 1H), 1.11 (d, 3H), 0.98 (d, 3H). MS m/z 419 (M+1).

### Example 139

### (8R)—N-(Cyclopropylmethyl)-N-{[4-(4-methyl-1-piperazinyl)-1H-benzimidazol-2-yl]methyl}-5,6,7,8-tetrahydro-8-quinolinamine

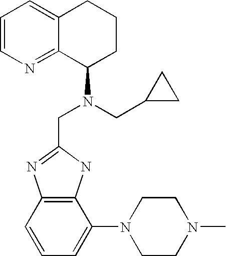

8R)—N-(cyclopropylmethyl)-N-{[4-(4-methyl-1-piperazinyl)-1H-benzimidazol-2-yl]methyl}-5,6,7,8-tetrahydro-8-quinolinamine was prepared in a similar manner to (8R)—N-methyl-N-{[4-(4-methyl-1-piperazinyl)-1H-benzimidazol-2-yl]methyl}-5,6,7,8-tetrahydro-8-quinolinamine from ethyl N-(cyclopropylmethyl)-N-[(8R)-5,6,7,8-tetrahydro-8-quinolinyl]glycinate (650 mg, 2.3 mmol) to afford a white solid (60 mg, 6%): ¹H-NMR (DMSO-d6) δ 8.56-8.55 (m, 1H), 7.55-7.52 (m, 1H), 7.25-7.21 (m, 1H), 7.10 (d, 1H), 7.00 (t, 1H), 6.46 (d, 1H), 4.20-4.00 (m, 3H), 3.55-3.40 (m, 2H), 2.91-2.63 (m, 2H), 2.63-2.56 (m, 8H), 2.34-2.10 (m, 4H), 2.03-1.83 (m, 2H), 1.76-1.55 (m, 1H), 0.90-0.77 (m, 1H), 0.33-0.30 (m, 2H), 0.04-0.02 (m, 2H). MS-m/z 431 (M+1).

### Example 140

### (8S)—N-(Cyclopropylmethyl)-N-{[4-(4-methyl-1-piperazinyl)-1H-benzimidazol-2-yl]methyl}-5,6,7,8-tetrahydro-8-quinolinamine

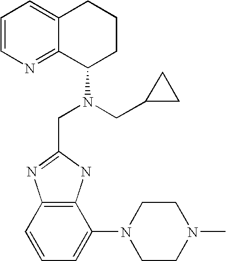

(8S)—N-(cyclopropylmethyl)-N-{[4-(4-methyl-1-piperazinyl)-1H-benzimidazol-2-yl]methyl}-5,6,7,8-tetrahydro-8-quinolinamine was prepared in a similar manner as described herein via reductive amination from (8S)—N-(cyclopropylmethyl)-5,6,7,8-tetrahydro-8-quinolinamine (25 mg, 0.13 mmol) and 4-(4-methyl-1-piperazinyl)-1H-benzimidazole-2-carbaldehyde (0.05 g, 0.2 mmol) to afford a white solid (20 mg, 35%). ¹H-NMR and MS data matched that of (8R)—N-(cyclopropylmethyl)-N-{[4-(4-methyl-1-piperazinyl)-1H-benzimidazol-2-yl]methyl}-5,6,7,8-tetrahydro-8-quinolinamine.

### Example 141

### (8S)—N-({4-[(8aR)-hexahydropyrrolo[1,2-a]pyrazin-2(1H)-yl]-1H-benzimidazol-2-yl}methyl)-N-methyl-5,6,7,8-tetrahydro-8-quinolinamine

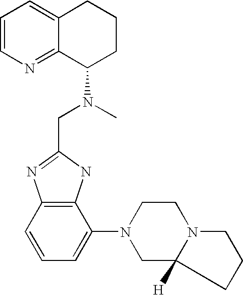

A) 3-Chloro 2-nitroaniline (0.25 g, 1.4 mmol) and (8aR)-octahydropyrrolo[1,2-a]pyrazine (0.5 g, 4.0 mmol) and N,N-diisopropylethylamine (0.75 mL, 4.0 mmol) were dissolved in DMF (2 mL), and the reaction mixture was heated for 4 h at 130° C. The reaction was cooled, concentrated and purified by column chromatography (1% to 10% 2M NH₃ in methanol/dichloromethane gradient) to afford a red solid (0.2 g, 54%): ¹H-NMR (DMSO-d₆) δ 7.09 (t, 1H), 6.50 (d, 1H), 6.36 (d, 1H), 5.83 (br s, 2H), 5.74 (s, 1H), 3.07 (m, 1H), 2.94 (m, 4H), 2.79 (t, 1H), 2.18-1.94 (m, 3H), 1.75-1.60 (m, 4H), 1.26 (m, 1H).

B) 3-[(8aR)-Hexahydropyrrolo[1,2-a]pyrazin-2(1H)-yl]-2-nitroaniline (0.55 g, 0.2 mmol) and palladium on carbon (10% w/w, 5.0 mg) were dissolved in ethanol (10 mL) and the reaction was stirred under a hydrogen atmosphere for 4 h. The reaction was filtered through celite, concentrated, placed under high vacuum and the resulting diamine used crude. This diamine and N-methyl-N-[(8S)-5,6,7,8-tetrahydro-8-quinolinyl]glycine were combined along with acetonitrile, N,N-diisopropylethylamine, bis(2-oxo-3-oxazolidinyl)phosphinic chloride, and stirred for 12 h at room temperature. The reaction was concentrated and diluted with ethyl acetate (200 mL). The organic phase was washed with sat. NaHCO₃ (5 mL), separated and the aqueous phase extracted with ethyl acetate (3×25 mL). The organic extracts were combined, dried over sodium sulfate, filtered and concentrated to a brown liquid. This brown liquid was treated with acetic acid to affect cyclization to benzimidazole in a similar manner as described herein. Concentration and purification by reverse phase HPLC according to the protocol described here-in afforded a orange solid (20 mg, 24%): ¹H-NMR (CD₃OD) δ 8.53 (d, 1H), 7.63 (d, 1H), 7.32-7.28 (m, 1H), 7.23-7.14 (m, 2H), 6.79 (d, 1H), 4.21-4.14 (m, 2H), 4.01-3.80 (m, 3H), 3.28-3.19 (m, 2H), 3.05-2.59 (m, 6H), 2.45-2.37 (m, 1H), 2.25-2.19 (m, 1H), 2.15 (s, 3H), 2.15-1.76 (m, 6H), 1.64-1.54 (m, 1H). MS m/z 417 (M+1).

### Example 142

### (8S)—N-Ethyl-N-({4-[(8aR)-hexahydropyrrolo[1,2-a]pyrazin-2(1H)-yl]-1H-benzimidazol-2-yl}methyl)-5,6,7,8-tetrahydro-8-quinolinamine

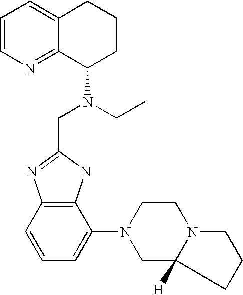

(8S)—N-Ethyl-N-({4-[(8aR)-hexahydropyrrolo[1,2-a]pyrazin-2(1H)-yl]-1H-benzimidazol-2-yl}methyl)-5,6,7,8-tetrahydro-8-quinolinamine was prepared in a similar manner to (8S)—N-({4-[(8aR)-hexahydropyrrolo[1,2-a]pyrazin-2(1H)-yl]-1H-benzimidazol-2-yl}methyl)-N-methyl-5,6,7,8-tetrahydro-8-quinolinamine from 3-[(8aR)-hexahydropyrrolo[1,2-a]pyrazin-2(1H)-yl]-2-nitroaniline (0.55 g, 0.2 mmol) and N-ethyl-N-[(8S)-5,6,7,8-tetrahydro-8-quinolinyl]glycine to afford an orange solid (20 mg, 23%). ¹H-NMR (CD₃OD) δ 8.54 (d, 1H), 7.60 (d, 1H), 7.29-7.13 (m, 3H), 6.78 (d, 1H), 4.20-4.13 (m, 1H), 4.13-3.82 (m, 4H), 3.29-3.20 (m, 2H), 3.06-2.38 (m, 9H), 2.32-2.25 (m, 1H), 2.13-1.89 (m, 5H), 1.81-1.54 (m, 2H), 0.96 (t, 3H). MS m/z 431 (M+1)

### Example 143

### (8S)—N-({4-[(8aR)-hexahydropyrrolo[1,2-a]pyrazin-2(1H)-yl]-1H-benzimidazol-2-yl}methyl)-N-propyl-5,6,7,8-tetrahydro-8-quinolinamine

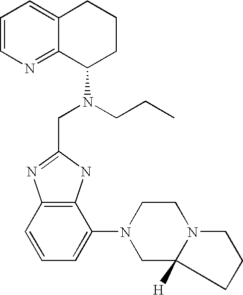

(8S)—N-({4-[(8aR)-hexahydropyrrolo[1,2-a]pyrazin-2(1H)-yl]-1H-benzimidazol-2-yl}methyl)-N-propyl-5,6,7,8-tetrahydro-8-quinolinamine was prepared in a similar manner to (8S)—N-({4-[(8aR)-hexahydropyrrolo[1,2-a]pyrazin-2(1H)-yl]-1H-benzimidazol-2-yl}methyl)-N-methyl-5,6,7,8-tetrahydro-8-quinolinamine to afford a tan solid (50 mg, 56%). ¹H-NMR (CD₃OD) δ 8.55 (d, 1H), 7.61 (d, 1H), 7.27 (dd, 1H), 7.22 (d, 1H), 7.16 (t, 1H), 6.78 (d, 1H), 4.18 (t, 1H), 4.07 (ABq, 2H), 4.06-3.80 (m, 2H), 3.26-3.18 (m, 2H), 3.05-2.53 (m, 7H), 2.44-2.25 (m, 3H), 2.13-1.89 (m, 5H), 1.82-1.72 (m, 1H), 1.63-1.53 (m, 1H), 1.37-1.28 (m, 2H), 0.74 (t, 3H). MS m/z 445 (M+1).

### Example 144

### (8S)—N-methyl-N-({4-[(1R,5R)-7-methyl-3,7-diazabicyclo[3.3.1]non-3-yl]-1H-benzimidazol-2-yl}methyl)-5,6,7,8-tetrahydro-8-quinolinamine

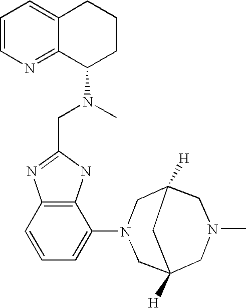

A) 1,1-Dimethylethyl (1S,5S)-7-[2-({methyl[(8S)-5,6,7,8-tetrahydro-8-quinolinyl]amino}methyl)-1H-benzimidazol-4-yl]-3,7-diazabicyclo[3.3.1]nonane-3-carboxylate was prepared in a similar manner as described herein from phenylmethyl N-methyl-N-[(8S)-5,6,7,8-tetrahydro-8-quinolinyl]glycinate (0.055 g, 0.2 mmol) and 1,1-dimethylethyl (1S,5S)-7-(3-amino-2-nitrophenyl)-3,7-diazabicyclo[3.3.1]nonane-3-carboxylate (0.1 g, 0.2 mmol) to yield a tan solid (110 mg, 71%): ¹H-NMR (CD₃OD) 6 ¹H-NMR (CD₃OD) δ 8.54-8.47 (m, 1H), 7.61 (d, 1H), 7.27 (dd, 1H), 7.11 (m, 2H), 6.69 (br s, 1H), 4.43-4.37 (m, 1H), 4.19-4.00 (m, 4H), 3.31-2.82 (m, 5H), 2.38-1.95 (m, 9H), 1.86-1.73 (m, 1H), 1.09 (s, 9H). MS m/z 517 (M+1).

(B) 1,1-Dimethylethyl (1S,5S)-7-[2-({methyl[(8S)-5,6,7,8-tetrahydro-8-quinolinyl]amino}methyl)-1H-benzimidazol-4-yl]-3,7-diazabicyclo[3.3.1]nonane-3-carboxylate (100 mg, 0.2 mmol) was dissolved in dichlormethane (2 mL) and trifluoroacetic acid (2 mL) and stirred for 3 h at room temperature. To the crude amine was added dichloroethane (5 mL), 37% formaldehyde (0.02 mL, 0.30 mmol), acetic acid (0.02 mL, 0.30 mmol) and sodium triacetoxyborohydride (0.60 g, 0.30 mmol). The reaction mixture was stirred 1 h and a solution of sat. NaHCO₃ (2.5 mL) was added and stirred for 15 min. The layers separated and the aqueous layer extracted with CH₂Cl₂ (3×5 mL), the organic layers combined, dried over Na₂SO₄, filtered, concentrated and purified using reverse phase HPLC (0% to 50% acetonitrile/water/0.1% trifluoroacetic acid gradient). The desired fractions were combined, NaCl added until the solution was saturated, neutralized and extracted with EtOAc (5×20 mL). The organic layers combined, dried over Na₂SO₄, filtered and concentrated to provide a white solid (20 mg, 24%). ¹H-NMR (DMSO-d6) δ 8.44-8.43 (m, 1H), 7.49 (d, 1H), 7.19-7.13 (m, 2H), 7.06-7.01 (m, 1H), 6.63-6.53 (m, 1H), 4.12-4.01 (m, 3H), 3.97-3.93 (m, 1H), 3.89-3.86 (m, 1H), 3.67-3.63 (m, 1H), 3.53-3.49 (m, 1H), 3.27-3.24 (m, 2H), 3.11-3.02 (m, 2H), 2.83-2.75 (m, 1H), 2.71-2.63 (m, 2H), 2.26-2.15 (m, 4H), 2.05-1.58 (m, 5H).

### Example 145

### N-Cyclopropyl-N-{[4-(4-methyl-1-piperazinyl)-1H-benzimidazol-2-yl]methyl}-5,6,7,8-tetrahydro-8-quinolinamine

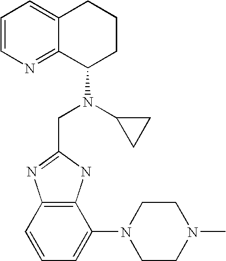

To a solution of N-cyclopropyl-5,6,7,8-tetrahydro-8-quinolinamine (0.075 g, 0.4 mmol) in dichloroethane (5 mL), acetic acid (0.035 mL, 0.6 mmol), 4-(4-methyl-1-piperazinyl)-1H-benzimidazole-2-carbaldehyde (0.15 g, 0.6 mmol) and sodium triacetoxyborohydride (0.13 g, 0.6 mmol) were added, the reaction stirred for 12 h and sat. NaHCO₃ (5 mL) added and stirred for 15 min. The aqueous layer was separated and extracted with CH₂Cl₂ (3×5 mL), the organic layers combined, dried over Na₂SO₄, filtered and concentrated. The product was purified using reverse phase HPLC (0% to 50% acetonitrile/water/0.1% trifluoroacetic acid gradient). The desired fractions were combined, NaCl added until the solution was saturated, neutralized and extracted with EtOAc (5×20 mL). The organic layers combined, dried over Na₂SO₄, filtered and concentrated to provide a white solid (30 mg, 18%): ¹H-NMR (CD₃OD) δ 8.51 (d, 1H), 7.59-7.56 (m, 1H), 7.27-7.12 (m, 3H), 6.76-6.73 (m, 1H), 4.22-4.04 (m, 3H), 3.45-3.36 (m, 4H), 2.99-2.74 (m, 6H), 2.47 (s, 3H), 2.35-2.21 (m, 3H), 2.19-2.05 (m, 1H), 1.89-1.73 (m, 1H), 0.40-0.10 (m, 3H), 0.04-0.11 (m, 1H). MS m/z 417.28 (M+1).

### Example 146

### (8S)—N-{(1S)-1-[4-(methyloxy)phenyl]ethyl}-N-{[4-(4-methyl-1-piperazinyl)-1H-benzimidazol-2-yl]methyl}-5,6,7,8-tetrahydro-8-quinolinamine

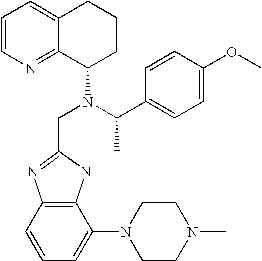

To a solution of (8S)—N-{(1S)-1-[4-(methyloxy)phenyl]ethyl}-5,6,7,8-tetrahydro-8-quinolinamine (0.175 g, 0.6 mmol), 4-(4-methyl-1-piperazinyl)-1H-benzimidazole-2-carbaldehyde (0.1 g, 0.4 mmol) and acetic acid (0.035 mL, 0.6 mmol) were added. Sodium triacetoxyborohydride (0.13 g, 0.6 mmol) was added in two portions over 1 h and the mixture-stirred for 12 h. Saturated NaHCO₃-(25 mL) added and stirred for 15 min. The aqueous layer was separated and extracted with CH₂Cl₂ (3×5 mL), the organic layers combined, dried over Na₂SO₄, filtered and concentrated. Purification by column chromatography (0% to 10% 2M NH₃ in methanol/dichloromethane gradient) afforded a brown oil (0.11 g, 54%). ¹H NMR (400 MHz, DMSO-d₆) d ppm 12.23 (br s, 1H) 8.54 (d, 1H) 7.45 (t, 3H) 7.17 (dd, 1H) 7.07 (d, 1H) 6.94 (t, 1H) 6.81 (d, 2H) 6.39 (d, 1H) 4.69 (dd, 1H) 3.89-3.96 (m, 1H) 3.74-3.90 (m, 2H) 3.68 (s, 3H) 3.32-3.51 (m, 4H) 2.62-2.79 (m, 1H) 2.52-2.62 (m, 1H) 2.41-2.48 (m, 4H) 2.20 (s, 3H) 2.07-2.17 (m, 1H) 1.70-1.90 (m, 2H) 1.35-1.50 (m, 1H) 1.15 (d, 3H). MS m/z 511 (M+1).

### Example 147

### (8S)—N-{[4-(4-Methyl-1-piperazinyl)-1H-benzimidazol-2-yl]methyl}-N-[2-(phenylmethyl)oxy]ethyl}-5,6,7,8-tetrahydro-8-quinolinamine

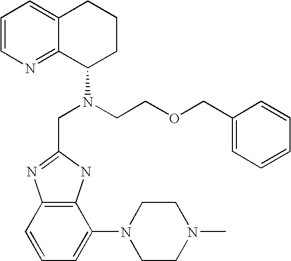

To (8S)—N-{(1S)-1-[4-(methyloxy)phenyl]ethyl}-5,6,7,8-tetrahydro-8-quinolinamine (0.25 g, 0.88 mmol) in dichloroethane (10 mL) was added acetic acid (0.075 mL, 1.3 mmol), [(phenylmethyl)oxy]acetaldehyde (0.135 mL, 1.3 mmol) and sodium triacetoxyborohydride (0.28 g, 1.3 mmol). The reaction was stirred for 12 h at room temperature, diluted with saturated aqueous sodium bicarbonate (10 mL) and extracted with CH₂Cl₂ (3×10 mL). The combined organic extracts were dried over sodium sulfate, filtered and concentrated. The crude tertiary amine was dissolved in dichloromethane (2 mL) and trifluoroacetic acid (2 mL) and stirred for 1 h. The reaction was concentrated and purified by column chromatography (0% to 10% 2M NH₃ in methanol/dichloromethane gradient) to afford the amine. Dichloroethane (5 mL), acetic acid (0.035 mL, 0.6 mmol), 4-(4-methyl-1-piperazinyl)-1H-benzimidazole-2-carbaldehyde (0.15 g, 0.6 mmol) and sodium triacetoxyborohydride (0.13 g, 0.6 mmol) were added, the reaction stirred for 12 h and sat. NaHCO₃ (5 mL) added and stirred for 15 min. The aqueous layer was separated and extracted with CH₂Cl₂ (3×5 mL), the organic layers combined, dried over Na₂SO₄, filtered and concentrated. The product was purified using reverse phase HPLC (0% to 50% acetonitrile/water/0.1% trifluoroacetic acid gradient). The desired fractions were combined, NaCl added until the solution was saturated, neutralized and extracted with EtOAc (5×20 mL). The organic layers combined, dried over Na₂SO₄, filtered and concentrated to provide a white solid (25 mg, 5%). ¹H-NMR (DMSO-d6) δ 12.3 (br s, 1H), 8.54 (d, 1H), 7.54 (d, 1H), 7.31-7.21 (m, 6H), 6.99 (m, 2H), 6.49 (dd, 1H), 4.37 (s, 2H), 4.20 (½ABq, 1H), 4.10 (m, 1H), 4.04 (½ ABq, 1H), 3.47 (m, 6H), 3.37-3.33 (m, 4H), 3.07-2.96 (m, 1H), 2.92-2.71 (m, 3H), 2.28 (s, 3H), 2.21-2.11 (m, 1H), 2.00-1.80 (m, 2H), 1.74-1.60 (m, 1H). MS m/z 511 (M+1).

### Example 148

### 2-{{[4-(4-Methyl-1-piperazinyl)-1H-benzimidazol-2-yl]methyl}[(8S)-5,6,7,8-tetrahydro-8-quinolinyl]amino}ethanol

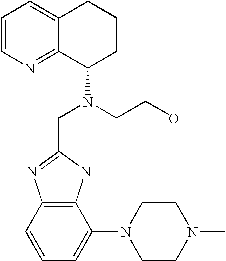

(8S)—N-{(1S)-1-[4-(Methyloxy)phenyl]ethyl}-N-{[4-(4-methyl-1-piperazinyl)-1H-benzimidazol-2-yl]methyl}-5,6,7,8-tetrahydro-8-quinolinamine (0.034 g) was dissolved in CH₂Cl₂ (2 mL) and trifluoroacetic acid (2 mL) and stirred for 4 h. The reaction was concentrated and purified by column chromatography (0% to 10% 2M NH₃ in methanol/dichloromethane gradient) to afford -(8S)—N-{[4-(4-methyl-1-piperazinyl)-1H-benzimidazol-2-yl]methyl}-5,6,7,8-tetrahydro-8-quinolinamine.

A portion of the amine (0.025 g, 0.06 mmol) was dissolved in dichloroethane (2 mL) and {[(1,1-dimethylethyl)(dimethyl)silyl]oxy}acetaldehyde (0.017 g, 0.1 mmol), acetic acid (0.005 mL, 0.1 mmol) and sodium triacetoxyborohydride (0.025 g, 0.1 mmol) were added. The reaction was stirred for 3 h, diluted with saturated aqueous sodium bicarbonate (10 mL) and extracted with CH₂Cl₂ (3×10 mL). The combined organic extracts were dried over sodium sulfate, filtered and concentrated. The crude tertiary amine was dissolved in THF (2 mL) and tetrabutylammonium fluoride (1.0 M, 0.25 mL) and stirred for 0.5 h. The reaction was concentrated and purified using reverse phase HPLC (0% to 50% acetonitrile/water/0.1% trifluoroacetic acid gradient). The desired fractions were lyophilized to provide the trifluoroacetate salt as a white solid (25 mg, 58%). ¹H-NMR (DMSO-d6) δ 9.86 (br s, 1H), 8.64 (d, 1H), 7.88 (d, 1H), 752 (d, 1H), 7.19 (t, 1H), 7.16 (t, 1H), 6.70 (dd, 1H), 4.72 (m, 1H), 4.51 (s, 2H), 4.27-4.16 (m, 2H), 3.70-3.63 (m, 1H), 3.58-3.51 (m, 4H), 3.29-3.18 (m, 3H), 3.04 (dd, 2H), 2.95-2.83 (m, 2H), 2.88 (s, 3H), 2.36 (m, 1H), 2.08-1.89 (m, 2H), 1.82-1.69 (m, 1H). MS m/z 421 (M+1).

### Example 149

### 3-{{[4-(4-Methyl-1-piperazinyl)-1H-benzimidazol-2-yl]methyl}[(8S)-5,6,7,8-tetrahydro-8-quinolinyl]amino}-1-propanol

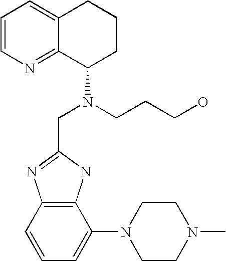

3-{{[4-(4-Methyl-1-piperazinyl)-1H-benzimidazol-2-yl]methyl}[(8S)-5,6,7,8-tetrahydro-8-quinolinyl]amino}-1-propanol was prepared in a similar manner to 2-{{[4-(4-methyl-1-piperazinyl)-1H-benzimidazol-2-yl]methyl}[(8S)-5,6,7,8-tetrahydro-8-quinolinyl]amino}ethanol from (8S)—N-{[4-(4-methyl-1-piperazinyl)-1H-benzimidazol-2-yl]methyl}-5,6,7,8-tetrahydro-8-quinolinamine (0.025 g, 0.06 mmol) and 3-{[(1,1-dimethylethyl)(dimethyl)silyl]oxy}propanal (0.019 g, 0.1 mmol) to afford a white solid (20 mg, 45%). ¹H-NMR (DMSO-d6) δ 9.92 (br s, 1H), 8.53 (d, 1H), 7.82 (d, 1H), 7.46 (d, 1H), 7.18 (t, 1H), 7.15 (t, 1H), 6.67 (dd, 1H), 4.85 (m, 1H), 4.54 (dd, 2H), 4.27 (m, 2H), 3.55 (m, 1H), 3.44 (m, 2H), 3.23 (m, 3H), 3.04 (m, 3H), 2.88 (s, 3H), 2.83 (m, 2H), 2.42-2.35 (m, 1H), 2.09-1.94 (m, 2H), 1.82-1.73 (m, 3H). MS m/z 435 (M+1).

### Example 150

### (8S)—N-{[4-(4-methyl-1-piperazinyl)-1H-benzimidazol-2-yl]methyl}-N-(phenylmethyl)-5,6,7,8-tetrahydro-8-quinolinamine

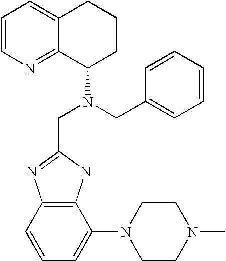

(8S)—N-{[4-(4-methyl-1-piperazinyl)-1H-benzimidazol-2-yl]methyl}-N-(phenylmethyl)-5,6,7,8-tetrahydro-8-quinolinamine was prepared in a similar manner to 2-{{[4-(4-methyl-1-piperazinyl)-1H-benzimidazol-2-yl]methyl}[(8S)-5,6,7,8-tetrahydro-8-quinolinyl]amino}ethanol from (8S)—N-{[4-(4-methyl-1-piperazinyl)-1H-benzimidazol-2-yl]methyl}-5,6,7,8-tetrahydro-8-quinolinamine (0.05 g, 0.12 mmol) and benzaldehyde (0.02 mL, 0.2 mmol) to afford a white solid (50 mg, 60%). ¹H-NMR (DMSO-d6) δ 9.92 (br s, 1H), 8.72 (d, 1H), 7.97 (d, 1H), 7.60 (t, 1H), 7.42 (d, 2H), 7.27-7.15 (m, 6H), 6.75 (d, 1H), 4.44 (m, 1H), 4.34 (½ABq, 1H), 4.22 (½ABq, 1H), 4.02 (m, 2H), 3.91 (ABq, 2H), 3.56 (m, 2H), 3.25 (m, 2H), 3.02 (m, 2H), 2.89 (s, 3H), 2.81 (m, 2H), 2.27 (m, 1H), 2.03 (m, 2H), 1.69 (m, 1H). MS m/z 467 (M+1).

### Example 151

### 1,1-Dimethylethyl 4-{1-methyl-2-[({[(phenylmethyl)oxy]carbonyl}amino)methyl]-1H-benzimidazol-4-yl}-1-piperazinecarboxylate and 1,1-dimethylethyl 4-{1-methyl-2-[({[(phenylmethyl)oxy]carbonyl}amino)methyl]-1H-benzimidazol-7-yl}-1-piperazinecarboxylate

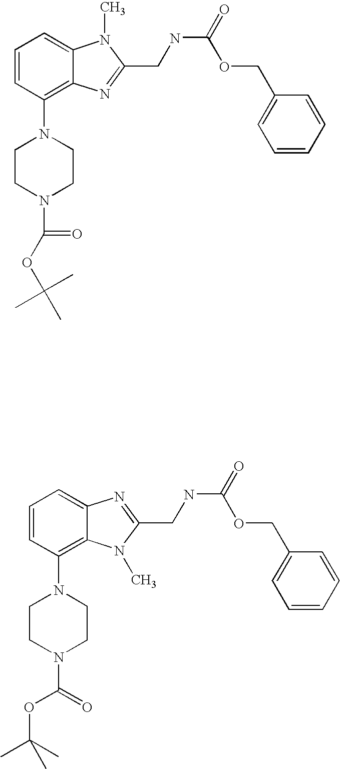

To a solution of 1,1-dimethylethyl 4-{2-[({[(phenylmethyl)oxy]carbonyl}amino)methyl]-1H-benzimidazol-4-yl}-1-piperazinecarboxylate (3.69 g, 7.93 mmol) in N,N-dimethylformamide (50 mL) was added iodomethane (1.35 g, 0.88 mL, 9.52 mmol) and cesium carbonate (3.88 g, 11.9 mmol). The reaction mixture was stirred at room temperature for 2 hours, diluted with ethyl acetate, washed with water and the organic layer was dried over sodium sulfate, filtered, and concentrated. The material was purified on silica (20% to 100% ethyl acetate/hexanes gradient) to provide both isomers as yellow solids.

1,1-Dimethylethyl 4-{1-methyl-2-[({[(phenylmethyl)oxy]carbonyl}amino)methyl]-1H-benzimidazol-4-yl}-1-piperazinecarboxylate (1.20 g, 32%). ¹H-NMR (CDCl₃): δ 7.36-7.31 (m, 5H), 7.19 (t, 1H), 6.94 (d, 1H), 6.64 (d, 1H), 5.14 (s, 2H), 4.66 (d, 2H), 3.75 (s, 3H), 3.69-3.67 (m, 4H), 3.42-3.39 (m, 4H), 1.47 (s, 9H).

1,1-Dimethylethyl 4-{1-methyl-2-[({[(phenylmethyl)oxy]carbonyl}amino)methyl]-1H-benzimidazol-7-yl}-1-piperazinecarboxylate (1.32 g, 36%). ¹H-NMR (CDCl₃) δ 7.49 (d, 1H), 7.34-7.28 (m, 5H), 7.22 (t, 1H), 7.03 (d, 1H), 5.10 (s, 2H), 4.69 (d, 2H), 4.21 (s, 3H), 4.19-4.05 (m, 2H), 3.12-3.06 (m, 4H), 2.89-2.83 (m, 2H) 1.48 (s, 9H).

### Example 152

### N-Methyl-N-{[1-methyl-4-(4-methyl-1-piperazinyl)-1H-benzimidazol-2-yl]methyl}-5,6,7,8-tetrahydro-8-quinolinamine

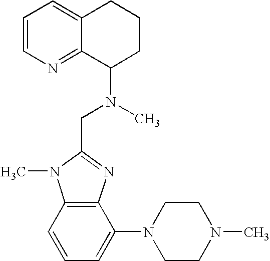

A) A solution 1,1-dimethylethyl 4-{1-methyl-2-[({[(phenylmethyl)oxy]carbonyl}amino)methyl]-1H-benzimidazol-4-yl}-1-piperazinecarboxylate (86 mg, 0.18 mmol) and palladium on carbon (10% w/w, catalytic) in ethanol (50 mL) was stirred under a hydrogen atmosphere for 60 h. The reaction was filtered through celite, and concentrated to provide the primary amine. The crude amine, 6,7-dihydro-8(5H)-quinolinone (26 mg, 0.18 mmol), acetic acid (catalytic), sodium triacetoxyborohydride (57 mg, 0.27 mmol) were dissolved in 1,2-dichloroethane (10 mL) and stirred for 16 h at room temperature. The reaction was diluted with ethyl acetate and saturated sodium bicarbonate, separated, and the organic phase dried over sodium sulfate and concentrated. The crude material was purified using reverse phase HPLC (0% to 75% acetonitrile/water/0.1% trifluoroacetic acid gradient) to provide 1,1-dimethylethyl 4-{1-methyl-2-[(5,6,7,8-tetrahydro-8-quinolinylamino)methyl]-1H-benzimidazol-4-yl}-1-piperazinecarboxylate (30 mg, 28%, clear oil) as the trifluoroacetate salt: ¹H-NMR (CDCl₃) δ 8.61 (d, 1H), 8.05 (d, 1H), 7.67-7.64 (m, 1H), 7.48 (t, 1H), 7.24 (d, 1H), 7.04 (d, 1H), 4.84 (d, 1H), 4.56 (d, 1H), 4.46-4.42 (m, 1H), 4.07 (s, 3H), 3.69-3.67 (m, 4H), 3.14-3.07 (m, 4H), 3.04-3.00 (m, 1H), 2.96-2.89 (m, 1H), 2.41-2.33 (m, 1H), 2.17-2.06 (m, 2H), 1.95-1.85 (m, 1H), 1.48 (s, 9H).

B) 1,1-Dimethylethyl 4-{1-methyl-2-[(5,6,7,8-tetrahydro-8-quinolinylamino)methyl]-1H-benzimidazol-4-yl}-1-piperazinecarboxylate bis(trifluoracetate) (30 mg, 0.05 mmol) was dissolved in dichlormethane (2 mL) and trifluoroacetic acid (2 mL) and stirred for 3 h at room temperature. The reaction was concentrated, dissolved in dichloroethane and formaldehyde (37% aqueous solution, 18 μL, 0.24 mmol) and sodium triacetoxyborohydride (51 mg, 0.24 mmol) were added. The reaction was stirred for 16 h at room temperature, concentrated and purified using reverse phase HPLC (0% to 70% acetonitrile/water/0.1% trifluoroacetic acid gradient) to provide a mixture of product and starting amine. The mixture was dissolved in dichloroethane and formaldehyde (37% aqueous solution, 18 μL, 0.24 mmol) and sodium triacetoxyborohydride (51 mg, 0.24 mmol) were added. The reaction was stirred for 16 h at room temperature, concentrated, purified using reverse phase HPLC (0% to 70% acetonitrile/water/0.1% trifluoroacetic acid gradient). Because the resulting product contained starting material, the amine was dissolved in dichloroethane (3 mL) and formaldehyde (37% aqueous solution, 18 μL, 0.24 mmol) and sodium triacetoxyborohydride (51 mg, 0.24 mmol) were added. The reaction was stirred for 16 h at room temperature, concentrated, purified using reverse phase HPLC (0% to 60% acetonitrile/water/0.1% trifluoroacetic acid gradient) and lyophilized from water to provide the product (23 mg, 62%, light tan solid) as the trifluoroacetate salt: ¹H-NMR (CD₃OD) δ 8.55 (d, 1H), 7.89 (d, 1H), 7.52-7.48 (m, 1H), 7.34-7.30 (m, 1H), 7.25 (d, 1H), 6.85 (d, 1H), 4.83-4.79 (m, 1H), 4.67 (d, 1H), 4.52 (d, 1H), 4.33-4.19 (m, 2H), 3.87 (s, 3H), 3.66-3.61 (m, 2H), 3.42-3.33 (m, 2H), 3.20-3.13 (m, 2H), 2.97-2.78 (m, 5H), 2.78 (s, 3H), 2.49-2.45 (m, 1H), 2.26-2.13 (m, 2H), 1.96-1.84 (m, 1H). MS m/z 405.23 (M+1).

### Example 153

### N-Methyl-N-{[1-methyl-7-(4-methyl-1-piperazinyl)-1H-benzimidazol-2-yl]methyl}-5,6,7,8-tetrahydro-8-quinolinamine

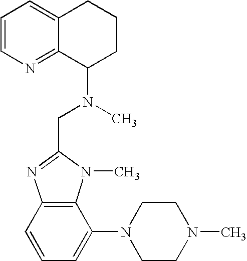

A) 1,1-Dimethylethyl 4-{1-methyl-2-[({[(phenylmethyl)oxy]carbonyl}amino)methyl]-1H-benzimidazol-7-yl}-1-piperazinecarboxylate (100 mg, 0.21 mmol) and palladium on carbon (10% w/w, catalytic) in ethanol (50 mL) was stirred under a hydrogen atmosphere for 16 h. The reaction was filtered through celite, and concentrated to provide the primary amine. The crude amine, 6,7-dihydro-8(5H)-quinolinone (31 mg, 0.21 mmol), acetic acid (catalytic) and sodium triacetoxyborohydride (68 mg, 0.32 mmol) were dissolved in 1,2-dichloroethane (10 mL) and stirred for 16 h at room temperature. The reaction was diluted with ethyl acetate, washed with saturated sodium bicarbonate, separated, and the organic phase dried over sodium sulfate and concentrated. The crude material was using reverse phase HPLC (0% to 70% acetonitrile/water/0.1% trifluoroacetic acid gradient) and lyophilized from water to provide Tert-butyl 4-{1-methyl-2-[(5,6,7,8-tetrahydro-8-quinolinylamino)methyl]-1H-benzimidazol-7-yl}-1-piperazinecarboxylate (79 mg, 53%) as the trifluoroacetate salt: ¹H-NMR (CDCl₃) δ 8.81 (d, 1H), 8.11 (d, 1H), 7.72-7.69 (m, 1H), 7.61-7.52 (m, 3H), 7.38-7.23 (m, 1H), 4.85 (d, 1H), 4.71 (s, 2H), 4.46 (s, 3H), 4.36-4.31 (m, 2H), 3.40-2.85 (m, 8H), 2.51-2.45 (m, 1H), 2.22-2.14 (m, 1H), 2.02-1.87 (m, 2H), 1.49 (s, 9H).

B) Tert-butyl 4-{1-methyl-2-[(5,6,7,8-tetrahydro-8-quinolinylamino)methyl]-1H-benzimidazol-7-yl}-1-piperazinecarboxylate bis(trifluoracetate) was dissolved in dichloromethane (2 mL) and trifluoroacetic acid (2 mL), stirred for 3 h at room temperature and concentrated. The crude-amine, formaldehyde (37% aqueous solution, 49 μL, 0.63 mmol) and sodium triacetoxyborohydride (134 mg, 0.63 mmol) were dissolved in dichloroethane (3 mL) and stirred for 16 h at room temperature. The reaction was concentrated and purified using reverse phase HPLC (0% to 60% acetonitrile/water/0.1% trifluoroacetic acid gradient). The amine was dissolved in dichloroethane (3 mL) and formaldehyde (37% aqueous solution, 49 μL, 0.63 mmol) and sodium triacetoxyborohydride (134 mg, 0.63 mmol) were added. The reaction was stirred for 16 h at room temperature, concentrated, purified using reverse phase HPLC (0% to 60% acetonitrile/water/0.1% trifluoroacetic acid gradient) and lyophilized from water to provide the product (12 mg, 15%, tan solid) as the trifluoroacetate salt: ¹H-NMR (CD₃OD) δ 8.65 (d, 1H), 8.05 (d, 1H), 7.65-7.60 (m, 2H), 7.41 (t, 1H), 7.31 (d, 1H), 4.60-4.56 (m, 1H), 4.52 (d, 1H), 4.38 (d, 1H), 4.23 (s, 3H), 3.68-3.62 (m, 2H), 3.51-3.40 (m, 4H), 3.26-3.19 (m, 2H), 3.01 (s, 3H), 3.00-2.96 (m, 2H), 2.55 (s, 3H), 2.43-2.37 (m, 1H), 2.25-2.09 (m, 4H), 1.95-1.87 (m, 1H). MS m/z 405.26 (M+1).

### Example 154

### N-Ethyl-N-{[1-methyl-7-(4-methyl-1-piperazinyl)-1H-benzimidazol-2-yl]methyl}-5,6,7,8-tetrahydro-8-quinolinamine

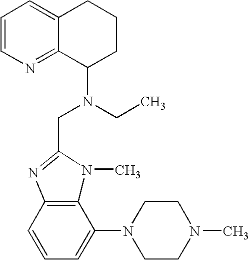

A) 1,1-Dimethylethyl 4-{1-methyl-2-[({[(phenylmethyl)oxy]carbonyl}amino)methyl]-1H-benzimidazol-7-yl}-1-piperazinecarboxylate (Boc protected Phenylmethyl {[1-methyl-7-(4-methyl-1-piperazinyl)-1H-benzimidazol-2-yl]methyl}carbamate) (1.36 g, 2.83 mmol) and palladium on carbon (10% w/w, catalytic) in ethanol (100 mL) was stirred under a hydrogen atmosphere for 16 h. The reaction was filtered through celite, and concentrated to provide the primary amine. The crude amine, 6,7-dihydro-8(5H)-quinolinone (416 mg, 2.83 mmol), acetic acid (255 mg, 4.25 mmol) and sodium triacetoxyborohydride (900 mg, 4.25 mmol) were dissolved in 1,2-dichloroethane (50 mL) and stirred for 16 h at room temperature. The reaction was diluted with dichloromethane (50 mL), washed with saturated sodium bicarbonate (100 mL) and brine (100 mL), separated, and the organic phase dried over sodium sulfate and concentrated. The crude material was purified on silica (1% to 10% 2M NH₃ in methanol/dichloromethane gradient) to provide Tert-butyl 4-{1-methyl-2-[(5,6,7,8-tetrahydro-8-quinolinylamino)methyl]-1H-benzimidazol-7-yl}-1-piperazinecarboxylate (1.09 g, 81%) as a tan foam: ¹H-NMR (CDCl₃) δ 8.37 (d, 1H), 7.49 (d, 1H), 7.38 (d, 1H), 7.13 (t, 1H), 7.09-7.06 (m, 1H), 6.94 (d, 1H), 4.28 (s, 2H), 4.22 (s, 3H), 4.15-4.06 (m, 2H), 3.97-3.93 (m, 1H), 3.14-3.04 (m, 4H), 2.88-2.72 (m, 4H), 2.25-2.17 (m, 1H), 2.05-1.85 (m, 2H), 1.80-1.71 (m, 1H). MS m/z 477.23 (M+1).

B) Tert-butyl 4-{1-methyl-2-[(5,6,7,8-tetrahydro-8-quinolinylamino)methyl]-1H-benzimidazol-7-yl}-1-piperazinecarboxylate (150 mg, 0.31 mmol), acetic acid (28 mg, 0.47 mmol), acetaldehyde (20 mg, 0.47 mmol) and sodium triacetoxyborohydride (100 mg, 0.47 mmol) were dissolved in dichloroethane (3 mL) and stirred for 16 h at room temperature. The reaction was diluted with dichloromethane (20 mL), washed with saturated sodium bicarbonate (20 mL), dried over sodium sulfate, filtered and concentrated. The crude amine dissolved in dichloromethane (2 mL) and trifluoroacetic acid (2 mL), stirred for 3 h at room temperature, concentrated, diluted with dichloromethane (20 mL), reconcentrated and dried under vacuum for 2 h. The crude amine, acetic acid (28 mg, 0.47 mmol), formaldehyde (37% aqueous solution, 35 μL, 0.47 mmol) and sodium triacetoxyborohydride (100 mg, 0.47 mmol) were dissolved in dichloroethane (3 mL) and stirred for 16 h at room temperature. The reaction was concentrated, purified using reverse phase HPLC (0% to 60% acetonitrile/water/0.1% trifluoroacetic acid gradient) and lyophilized from water to provide the product (201 mg, 85%, white solid) as the trifluoroacetate salt: ¹H-NMR (CD₃OD) δ 8.74 (d, 1H), 8.15 (d, 1H), 7.73-7.66 (m, 2H), 7.47 (t, 1H), 7.37 (d, 1H), 4.60-4.50 (m, 2H), 4.38 (d, 1H), 4.27 (s, 3H), 3.69-3.64 (m, 2H), 3.54-3.39 (m, 4H), 3.27-3.20 (m, 2H), 3.02-2.93 (m, 6H), 2.85-2.79 (m, 1H), 2.47-2.41 (m, 1H), 2.25-2.19 (m, 1H), 2.16-2.07 (m, 1H), 1.94-1.85 (m, 1H), 1.18 (t, 3H). MS m/z 419.20 (M+1).

### Example 155

### N-{[1-Methyl-7-(4-methyl-1-piperazinyl)-1H-benzimidazol-2-yl]methyl}-N-propyl-5,6,7,8-tetrahydro-8-quinolinamine

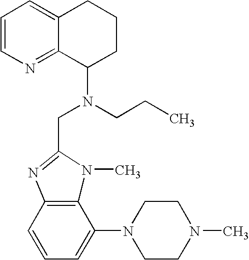

Tert-butyl 4-{1-methyl-2-[(5,6,7,8-tetrahydro-8-quinolinylamino)methyl]-1H-benzimidazol-7-yl}-1-piperazinecarboxylate (150 mg, 0.31 mmol), acetic acid (28 mg, 0.47 mmol), proprionaldehyde (27 mg, 0.47 mmol) and sodium triacetoxyborohydride (100 mg, 0.47 mmol) were dissolved in dichloroethane (3 mL) and stirred for 16 h at room temperature. The reaction was diluted with dichloromethane (20 mL), washed with saturated sodium bicarbonate (20 mL), dried over sodium sulfate, filtered and concentrated. The crude amine dissolved in dichloromethane (2 mL) and trifluoroacetic acid (1 mL), stirred for 3 h at room temperature, concentrated, diluted with dichloromethane (20 mL), reconcentrated and dried under vacuum for 2 h. The crude amine, acetic acid (28 mg, 0.47 mmol), formaldehyde (37% aqueous solution, 35 μL, 0.47 mmol) and sodium triacetoxyborohydride (100 mg, 0.47 mmol) were dissolved in dichloroethane (3 mL) and stirred for 16 h at room temperature. The reaction was concentrated, purified using reverse phase HPLC (0% to 60% acetonitrile/water/0.1% trifluoroacetic acid gradient) and lyophilized from water to provide the product (192 mg, 80%, white solid) as the trifluoroacetate salt: ¹H-NMR (CD₃OD) δ 8.79 (d, 1H), 8.25 (m, 1H), 7.82-7.79 (m, 1H), 7.70 (d, 1H), 7.52 (t, 1H), 7.41 (d, 1H), 4.58-4.51 (m, 2H), 4.36 (d, 1H), 4.30 (s, 3H), 3.68-3.65 (m, 2H), 3.54-3.39 (m, 4H), 3.26-3.23 (m, 2H), 3.05-3.02 (m, 5H), 2.86-2.79 (m, 1H), 2.61-2.53 (m, 1H), 2.49-2.44 (m, 1H), 2.24-2.19 (m, 1H), 2.19-2.15 (m, 1H), 2.06-1.84 (m, 1H), 1.64-1.55 (m, 2H), 0.85 (t, 3H). MS m/z 433.27 (M+1).

### Example 156

### N-(Cyclopropylmethyl)-N-{[1-methyl-7-(4-methyl-1-piperazinyl)-1H-benzimidazol-2-yl]methyl}-5,6,7,8-tetrahydro-8-quinolinamine

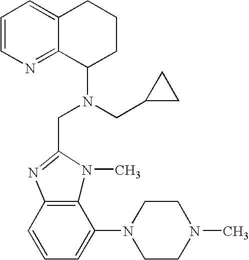

Tert-butyl 4-{1-methyl-2-[(5,6,7,8-tetrahydro-8-quinolinylamino)methyl]-1H-benzimidazol-7-yl}-1-piperazinecarboxylate (150 mg, 0.31 mmol), acetic acid (28 mg, 0.47 mmol), cyclopropylcarboxaldehyde (33 mg, 0.47 mmol) and sodium triacetoxyborohydride (100 mg, 0.47 mmol) were dissolved in dichloroethane (3 mL) and stirred for 16 h at room temperature. The reaction was diluted with dichloromethane (20 mL), washed with saturated sodium bicarbonate (20 mL), dried over sodium sulfate, filtered and concentrated. The crude amine dissolved in dichloromethane (2 mL) and trifluoroacetic acid (1 mL), stirred for 3 h at room temperature, concentrated, diluted with dichloromethane (20 mL), reconcentrated and dried under vacuum for 2 h. The crude amine, acetic acid (28 mg, 0.47 mmol), formaldehyde (37% aqueous solution, 35 μL, 0.47 mmol) and sodium triacetoxyborohydride (100 mg, 0.47 mmol) were dissolved in dichloroethane (3 mL) and stirred for 16 h at room temperature. The reaction was concentrated, purified using reverse phase HPLC (0% to 60% acetonitrile/water/0.1% trifluoroacetic acid gradient) and lyophilized from water to provide the product as the trifluoroacetate salt (164 mg, 67%, white solid): ¹H-NMR (CD₃OD) δ 8.78 (d, 1H), 8.24 (d, 1H), 7.80-7.77 (m, 1H), 7.70 (d, 1H), 7.52 (t, 1H), 7.41 (d, 1H), 4.74-4.70 (m, 1H), 4.57-4.44 (m, 2H), 4.32 (s, 3H), 3.68-3.65 (m, 2H), 3.52-3.38 (m, 4H), 3.28-3.19 (m, 2H), 3.05-3.01 (m, 5H), 2.77-2.72 (m, 1H), 2.62-2.57 (m, 1H), 2.47-2.41 (m, 1H), 2.24-2.19 (m, 1H), 2.15-2.06 (m, 1H), 1.96-1.85 (m, 1H), 0.98-0.97 (m, 1H), 0.38-0.35 (m, 2H), 0.20-0.09 (m, 2H). MS m/z 445.24 (M+1).

### Example 157

### Tert-butyl 4-{2-[(acetyloxy)methyl]-1H-benzimidazol-4-yl}-1-piperazinecarboxylate

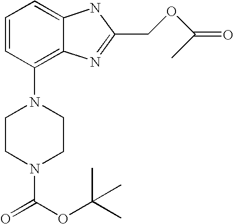

A) 3-Chloro 2-nitroaniline (1.0 g, 5.8 mmol) and 1-boc-piperazine (5.0 g, 26.8 mmol) were dissolved in N,N-diisopropylethylamine (2.25 g, 3.0 mL, 17.4 mmol) and the reaction mixture was heated for 16 h at 130° C. The reaction was cooled, concentrated and purified on silica (10% to 40% ethylacetate/hexanes gradient) to provide tert-butyl 4-(3-amino-2-nitrophenyl)-1-piperazinecarboxylate (1.58 g, 85%) as a red solid: ¹H-NMR (DMSO-d₆) δ 7.10 (t, 1H), 6.55 (d, 1H), 6.38 (d, 1H), 5.85 (br s, 2H), 3.34-3.32 (m, 4H), 2.79-2.77 (m, 4H), 1.38 (s, 9H).

B) Tert-butyl 4-(3-amino-2-nitrophenyl)-1-piperazinecarboxylate (21.5 g, 66.7 mmol) and palladium on carbon (10% w/w, catalytic) were dissolved in ethanol (500 mL) and the reaction was stirred under a hydrogen atmosphere for 16 h. The reaction was filtered through celite, concentrated and purified on silica (2% to 20% 2 M NH₃ in methanol/dichloromethane gradient) to provide tert-butyl 4-(2,3-diaminophenyl)-1-piperazinecarboxylate (17.5 g, 90%) as a brown solid: ¹H-NMR (DMSO-d₆) δ 6.39-6.32 (m, 2H), 6.30-6.27 (m, 1H), 4.44 (br s, 2H), 4.18 (br s, 2H), 3.48-3.41 (m, 4H), 2.70-2.62 (m, 4H), 9.28 (s, 9H).

C) Tert-butyl 4-(2,3-diaminophenyl)-1-piperazinecarboxylate (17.0 g, 58.2 mmol), acetoxyacetic acid (6.36 g, 53.9 mmol), bis(2-oxo-3-oxazolidinyl)phosphinic chloride (19.1 g, 74.9 mmol), and N,N-diisopropylethylamine (11.6 g, 89.9 mmol) were dissolved in acetonitrile (300 mL) and the reaction was stirred for 2 h at room temperature. The reaction was concentrated to 150 mL and diluted with ethyl acetate (200 mL). The organic phase was washed with water (200 mL) and the aqueous phase was extracted with ethyl acetate (200 mL) and chloroform/isopropanol (3:1, 300 mL). The organic extracts were combined, dried over sodium sulfate, filtered and concentrated to a brown solid. The crude-amide was dissolved in acetic acid (100 mL), heated at 70° C. for 2 h, cooled and concentrated. The crude material was diluted with dichloromethane (200 mL) and washed with saturated sodium bicarbonate (200 mL). The aqueous phase was extracted with dichloromethane (100 mL) and the organic extracts were combined, dried over sodium sulfate, filtered and concentrated to a red oily foam. The crude acetate was purified on silica (1% to 10% 2M NH₃ in methanol/dichloromethane gradient) to provide the product (17.10 g, 78%) as a red foam: ¹H-NMR (DMSO-d₆) δ 12.44 (s, 1H), 7.07-6.98 (m, 2H), 6.49 (d, 1H), 5.20 (s, 2H), 3.55-3.36 (m, 8H), 2.09 (s, 3H), 1.40 (s, 9H).

### Example 158

### Tert-butyl 4-{2-[(acetyloxy)methyl]-1-methyl-1H-benzimidazol-4-yl}-1-piperazinecarboxylate and Tert-butyl 4-{2-[(acetyloxy)methyl]-1-methyl-1H-benzimidazol-7-yl}-1-piperazinecarboxylate

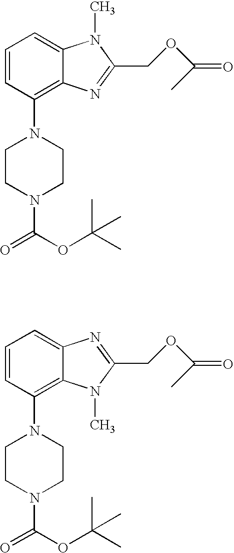

A slurry of tert-butyl 4-{2-[(acetyloxy)methyl]-1H-benzimidazol-4-yl}-1-piperazinecarboxylate (1.16 g, 3.10 mmol), cesium carbonate (1.52 g, 4.65 mmol) and iodomethane (528 mg, 3.72 mmol) in N,N-dimethylformamide (20 mL) was stirred at room temperature for 2 h, dilute with ethyl acetate and water and the phases separated. The organic phase was dried over sodium sulfate, filtered, concentrated and purified on silica (0% to 100% ethyl acetate/hexanes gradient) to provide the products as red solids. Structural assignments confirmed by H-nOe: Tert-butyl-4-{2-[(acetyloxy)methyl]-1-methyl-1H-benzimidazol-4-yl}-1-piperazinecarboxylate (570 mg, 48%). ¹H-NMR (CDCl₃): δ 7.20 (t, 1H), 6.94 (d, 1H), 6.65-6.60 (d, 1H), 5.35 (s, 2H), 3.20 (s, 3H), 3.69-3.67 (m, 4H), 3.48-3.46 (m, 4H), 2.11 (s, 3H), 1.47 (s, 9H). MS m/z 389 (M+1).

Tert-butyl 4-{2-[(acetyloxy)methyl]-1-methyl-1H-benzimidazol-7-yl}-1-piperazinecarboxylate (494 mg, 41%). ¹H-NMR (CDCl₃) δ 7.55 (d, 1H), 7.19 (t, 1H), 7.02 (d, 1H), 5.36 (s, 2H), 4.19-4.10 (m, 5H), 3.14-3.05 (m, 4H), 2.90-2.83 (m, 2H), 2.14 (s, 3H), 1.48 (s, 9H). MS m/z 389 (M+1).

### Example 159

### (8S)—N-methyl-N-{[1-methyl-7-(1-piperazinyl)-1H-benzimidazol-2-yl]methyl}-5,6,7,8-tetrahydro-8-quinolinamine

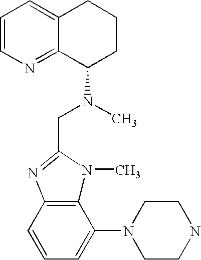

A) To a solution of tert-butyl 4-{2-[(acetyloxy)methyl]-1-methyl-1H-benzimidazol-7-yl}-1-piperazinecarboxylate (3.8 g, 9.8 mmol) in methanol (100 mL) was added cesium carbonate (catalytic). The reaction was stirred at room temperature for 2 h, concentrated and purified on silica (0% to 10% 2M NH₃ in methanol/dichloromethane gradient) to provide tert-butyl 4-[2-(hydroxymethyl)-1-methyl-1H-benzimidazol-7-yl]-1-piperazinecarboxylate (3.2 g, 95%) as a light tan foam: ¹H-NMR (DMSO-d₆): δ 7.30 (d, 1H), 7.05 (t, 1H), 6.94 (d, 1H), 5.49 (t, 1H), 4.66 (d, 2H), 4.10 (s, 3H), 4.02-3.92 (m, 2H), 3.14-3.05 (m, 4H), 2.74-2.67 (m, 2H), 1.41 (s, 9H). MS m/z 347 (M+1).

B) A slurry of tert-butyl 4-[2-(hydroxymethyl)-1-methyl-1H-benzimidazol-7-yl]-1-piperazinecarboxylate (1.0 g, 2.9 mmol) and manganese dioxide (85% w/w, 2.95 g, 29 mmol) in acetonitrile (200 mL) was stirred for 16 h at room temperature. The reaction was heated to 60° C. and filtered through celite. The celite pad was rinsed with an additional portion of hot acetonitrile (200 mL). The filtrate was concentrated to provide tert-butyl 4-(2-formyl-1-methyl-1H-benzimidazol-7-yl)-1-piperazinecarboxylate (820 mg, 82%) as an orange foam: ¹H-NMR (CDCl₃): δ 10.11 (s, 1H), 7.67 (d, 1H), 7.29 (t, 1H), 7.11 (d, 1H), 4.51 (s, 3H), 4.20-4.12 (m, 2H), 3.18-3.10 (m, 4H), 2.90-2.83 (m, 2H), 1.50 (s, 9H). MS m/z 345 (M+1).

C) To a solution of tert-butyl 4-(2-formyl-1-methyl-1H-benzimidazol-7-yl)-1-piperazinecarboxylate (165 mg, 0.48 mmol) in dichloroethane (10 mL) was added (8S)—N-methyl-5,6,7,8-tetrahydro-8-quinolinamine (89 mg, 0.55 mmol), acetic acid (43 mg, 0.72 mmol) and sodium triacetoxyborohydride (153 mg, 0.72 mmol). The reaction was stirred at room temperature for 2 h, washed with saturated sodium bicarbonate, dried over sodium sulfate, filtered and concentrated. The crude material was purified on silica (1% to 10% 2M NH₃ in methanol/dichloromethane gradient) to provide tert-butyl 4-[1-methyl-2-({methyl[(8S)-5,6,7,8-tetrahydro-8-quinolinyl]amino}methyl)-1H-benzimidazol-7-yl]-1-piperazinecarboxylate (194 mg, 82%): ¹H-NMR (CD₃OD) δ 8.36 (d, 1H), 7.46 (d, 1H), 7.30 (d, 1H), 7.14-7.08 (m, 2H), 6.99 (m, 1H), 4.16 (s, 3H), 4.14-4.06 (m, 2H), 4.03-3.97 (m, 2H), 3.90 (d, 1H), 3.29 (s, 3H), 3.25-3.12 (m, 4H), 2.91-2.70 (m, 4H), 2.25 (s, 3H), 2.15-2.03 (m, 3H), 1.77-1.66 (m, 1H), 1.48 (s, 9H). MS m/z 491.9 (M+1).

D) A solution of tert-butyl 4-[1-methyl-2-({methyl[(8S)-5,6,7,8-tetrahydro-8-quinolinyl]amino}methyl)-1H-benzimidazol-7-yl]-1-piperazinecarboxylate (184 mg, 0.38 mmol) in dichloromethane (5 mL) and trifluoroacetic acid (2 mL) was stirred for 2 h at room temperature. The reaction was concentrated to afford the trifluoroacetate salt: ¹H-NMR (CD₃OD) δ 8.76 (d, 1H), 8.29 (d, 1H), 7.85-7.81 (m, 1H), 7.68 (d, 1H), 7.54 (t, 1H), 7.43 (d, 1H), 4.60-4.53 (m, 2H), 4.38 (d, 1H), 4.32 (s, 3H), 3.55-3.41 (m, 6H), 3.28-3.21 (m, 2H), 3.07-3.01 (m, 2H), 2.47 (s, 3H), 2.44-2.39 (m, 1H), 2.30-2.08 (m, 2H), 2.00-1.90 (m, 1H). MS m/z 391 (M+1).

### Example 160

### (8S)—N-Methyl-N-{[1-methyl-7-(4-methyl-1-piperazinyl)-1H-benzimidazol-2-yl]methyl}-5,6,7,8-tetrahydro-8-quinolinamine

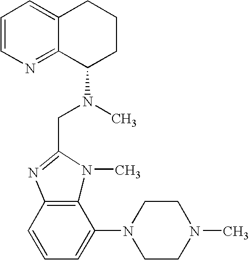

C) (8S)—N-methyl-N-{[1-methyl-7-(1-piperazinyl)-1H-benzimidazol-2-yl]methyl}-5,6,7,8-tetrahydro-8-quinolinamine trifluoroacetate salt (260 mg, 0.35 mmol), acetic acid (34 mg, 0.57 mmol), formaldehyde (37% aqueous solution, 0.043 mL, 0.57 mmol) and sodium triacetoxyborohydride (121 mg, 0.57 mmol) were dissolved in dichloroethane (5 mL) and stirred for 3 h at room temperature, concentrated, neutralized with 2M NH₃ in methanol, concentrated and purified on silica (0% to 10% 2M NH₃ in methanol/dichloromethane gradient) to provide the product (89 mg, 63%) as a white solid. Structural assignment confirmed by NMR: ¹H-NMR (CD₃OD) δ 8.37 (d, 1H), 7.48 (d, 1H), 7.31 (d, 1H), 7.16-7.10 (m, 2H), 7.03 (d, 1H), 4.16 (s, 3H), 4.04-3.99 (m, 2H), 3.91 (d, 1H), 3.17-3.09 (m, 2H), 3.05-2.85 (m, 5H), 2.77-2.70 (m, 1H), 2.51-2.43 (m, 2H), 2.40 (s, 3H), 2.27 (s, 3H), 2.17-2.06 (m, 3H), 1.77-1.68 (m, 1H). ¹³C-NMR (DMSO-d₆) δ 157, 153, 146, 143, 140, 136, 134, 130, 122, 121, 115, 113, 62, 54, 53, 52, 45, 31, 28, 26, 21. MS m/z 405 (M+1). HRMS calcd for C24H32N6 (M+1): 405.2761. Found: 405.2760. Anal. Calcd for C24H31N6.0.37H20: C, 70.10; H, 8.02; N, 20.44; O, 1.44. Found: C, 70.18; H, 8.03; N, 20.42.

### Example 161

### (8R)—N-Methyl-N-{[1-methyl-7-(4-methyl-1-piperazinyl)-1H-benzimidazol-2-yl]methyl}-5,6,7,8-tetrahydro-8-quinolinamine

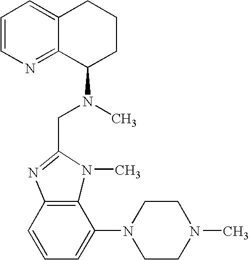

A) To a solution of tert-butyl 4-(2-formyl-1-methyl-1H-benzimidazol-7-yl)-1-piperazinecarboxylate (50 mg, 0.15 mmol) in dichloroethane (1 mL) was added (8R)—N-{(1R)-1-[4-(methyloxy)phenyl]ethyl}-5,6,7,8-tetrahydro-8-quinolinamine (49 mg, 0.17 mmol), acetic acid (14 mg, 0.23 mmol) and sodium triacetoxyborohydride (49 mg, 0.23 mmol). The reaction was stirred at room temperature for 16 h, diluted with dichloromethane, washed with saturated sodium bicarbonate and concentrated. The crude material was purified on silica (0% to 10% 2M NH₃ in methanol/dichloromethane gradient) to provide tert-butyl 4-[1-methyl-2-({{(1R)-1-[4-(methyloxy)phenyl]ethyl}[(8R)-5,6,7,8-tetrahydro-8-quinolinyl]amino}methyl)-1H-benzimidazol-7-yl]-1-piperazinecarboxylate (30 mg, 41%): ¹H-NMR (CD₃OD) δ 8.80 (d, 1H), 8.28-8.24 (m, 1H), 7.82-7.76 (m, 1H), 7.57-7.54 (m, 1H), 7.45 (t, 1H), 7.31-7.26 (m, 3H), 6.45 (d, 1H), 5.01 (m, 1H), 4.26 (d, 1H), 4.18-4.06 (m, 3H), 3.98-3.94 (m, 1H), 3.92 (s, 3H), 3.38 (s, 3H), 3.26-2.71 (m, 8H), 2.63-2.55 (m, 1H), 2.29-2.12 (m, 2H), 2.03-1.90 (m, 1H), 1.69 (d, 3H), 1.49 (s, 9H).

B) A solution of tert-butyl 4-[1-methyl-2-({{(1R)-1-[4-(methyloxy)phenyl]ethyl}[(8R)-5,6,7,8-tetrahydro-8-quinolinyl]amino}methyl)-1H-benzimidazol-7-yl]-1-piperazinecarboxylate (30 mg, 0.05 mmol) in dichloromethane (2 mL) and trifluoroacetic acid (1 mL) was stirred for 2 h at room temperature. The reaction was concentrated and dried under vacuum for 2 h. The crude amine, formaldehyde (37% aqueous solution, 15 μL, 0.20 mmol) and sodium triacetoxyborohydride (32 mg, 0.20 mmol) were dissolved in dichloroethane (1 mL) and stirred for 60 h at room temperature. Additional portions of formaldehyde (37% aqueous solution, 15 μL, 0.20 mmol) and sodium triacetoxyborohydride (32 mg, 0.20 mmol) were added and the reaction was stirred for 16 h at room temperature. The reaction was concentrated, purified using reverse phase HPLC (0% to 50% acetonitrile/water/0.1% trifluoroacetic acid gradient) and lyophilized from water to provide the product as the trifluoroacetate salt (8.8 mg, 24%, white solid): ¹H-NMR (CD₃OD) δ 8.74 (d, 1H), 8.24 (d, -1H), 7.79 (t, 1H), 7.67 (d, 1H), 7.51 (t, 1H), 7.41 (d, 1H), 4.61-4.57 (m, 1H), 4.54 (d, 1H), 4.39 (d, 1H), 4.30 (s, 3H), 3.68-3.65 (m, 2H), 3.52-3.44 (m, 4H), 3.28-3.24 (m, 2H), 3.03-3.01 (m, 5H), 2.49 (s, 3H), 2.44-2.39 (m, 1H), 2.26-2.09 (m, 2H), 1.99-1.88 (m, 1H). MS m/z 405.40 (M+1).

### Example 162

### [4-(4-Methyl-1-piperazinyl)-1H-benzimidazol-2-yl]methyl acetate

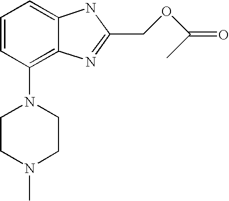

A) 3-Chloro 2-nitroaniline (4.0 g, 23.2 mmol) was dissolved in 1-methyl piperazine (20 mL) and the reaction mixture was heated for 2 h at 140° C. The reaction was cooled, concentrated and purified on silica (5% to 20% 2 M NH₃ in methanol/dichloromethane) to provide 3-(4-methyl-1-piperazinyl)-2-nitroaniline (5.40 g, 99%) as a red solid: ¹H-NMR (CD₃OD) δ 7.13 (t, 1H), 6.56 (d, 1H), 6.45 (d, 1H), 2.99-2.96 (m, 4H), 2.55-2.53 (m, 4H), 2.32 (s, 3H).

B) 3-(4-Methyl-1-piperazinyl)-2-nitroaniline (5.40 g, 23.0 mmol) and palladium on carbon (10% w/w, catalytic) were dissolved in ethanol (200 mL) and the reaction was stirred under a hydrogen atmosphere for 60 h. The reaction was filtered through celite, concentrated to provide 3-(4-methyl-1-piperazinyl)-1,2-benzenediamine (4.60 g, 97%) as a brown solid: ¹H-NMR (CD₃OD) δ 6.59-6.50 (m, 3H), 2.88-2.84 (m, 4H), 2.71-2.54 (m, 4H), 2.34 (s, 3H).

C) 3-(4-Methyl-1-piperazinyl)-1,2-benzenediamine (2.40 g, 11.6 mmol), acetoxyacetic acid (1.23 g, 10.4 mmol), bis(2-oxo-3-oxazolidinyl)phosphinic chloride (3.53 g, 13.9 mmol), and N,N-diisopropylethylamine (1.80 g, 13.9 mmol) were dissolved in acetonitrile (30 mL) and the reaction was stirred for 2 h at room temperature. The reaction was concentrated and the crude amide was dissolved in acetic acid (30 mL), heated at 70° C. for 2 h, cooled and concentrated. The crude material was diluted with dichloromethane and 2 M NH₃ in methanol. The resulting precipitate was filtered off and the filtrate was concentrated. The crude acetate was purified on silica (0% to 10% 2M NH₃ in methanol/dichloromethane gradient) to provide the product (2.60 g, 78%) as a red foam: ¹H-NMR (CD₃OD) δ 7.22 (t, 1H), 7.12 (d, 1-H), 6.70 (d, 1H), 5.37 (s, 2H), 3.49-3.38 (m, 4H), 2.73-2.71 (m, 4H), 2.37 (s, 3H), 2.11 (s, 3H). MS m/z 289.12 (M+1).

### Example 163

### [1-ethyl-7-(4-methyl-1-piperazinyl)-1H-benzimidazol-2-yl]methyl acetate and [1-ethyl-4-(4-methyl-1-piperazinyl)-1H-benzimidazol-2-yl]methyl acetate

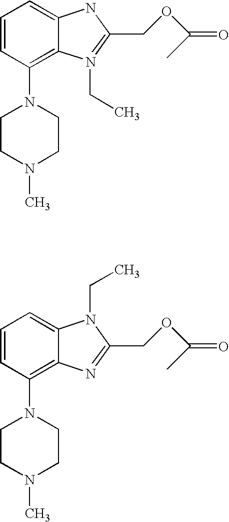

A slurry of [4-(4-methyl-1-piperazinyl)-1H-benzimidazol-2-yl]methyl acetate (1.13 g, 3.92 mmol), cesium carbonate (1.92 g, 5.88 mmol) and iodoethane (733 mg, 4.70 mmol) in N,N-dimethylformamide (10 mL) was stirred at room temperature for 2 h, dilute with water (50 mL), washed with ethyl acetate (3×50 mL) and the phases separated. The organic phase was dried over sodium sulfate, filtered, concentrated and purified on silica (0% to 20% 2 M NH₃ in methanol/dichloromethane gradient) to provide the products as white solids:

[1-ethyl-4-(4-methyl-1-piperazinyl)-1H-benzimidazol-2-yl]methyl acetate (240 mg, 19%). ¹H-NMR (CD₃OD): δ 7.45-7.43 (m, 1H), 7.24-7.22 (m, 2H), 5.38 (s, 2H), 4.59 (q, 2H), 3.11-3.08 (m, 4H), 2.98-2.95 (m, 2H), 2.47-2.41 (m, 2H), 2.40 (s, 3H), 2.14 (s, 3H), 1.41 (t, 3H).

[1-ethyl-7-(4-methyl-1-piperazinyl)-1H-benzimidazol-2-yl]methyl acetate (460 mg, 37%). ¹H-NMR (CD₃OD) δ 7.22 (t, 3H), 7.12 (d, 1H), 6.70 (d, 1H), 5.37 (s, 2H), 4.30 (q, 2H), 3.48-3.38 (m, 4H), 2.73-2.71 (m, 4H), 2.37 (s, 3H), 2.11 (s, 3H), 1.42 (t, 3H).

### Example 164

### (8S)—N-{[1-ethyl-7-(4-methyl-1-piperazinyl)-1H-benzimidazol-2-yl]methyl}-N-methyl-5,6,7,8-tetrahydro-8-quinolinamine

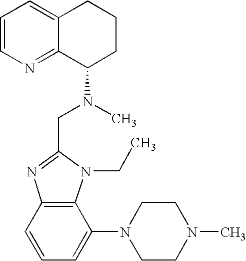

A) To a solution of [1-ethyl-7-(4-methyl-1-piperazinyl)-1H-benzimidazol-2-yl]methyl acetate (150 mg, 0.48 mmol) in methanol (5 mL) was added cesium carbonate (catalytic). The reaction was stirred at room temperature for 2 h, concentrated and purified on silica (0% to 10% 2M NH₃ in methanol/dichloromethane gradient) to provide the primary alcohol. The alcohol and manganese dioxide (85% w/w, 484 mg, 4.83 mmol) in acetonitrile (10 mL) was for 16 h at room temperature. The reaction was heated to 60° C. and filtered through celite. The celite pad was rinsed with an additional portion of hot acetonitrile and the filtrate was concentrated to provide 1-ethyl-7-(4-methyl-1-piperazinyl)-1H-benzimidazole-2-carbaldehyde (97 mg, 74%) as a white solid: ¹H-NMR (CDCl₃): 10.10 (s, 1H), 7.70 (d, 1H), 7.34-7.28 (m, 2H), 5.05 (q, 2H), 3.18-3.04 (m, 4H), 3.00-2.89 (m, 2H), 2.48-2.30 (m, 5H), 1.40 (t, 3H). MS m/z 273.19 (M+1).

B) A solution of 1-ethyl-7-(4-methyl-1-piperazinyl)-1H-benzimidazole-2-carbaldehyde (24 mg, 0.09 mmol), (8S)—N-methyl-5,6,7,8-tetrahydro-8-quinolinamine (29 mg, 0.18 mmol), acetic acid (catalytic), and sodium triacetoxyborohydride (29 mg, 0.14 mmol) in dichloroethane (1 mL) was stirred for 3 h at room temperature. The reaction was concentrated, purified using reverse phase HPLC (0% to 40% acetonitrile/water/0.1% trifluoroacetic acid gradient) and lyophilized from water to provide the product as the trifluoroacetate salt (22.8 mg, 34%, white solid): ¹H-NMR (CD₃OD) δ 8.80 (d, 1H), 8.36 (d, 1H), 7.91-7.88 (m, 1H), 7.78 (d, 1H), 7.64-7.58 (m, 2H), 4.81-4.74 (m, 2H), 4.63-4.57 (m, 2H), 4.45 (d, 1H), 3.70-3.65 (m, 2H), 3.45-3.34 (m, 6H), 3.07-3.04 (m, 5H), 2.50-2.42 (m, 4H), 2.28-2.21 (m, 1H), 2.18-2.09 (m, 1H), 2.00-1.90 (m, 1H), 1.53 (t, 3H). MS m/z 419.23 (M+1).

### Example 165

### (8S)—N-{[1-Ethyl-7-(4-methyl-1-piperazinyl)-1H-benzimidazol-2-yl]methyl}-N-(Phenylmethyl)-5,6,7,8-tetrahydro-8-quinolinamine

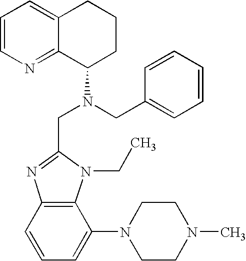

A solution of 1-ethyl-7-(4-methyl-1-piperazinyl)-1H-benzimidazole-2-carbaldehyde (24 mg, 0.09 mmol), (8S)—N-(phenylmethyl)-5,6,7,8-tetrahydro-8-quinolinamine (43 mg, 0.18 mmol), acetic acid (catalytic), and sodium triacetoxyborohydride (30 mg, 0.14 mmol) in dichloroethane (1 mL) was stirred for 16 h at room temperature. The reaction was concentrated, purified using reverse phase HPLC (0% to 40% acetonitrile/water/0.1% trifluoroacetic acid gradient) and lyophilized from water to provide the product as the trifluoroacetate salt (13 mg, 17%, white solid): ¹H-NMR (CD₃OD) δ 8.88 (d, 1H), 8.19 (d, 1H), 7.79-7.76 (m, 1H), 7.69 (d, 1H), 7.54-7.45 (m, 2H), 7.34 (d, 2H), 7.10 (t, 2H), 6.99 (d, 1H), 4.64-4.57 (m, 4H), 4.40 (d, 1H), 3.85 (q, 2H), 3.70-3.64 (m, 2H), 3.40-3.25 (m, 6H), 3.03 (s, 3H), 3.02-2.97 (m, 2H), 2.52-2.44 (m, 1H), 2.36-2.17 (m, 2H), 2.15 (s, 3H), 1.91-1.86 (m, 1H), 1.39 (t, 3H). MS m/z 517.29 (M+Na).

### Example 166

### (8S)—N-Ethyl-N-{[1-ethyl-7-(4-methyl-1-piperazinyl)-1H-benzimidazol-2-yl]methyl}-5,6,7,8-tetrahydro-8-quinolinamine

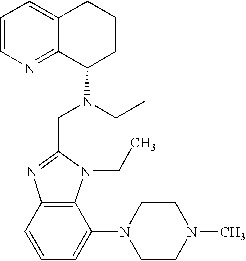

A solution of 1-ethyl-7-(4-methyl-1-piperazinyl)-1H-benzimidazole-2-carbaldehyde (24 mg, 0.09 mmol), (8S)—N-(ethyl)-5,6,7,8-tetrahydro-8-quinolinamine (43 32 mg, 0.18 mmol), acetic acid (catalytic), and sodium triacetoxyborohydride (30 mg, 0.14 mmol) in dichloroethane (1 mL) was stirred for 16 h at room temperature. The reaction was concentrated, purified using reverse phase HPLC (0% to 40% acetonitrile/water/0.1% trifluoroacetic acid gradient) and lyophilized from water to provide the product as the trifluoroacetate salt (1.8 mg, 3%, white solid): ¹H-NMR (CD₃OD) δ 8.77 (d, 1H), 8.21 (d, 1H), 7.79-7.75 (m, 2H), 7.55-7.49 (m, 2H), 4.76-4.67 (m, 2H), 4.60-4.55 (m, 2H), 4.42 (d, 1H), 3.71-3.66 (m, 2H), 3.62-3.34 (m, 7H), 3.04-2.91 (m, 5H), 2.84-2.72 (m, 1H), 2.47-2.42 (m, 1H), 2.25-2.19 (m, 1H), 2.15-2.10 (m, 1H), 1.98-1.86 (m, 1H), 1.49 (t, 3H), 1.15 (t, 3H). MS m/z 455.27 (M+Na).

### Example 167

### (8S)—N-Ethyl-N-{[1-methyl-7-(4-methyl-1-piperazinyl)-1H-benzimidazol-2-yl]methyl}-5,6,7,8-tetrahydro-8-quinolinamine

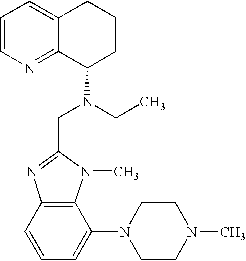

A) To a solution of tert-butyl 4-(2-formyl-1-methyl-1H-benzimidazol-7-yl)-1-piperazinecarboxylate (60 mg, 0.17 mmol), (8S)—N-ethyl-5,6,7,8-tetrahydro-8-quinolinamine (61 mg, 0.34 mmol) and acetic acid (31 mg, 0.51 mmol)) in dichloroethane (2 mL) was added sodium triacetoxyborohydride (108 mg, 0.51 mmol) portionwise over 30 min. The reaction was stirred at room temperature for 16 h, concentrated and purified on silica (0% to 10% 2M NH₃ in methanol/dichloromethane gradient) to provide tert-butyl 4-[2-({ethyl[(8S)-5,6,7,8-tetrahydro-8-quinolinyl]amino}methyl)-1-methyl-1H-benzimidazol-7-yl]-1-piperazinecarboxylate (82 mg, 96%): ¹H-NMR (CD₃OD) 8.39 (d, 1H), 7.46 (d, 1H), 7.38-7.33 (m, 1H), 7.17-7.11 (m, 2H), 7.04 (t, 1H), 4.22 (s, 3H), 4.17-4.09 (m, 5H), 3.22-3.08 (m, 4H), 2.90-2.68 (m, 6H), 2.20-2.03 (m, 3H), 1.76-1.57 (m, 1H), 1.49 (s, 9H), 1.04 (t, 3H).

B) A solution of tert-butyl 4-[2-({ethyl[(8S)-5,6,7,8-tetrahydro-8-quinolinyl]amino}methyl)-1-methyl-1H-benzimidazol-7-yl]-1-piperazinecarboxylate (82 mg, 0.16 mmol) in dichloromethane (2 mL) and trifluoroacetic acid (2 mL) was stirred for 2 h at room temperature. The reaction was concentrated, the crude material was dissolved

In dichloroethane (2 mL) and formaldehyde (37% aqueous solution, 26 μL, 0.34 mmol) and sodium triacetoxyborohydride (72 mg, 0.34 mmol) were added. The reaction was stirred for 16 h at room temperature, concentrated, and purified using reverse phase HPLC (0% to 40% acetonitrile/water/0.1% trifluoroacetic acid gradient) and lyophilized from water to provide the product as the trifluoroacetate salt (53 mg, 41%, light tan solid): ¹H-NMR (CD₃OD) δ 8.78 (d, 1H), 8.27 (d, 1H), 7.83-7.80 (m, 1H), 7.71 (d, 1H), 7.53 (t, 1H), 7.42 (d, 1H), 4.61-4.50 (m, 2H), 4.35 (d, 1H), 4.30 (s, 3H), 3.68-3.65 (m, 2H), 3.52-3.42 (m, 4H), 3.25-3.21 (m, 2H), 3.02-2.98 (m, 5H), 2.96-2.89 (m, 1H), 2.77-2.71 (m, 1H), 2.49-2.43 (m, 1H), 2.25-2.21 (m, 1H), 2.14-2.04 (m, 1H), 1.96-1.85 (m, 1H), 1.18 (t, 3H). MS m/z 419.30 (M+1).

### Example 168

### (8S)—N-{[1-Methyl-7-(4-methyl-1-piperazinyl)-1H-benzimidazol-2-yl]methyl}-N-propyl-5,6,7,8-tetrahydro-8-quinolinamine

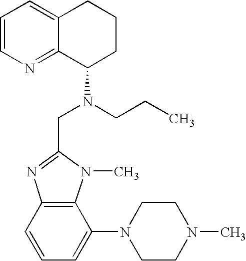

A) To a solution of tert-butyl 4-(2-formyl-1-methyl-1H-benzimidazol-7-yl)-1-piperazinecarboxylate (60 mg, 0.17 mmol), (8S)—N-propyl-5,6,7,8-tetrahydro-8-quinolinamine (65 mg, 0.34 mmol) and acetic acid (31 mg, 0.51 mmol)) in dichloroethane (2 mL) was added sodium triacetoxyborohydride (108 mg, 0.51 mmol) portionwise over 30 min. The reaction was stirred at room temperature for 16 h, concentrated and purified on silica (0% to 10% 2M NH₃ in methanol/dichloromethane gradient) to provide tert-butyl 4-[2-({propyl[(8S)-5,6,7,8-tetrahydro-8-quinolinyl]amino}methyl)-1-methyl-1H-benzimidazol-7-yl]-1-piperazinecarboxylate (121 mg, >99%): ¹H-NMR (CD₃OD) 8.36 (d, 1H), 7.40 (d, 1H), 7.31 (d, 1H), 7.13-7.06 (m, 2H), 7.00 (d, 1H), 4.25 (s, 3H), 4.16-4.04 (m, 5H), 3.26-3.08 (m, 4H), 2.91-2.66 (m, 4H), 2.60-2.55 (m, 2H), 2.17-2.00 (m, 3H), 1.73-1.61 (m, 1H), 1.49 (s, 9H), 1.41-1.28 (m, 2H), 0.74 (t, 3H).

B) A solution of tert-butyl 4-[2-({propyl[(8S)-5,6,7,8-tetrahydro-8-quinolinyl]amino}methyl)-1-methyl-1H-benzimidazol-7-yl]-1-piperazinecarboxylate (88 mg, 0.17 mmol) in dichloromethane (2 mL) and trifluoroacetic acid (2 mL) was stirred for 2 h at room temperature. The reaction was concentrated, the crude material was dissolved In dichloroethane (2 mL) and formaldehyde (37% aqueous solution, 26 μL, 0.34 mmol) and sodium triacetoxyborohydride (72 mg, 0.34 mmol) were added. The reaction was stirred for 16 h at room temperature, concentrated, and purified using reverse phase HPLC (0% to 40% acetonitrile/water/0.1% trifluoroacetic acid gradient) and lyophilized from water to provide the product as the trifluoroacetate salt (8 mg, 6%, light tan solid): ¹H-NMR (CD₃OD) δ 8.77 (d, 1H), 8.22 (d, 1H), 7.79-7.76 (m, 1H), 7.69 (d, 1H), 7.50 (t, 1H), 7.39 (d, 1H), 4.55-4.51 (m, 2H), 4.33-4.28 (m, 1H), 4.28 (s, 3H), 3.67-3.63 (m, 2H), 3.51-3.41 (m, 4H), 3.03-2.98 (m, 4H), 2.86-2.77 (m, 1H), 2.64 (s, 3H), 2.61-2.52 (m, 1H), 2.47-2.40 (m, 1H), 2.23-2.05 (m, 2H), 1.93-1.82 (m, 1H), 1.62-1.53 (m, 2H), 0.84 (t, 3H). MS m/z 433.31 (M+1).

### Example 169

### (8S)—N-{[1-Methyl-7-(4-methyl-1-piperazinyl)-1H-benzimidazol-2-yl]methyl}-N-(Phenylmethyl)-5,6,7,8-tetrahydro-8-quinolinamine

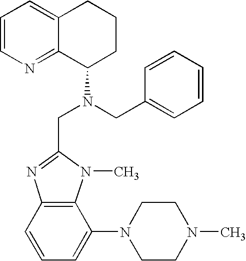

To a solution of tert-butyl 4-(2-formyl-1-methyl-1H-benzimidazol-7-yl)-1-piperazinecarboxylate (60 mg, 0.17 mmol), (8S)—N-(phenylmethyl)-5,6,7,8-tetrahydro-8-quinolinamine (81 mg, 0.34 mmol) and acetic acid (31 mg, 0.51 mmol)) in dichloroethane (2 mL) was added sodium triacetoxyborohydride (108 mg, 0.51 mmol) portionwise over 30 min. The reaction was stirred at room temperature for 16 h, concentrated and purified on silica (0% to 10% 2M NH₃ in methanol/dichloromethane gradient) to provide a mixture of the product (1,1-dimethylethyl 4-[1-methyl-2-({(phenylmethyl)[(8S)-5,6,7,8-tetrahydro-8-quinolinyl]amino}methyl)-1H-benzimidazol-7-yl]-1-piperazinecarboxylate) and the starting amine. A solution of the mixture in dichloromethane (2 mL) and trifluoroacetic acid (2 mL) was stirred for 2 h at room temperature. The reaction was concentrated, the crude material was dissolved In dichloroethane (2 mL) and formaldehyde (37% aqueous solution, 26 μL, 0.34 mmol) and sodium triacetoxyborohydride (72 mg, 0.34 mmol) were added. The reaction was stirred for 16 h at room temperature, concentrated, and purified using reverse phase HPLC (0% to 40% acetonitrile/water/0.1% trifluoroacetic acid gradient) and lyophilized from water to provide the product as the trifluoroacetate salt (30 mg, 21%, light tan solid): ¹H-NMR (CD₃OD) δ 8.86 (d, 1H), 8.16 (d, 1H), 7.76-7.73 (m, 1H), 7.60 (d, 1H), 7.46 (t, 1H), 7.35-7.32 (m, 3H), 7.09 (t, 2H), 6.96 (d, 1H), 4.64-4.59 (m, 1H) m 4.55 (d, 1H), 4.35 (d, 1H), 4.17 (s, 3H), 3.84 (q, 2H), 3.67-3.64 (m, 2H), 3.64-3.19 (m, 6H), 3.02 (s, 3H), 3.02-2.94 (m, 2H), 2.51-2.43 (m, 1H), 2.33-2.18 (m, 2H), 1.93-1.82 (m, 1H). MS m/z 481.31 (M+1).

### Example 170

### N-{[5-Chloro-4-(4-methyl-1-piperazinyl)-1H-benzimidazol-2-yl]methyl}-N-methyl-5,6,7,8-tetrahydro-8-quinolinamine

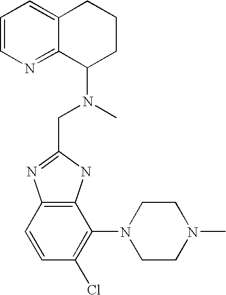

### a) 4-Chloro-3-(4-methyl-1-piperazinyl)-2-nitroaniline

To a stirred solution of 3-(4-methyl-1-piperazinyl)-2-nitroaniline (0.50 g, 2.1 mmol) in 30 mL of isopropanol at 60° C. was added N-chlorosuccinimide (0.31 g, 2.3 mmol). The resulting solution was heated to reflux for 20 minutes and then cooled to RT. After concentrating the solution to dryness at reduced pressure, the residue was dissolved in EtOAc. The solution was washed with aqueous brine (2×), dried over Na₂SO₄, and concentrated to dryness by rotary evaporation. The crude product was subjected to flash chromatography (silica gel, gradient of EtOAc to 8:2 EtOAc:MeOH) to afford 0.29 g (51%) of 4-chloro-3-(4-methyl-1-piperazinyl)-2-nitroaniline as the earlier eluting component of a two compound mixture. ¹H-NMR (CDCl₃) δ 7.24 (d, 1H), 6.50 (d, 1H), 4.62 (br s, 2H), 3.19 (br s, 4H), 2.57 (br s, 4H), 2.38 (s, 3H). MS m/z 271 (M+1).

### b) 4-Chloro-3-(4-methyl-1-piperazinyl)-1,2-benzenediamine

To a stirred mixture of 4-chloro-3-(4-methyl-1-piperazinyl)-2-nitroaniline (0.12 g, 0.45 mmol) and anhydrous nickel (II) chloride (0.12 g, 0.89 mmol) in 12 mL of anhydrous EtOH was added sodium borohydride (0.10 g, 2.7 mmol). After stirring at RT for 30 minutes the black mixture was filtered through celite to remove solids and the filtrate concentrated to dryness at reduced pressure. The residue was subjected to flash chromatography (silica gel, gradient of dichloromethane to 9:1 dichloromethane:2M NH₃ in MeOH) to afford 84 mg (74%) of 4-chloro-3-(4-methyl-1-piperazinyl)-1,2-benzenediamine. ¹H-NMR (CDCl₃) δ6.59 (d, 1H), 6.48 (d, 1H), 4.12 (br s, 2H), 3.78-3.69 (m, 2H), 3.34 (br s, 2H), 2.88 (d, 2H), 2.73 (d, 2H), 2.38 (s, 3H), 2.28-2.17 (m, 2H). MS m/z 241 (M+1).

### c) N-{[5-Chloro-4-(4-methyl-1-piperazinyl)-1H-benzimidazol-2-yl]methyl}-N-methyl-5,6,7,8-tetrahydro-8-quinolinamine

In a manner similar to the procedure described herein for the synthesis of (8R)—N-methyl-N-{[4-(4-methyl-1-piperazinyl)-1H-benzimidazol-2-yl]methyl}-5,6,7,8-tetrahydro-8-quinolinamine, 4-chloro-3-(4-methyl-1-piperazinyl)-1,2-benzenediamine (0.12 g, 0.50 mmol) was subjected to BOP-chloride mediated coupling with N-methyl-1-N-(5,6,7,8-tetrahydro-8-quinolinyl)glycine (0.17 g, 0.75 mmol) followed by acetic acid induced cyclization to afford, following reverse phase HPLC purification (C8, gradient elution of H₂O/0.1% TFA to 100% MeCN over 40 minutes), 94 mg (44%) of N-{[5-chloro-4-(4-methyl-1-piperazinyl)-1H-benzimidazol-2-yl]methyl}-N-methyl-5,6,7,8-tetrahydro-8-quinolinamine as a light yellow foam. ¹H-NMR (DMSO-d₆, 100° C.): δ 12.16 (br s, 1H), 8.50 (d, 1H), 7.49 (d, 1H), 7.23-7.14 (m, 2H), 7.10 (d, 1H), 4.08-3.90 (m, 3H), 3.45-3.34 (m, 4H), 2.83 (m, 1H), 2.71 (m, 1H), 2.58-2.46 (m, 4H), 2.34 (s, 3H), 2.29 (s, 3H), 2.10-1.88 (m, 3H), 1.68 (m, 1H). MS m/z 425 (M+1).

### Example 171

### N-{[4-Chloro-7-(4-methyl-1-piperazinyl)-1H-benzimidazol-2-yl]methyl}-N-methyl-5,6,7,8-tetrahydro-8-quinolinamine

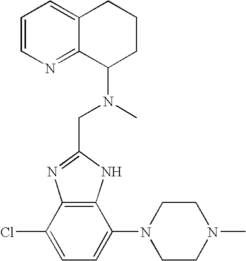

### a) 6-Chloro-3-(4-methyl-1-piperazinyl)-2-nitroaniline

To a stirred solution of 3-(4-methyl-1-piperazinyl)-2-nitroaniline (0.50 g, 2.1 mmol) in 30 mL of isopropanol at 60° C. was added N-chlorosuccinimide (0.31 g, 2.3 mmol). The resulting solution was heated to reflux for 20 minutes and then cooled to RT. After concentrating the solution to dryness at reduced pressure, the residue was dissolved in EtOAc. The solution was washed with aqueous brine (2×), dried over Na₂SO₄, and concentrated to dryness by rotary evaporation. The crude product was subjected to flash chromatography (silica gel, gradient of EtOAc to 8:2 EtOAc:MeOH) to afford 0.15 g (26%) of 6-chloro-3-(4-methyl-1-piperazinyl)-2-nitroaniline as the later eluting component of a two compound mixture. ¹H-NMR (CDCl₃) δ 7.31 (d, 1H), 6.44 (d, 1H), 5.19 (br s, 2H), 3.10-3.02 (m, 4H), 2.63-2.52 (m, 4H), 2.38 (s, 3H). MS m/z 271 (M+1).

### b) N-{[4-Chloro-7-(4-methyl-1-piperazinyl)-1H-benzimidazol-2-yl]methyl}-N-methyl-5,6,7,8-tetrahydro-8-quinolinamine

In a manner similar to the sequence described herein for the synthesis of N-{[5-chloro-4-(4-methyl-1-piperazinyl)-1H-benzimidazol-2-yl]methyl}-N-methyl-5,6,7,8-tetrahydro-8-quinolinamine, 6-chloro-3-(4-methyl-1-piperazinyl)-2-nitroaniline (0.15 g, 0.54 mmol) was converted, in three steps, to N-{[4-chloro-7-(4-methyl-1-piperazinyl)-1H-benzimidazol-2-yl]methyl}-N-methyl-5,6,7,8-tetrahydro-8-quinolinamine which was obtained as a light yellow foam in 12% overall yield. ¹H-NMR (CD₃OD): δ 8.51 (d, 1H), 7.58 (d, 1H), 7.26 (dd, 1H), 7.12 (d, 1H), 6.68 (d, 1H), 4.20-4.11 (m, 2H), 3.96 (d, 1H), 3.38-3.24 (br s, 4H), 2.93-2.68 (m, 6H), 2.39 (s, 3H), 2.20 (m, 1H), 2.11-1.90 (m, 5H), 1.76 (m, 1H). MS m/z 425 (M+1).

### Example 172

### N-Methyl-N-{(1R)-1-[4-(4-methyl-1-piperazinyl)-1H-benzimidazol-2-yl]ethyl}-5,6,7,8-tetrahydro-8-quinolinamine

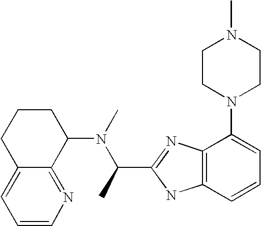

N-Methyl-N-{(1R)-1-[4-(4-methyl-1-piperazinyl)-1H-benzimidazol-2-yl]ethyl}-5,6,7,8-tetrahydro-8-quinolinamine was prepared in a similar manner to N-(1-methylethyl)-N-{[4-(4-methyl-1-piperazinyl)-1H-benzimidazol-2-yl]methyl}-5,6,7,8-tetrahydro-8-quinolinamine as a mixture of diastereomers. The R and S isomers of the undefined stereogenic center can be separated using chiral chromatography or by supercritical fluid chromatography, SFC conditions: Chiralpcel OD-H (3 cm), 1500 psi, 27 deg C., 2 ml/min, 20% methanol (0.5% DIPEA):

Isomer #1: ¹H-NMR (DMSO-d₆) δ 9.96 (br s, 1H), 8.62 (d, 1H), 7.94 (d, 1H), 7.53 (dd, 1H), 7.23 (m, 2H), 6.71 (dd, 1H), 5.03 (dd, 1H), 4.83 (dd, 1H), 4.48 (d, 1H), 4.30 (d, 1H), 3.58 (m, 2H), 3.29 (m, 2H), 3.20-3.01 (m, 2H), 2.93 (s, 3H), 2.83 (m, 2H), 2.68 (s, 3H), 2.11-1.92 (m, 2H), 1.88-1.72 (m, 2H), 1.84 (d, 3H). MS m/z 405 (M+1).

Isomer #2: ¹H-NMR (DMSO-d₆) δ 10.2 (br s, 1H), 8.62 (d, 1H), 7.85 (d, 1H), 7.60 (dd, 1H), 7.25 (m, 2H), 6.77 (d, 1H), 5.02 (dd, 1H), 4.86 (dd, 1H), 4.23 (t, 2H), 3.60 (m, 2H), 3.28 (m, 2H), 3.11 (m, 2H), 2.93 (s, 3H), 2.86 (m, 2H), 2.55 (s, 3H), 2.16 (m, 1H), 2.10-1.98 (m, 2H), 1.89-1.81 (m, 2H), 1.80 (d, 3H). MS m/z 405 (M+1).

### Example 173

### (2R)-2-[{[4-(methyloxy)phenyl]methyl}(5,6,7,8-tetrahydro-8-quinolinyl)amino]-2-[4-(4-methyl-1-piperazinyl)-1H-benzimidazol-2-yl]ethanol

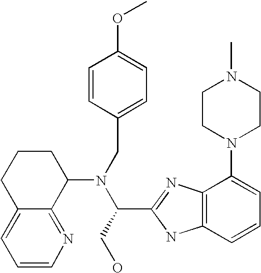

Phenylmethyl N-{[4-(methyloxy)phenyl]methyl}-N-(5,6,7,8-tetrahydro-8-quinolinyl)-L-serinate was prepared in a similar manner to N-(1-methylethyl)-N-{[4-(4-methyl-1-piperazinyl)-1H-benzimidazol-2-yl]methyl}-5,6,7,8-tetrahydro-8-quinolinamine by sequential reductive aminations of 6,7-dihydro-8(5H)-quinolinone with (L)-serine benzyl ester and p-anisaldehyde to an oil as a mixture of diastereomers. The intermediate was carried forward in a similar manner to N-(1-methylethyl)-N-{[4-(4-methyl-1-piperazinyl)-1H-benzimidazol-2-yl]methyl}-5,6,7,8-tetrahydro-8-quinolinamine via reduction of the benzyl ester, coupling with 3-(4-methyl-1-piperazinyl)-1,2-benzenediamine and acid catalyzed cyclization to afford phenylmethyl N-{[4-(methyloxy)phenyl]methyl}-N-(5,6,7,8-tetrahydro-8-quinolinyl)-L-serinate as a trifluoroacetate salt: ¹H-NMR (CD₃OD) δ 8.77 (d, 1H), 7.99 (d, 1H), 7.64 (dd, 1H), 7.44 (d, 1H), 7.36 (t, 1H), 7.04 (m, 3H), 6.59 (d, 2H), 4.54 (t, 1H), 4.27 (m, 2H), 3.89 (d, 1H), 3.76 (m, 3H), 3.66 (m, 2H), 3.58 (s, 3H), 3.46-3.33 (m, 2H), 3.21-3.11 (m, 3H), 3.01 (s, 3H), 2.87 (m, 2H), 2.37-2.30 (m, 2H), 2.16-2.08 (m, 1H), 1.92-1.78 (m, 1H). MS m/z 527 (M+1).

### Example 174

### (2R)-2-[4-(4-methyl-1-piperazinyl)-1H-benzimidazol-2-yl]-2-(5,6,7,8-tetrahydro-8-quinolinylamino)ethanol

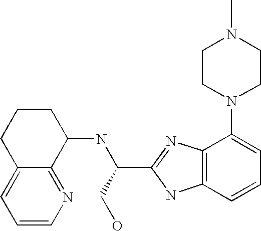

Phenylmethyl N-{[4-(methyloxy)phenyl]methyl}-N-(5,6,7,8-tetrahydro-8-quinolinyl)-L-serinate (0.07 g, 0.13 mmol) was dissolved in methylene chloride (1 ml) and trifluoracetic acid (1 ml) added and the mixture stirred for 4 h. The solvent evaporated and reaction mixture purified by RPHPLC and the desired fraction neutralized as described here within to afford a white solid (19 mg, 5%): ¹H-NMR (CD₃OD) δ 8.37 (d, 1H), 7.50 (d, 1H), 7.18 (t, 1H), 7.10 (d, 2H), 6.68 (s, 1H), 4.35 (t, 1H), 3.95 (m, 2H), 3.83 (m, 1H), 3.46 (m, 2H), 2.72 (m, 8H), 2.37 (s, 3H), 1.89 (m, 1H), 1.78 (m, 1H), 1.58 (m, 2H). MS m/z 407 (M+1).

### Example 175

### (2R)-2-[4-(4-Methyl-1-piperazinyl)-1H-benzimidazol-2-yl]-2-[methyl(5,6,7,8-tetrahydro-8-quinolinyl)amino]ethanol

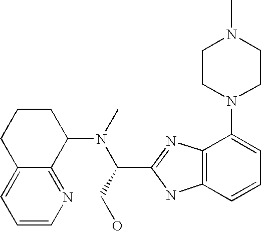

(2R)-2-[4-(4-methyl-1-piperazinyl)-1H-benzimidazol-2-yl]-2-[methyl(5,6,7,8-tetrahydro-8-quinolinyl)amino]ethanol was prepared from (2R)-2-[4-(4-methyl-1-piperazinyl)-1H-benzimidazol-2-yl]-2-(5,6,7,8-tetrahydro-8-quinolinylamino)ethanol (0.01 g, 0.02 mmol) via reductive amination with formaldehyde (0.003 mL, 0.03 mmol) in a similar manner described here within to give a white solid (0.01 g, 95%): ¹H-NMR (CD₃OD) δ 8.47 (d, 1H), 7.56 (d, 1H), 7.24 (dd, 1H), 7.10 (t, 2H), 6.68 (s, 1H), 4.36 (m, 1H), 4.25 (m, 1H), 4.09 (m, 2H), 2.89-2.81 (m, 2H), 2.74 (m, 8H), 2.38 (s, 3H), 2.13-1.99 (m, 3H), 2.02 (s, 3H), 1.77 (m, 1H). MS m/z 421 (M+1).

### Example 176

### N-Methyl-N-{(1R)-1-[4-(4-methyl-1-piperazinyl)-1H-benzimidazol-2-yl]-2-[(phenylmethyl)oxy]ethyl}-5,6,7,8-tetrahydro-8-quinolinamine

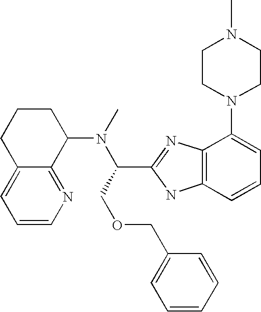

N-Methyl-N-{(1R)-1-[4-(4-methyl-1-piperazinyl)-1H-benzimidazol-2-yl]-2-[(phenylmethyl)oxy]ethyl}-5,6,7,8-tetrahydro-8-quinolinamine was prepared in a similar manner to (2R)-2-[4-(4-methyl-1-piperazinyl)-1H-benzimidazol-2-yl]-2-[methyl(5,6,7,8-tetrahydro-8-quinolinyl)amino]ethanol from O-(phenylmethyl)-N-{[(phenylmethyl)oxy]carbonyl}-L-serine to give a light yellow solid: ¹H-NMR (CD₃OD) δ13.9-13.5 (br s, 1H), 8.68 (d, 1H), 7.60 (d, 1H), 7.36 (m, 6H), 7.17-7.01 (m, 2H), 6.49 (s, 1H), 4.61 (ABq, 2H), 4.35-4.15 (m, 2H), 4.04-3.89 (m, 2H), 3.53 (m, 4H), 2.90-2.70 (m, 2H), 2.60 (m, 4H), 2.30 (s, 3H), 2.20 (s, 3H), 2.13-1.87 (m, 3H), 1.73-1.60 (m, 1H). MS m/z 511 (M+1).

### Example 177

### N-Methyl-N-{(1S)-1-[4-(4-methyl-1-piperazinyl)-1H-benzimidazol-2-yl]-2-[(phenylmethyl)oxy]ethyl}-5,6,7,8-tetrahydro-8-quinolinamine

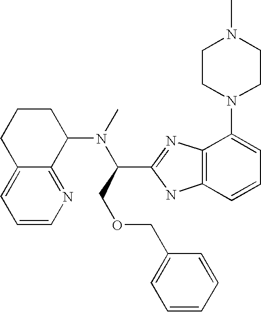

N-Methyl-N-{(1S)-1-[4-(4-methyl-1-piperazinyl)-1H-benzimidazol-2-yl]-2-[(phenylmethyl)oxy]ethyl}-5,6,7,8-tetrahydro-8-quinolinamine was prepared in a similar manner to (2R)-2-[4-(4-methyl-1-piperazinyl)-1H-benzimidazol-2-yl]-2-[methyl(5,6,7,8-tetrahydro-8-quinolinyl)amino]ethanol from O-(phenylmethyl)-N-{[(phenylmethyl)oxy]carbonyl}-L-serine to give a light yellow solid. Analytical data matched that of N-methyl-N-{(1R)-1-[4-(4-methyl-1-piperazinyl)-1H-benzimidazol-2-yl]-2-[(phenylmethyl)oxy]ethyl}-5,6,7,8-tetrahydro-8-quinolinamine.

### Example 178

### 2-{{[1-Methyl-7-(1-piperazinyl)-1H-benzimidazol-2-yl]methyl}[(8S)-5,6,7,8-tetrahydro-8-quinolinyl]amino}ethanol

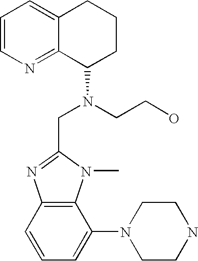

(8S)-5,6,7,8-Tetrahydro-8-quinolinamine (0.3 g, 0.58 mmol), 1,1-dimethylethyl 4-(2-formyl-1-methyl-1H-benzimidazol-7-yl)-1-piperazinecarboxylate (0.2 g, 1.3 mmol) and acetic acid (0.075 mL, 1.3 mmol) were added to dichloroethane (10 mL). Sodium triacetoxyborohydride (0.275 g, 1.3 mol) was added in two equal portions over 2 h and the solution stirred for 16 h. A saturated solution of NaHCO₃ (3 mL) was added and the solution stirred for 15 min. The organic layer was extracted 3×10 mL combined dried over Na₂SO₄, filtered and evaporated. Purification by column chromatography (1%-5% 2N NH₃/methanol in methylene chloride afford the amine. Reductive amination with {[(1,1-dimethylethyl)(dimethyl)silyl]oxy}acetaldehyde (0.07 g, 0.39 mmol) and a portion of the amine (0.125 g, 0.26 mmol) followed by acid (4N HCl) catalyzed cleavage of the Boc and tertbutyldimethyl silyl groups afforded the amine. Purification by reverse phase HPLC and neutralization of the desired fractions afford the product as a white solid (15 mg, 13%): ¹H-NMR (CD₃OD) δ 8.32 (d, 1H), 7.40 (d, 1H), 7.27 (d, 1H), 7.12-7.05 (m, 2H), 6.99 (d, 1H), 4.27 (s, 3H), 4.11 (ABq, 2H), 4.03 (dd, 1H), 3.47 (m, 1H), 3.36 (m, 1H), 3.13-3.00 (m, 6H), 2.82 (m, 5H), 2.70 (m, 1H), 2.27 (m, 1H), 2.06 (m, 2H), 1.70 (m, 1H). MS m/z 421 (M+1).

### Example 179

### 3-{{[1-Methyl-7-(1-piperazinyl)-1H-benzimidazol-2-yl]methyl}[(8S)-5,6,7,8-tetrahydro-8-quinolinyl]amino-}1-propanol

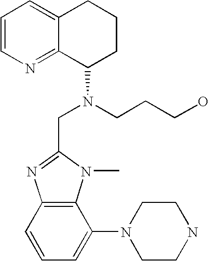

3-{{[1-Methyl-7-(1-piperazinyl)-1H-benzimidazol-2-yl]methyl}[(8S)-5,6,7,8-tetrahydro-8-quinolinyl]amino}-1-propanol was prepared in a similar manner to 2-{{[1-methyl-7-(1-piperazinyl)-1H-benzimidazol-2-yl]methyl}[(8S)-5,6,7,8-tetrahydro-8-quinolinyl]amino}ethanol from 1,1-dimethylethyl 4-(2-formyl-1-methyl-1H-benzimidazol-7-yl)-1-piperazinecarboxylate and 3-{[(1,1-dimethylethyl)(dimethyl)silyl]oxy}propanal: ¹H-NMR (CD₃OD) δ 8.32 (d, 1H), 7.38 (d, 1H), 7.28 (d, 1H), 7.11 (t, 1H), 7.05 (dd, 1H), 7.00 (d, 1H), 4.24 (s, 3H), 4.08 (m, 1H), 4.03 (ABq, 2H), 4.03, 3.54 (m, 1H), 3.45 (m, 1H), 3.16 (m, 2H), 3.03 (m, 4H), 2.86 (m, 5H), 2.70 (m, 1H), 2.22 (m, 1H), 2.14-2.05 (m, 2H), 1.79-1.68 (m, 2H), 1.54-1.49 (m, 1H). MS m/z 435 (M+1).

### Example 180

### 2-{{[1-Methyl-7-(4-methyl-1-piperazinyl)-1H-benzimidazol-2-yl]methyl}[(8S)-5,6,7,8-tetrahydro-8-quinolinyl]amino}ethanol

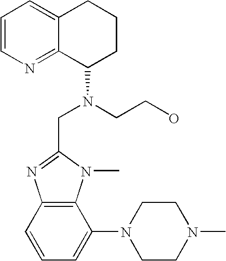

(2-{{[1-Methyl-7-(4-methyl-1-piperazinyl)-1H-benzimidazol-2-yl]methyl}[(8S)-5,6,7,8-tetrahydro-8-quinolinyl]amino}ethanol (0.035 g, 0.08 mmol), formaldehyde (0.01 mL, 0.12 mmol), acetic acid (0.008 mL, 1.2 mmol) were added to dichloroethane (10 mL). Sodium triacetoxyborohydride (0.275 g, 1.2 mol) was added and the solution stirred for 2 h. A saturated solution of NaHCO₃ (1 mL) was added, the solution stirred for 5 min and evaporated to dryness. Purification by reverse phase HPLC and neutralization of the desired fractions afford the product as a white solid (9 mg, 25%): ¹H-NMR (CD₃OD) δ 8.33 (d, 1H), 7.39 (d, 1H), 7.28 (d, 1H), 7.11 (d, 1H), 7.06 (dd, 1H), 7.01 (d, 1H), 4.26 (s, 3H), 4.12 (ABq, 2H), 4.03 (dd, 1H), 3.47 (m, 1H), 3.39 (m, 1H), 3.13 (m, 2H), 2.94 (m, 4H), 2.82 (m, 2H), 2.71 (m, 2H), 2.47 (m, 2H), 2.40 (s, 3H), 2.27 (m, 1H), 2.06 (m, 2H), 1.70 (m, 1H). MS m/z 435 (M+1).

### Example 181

### 3-{{[1-Methyl-7-(4-methyl-1-piperazinyl)-1H-benzimidazol-2-yl]methyl}[(8S)-5,6,7,8-tetrahydro-8-quinolinyl]amino}-1-propanol

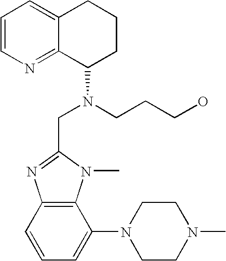

3-{{[1-Methyl-7-(4-methyl-1-piperazinyl)-1H-benzimidazol-2-yl]methyl}[(8S)-5,6,7,8-tetrahydro-8-quinolinyl]amino}-1-propanol was prepared in a similar manner to 2-{{[1-methyl-7-(4-methyl-1-piperazinyl)-1H-benzimidazol-2-yl]methyl}[(8S)-5,6,7,8-tetrahydro-8-quinolinyl]amino}ethanol from 3-{{[1-methyl-7-(1-piperazinyl)-1H-benzimidazol-2-yl]methyl}[(8S)-5,6,7,8-tetrahydro-8-quinolinyl]amino}-1-propanol: ¹H-NMR (CD₃OD) δ 8.32 (d, 1H), 7.38 (d, 1H), 7.29 (d, 1H), 7.11 (t, 1H), 7.06 (dd, 1H), 7.02 (d, 1H), 4.23 (s, 3H), 4.08 (m, 1H), 4.03 (ABq, 2H), 3.54 (m, 1H), 3.46 (m, 1H), 3.16 (m, 2H), 2.94 (m, 4H), 2.85 (m, 2H), 2.72 (m, 2H), 2.47 (m, 2H), 2.40 (s, 3H), 2.27-2.20 (m, 1H), 2.15-2.04 (m, 2H), 1.77-1.66 (m, 2H), 1.55-1.48 (m, 1H). MS m/z 449 (M+1).

### Example 182

### N-{[4-(4-Methyl-1-piperazinyl)-1H-benzimidazol-2-yl]methyl}-6,7-dihydro-5H-cyclopenta[b]pyridin-7-amine

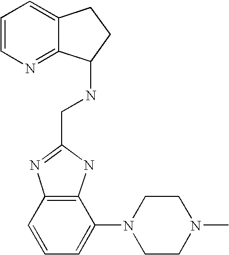

N-{[4-(4-Methyl-1-piperazinyl)-1H-benzimidazol-2-yl]methyl}-6,7-dihydro-5H-cyclopenta[b]pyridin-7-amine was prepared in a similar manner to 2-{{[1-methyl-7-(4-methyl-1-piperazinyl)-1H-benzimidazol-2-yl]methyl}[(8S)-5,6,7,8-tetrahydro-8-quinolinyl]amino}ethanol from phenylmethyl {[4-(4-methyl-1-piperazinyl)-1H-benzimidazol-2-yl]methyl}carbamate and 5,6-dihydro-7H-cyclopenta[b]pyridin-7-one to afford a tan solid: ¹H-NMR (CD₃OD) δ 8.32 (d, 1H), 7.38 (d, 1H), 7.29 (d, 1H), 7.11 (t, 1H), 7.02 (t, 1H), 6.71 (d, 1H), 4.22 (m, 3H), 3.42 (m, 4H), 3.08 (m, 4H), 3.03 (m, 1H), 2.86 (m, 1H), 2.65 (s, 3H), 2.50 (m, 2H), 1.94 (m, 1H), MS m/z 363 (M+1).

### Example 183

### N-Methyl-N-{[4-(4-methyl-1-piperazinyl)-1H-benzimidazol-2-yl]methyl}-6,7-dihydro-5H-cyclopenta[b]pyridin-7-amine

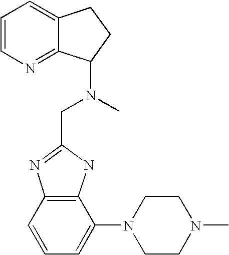

N-Methyl-N-{[4-(4-methyl-1-piperazinyl)-1H-benzimidazol-2-yl]methyl}-6,7-dihydro-5H-cyclopenta[b]pyridin-7-amine was prepared from N-{[4-(4-Methyl-1-piperazinyl)-1H-benzimidazol-2-yl]methyl}-6,7-dihydro-5H-cyclopenta[b]pyridin-7-amine by reductive amination as described here within to afford a white solid: ¹H-NMR (CD₃OD) δ 8.43 (d, 1H), 7.69 (d, 1H), 7.25 (dd, 1H), 7.14 (t, 1H), 7.10 (t, 1H), 6.68 (d, 1H), 4.51 (t, 1H), 4.00 (½ABq, 1H), 3.83 (½ABq, 1H), 3.32 (m, 4H), 2.98 (m, 1H), 2.88 (m, 1H), 2.40 (m, 4H), 2.77 (dd, 2H), 2.13 (s, 3H). MS m/z 377 (M+1).

### Example 184

### N-{[4-(4-methyl-1-piperazinyl)-1H-benzimidazol-2-yl]methyl}-6,7,8,9-tetrahydro-5H-cyclohepta[b]pyridin-9-amine

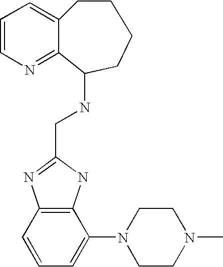

N-{[4-(4-methyl-1-piperazinyl)-1H-benzimidazol-2-yl]methyl}-6,7,8,9-tetrahydro-5H-cyclohepta[b]pyridin-9-amine was prepared in a similar manner to 2-{{[1-methyl-7-(4-methyl-1-piperazinyl)-1H-benzimidazol-2-yl]methyl}[(8S)-5,6,7,8-tetrahydro-8-quinolinyl]amino}ethanol from phenylmethyl {[4-(4-methyl-1-piperazinyl)-1H-benzimidazol-2-yl]methyl}carbamate and 5,6,7,8-tetrahydro-9H-cyclohepta[b]pyridin-9-one to afford a white solid: ¹H-NMR (CD₃OD) δ 8.36 (d, 1H), 7.58 (d, 1H), 7.22 (d, 1H), 7.17 (m, 2H), 6.72 (d, 1H), 4.48 (d, 1H), 4.34 (ABq, 2H), 3.53 (m, 4H), 3.22 (m, 4H), 2.92 (m, 1H), 2.82 (m, 1H), 2.78 (s, 3H), 2.20-2.09 (m, 2H), 1.89 (m, 2H), 1.66 (m, 1H), 1.39 (m, 1H). MS m/z 391 (M+1).

### Example 185

### N-methyl-N-{[4-(4-methyl-1-piperazinyl)-1H-benzimidazol-2-yl]methyl}-6,7,8,9-tetrahydro-5H-cyclohepta[b]pyridin-9-amine

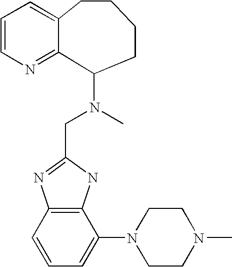

N-methyl-N-{[4-(4-methyl-1-piperazinyl)-1H-benzimidazol-2-yl]methyl}-6,7,8,9-tetrahydro-5H-cyclohepta[b]pyridin-9-amine was prepared from N-{[4-(4-methyl-1-piperazinyl)-1H-benzimidazol-2-yl]methyl}-6,7,8,9-tetrahydro-5H-cyclohepta[b]pyridin-9-amine by reductive amination as described here within to afford a white solid: ¹H-NMR (CD₃OD) δ 8.28 (d, 1H), 7.51 (d, 1H), 7.18 (m, 2H), 7.10 (t, 1H), 6.68 (d, 1H), 3.87 (½ABq, 1H), 3.77 (m, 1H), 3.69 (½ABq, 1H), 3.52 (m, 1H), 3.33 (m, 4H), 2.80 (m, 4H), 2.61 (m, 1H), 2.43 (s, 3H), 2.27-2.14 (m, 2H), 2.22 (s, 3H), 2.90 (m, 1H), 1.94 (m, 1H), 1.78 (m, 2H), 1.47 (m, 1H). MS m/z 405 (M+1).

### Biological Section

Fusion Assay

Plasmid Generation

The complete coding sequences of HIV-1 tat (GenBank Accession No. X07861) and rev (GenBank Accession No. M34378) were cloned into pcDNA3.1 expression vectors containing G418 and hygromycin resistance genes, respectively. The complete coding sequence of the HIV-1 (HXB2 strain) gp160 envelope gene (nucleotide bases 6225-8795 of GenBank Accession No. K03455) was cloned into plasmid pCRII-TOPO. The three HIV genes were additionally inserted into the baculovirus shuttle vector, pFastBacMam1, under the transcriptional control of the CMV promoter. A construction of the pHIV-I LTR containing mutated NFkB sequences linked to the luciferase reporter gene was prepared by digesting pcDNA3.1, containing the G418 resistance gene, with Nru I and Bam HI to remove the CMV promoter. LTR-luc was then cloned into the Nru I/Bam HI sites of the plasmid vector. Plasmid preparations were performed after the plasmids were amplified in Escherichia coli strain DH5-alpha. The fidelity of the inserted sequences was confirmed by double-strand nucleotide sequencing using an ABI Prism Model 377 automated sequencer.

BacMam Baculovirus Generation

Recombinant BacMam baculoviruses were constructed from pFastBacMam shuttle plasmids by using the bacterial cell-based Bac-to-Bac system. Viruses were propagated in Sf9 (Spodoptera frugiperda) cells cultured in Hink's TNM-FH Insect media supplemented with 10% (v/v) fetal bovine serum and 0.1% (v/v) pluronic F-68 according to established protocols.

Cell Culture

Human osteosarcoma (HOS) cells that naturally express human CXCR4 were transfected with human CCR5, human CD4 and the pHIV-LTR-luciferase plasmid using FuGENE 6 transfection reagent. Single cells were isolated and grown under selection condition in order to generate a stable HOS (hCXCR4/hCCR5/hCD4/pHIV-LTR-luciferase) clonal cell line. The cells were maintained in Dulbeccos modified Eagles media supplemented with 10% fetal calf serum (FCS), G418 (400 ug/ml), puromycin (1 ug/ml), mycophenolic acid (40 ug/ml), xanthine (250 ug/ml) and hypoxanthine (13.5 ug/ml) to maintain a selection pressure for cells expressing the LTR-luciferase, hCCR5 and hCD4, respectively. Human embryonic kidney (HEK-293) cells stably transfected to express the human macrophage scavenging receptor (Class A, type 1; GenBank Accession No. D90187), were maintained in DMEM/F-12 media (1:1) supplemented with 10% FCS and 1.5 ug/ml puromycin. The expression of this receptor by the HEK-293 cells enhances their ability to stick to tissue culture treated plasticware.

Transduction of HEK-293 Cells

HEK-293 cells were harvested using enzyme-free cell dissociation buffer. The cells were resuspended in DMEM/F-12 media supplemented with 10% FCS and 1.5 ug/ml and counted. Tranductions were performed by direct addition of BacMam baculovirus containing insect cell media to cells. The cells were simultaneously transduced with BacMam baculovirus expressing HIV-1 tat, HIV-1 rev and HIV-1 gp160 (from the HXB2 HIV strain). Routinely an MOI of 10 of each virus was added to the media containing the cells. 2 mM butyric acid was also added to the cells at this stage to increase protein expression in transduced cells. The cells were subsequently mixed and seeded into a flask at 30 million cells per T225. The cells were incubated at 37° C., 5% CO₂, 95% humidity for 24 h to allow for protein expression.

Cell/Cell Fusion Assay Format

HEK and HOS cells were harvested in DMEM/F-12 media containing 2% FCS and DMEM media containing 2% FCS, respectively, with no selection agents added. Compounds were plated as 1 ul spots in 100% DMSO on a 96-well CulturPlate plates. HOS cells (50 ul) were added first to the wells, followed immediately by the HEK cells (50 ul). The final concentration of each cell type was 20,000 cells per well. Following these additions, the cells were returned to a tissue culture incubator (37° C.; 5% CO₂/95% air) for an additional 24 h.

Measurement of Luciferase Production

Following the 24 h incubation, total cellular luciferase activity was measured using the LucLite Plus assay kit (Packard, Meridien, Conn.). In brief, 100 ul of this reagent was added to each well. The plates were sealed and mixed. The plates were dark adapted for approximately 10 min prior to the luminescence being read on a Packard TopCount.

Functional Assay

Cell Culture

Human embryonic kidney (HEK-293) cells were maintained and harvested as described above. Cells were plated in 96-well, black clear bottom, poly-lysine coated plates at a concentration of 40,000 cells per well in a final volume of 100 ul containing human CXCR4BacMam (MOI=25) and Gqi5 BacMam (MOI=12.5). The cells were incubated at 37° C., 5% CO₂, 95% humidity for 24 h to allow for protein expression.

Functional FLIPR Assay

After the required incubation time the cells were washed once with 50 ul of fresh serum-free DMEM/F12 media containing probenicid. 50 ul of dye solution was then added to the cells (Calcium Plus Assay Kit Dye; Molecular Devices) was dissolved in 200 ml of the above probenicid/BSA containing media and incubated for 1 h. Cell plates were transferred to a Fluorometric Imaging Plate Reader (FLIPR). Upon addition the effect of the compounds on the change in [Ca²⁺]ᵢ was examined to determine if the compounds were agonists or antagonists (ability to block SDF-1 alpha activity) at the CXCR4 receptor. IC₅₀ values are determined and pK_{b} values are calculated using the Leff and Dougall equation: K_{B}=IC₅₀/((2+([agonist]/EC₅₀ˆb)ˆ1/b−1) Where IC₅₀ is that defined by the antagonist concentration-response curve [agonist] is the EC₈₀ concentration of agonist used EC₅₀ is that defined by the agonist concentration-response curve b is the slope of the agonist concentration-response curve.

HOS HIV-1 Infectivity Assay

HIV Virus Preparation

Compounds were profiled against two HIV-1 viruses, the M-tropic (CCR5 utilizing) Ba-L strain and the T-tropic (CXCR4 utilizing) IIIB strain. Both viruses were propagated in human peripheral blood lymphocytes. Compounds were tested for there ability to block infection of the HOS cell line (expressing hCXCR4/hCCR5/hCD4/pHIV-LTR-luciferase) by either HIV-1 Ba-L or HIV-1 IIIB. Compound cytotoxicity was also examined in the absence of virus addition.

HOS HIV-1 Infectivity Assay Format

HOS cells (expressing hCXCR4/hCCR5/hCD4/pHIV-LTR-luciferase) were harvested and diluted in Dulbeccos modified Eagles media supplemented with 2% FCS and non-essential amino acid to a concentration of 60,000 cells/ml. The cells were plated into 96-well plates (100 ul per well) and the plates were placed in a tissue culture incubator (37° C.; 5% CO₂/95% air) for a period of 24 h.

Subsequently, 50 ul of the desired drug solution (4 times the final concentration) was added to each well and the plates were returned to the tissue culture incubator (37° C.; 5% CO₂/95% air) for 1 h. Following this incubation 50 ul of diluted virus was added to each well (approximately 2 million RLU per well of virus). The plates were returned to the tissue culture incubator (37° C.; 5% CO₂/95% air) and were incubated for a further 96 h.

Following this incubation the endpoint for the virally infected cultures was quantified following addition of Steady-Glo Luciferase assay system reagent (Promega, Madison, Wis.). Cell viability or non-infected cultures was measured using a CellTiter-Glo luminescent cell viability assay system (Promega, Madison, Wis.). All luminescent readouts are performed on a Topcount luminescence detector (Packard, Meridien, Conn.).

TABLE 1

| | | Functional | Fusion | | |
|---|---|---|---|---|---|
| | | assay | assay | Cytotox | HOS (3B) |
| Example | Structure | (pIC50) | (pIC50) | (pIC50) | (μM) |
| 7 | 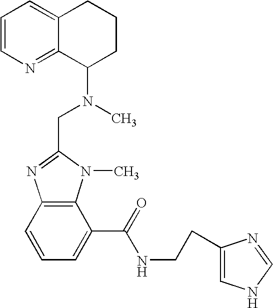 | 8.07 (n = 1) | 7.26 (n = 2) | <4.00 (n = 1) | 0.008 (n = 2) |
| 8 | 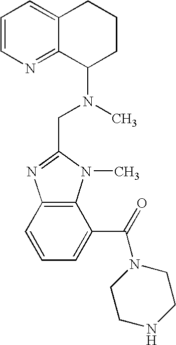 | 7.47 (n = 1) | 7.27 (n = 2) | <4.00 (n = 1) | 0.043 (n = 2) |
| 9 | 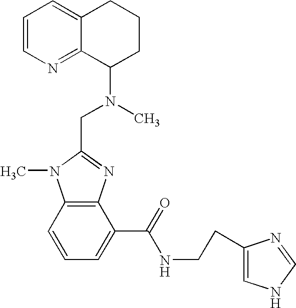 | 7.04 (n = 1) | 7.12 (n = 2) | <4.00 (n = 1) | 0.056 (n = 2) |
| 10 | 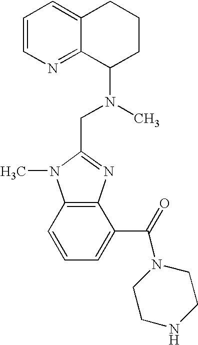 | 5.85 (n = 1) | 5.72 (n = 2) | <4.00 (n = 1) | 0.84 (n = 2) |
| 11 | 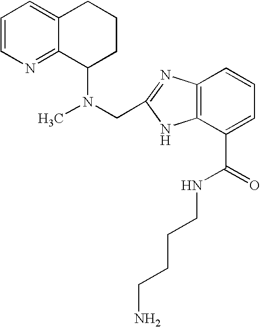 | 6.59 (n = 1) | 6.13 (n = 2) | <4.00 (n = 1) | 0.395 (n = 3) |
| 12 | 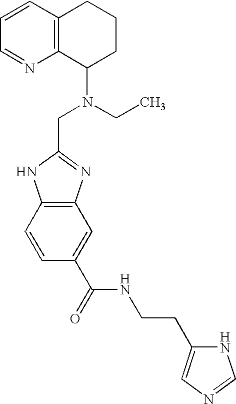 | 6.35 (n = 1) | 5.93 (n = 2) | <4.00 (n = 1) | 0.179 (n = 5) |
| 13 | 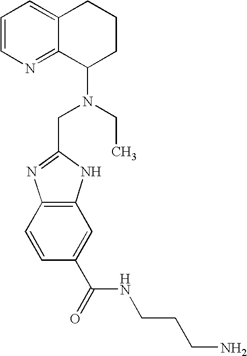 | 6.38 (n = 1) | 5.76 (n = 2) | <4.00 (n = 1) | 0.594 (n = 2) |
| 14 | 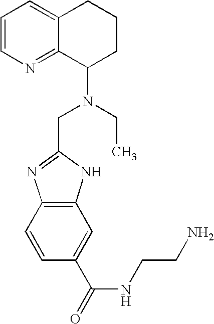 | 6.47 (n = 1) | 5.59 (n = 2) | <4.00 (n = 1) | 0.440 (n = 2) |
| 15 | 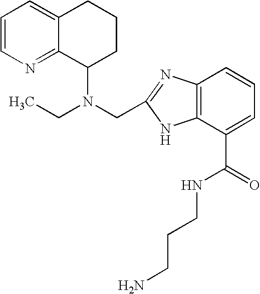 | 6.92 (n = 1) | 6.45 (n = 2) | <4.00 (n = 1) | 0.141 (n = 3) |
| 16 | 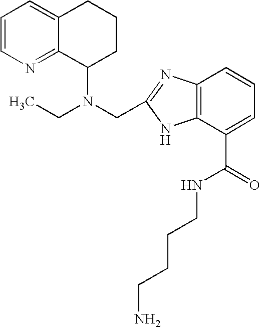 | 6.54 (n = 1) | 6.14 (n = 2) | <4.00 (n = 1) | 0.238 (n = 3) |
| 17 | 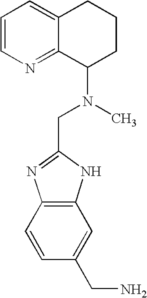 | 6.59 (n = 1) | 5.89 (n = 1) | <4.00 (n = 1) | 1.36 (n = 2) |
| 18 | 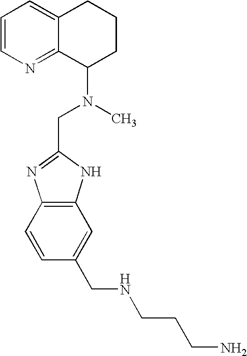 | 6.82 (n = 1) | 6.33 (n = 2) | <4.00 (n = 1) | 0.160 (n = 1) |
| 19 | 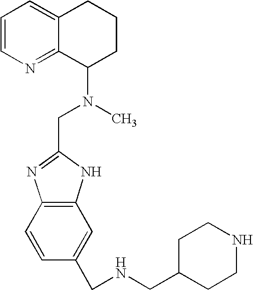 | 6.60 (n = 1) | 6.30 (n = 2) | <4.00 (n = 1) | 0.167 (n = 2) |
| 20 | 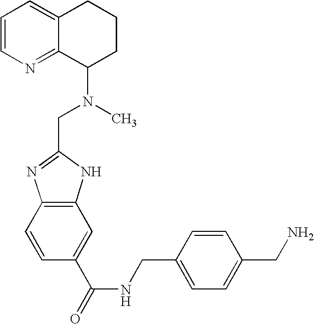 | <4.00 (n = 1) | 5.25 (n = 2) | <4.00 (n = 1) | 2.35 (n = 2) |
| 21 | 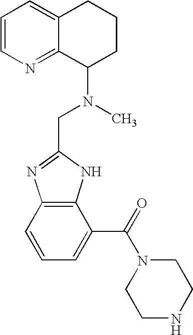 | 7.80 (n = 1) | 7.22 (n = 2) | <4.00 (n = 1) | 0.046 (n = 2) |
| 22 | 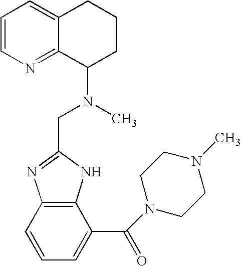 | 6.77 (n = 1) | 5.98 (n = 2) | <4.00 (n = 1) | 0.331 (n = 2) |
| 23 | 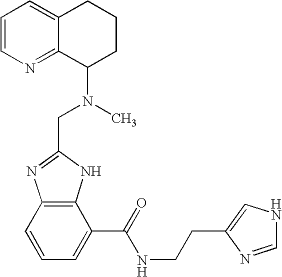 | 7.87 (n = 1) | 6.80 (n = 2) | <4.00 (n = 1) | 0.018 (n = 2) |
| 24 | 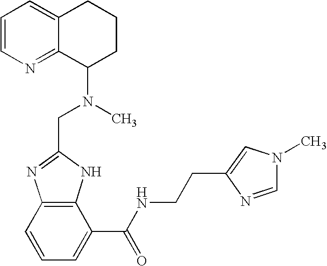 | 7.91 (n = 1) | 6.84 (n = 2) | <4.00 (n = 1) | 0.022 (n = 1) |
| 25 | 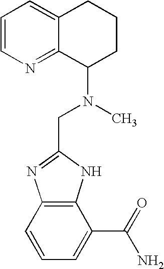 | 6.76 (n = 1) | 6.56 (n = 2) | <4.00 (n = 1) | 0.143 (n = 2) |
| 26 | 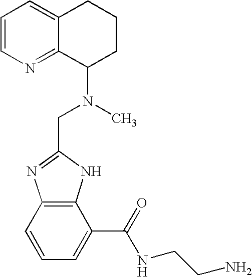 | 7.68 (n = 1) | 7.31 (n = 2) | <4.00 (n = 1) | 0.027 (n = 2) |
| 27 | 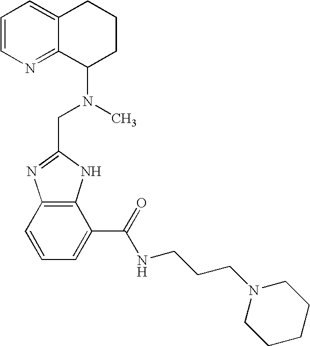 | 6.75 (n = 1) | 6.65 (n = 2) | <4.00 (n = 1) | 0.062 (n = 2) |
| 28 | 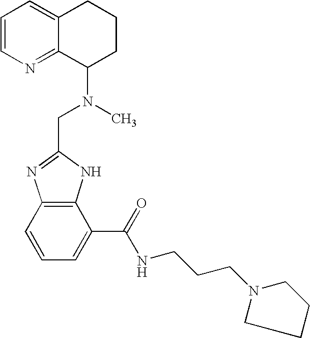 | 7.24 (n = 1) | 6.83 (n = 2) | <4.00 (n = 1) | 0.044 (n = 2) |
| 29 | 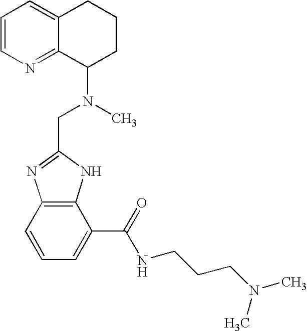 | 6.88 (n = 1) | 6.59 (n = 2) | <4.00 (n = 1) | 0.081 (n = 2) |
| 30 | 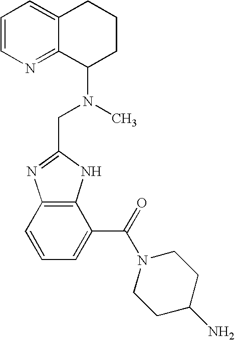 | 5.70 (n = 1) | 6.39 (n = 2) | <4.00 (n = 1) | 0.075 (n = 2) |
| 31 | 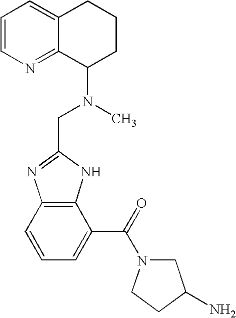 | 7.91 (n = 1) | 7.31 (n = 2) | <4.00 (n = 1) | 0.022 (n = 2) |
| 32 | 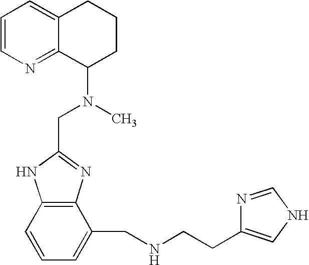 | 7.18 (n = 1) | 7.07 (n = 1) | 5.12 (n = 1) | 0.045 (n = 2) |
| 33 | 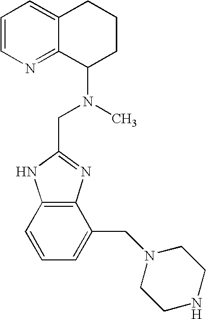 | 7.82 (n = 1) | 6.79 (n = 2) | <4.00 (n = 1) | 0.013 (n = 1) |
| 34 | 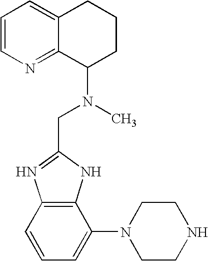 | 8.09 (n = 1) | 8.26 (n = 2) | <4.00 (n = 1) | 0.0062 (n = 2) |
| 35 | 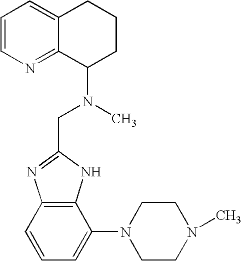 | 8.23 (n = 1) | 9.41 (n = 2) | <4.00 (n = 1) | 0.0016 (n = 1) |
| 36 | 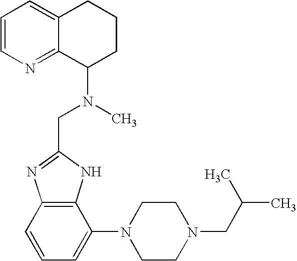 | 6.11 (n = 1) 8.08 (n = 1) | 5.27 (n = 2) 7.66 (n = 1) | <4.00 (n = 1) | 0.05 (n = 2) |

Compounds of the present invention demonstrate desired potency. For example, compounds of the present invention demonstrate desired potency, below 100 nm. Moreover, compounds of the present invention are believed to provide a desired pK profile. Also, compounds of the present invention are believed to provide a desired secondary biological profile. Compounds active in HOS assay were also active in the fusion assay. Compounds exhibited separation between activity and cytotoxicity in the described assays.

Activity of various compounds of the present invention are included in Table 2.

TABLE 2

| | | Activity |
|---|---|---|
| Example | Structure | Level* |
| 7 | 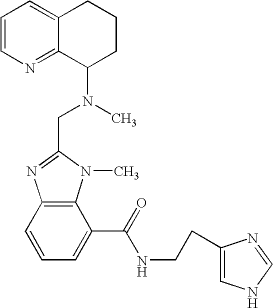 | A |
| 8 | 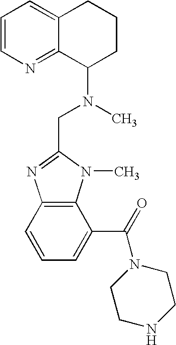 | A |
| 9 | 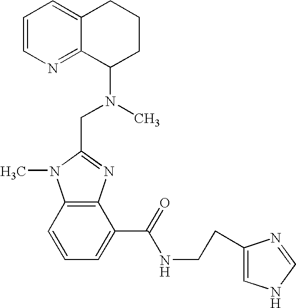 | A |
| 10 | 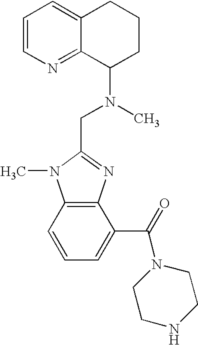 | C |
| 11 | 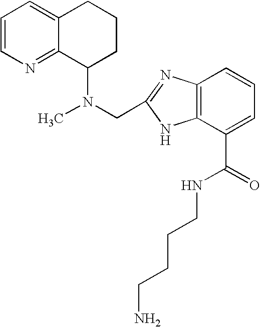 | B |
| 12 | 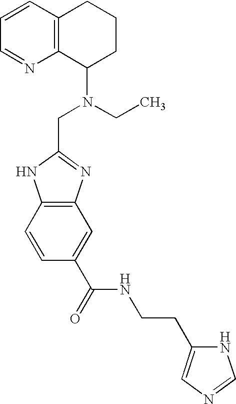 | B |
| 13 | 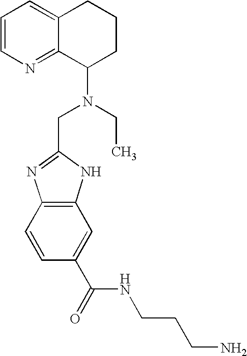 | C |
| 14 | 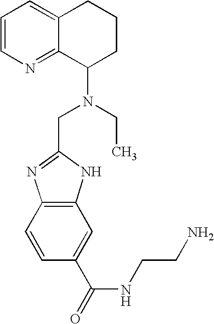 | B |
| 15 | 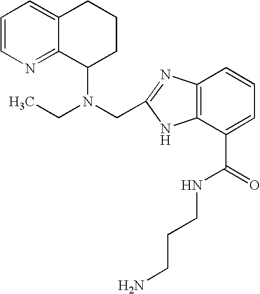 | B |
| 16 | 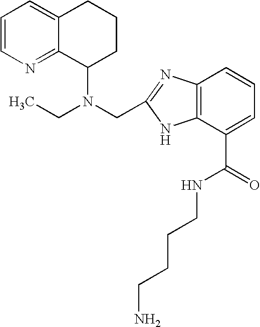 | B |
| 17 | 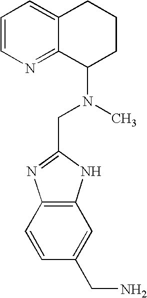 | C |
| 18 | 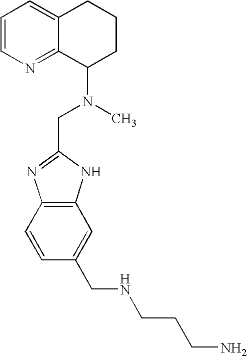 | B |
| 19 | 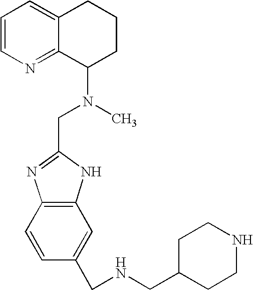 | B |
| 20 | 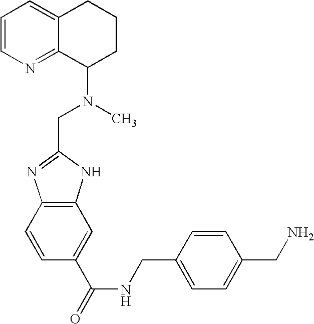 | C |
| 21 | 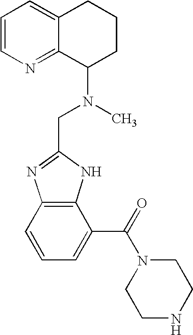 | A |
| 22 | 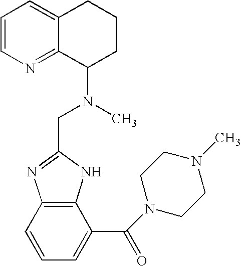 | B |
| 23 | 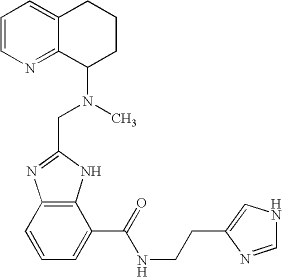 | A |
| 24 | 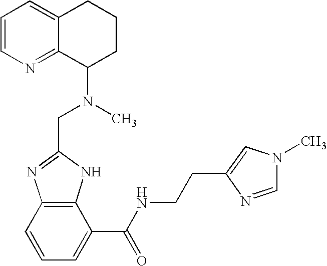 | A |
| 25 | 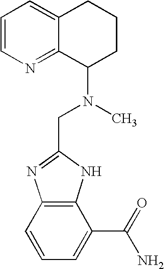 | B |
| 26 | 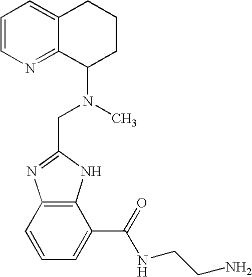 | A |
| 27 | 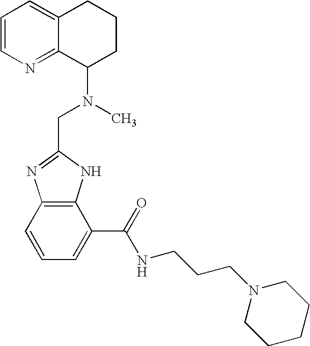 | A |
| 28 | 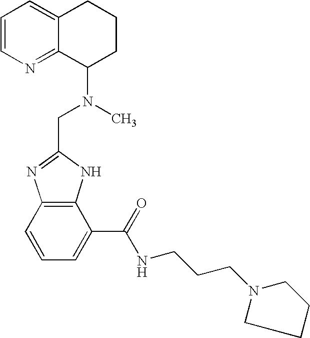 | A |
| 29 | 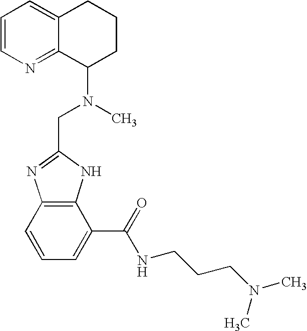 | A |
| 30 | 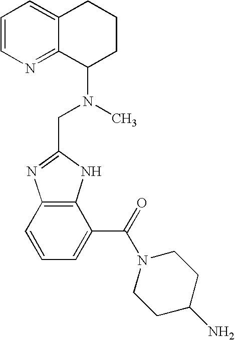 | A |
| 31 | 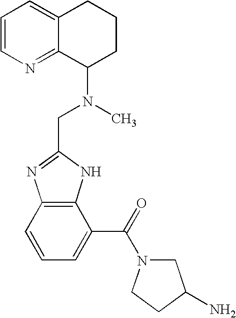 | A |
| 32 | 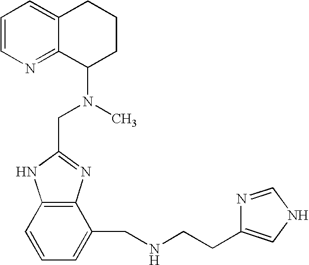 | A |
| 33 | 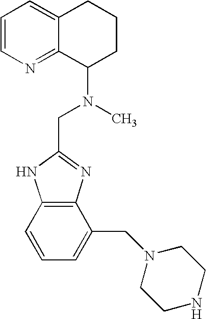 | A |
| 35 | 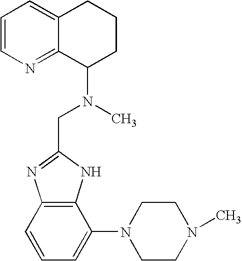 | A |
| 36 | 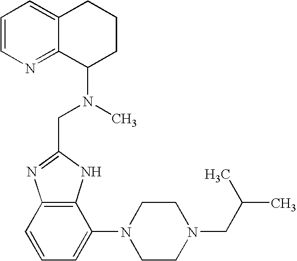 | A |
| 37 | 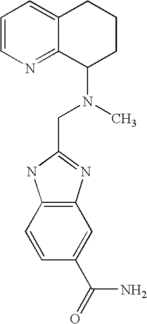 | A |
| 38 | 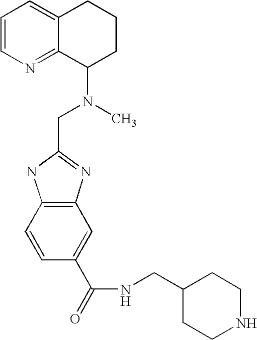 | C |
| 39 | 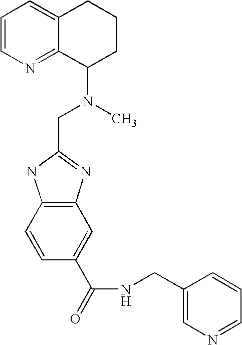 | C |
| 40 | 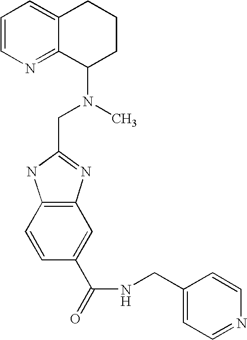 | C |
| 41 | 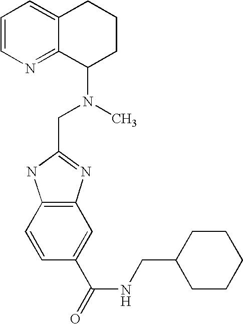 | C |
| 42 | 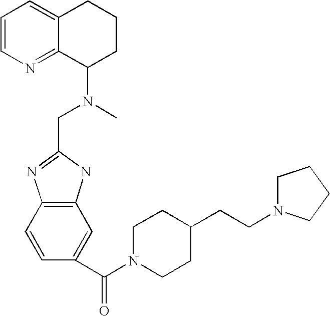 | C |
| 43 | 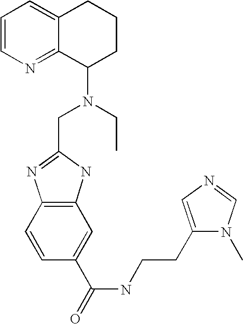 | B |
| 44 | 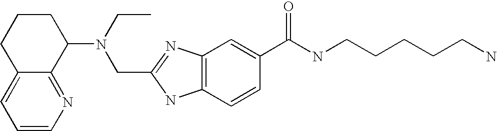 | C |
| 46 | 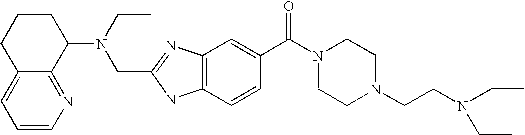 | C |
| 48 | 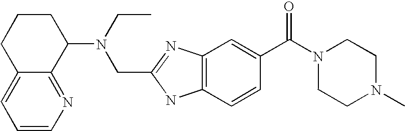 | B |
| 49 | 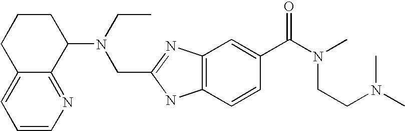 | C |
| 50 | 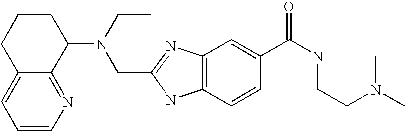 | B |
| 51 | 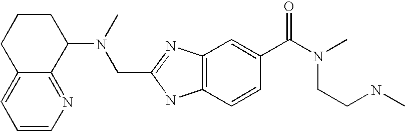 | A |
| 52 | 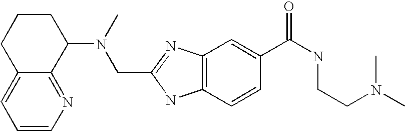 | A |
| 53 | 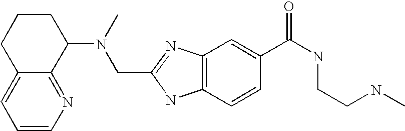 | A |
| 54 | 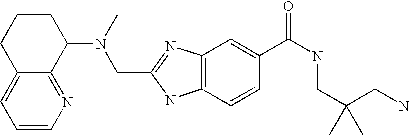 | B |
| 55 | 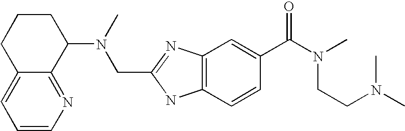 | A |
| 56 | 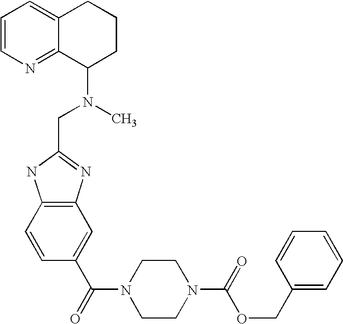 | C |
| 57 | 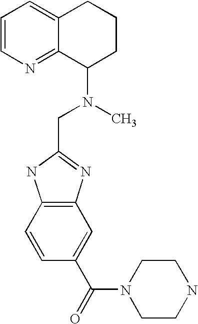 | C |
| 58 | 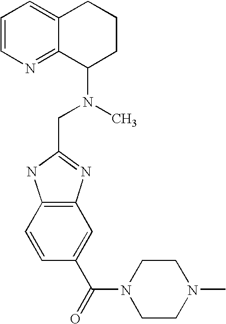 | B |
| 59 | 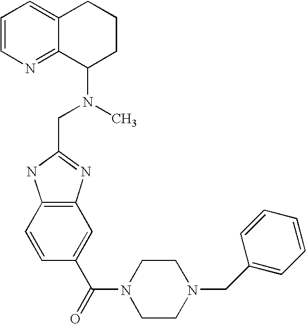 | C |
| 61 | 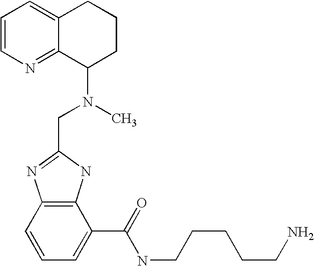 | B |
| 62 | 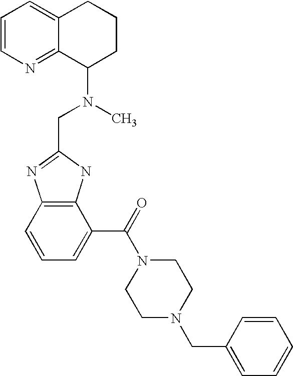 | C |
| 64 | 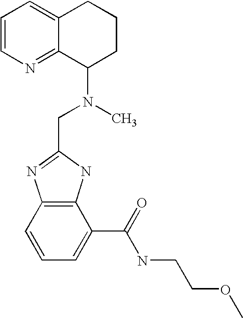 | C |
| 67 | 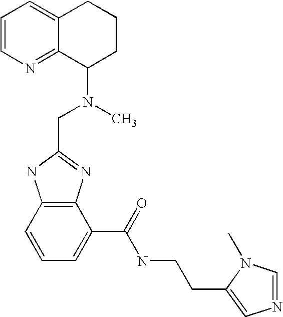 | B |
| 68 | 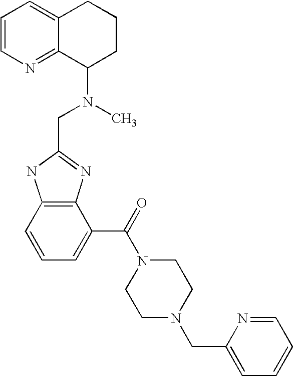 | C |
| 69 | 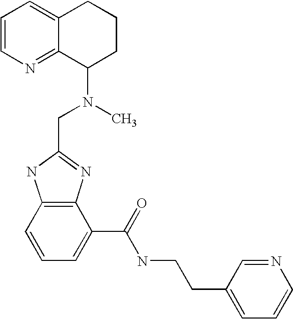 | C |
| 71 | 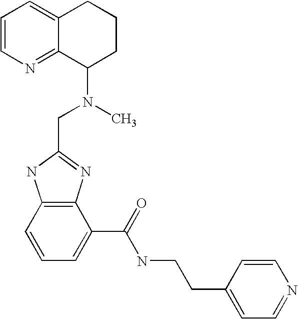 | C |
| 73 | 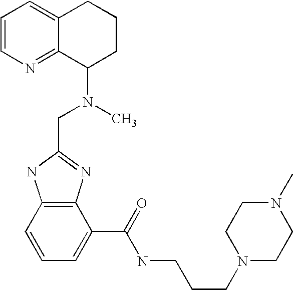 | B |
| 74 | 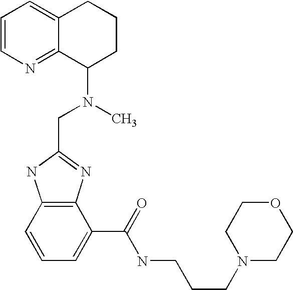 | A |
| 75 | 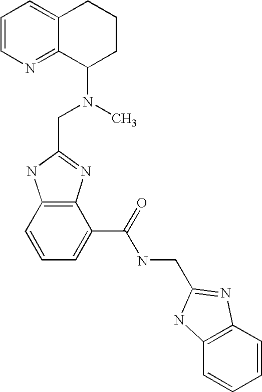 | A |
| 76 | 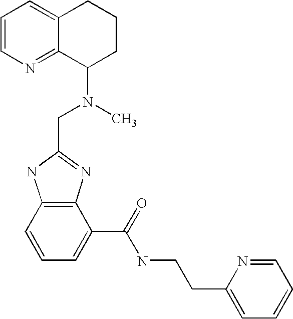 | B |
| 77 | 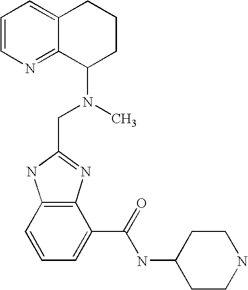 | B |
| 78 | 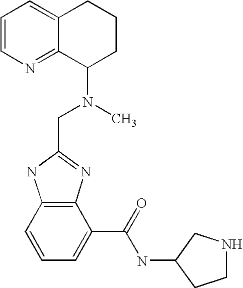 | A |
| 79 | 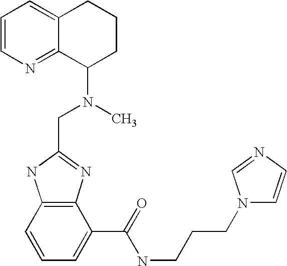 | A |
| 80 | 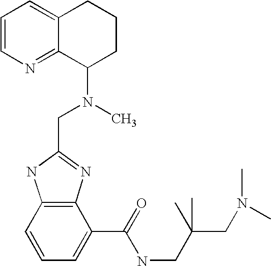 | B |
| 81 | 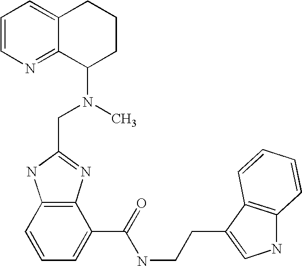 | C |
| 82 | 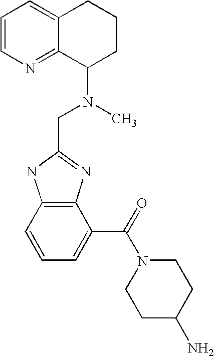 | A |
| 83 | 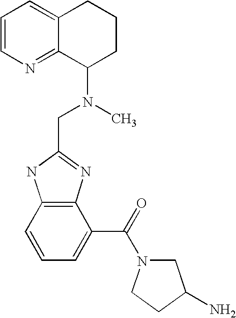 | A |
| 84 | 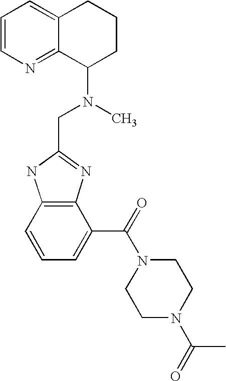 | B |
| 85 | 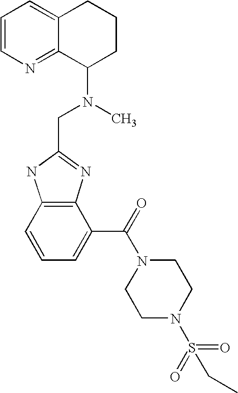 | B |
| 86 | 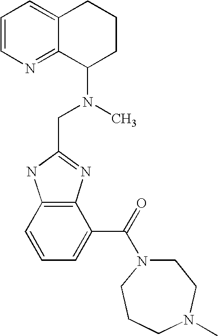 | A |
| 87 | 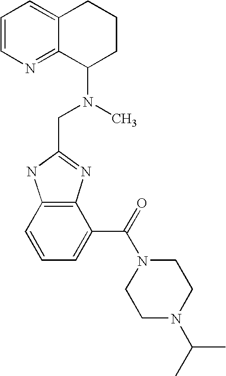 | B |
| 88 | 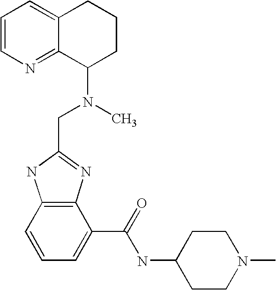 | B |
| 89 | 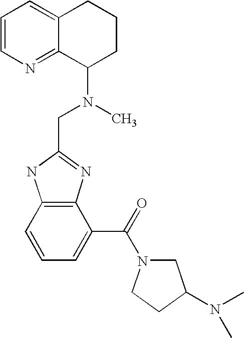 | B |
| 90 | 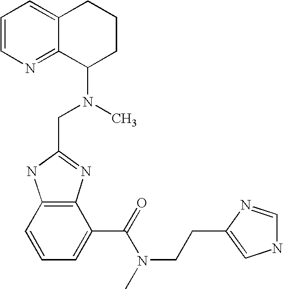 | A |
| 91 | 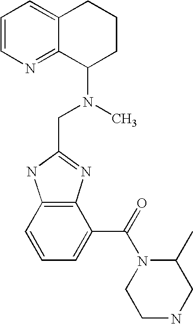 | A |
| 92 | 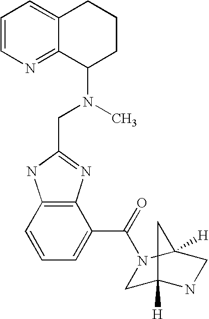 | A |
| 93 | 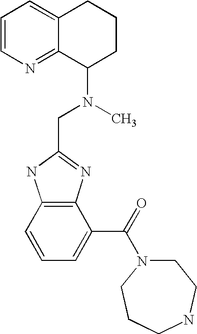 | A |
| 94 | 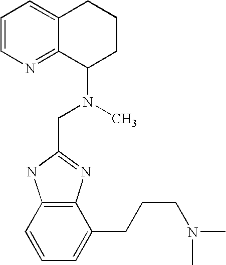 | A |
| 95 | 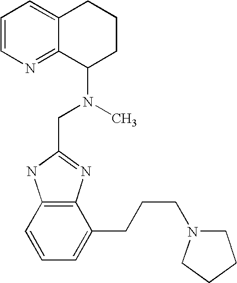 | A |
| 96 | 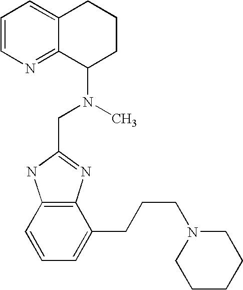 | A |
| 97 | 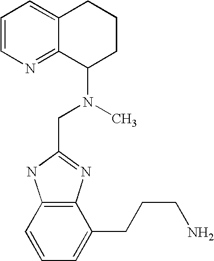 | A |
| 99 | 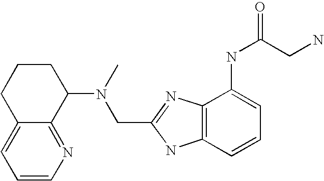 | B |
| 100 | 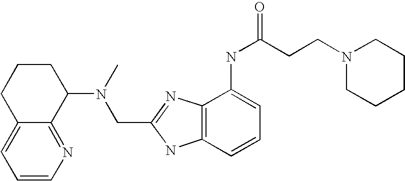 | B |
| 101 | 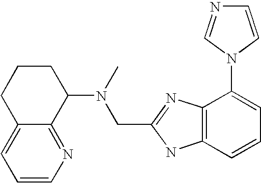 | B |
| 102 | 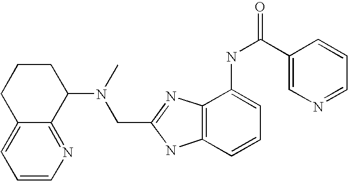 | B |
| 103 | 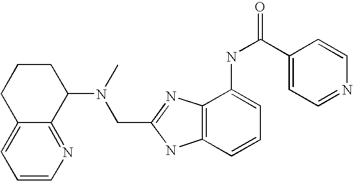 | C |
| 104 | 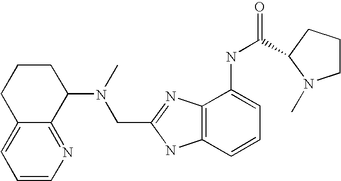 | B |
| 105 | 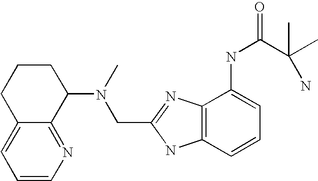 | B |
| 106 | 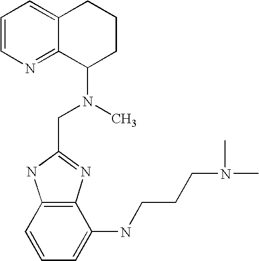 | B |
| 107 | 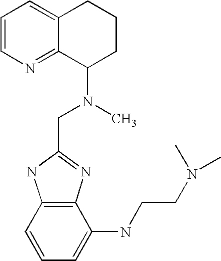 | A |
| 108 | 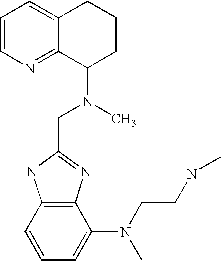 | A |
| 109 | 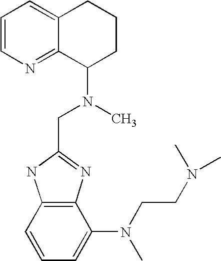 | A |
| 110 | 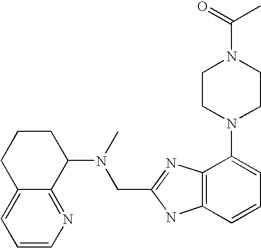 | B |
| 111 | 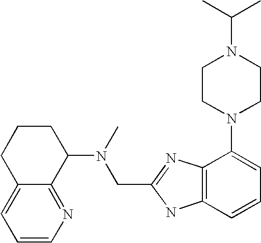 | A |
| 112 | 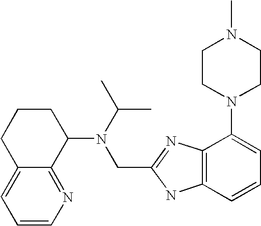 | A |
| 113 | 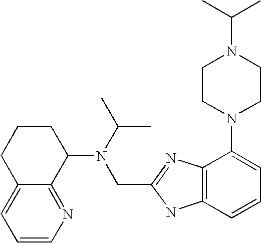 | A |
| 114 | 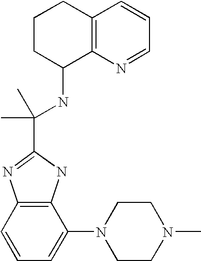 | A |
| 115 | 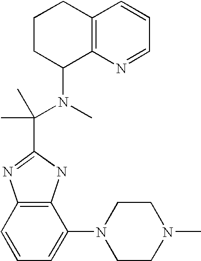 | A |
| 116 | 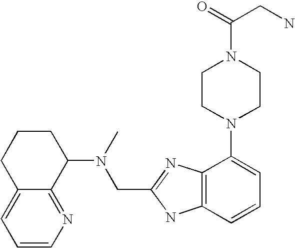 | A |
| 117 | 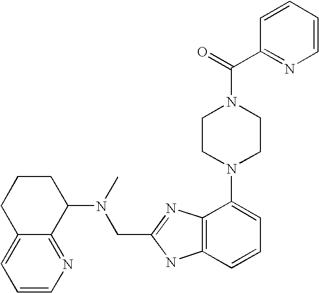 | B |
| 133A | 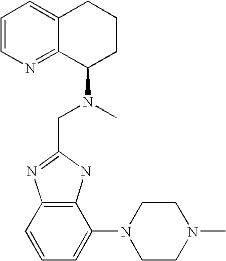 | A |
| 133B | 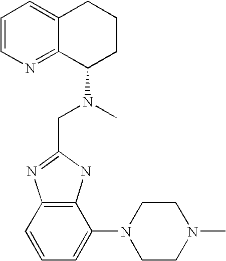 | A |
| 134 | 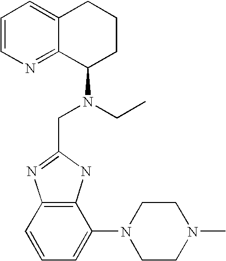 | A |
| 135 | 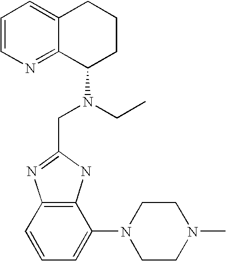 | A |
| 136 | 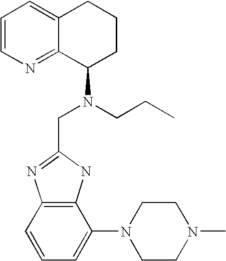 | A |
| 137 | 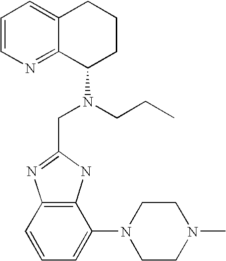 | A |
| 138 | 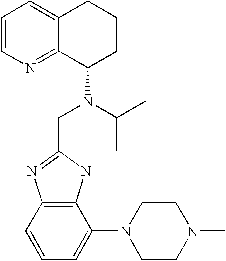 | A |
| 139 | 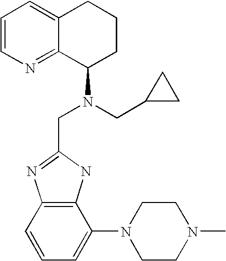 | A |
| 140 | 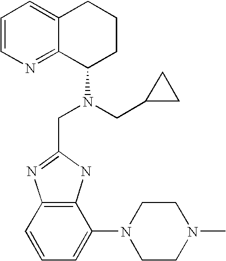 | A |
| 141 | 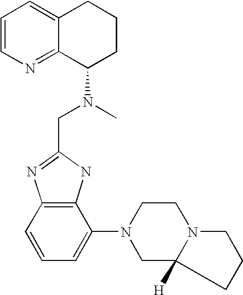 | A |
| 142 | 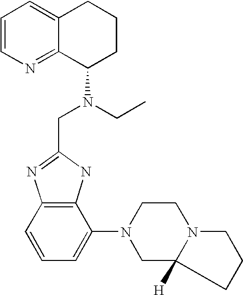 | A |
| 143 | 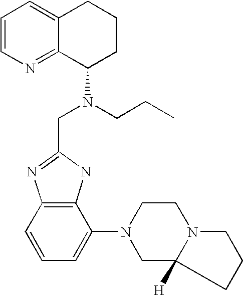 | A |
| 144 | 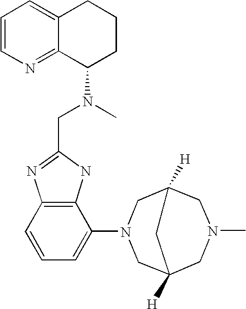 | A |
| 145 | 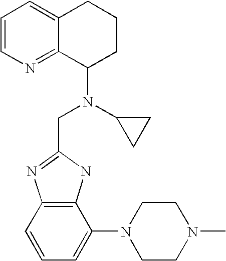 | A |
| 146 | 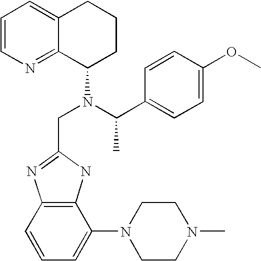 | A |
| 147 | 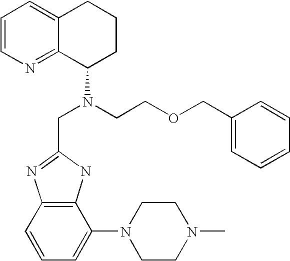 | A |
| 148 | 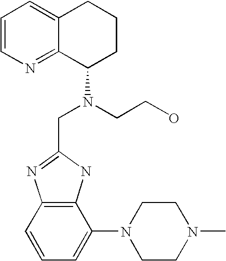 | A |
| 149 | 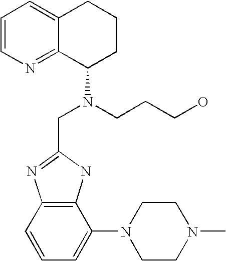 | A |
| 150 | 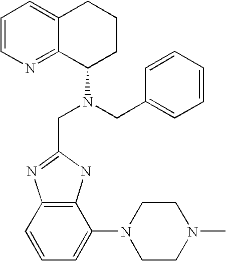 | A |
| 152 | 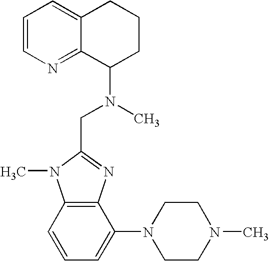 | B |
| 153 | 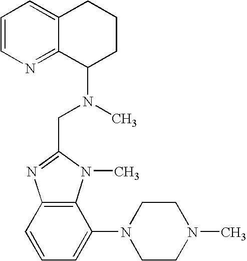 | A |
| 154 | 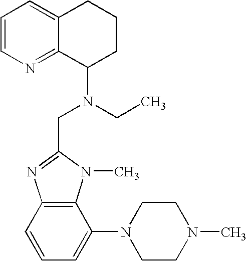 | A |
| 155 | 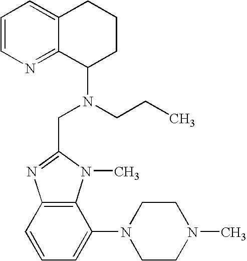 | A |
| 156 | 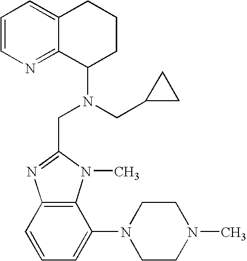 | A |
| 159 | 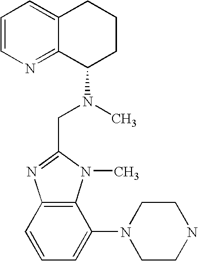 | A |
| 160 | 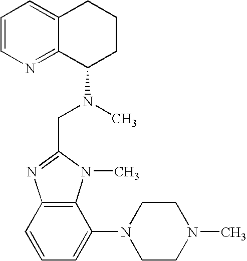 | A |
| 161 | 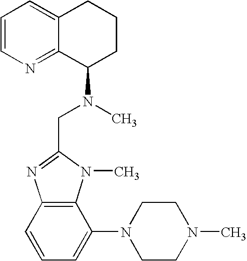 | A |
| 164 | 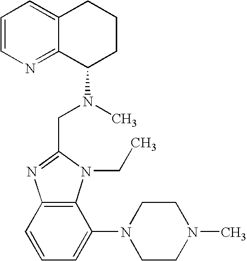 | A |
| 165 | 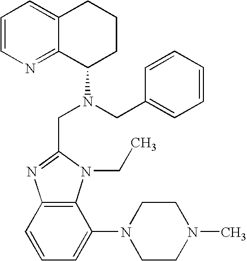 | A |
| 166 | 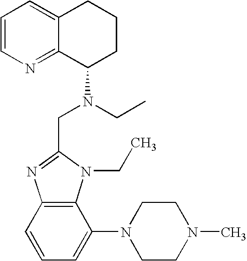 | A |
| 167 | 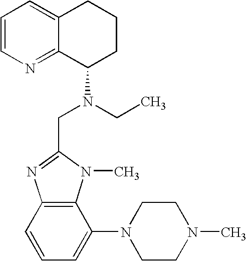 | A |
| 168 | 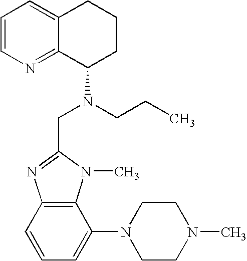 | A |
| 169 | 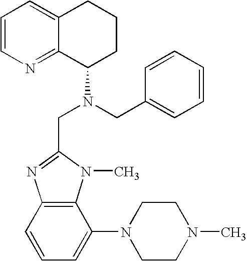 | A |
| 170 | 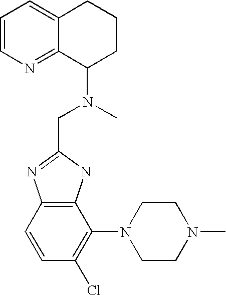 | A |
| 171 | 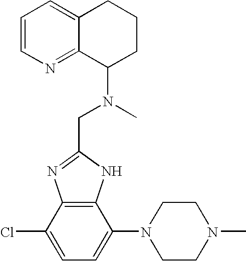 | A |
| 172 | 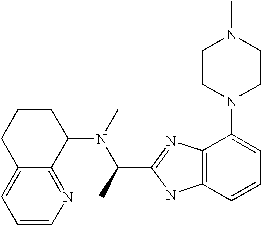 | A |
| 176 | 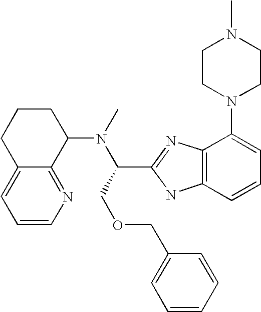 | A |
| 177 | 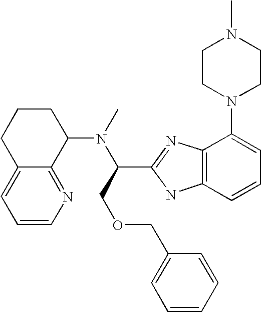 | A |
| 178 | 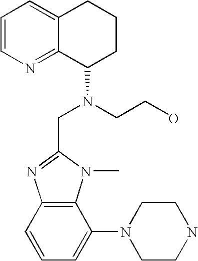 | A |
| 179 | 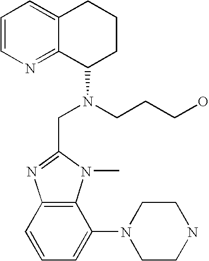 | A |
| 180 | 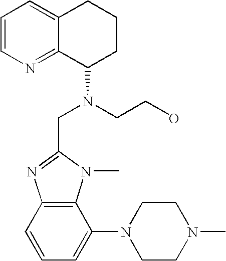 | A |
| 181 | 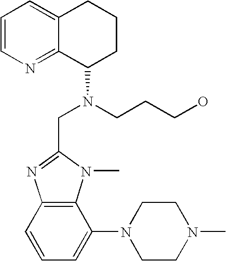 | A |
| 182 | 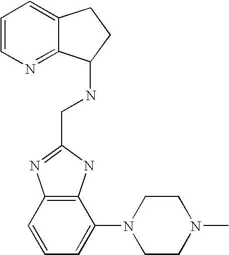 | B |
| 183 | 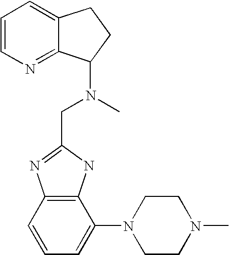 | A |
| 184 | 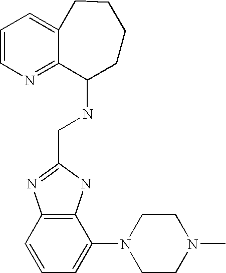 | A |
| 185 | 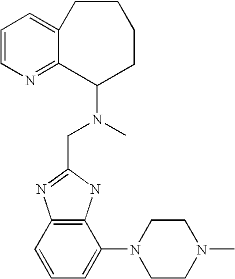 | A |

*“A” indicates compounds with activity in the range of less than 100 nM as determined by the infectivity assay.
“B” indicates compounds with activity in the range of between 100 nM and 500 nM as determined by the infectivity assay.
“C” indicates compounds with activity in the range of between 500 nM and 10 μM as determined by the infectivity assay.

Test compounds were employed in free or salt form.

All research complied with the principles of laboratory animal care (NIH publication No. 85-23, revised 1985) and GlaxoSmithKline policy on animal use.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of formula (I): 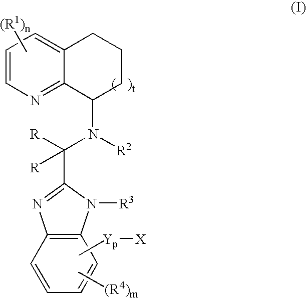
wherein:
t is 0, 1, or 2;
each R independently is H, alkyl, alkenyl, alkynyl, haloalkyl, cycloalkyl, —R^{a}Ay, —R^{a}OR⁵, or —R^{a}S(O)_{q}R⁵;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from a group consisting of H, optionally substituted alkyl, haloalkyl, cycloalkyl, alkenyl, alkynyl, —R^{a}Ay, —R^{a}OR⁵, —R^{a}S(O)_{q}R⁵ wherein R² is not amine or alkylamine, or substituted with amine or alkylamine;
R³ is H, optionally substituted alkyl, haloalkyl, cycloalkyl, alkenyl, alkynyl, —R^{a}Ay, —R^{a}OR⁵, or —R^{a}S(O)_{q}R⁵ wherein when p is 0, R³ is not amine or alkylamine, or substituted with amine or alkylamine;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido,
m is 0, 1, or 2;
each R⁵ independently is H, alkyl, alkenyl, alkynyl, cycloalkyl, —R^{a}Ay, or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, S(O)_{q}NR¹⁰, or —NR¹⁰S(O)_{q}—;
X is —N(R¹⁰)₂—R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay or -HetR^{a}Het, wherein when p is 0 then X is not —N(R¹⁰)₂;
each R^{a} independently is an optionally substituted alkylene, cycloalkylene, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or —R^{a}Het
each of R⁶ and R⁷ independently are selected from H, alkyl, alkenyl, alkynyl, cycloalkyl, cycloalkenyl, —R^{a}cycloalkyl, —R^{a}OH, —R^{a}OR⁵, —R^{a}NR⁸R⁹, -Ay, -Het —R^{a}Ay, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted 4-, 5-, or 6-membered heterocyclyl or heteroaryl group; or a pharmaceutically acceptable salt or ester thereof.

2. The compound of claim 1 wherein -Het is optionally substituted with at least one of alkyl, —(C═O)alkyl, alkoxy, hydroxyl, halogen, cycloalkyl, cycloalkoxy, cyano, amide, amino, or alkylamino.

3. The compound of claim 1 wherein t is 0.

4. The compound of claim 1 wherein t is 1.

5. The compound of claim 1 wherein t is 2.

6. The compound of claim 1 wherein R is H or alkyl.

7. The compound of claim 3 wherein R is H.

8. The compound of claim 1 wherein n is 0.

9. The compound of claim 1 wherein n is 1 and R¹ is halogen, haloalkyl, alkyl, OR¹⁰, NR⁶R⁷, CO₂R¹⁰, CONR⁶R⁷, or cyano.

10. The compound of claim 1 wherein R² is H, optionally substituted alkyl, haloalkyl, or cycloalkyl and wherein R² is not substituted with amine or alkylamine.

11. The compound of claim 10 wherein R² is alkyl optionally substituted with cycloalkyl.

12. The compound of claim 10 wherein R² is a branched chain alkyl.

13. The compound of claim 10 wherein R² is optionally substituted alkyl, haloalkyl, or cycloalkyl and wherein R² is not substituted with amine or alkylamine.

14. The compound of claim 1 wherein R³ is H, optionally substituted alkyl, haloalkyl, cycloalkyl, alkenyl, or alkynyl and wherein when p is 0, R³ is not substituted with amine or alkylamine.

15. The compound of claim 14 wherein R³ is H, optionally substituted alkyl, haloalkyl, or cycloalkyl and wherein when p is 0, R³ is not substituted with amine or alkylamine.

16. The compound of claim 14 wherein R³ is H or optionally substituted alkyl and wherein when p is 0, R³ is not substituted with amine or alkylamine.

17. The compound of claim 1 wherein R³ is H.

18. The compound of claim 1 wherein R³ is optionally substituted alkyl.

19. The compound of claim 14 wherein R³ is a branched chain alkyl.

20. The compound of claim 1 wherein m is 0.

21. The compound of claim 1 wherein m is 1 or 2.

22. The compound of claim 21 wherein m is 1.

23. The compound of claim 22 wherein R⁴ is halogen, haloalkyl, alkyl, OR¹⁰, NR⁶R⁷, CO₂R¹⁰, CONR⁶R⁷, or cyano.

24. The compound of claim 1 wherein R^{a} is alkylene or cycloalkylene, optionally substituted with at least one of alkyl, hydroxyl or oxo.

25. The compound of claim 1 wherein p is 0 and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂.

26. The compound of claim 25 wherein X is —R^{a}N(R¹⁰)₂, -Het, —R^{a}Het, -HetN(R¹⁰)₂, —R^{a}HetN(R¹⁰)₂, or -HetR^{a}N(R¹⁰)₂.

27. The compound of claim 25 wherein X is -Het, optionally substituted with at least one of alkyl, —(C═O)alkyl, alkoxy or hydroxyl.

28. The compound of claim 1 wherein each R is H; R² is alkyl, haloalkyl, or cycloalkyl; R³ is alkyl, haloalkyl, or cycloalkyl; n is 0; m is 0; p is 0; X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂; R^{a} is an optionally substituted alkylene, cycloalkylene, alkenylene, cycloalkenylene, or alkynylene; and R¹⁰ is H or alkyl.

29. The compound of claim 28 wherein X is -Het or —R^{a}Het and -Het is optionally substituted with at least one alkyl.

30. The compound of claim 29 wherein -Het is substituted with a branched chain alkyl.

31. The compound of claim 1 wherein
p is 1;
Y is C(O), —N(R¹⁰)—, —O—, —S—, —C(O)NR¹⁰—, —NR¹⁰CO—, or —S(O)_{q}NR¹⁰—;
X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂; and
-Het is optionally substituted with at least one of alkyl, —(C═O)alkyl, alkoxy, hydroxyl.

32. The compound of claim 31 wherein p is 1; Y is —C(O) or —C(O)NR10; X is —R^{a}Het or -Het; and -Het is optionally substituted with at least one alkyl.

33. The compound of claim 32 wherein -Het is substituted with a branched chain alkyl.

34. The compound of claim 1 wherein -Het is piperidine, piperazine, azetidine, pyrrolidine, imidazole, or pyridine.

35. The compound of claim 1 wherein the substituent —(Y)ₚ—X is located on the depicted benzimidazole ring as in formula (I-A): 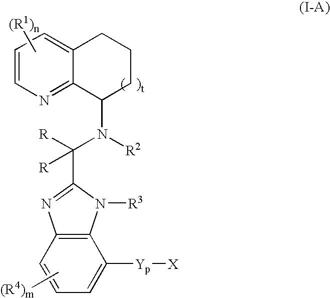
wherein all variables are as defined with respect to formula (I); or a pharmaceutically acceptable salt or ester thereof.

36. The compound of claim 35 wherein each R is H; R² is alkyl or cycloalkyl; R³ is alkyl or cycloalkyl; n is 0; m is 0; p is 0; X is —R^{a}N(R¹⁰)₂, —-AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂; R^{a} is alkylene, cycloalkylene, alkenylene, cycloalkenylene, or alkynylene; and R¹⁰ is H or alkyl.

37. The compound of claim 35 wherein X is -Het, —R^{a}Het, or HetR^{a}N(R¹⁰)₂.

38. The compound of claim 37 wherein X is -Het, optionally substituted with at least one of alkyl, —(C═O)alkyl, alkoxy or hydroxyl.

39. The compound of claim 38 wherein -Het is substituted with a branched chain alkyl.

40. A compound of claim 1 selected from the group consisting of
N-[2-(1H-Imidazol-4-yl)ethyl]-1-methyl-2-{[methyl(5,6,7,8-tetrahydro-8-quinolinyl)amino]methyl}-1H-benzimidazole-7-carboxamide;
N-[2-(1H-Imidazol-4-yl)ethyl]-1-methyl-2-{[methyl(5,6,7,8-tetrahydro-8-quinolinyl)amino]methyl}-1H-benzimidazole-4-carboxamide;
N-[2-(1H-Imidazol-4-yl)ethyl]-1-methyl-2-{[methyl(5,6,7,8-tetrahydro-8-quinolinyl)amino]methyl}-1H-benzimidazole-4-carboxamide;
N-[2-(1-Methyl-1H-imidazol-4-yl)ethyl]-2-{[methyl(5,6,7,8-tetrahydro-8-quinolinyl)amino]methyl}-1H-benzimidazole-4-carboxamide;
N-(2-Aminoethyl)-2-{[methyl(5,6,7,8-tetrahydro-8-quinolinyl)amino]methyl}-1H-benzimidazole-4-carboxamide;
2-{[Methyl(5,6,7,8-tetrahydro-8-quinolinyl)amino]methyl}-N-[2-(1-piperidinyl)propyl]-1H-benzimidazole-4-carboxamide;
2-{[Methyl(5,6,7,8-tetrahydro-8-quinolinyl)amino]methyl}-N-[3-(1-pyrrolidinyl)propyl]-1H-benzimidazole-4-carboxamide;
N-[3-(Dimethylamino)propyl]-2-{[methyl(5,6,7,8-tetrahydro-8-quinolinyl)amino]methyl}-1H-benzimidazole-4-carboxamide;
N-({4-[(4-Amino-1-piperidinyl)carbonyl]-1H-benzimidazol-2-yl}methyl)-N-methyl-5,6,7,8-tetrahydro-8-quinolinamine;
N-({4-[(3-Amino-1-pyrrolidinyl)carbonyl]-1H-benzimidazol-2-yl}methyl)-N-methyl-5,6,7,8-tetrahydro-8-quinolinamine;
N-{[4-({[2-(1H-imidazol-4-yl)ethyl]amino}methyl)-1H-benzimidazol-2-yl]methyl}-N-methyl-5,6,7,8-tetrahydro-8-quinolinamine;
N-Methyl-N-{[4-(1-piperazinylmethyl)-1H-benzimidazol-2-yl]methyl}-5,6,7,8-tetrahydro-8-quinolinamine;
N-methyl-N-({4-[4-(2-methylpropyl)-1-piperazinyl]-1H-benzimidazol-2-yl}methyl)-5,6,7,8-tetrahydro-8-quinolinamine;
2-{[Methyl(5,6,7,8-tetrahydroquinolin-8-yl)amino]methyl}-1H-benzimidazole-5-carboxamide;
N-Methyl-N-[2-(methylamino)ethyl]-2-{[methyl(5,6,7,8-tetrahydro-8-quinolinyl)amino]methyl}-1H-benzimidazole-5-carboxamide;
N-[2-(Dimethylamino)ethyl]-2-{[methyl(5,6,7,8-tetrahydro-8-quinolinyl)amino]methyl}-1H-benzimidazole-5-carboxamide;
N-[2-(Methylamino)ethyl]-2-{[methyl(5,6,7,8-tetrahydro-8-quinolinyl)amino]methyl}-1H-benzimidazole-5-carboxamide,
N-[2-(Dimethylamino)ethyl]-N-methyl-2-{[methyl(5,6,7,8-tetrahydro-8-quinolinyl)amino]methyl}-1H-benzimidazole-5-carboxamide;
N-[2-(1H-imidazol-4-yl)ethyl]-N-methyl-2-{[methyl(5,6,7,8-tetrahydro-8-quinolinyl)amino]methyl}-1H-benzimidazole-4-carboxamide;
N-methyl-N-({4-[(2-methyl-1-piperazinyl)carbonyl]-1H-benzimidazol-2-yl}methyl)-5,6,7,8-tetrahydro-8-quinolinamine;
N-({4-[(1S,4S)-2,5-diazabicyclo[2.2.1]hept-2-ylcarbonyl]-1H-benzimidazol-2-yl}methyl)-N-methyl-5,6,7,8-tetrahydro-8-quinolinamine;
N-{[4-(hexahydro-1H-1,4-diazepin-1-ylcarbonyl)-1H-benzimidazol-2-yl]methyl}-N-methyl-5,6,7,8-tetrahydro-8-quinolinamine;
N-({4-[3-(Dimethylamino)propyl]-1H-benzimidazol-2-yl}methyl)-N-methyl-5,6,7,8-tetrahydro-8-quinolinamine;
N-Methyl-N-({4-[3-(1-pyrrolidinyl)propyl]-1H-benzimidazol-2-yl}methyl)-5,6,7,8-tetrahydro-8-quinolinamine;
N-methyl-N-({4-[3-(1-piperidinyl)propyl]-1H-benzimidazol-2-yl}methyl)-5,6,7,8-tetrahydro-8-quinolinamine;
N-{[4-(3-aminopropyl)-1H-benzimidazol-2-yl]methyl}-N-methyl-5,6,7,8-tetrahydro-8-quinolinamine;
2-{[Methyl(5,6,7,8-tetrahydro-8-quinolinyl)amino]methyl}-N-[3-(4-morpholinyl)propyl]-1H-benzimidazole-4-carboxamide;
N-(1H-Benzimidazol-2-ylmethyl)-2-{[methyl(5,6,7,8-tetrahydro-8-quinolinyl)amino]methyl}-1H-benzimidazole-4-carboxamide;
2-{[Methyl(5,6,7,8-tetrahydro-8-quinolinyl)amino]methyl}-N-3-pyrrolidinyl-1H-benzimidazole-4-carboxamide;
N-[3-(1H-Imidazol-1-yl)propyl]-2-{[methyl(5,6,7,8-tetrahydro-8-quinolinyl)amino]methyl}-1H-benzimidazole-4-carboxamide;
N-({4-[(4-Amino-1-piperidinyl)carbonyl]-1H-benzimidazol-2-yl}methyl)-N-methyl-5,6,7,8-tetrahydro-8-quinolinamine;
N-({4-[(3-amino-1-pyrrolidinyl)carbonyl]-1H-benzimidazol-2-yl}methyl)-N-methyl-5,6,7,8-tetrahydro-8-quinolinamine;
N-Methyl-N-({4-[(4-methylhexahydro-1H-1,4-diazepin-1-yl)carbonyl]-1H-benzimidazol-2-yl}methyl)-5,6,7,8-tetrahydro-8-quinolinamine;
[2-(Dimethylamino)ethyl](2-{[methyl(5,6,7,8-tetrahydro-8-quinolinyl)amino]methyl}-1H-benzimidazol-4-yl)amine;
Methyl[2-(methylamino)ethyl](2-{[methyl(5,6,7,8-tetrahydro-8-quinolinyl)amino]methyl}-1H-benzimidazol-4-yl)amine;
[2-(Dimethylamino)ethyl]methyl(2-{[methyl(5,6,7,8-tetrahydro-8-quinolinyl)amino]methyl}-1H-benzimidazol-4-yl)amine;
N-Methyl-N-({4-[4-(1-methylethyl)-1-piperazinyl]-1H-benzimidazol-2-yl}methyl)-5,6,7,8-tetrahydro-8-quinolinamine;
N-(1-Methylethyl)-N-{[4-(4-methyl-1-piperazinyl)-1H-benzimidazol-2-yl]methyl}-5,6,7,8-tetrahydro-8-quinolinamine;
N-(1-methylethyl)-N-({4-[4-(1-methylethyl)-1-piperazinyl]-1H-benzimidazol-2-yl}methyl)-5,6,7,8-tetrahydro-8-quinolinamine;
N-{1-methyl-1-[4-(4-methyl-1-piperazinyl)-1H-benzimidazol-2-yl]ethyl}-5,6,7,8-tetrahydro-8-quinolinamine;
N-Methyl-N-{1-methyl-1-[4-(4-methyl-1-piperazinyl)-1H-benzimidazol-2-yl]ethyl}-5,6,7,8-tetrahydro-8-quinolinamine;
N-({4-[4-(Aminoacetyl)-1-piperazinyl]-1H-benzimidazol-2-yl}methyl)-N-methyl-5,6,7,8-tetrahydro-8-quinolinamine;
(8R)—N-Methyl-N-{[4-(4-methyl-1-piperazinyl)-1H-benzimidazol-2-yl]methyl}-5,6,7,8-tetrahydro-8-quinolinamine;
(8S)—N-Methyl-N-{[4-(4-methyl-1-piperazinyl)-1H-benzimidazol-2-yl]methyl}-5,6,7,8-tetrahydro-8-quinolinamine;
(8R)—N-Ethyl-N-{[4-(4-methyl-1-piperazinyl)-1H-benzimidazol-2-yl]methyl}-5,6,7,8-tetrahydro-8-quinolinamine;
(8S)—N-Ethyl-N-{[4-(4-methyl-1-piperazinyl)-1H-benzimidazol-2-yl]methyl}-5,6,7,8-tetrahydro-8-quinolinamine;
(8R)—N-{[4-(4-methyl-1-piperazinyl)-1H-benzimidazol-2-yl]methyl}-N-propyl-5,6,7,8-tetrahydro-8-quinolinamine;
(8S)—N-{[4-(4-methyl-1-piperazinyl)-1H-benzimidazol-2-yl]methyl}-N-propyl-5,6,7,8-tetrahydro-8-quinolinamine;
(8S)—N-(1-Methylethyl)-N-{[4-(4-methyl-1-piperazinyl)-1H-benzimidazol-2-yl]methyl}-5,6,7,8-tetrahydro-8-quinolinamine;
(8R)—N-(Cyclopropylmethyl)-N-{[4-(4-methyl-1-piperazinyl)-1H-benzimidazol-2-yl]methyl}-5,6,7,8-tetrahydro-8-quinolinamine;
(8S)—N-(Cyclopropylmethyl)-N-{[4-(4-methyl-1-piperazinyl)-1H-benzimidazol-2-yl]methyl}-5,6,7,8-tetrahydro-8-quinolinamine;
(8S)—N-({4-[(8aR)-hexahydropyrrolo[1,2-a]pyrazin-2(1H)-yl]-1H-benzimidazol-2-yl}methyl)-N-methyl-5,6,7,8-tetrahydro-8-quinolinamine;
(8S)—N-Ethyl-N-({4-[(8aR)-hexahydropyrrolo[1,2-a]pyrazin-2(1H)-yl]-1H-benzimidazol-2-yl}methyl)-5,6,7,8-tetrahydro-8-quinolinamine;
(8S)—N-({4-[(8aR)-hexahydropyrrolo[1,2-a]pyrazin-2(1H)-yl]-1H-benzimidazol-2-yl}methyl)-N-propyl-5,6,7,8-tetrahydro-8-quinolinamine;
(8S)—N-methyl-N-({4-[(1R,5R)-7-methyl-3,7-diazabicyclo[3.3.1]non-3-yl]-1H-benzimidazol-2-yl}methyl)-5,6,7,8-tetrahydro-8-quinolinamine;
N-Cyclopropyl-N-{[4-(4-methyl-1-piperazinyl)-1H-benzimidazol-2-yl]methyl}-5,6,7,8-tetrahydro-8-quinolinamine;
(8S)—N-{(1S)-1-[4-(methyloxy)phenyl]ethyl}-N-{[4-(4-methyl-1-piperazinyl)-1H-benzimidazol-2-yl]methyl}-5,6,7,8-tetrahydro-8-quinolinamine;
(8S)—N-{[4-(4-Methyl-1-piperazinyl)-1H-benzimidazol-2-yl]methyl}-N-{2-[(phenylmethyl)oxy]ethyl}-5,6,7,8-tetrahydro-8-quinolinamine;
2-{{[4-(4-Methyl-1-piperazinyl)-1H-benzimidazol-2-yl]methyl}[(8S)-5,6,7,8-tetrahydro-8-quinolinyl]amino}ethanol;
3-{{[4-(4-Methyl-1-piperazinyl)-1H-benzimidazol-2-yl]methyl}[(8S)-5,6,7,8-tetrahydro-8-quinolinyl]amino}-1-propanol;
(8S)—N-{[4-(4-methyl-1-piperazinyl)-1H-benzimidazol-2-yl]methyl}-N-(phenylmethyl)-5,6,7,8-tetrahydro-8-quinolinamine;
N-Methyl-N-{[1-methyl-7-(4-methyl-1-piperazinyl)-1H-benzimidazol-2-yl]methyl}-5,6,7,8-tetrahydro-8-quinolinamine;
N-Ethyl-N-{[1-methyl-7-(4-methyl-1-piperazinyl)-1H-benzimidazol-2-yl]methyl}-5,6,7,8-tetrahydro-8-quinolinamine,
N-{[1-Methyl-7-(4-methyl-1-piperazinyl)-1H-benzimidazol-2-yl]methyl}-N-propyl-5,6,7,8-tetrahydro-8-quinolinamine;
N-(Cyclopropylmethyl)-N-{[1-methyl-7-(4-methyl-1-piperazinyl)-1H-benzimidazol-2-yl]methyl}-5,6,7,8-tetrahydro-8-quinolinamine;
(8S)—N-methyl-N-{[1-methyl-7-(1-piperazinyl)-1H-benzimidazol-2-yl]methyl}-5,6,7,8-tetrahydro-8-quinolinamine;
(8S)—N-Methyl-N-{[1-methyl-7-(4-methyl-1-piperazinyl)-1H-benzimidazol-2-yl]methyl}-5,6,7,8-tetrahydro-8-quinolinamine;
(8S)—N-{[1-ethyl-7-(4-methyl-1-piperazinyl)-1H-benzimidazol-2-yl]methyl}-N-methyl-5,6,7,8-tetrahydro-8-quinolinamine;
(8S)—N-{[1-Ethyl-7-(4-methyl-1-piperazinyl)-1H-benzimidazol-2-yl]methyl}-N-(phenylmethyl)-5,6,7,8-tetrahydro-8-quinolinamine;
(8S)—N-Ethyl-N-{[1-ethyl-7-(4-methyl-1-piperazinyl)-1H-benzimidazol-2-yl]methyl}-5,6,7,8-tetrahydro-8-quinolinamine;
N-{[5-Chloro-4-(4-methyl-1-piperazinyl)-1H-benzimidazol-2-yl]methyl}-N-methyl-5,6,7,8-tetrahydro-8-quinolinamine;
N-{[4-Chloro-7-(4-methyl-1-piperazinyl)-1H-benzimidazol-2-yl]methyl}-N-methyl-5,6,7,8-tetrahydro-8-quinolinamine;
N-Methyl-N-{(1R)-1-[4-(4-methyl-1-piperazinyl)-1H-benzimidazol-2-yl]ethyl}-5,6,7,8-tetrahydro-8-quinolinamine;
N-Methyl-N-{(1R)-1-[4-(4-methyl-1-piperazinyl)-1H-benzimidazol-2-yl]-2-[(phenylmethyl)oxy]ethyl}-5,6,7,8-tetrahydro-8-quinolinamine;
N-Methyl-N-{(1S)-1-[4-(4-methyl-1-piperazinyl)-1H-benzimidazol-2-yl]-2-[(phenylmethyl)oxy]ethyl}-5,6,7,8-tetrahydro-8-quinolinamine;
N-Methyl-N-{[4-(1-piperazinylcarbonyl)-1H-benzimidazol-2-yl]methyl}-5,6,7,8-tetrahydro-8-quinolinamine;
N-[2-(1H-Imidazol-4-yl)ethyl]-2-{[methyl(5,6,7,8-tetrahydro-8-quinolinyl)amino]methyl}-1H-benzimidazole-4-carboxamide;
(8R)—N-Methyl-N-{[1-methyl-7-(4-methyl-1-piperazinyl)-1H-benzimidazol-2-yl]methyl}-5,6,7,8-tetrahydro-8-quinolinamine;
2-{{[1-Methyl-7-(1-piperazinyl)-1H-benzimidazol-2-yl]methyl}[(8S)-5,6,7,8-tetrahydro-8-quinolinyl]amino}ethanol;
3-{{[1-Methyl-7-(1-piperazinyl)-1H-benzimidazol-2-yl]methyl}[(8S)-5,6,7,8-tetrahydro-8-quinolinyl]amino}-1-propanol;
2-{{[1-Methyl-7-(4-methyl-1-piperazinyl)-1H-benzimidazol-2-yl]methyl}[(8S)-5,6,7,8-tetrahydro-8-quinolinyl]amino}ethanol;
3-{{[1-Methyl-7-(4-methyl-1-piperazinyl)-1H-benzimidazol-2-yl]methyl}[(8S)-5,6,7,8-tetrahydro-8-quinolinyl]amino}-1-propanol;
N-Methyl-N-{[4-(4-methyl-1-piperazinyl)-1H-benzimidazol-2-yl]methyl}-6,7-dihydro-5H-cyclopenta[b]pyridin-7-amine;
N-{[4-(4-methyl-1-piperazinyl)-1H-benzimidazol-2-yl]methyl}-6,7,8,9-tetrahydro-5H-cyclohepta[b]pyridin-9-amine;
N-methyl-N-{[4-(4-methyl-1-piperazinyl)-1H-benzimidazol-2-yl]methyl}-6,7,8,9-tetrahydro-5H-cyclohepta[b]pyridin-9-amine;
and pharmaceutically acceptable salts or esters thereof.

41. A compound selected from the group consisting of:
N-methyl-N-{[4-(1-piperazinyl)-1H-benzimidazol-2-yl]methyl}-5,6,7,8-tetrahydro-8-quinolinamine;
N-methyl-N-{[4-(4-methyl-1-piperazinyl)-1H-benzimidazol-2-yl]methyl}-5,6,7,8-tetrahydro-8-quinolinamine;
N-methyl-N-({4-[4-(2-methylpropyl)-1-piperazinyl]-1H-benzimidazol-2-yl}methyl)-5,6,7,8-tetrahydro-8-quinolinamine;
N-Methyl-N-({4-[4-(1-methylethyl)-1-piperazinyl]-1H-benzimidazol-2-yl}methyl)-5,6,7,8-tetrahydro-8-quinolinamine;
N-(1-Methylethyl)-N-{[4-(4-methyl-1-piperazinyl)-1H-benzimidazol-2-yl]methyl}-5,6,7,8-tetrahydro-8-quinolinamine;
(8R)—N-Methyl-N-{[4-(4-methyl-1-piperazinyl)-1H-benzimidazol-2-yl]methyl}-5,6,7,8-tetrahydro-8-quinolinamine;
(8S)—N-Methyl-N-{[4-(4-methyl-1-piperazinyl)-1H-benzimidazol-2-yl]methyl}-5,6,7,8-tetrahydro-8-quinolinamine;
(8S)—N-Ethyl-N-{[4-(4-methyl-1-piperazinyl)-1H-benzimidazol-2-yl]methyl}-5,6,7,8-tetrahydro-8-quinolinamine;
(8S)—N-{[4-(4-methyl-1-piperazinyl)-1H-benzimidazol-2-yl]methyl}-N-propyl-5,6,7,8-tetrahydro-8-quinolinamine;
(8S)—N-(1-Methylethyl)-N-{[4-(4-methyl-1-piperazinyl)-1H-benzimidazol-2-yl]methyl}-5,6,7,8-tetrahydro-8-quinolinamine;
(8S)—N-(Cyclopropylmethyl)-N-{[4-(4-methyl-1-piperazinyl)-1H-benzimidazol-2-yl]methyl}-5,6,7,8-tetrahydro-8-quinolinamine;
(8S)—N-({4-[(8aR)-hexahydropyrrolo[1,2-a]pyrazin-2(1H)-yl]-1H-benzimidazol-2-yl}methyl)-N-methyl-5 ,6,7,8-tetrahydro-8-quinolinamine;
(8S)—N-Ethyl-N-({4-[(8aR)-hexahydropyrrolo[1,2-a]pyrazin-2(1H)-yl]-1H-benzimidazol-2-yl}methyl)-5,6,7,8-tetrahydro-8-quinolinamine;
(8S)—N-({4-[(8aR)-hexahydropyrrolo[1,2-a]pyrazin-2(1H)-yl]-1H-benzimidazol-2-yl}methyl)-N-propyl-5,6,7,8-tetrahydro-8-quinolinamine;
2-{{[4-(4-Methyl-1-piperazinyl)-1H-benzimidazol-2-yl]methyl}[(8S)-5,6,7,8-tetrahydro-8-quinolinyl]amino}ethanol;
3-{{[4-(4-Methyl-1-piperazinyl)-1H-benzimidazol-2-yl]methyl}[(8S)-5,6,7,8-tetrahydro-8-quinolinyl]amino}-1-propanol;
(8S)—N-{[4-(4-methyl-1-piperazinyl)-1H-benzimidazol-2-yl]methyl}-N-(phenylmethyl)-5,6,7,8-tetrahydro-8-quinolinamine;
N-Methyl-N-{[1-methyl-7-(4-methyl-1-piperazinyl)-1H-benzimidazol-2-yl]methyl}-5,6,7,8-tetrahydro-8-quinolinamine;
N-Ethyl-N-{[1-methyl-7-(4-methyl-1-piperazinyl)-1H-benzimidazol-2-yl]methyl}-5,6,7,8-tetrahydro-8-quinolinamine;
N-{[1-Methyl-7-(4-methyl-1-piperazinyl)-1H-benzimidazol-2-yl]methyl}-N-propyl-5,6,7,8-tetrahydro-8-quinolinamine;
N-(Cyclopropylmethyl)-N-{[1-methyl-7-(4-methyl-1-piperazinyl)-1H-benzimidazol-2-yl]methyl}-5,6,7,8-tetrahydro-8-quinolinamine;
(8S)—N-methyl-N-{[1-methyl-7-(1-piperazinyl)-1H-benzimidazol-2-yl]methyl}-5,6,7,8-tetrahydro-8-quinolinamine;
(8S)—N-Methyl-N-{[1-methyl-7-(4-methyl-1-piperazinyl)-1H-benzimidazol-2-yl]methyl}-5,6,7,8-tetrahydro-8-quinolinamine;
(8S)—N-{[1-ethyl-7-(4-methyl-1-piperazinyl)-1H-benzimidazol-2-yl]methyl}-N-methyl-5,6,7,8-tetrahydro-8-quinolinamine;
(8S)—N-{[1-Ethyl-7-(4-methyl-1-piperazinyl)-1H-benzimidazol-2-yl]methyl}-N-(phenylmethyl)-5,6,7,8-tetrahydro-8-quinolinamine;
(8S)—N-Ethyl-N-{[1-ethyl-7-(4-methyl-1-piperazinyl)-1H-benzimidazol-2-yl]methyl}-5,6,7,8-tetrahydro-8-quinolinamine;
(8S)—N-Ethyl-N-{[1-methyl-7-(4-methyl-1-piperazinyl)-1H-benzimidazol-2-yl]methyl}-5,6,7,8-tetrahydro-8-quinolinamine;
(8S)—N-{[1-Methyl-7-(4-methyl-1-piperazinyl)-1H-benzimidazol-2-yl]methyl}-N-propyl-5,6,7,8-tetrahydro-8-quinolinamine;
(8S)—N-{[1-Methyl-7-(4-methyl-1-piperazinyl)-1H-benzimidazol-2-yl]methyl}-N-(phenylmethyl)-5,6,7,8-tetrahydro-8-quinolinamine;
(8R)—N-Methyl-N-{[1-methyl-7-(4-methyl-1-piperazinyl)-1H-benzimidazol-2-yl]methyl}-5,6,7,8-tetrahydro-8-quinolinamine;
2-{{[1-Methyl-7-(4-methyl-1-piperazinyl)-1H-benzimidazol-2-yl]methyl}[(8S)-5,6,7,8-tetrahydro-8-quinolinyl]amino}ethanol;
3-{{[1-Methyl-7-(4-methyl-1-piperazinyl)-1H-benzimidazol-2-yl]methyl}[(8S)-5,6,7,8-tetrahydro-8-quinolinyl]amino}-1-propanol;
N-Methyl-N-{[4-(4-methyl-1-piperazinyl)-1H-benzimidazol-2-yl]methyl}-6,7-dihydro-5H-cyclopenta[b]pyridin-7-amine;
N-methyl-N-{[4-(4-methyl-1-piperazinyl)-1H-benzimidazol-2-yl]methyl}-6,7,8,9-tetrahydro-5H-cyclohepta[b]pyridin-9-amine;
and pharmaceutically acceptable salts or esters thereof.

42. A compound selected from the group consisting of:
N-Methyl-N-{[1-methyl-7-(4-methyl-1-piperazinyl)-1H-benzimidazol-2-yl]methyl}-5,6,7,8-tetrahydro-8-quinolinamine;
N-Ethyl-N-{[1-methyl-7-(4-methyl-1-piperazinyl)-1H-benzimidazol-2-yl]methyl}-5,6,7,8-tetrahydro-8-quinolinamine;
N-{[1-Methyl-7-(4-methyl-1-piperazinyl)-1H-benzimidazol-2-yl]methyl}-N-propyl-5,6,7,8-tetrahydro-8-quinolinamine;
N-(Cyclopropylmethyl)-N-{[1-methyl-7-(4-methyl-1-piperazinyl)-1H-benzimidazol-2-yl]methyl}-5,6,7,8-tetrahydro-8-quinolinamine;
(8S)—N-methyl-N-{[1-methyl-7-(1-piperazinyl)-1H-benzimidazol-2-yl]methyl}-5,6,7,8-tetrahydro-8-quinolinamine;
(8S)—N-Methyl-N-{[1-methyl-7-(4-methyl-1-piperazinyl)-1H-benzimidazol-2-yl]methyl}-5,6,7,8-tetrahydro-8-quinolinamine;
(8S)—N-{[1-ethyl-7-(4-methyl-1-piperazinyl)-1H-benzimidazol-2-yl]methyl}-N-methyl-5,6,7,8-tetrahydro-8-quinolinamine;
(8S)—N-{[1-Ethyl-7-(4-methyl-1-piperazinyl)-1H-benzimidazol-2-yl]methyl}-N-(phenylmethyl)-5,6,7,8-tetrahydro-8-quinolinamine;
(8S)—N-Ethyl-N-{[1-ethyl-7-(4-methyl-1-piperazinyl)-1H-benzimidazol-2-yl]methyl}-5,6,7,8-tetrahydro-8-quinolinamine;
(8S)—N-Ethyl-N-{[1-methyl-7-(4-methyl-1-piperazinyl)-1H-benzimidazol-2-yl]methyl}-5,6,7,8-tetrahydro-8-quinolinamine;
(8S)—N-{[1-Methyl-7-(4-methyl-1-piperazinyl)-1H-benzimidazol-2-yl]methyl}-N-propyl-5,6,7,8-tetrahydro-8-quinolinamine; and
(8S)—N-{[1-Methyl-7-(4-methyl-1-piperazinyl)-1H-benzimidazol-2-yl]methyl}-N-(phenylmethyl)-5,6,7,8-tetrahydro-8-quinolinamine;
(8R)—N-Methyl-N-{[1-methyl-7-(4-methyl-1-piperazinyl)-1H-benzimidazol-2-yl]methyl}-5,6,7,8-tetrahydro-8-quinolinamine;
2-{{[1-Methyl-7-(4-methyl-1-piperazinyl)-1H-benzimidazol-2-yl]methyl}[(8S)-5,6,7,8-tetrahydro-8-quinolinyl]amino}ethanol;
3-{{[1-Methyl-7-(4-methyl-1-piperazinyl)-1H-benzimidazol-2-yl]methyl}[(8S)-5,6,7,8-tetrahydro-8-quinolinyl]amino}-1-propanol;
and pharmaceutically acceptable salts or esters thereof.

43. (canceled)

44. A pharmaceutical composition comprising a compound according to claim 1, and a pharmaceutically acceptable carrier.

45. A compound according to claim 1 for use as an active therapeutic substance.

46. A compound according to claim 1 for use in the treatment or prophylaxis of diseases and conditions caused by inappropriate activity of CXCR4.

47. A compound according to claim 1 for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers.

48. The compound of claim 47 wherein the condition or disease is HIV infection, rheumatoid arthritis, inflammation, or cancer.

49. The compound of claim 47 wherein the condition or disease is HIV infection.

50. Use of a compound according to claim 1 in the manufacture of a medicament for use in the treatment or prophylaxis of a condition or disease modulated by a chemokine receptor.

51. Use of a compound according to claim 50 wherein the chemokine receptor is CXCR4.

52. Use of a compound according to claim 1 in the manufacture of a medicament for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers.

53. Use of a compound as in claim 52 wherein the condition or disorder is HIV infection, rheumatoid arthritis, inflammation, or cancer.

54. Use of a compound as in claim 52 wherein the condition is HIV infection.

55. A method for the treatment or prophylaxis of a condition or disease modulated by a chemokine receptor comprising the administration of a compound according to claim 1.

56. The method of claim 55 wherein the chemokine receptor is CXCR4.

57. A method for the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers comprising the administration of a compound according to claim 1.

58. A method for the treatment or prophylaxis of HIV infection rheumatoid arthritis, inflammation, or cancer comprising the administration of a compound according to claim 1.

59. A method for the treatment or prophylaxis of HIV infection comprising the administration of a compound according to claim 1.

60. A method of treatment or prevention of a viral infection in a human comprising administering to said human a composition comprising a compound according to claim 1 and another therapeutic agent.

61. A composition according to claim 44, wherein said composition comprises at least one additional therapeutic agent selected from the group consisting of nucleotide reverse transcriptase inhibitors such as zidovudine, didanosine, lamivudine, zalcitabine, abacavir, stavidine, adefovir, adefovir dipivoxil, fozivudine, todoxil, and similar agents; non-nucleotide reverse transcriptase inhibitors (including an agent having anti-oxidation activity such as immunocal, oltipraz, etc.) such as nevirapine, delavirdine, efavirenz, loviride, immunocal, oltipraz, and similar agents; protease inhibitors such as saquinavir, ritonavir, indinavir, nelfinavir, aprenavir, palinavir, lasinavir, and similar agents; entry inhibitors such as T-20, T-1249, PRO-542, PRO-140, TNX-355, BMS-806, 5-Helix and similar agents; Integrase inhibitors such as L-870, 180 and similar agents; budding inhibitors such as PA-344 and PA-457, and similar agents; and other CXCR4 and/or CCR5 inhibitors such as Sch-C, Sch-D, TAK779, UK 427, 857, TAK449, and similar agents.

62. A method according to claim 60, wherein said therapeutic agent is selected from the group consisting of nucleotide reverse transcriptase inhibitors such as zidovudine, didanosine, lamivudine, zalcitabine, abacavir, stavidine, adefovir, adefovir dipivoxil, fozivudine, todoxil, and similar agents; non-nucleotide reverse transcriptase inhibitors (including an agent having anti-oxidation activity such as immunocal, oltipraz, etc.) such as nevirapine, delavirdine, efavirenz, loviride, immunocal, oltipraz, and similar agents; protease inhibitors such as saquinavir, ritonavir, indinavir, nelfinavir, aprenavir, palinavir, lasinavir, and similar agents; entry inhibitors such as T-20, T-1249, PRO-542, PRO-140, TNX-355, BMS-806, 5-Helix and similar agents; Integrase inhibitors such as L-870, 180 and similar agents; budding inhibitors such as PA-344 and PA-457, and similar agents; and other CXCR4 and/or CCR5 inhibitors such as Sch-C, Sch-D, TAK779, UK 427, 857, TAK449, and similar agents.

63. A process for the preparation of a compound of formula (I) 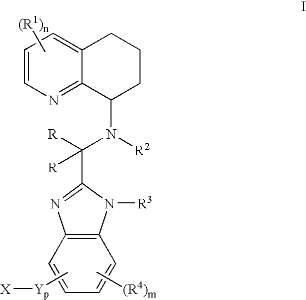
wherein
t is 1;
each R is H;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2, and, as shown, R¹ can be substituted throughout the depicted tetrahydroquinoline;
R² is selected from a group consisting of H, alkyl, haloalkyl, cycloalkyl, alkenyl, alkynyl, —R^{a}Ay, —R^{a}OR⁵, —R^{a}S(O)_{q}R⁵; wherein R² is not amine or alkylamine, or substituted with amine or alkylamine;
R³ is H, alkyl, haloalkyl, cycloalkyl, alkenyl, alkynyl, —R^{a}Ay, —R^{a}OR⁵, or —R^{a}S(O)_{q}R⁵;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, —S(O)_{q}NR¹⁰—, or —NR¹⁰S(O)_{q}—;
X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het, wherein when p is 0 then X is not —N(R¹⁰)₂, or —R^{a}N(R¹⁰)₂;
each R^{a} independently is alkylene, cycloalkylene, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or —R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁵, —R^{a}NR⁸R⁹, -Ay, -Het, —R^{a}Ay, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted 4-, 5-, or 6-membered heterocyclyl or heteroaryl group;
comprising reacting a compound of formula (II) 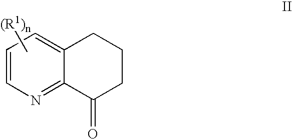
with a compound of formula (IV) 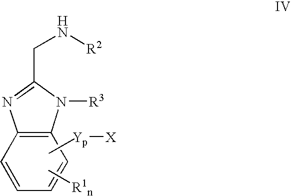
under reductive amination conditions to form a compound of formula (I).

64. A process for the preparation of a compound of formula (I) 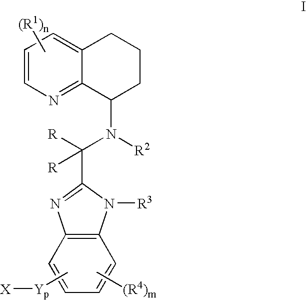
wherein
t is 1;
each R is H;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2, and, as shown, R¹ can be substituted throughout the depicted tetrahydroquinoline;
R² is selected from a group consisting of H, alkyl, haloalkyl, cycloalkyl, alkenyl, alkynyl, —R^{a}Ay, —R^{a}OR⁵, —R^{a}S(O)_{q}R⁵; wherein R² is not amine or alkylamine, or substituted with amine or alkylamine;
R³ is H, alkyl, haloalkyl, cycloalkyl, alkenyl, alkynyl, —R^{a}Ay, —R^{a}OR⁵, or —R^{a}S(O)_{q}R⁵;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, S(O)_{q}NR¹⁰—, or —NR¹⁰S(O)_{q}—;
X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het, wherein when p is 0 then X is not —N(R¹⁰)₂, or —R^{a}N(R¹⁰)₂;
each R^{a} independently is alkylene, cycloalkylene, alkenylene, cycloalkenylene, or alkynylene;
each R¹⁰ independently is H, alkyl, cycloalkyl, alkenyl, alkynyl, cycloalkenyl, —R^{a}cycloalkyl —R^{a}OH, —R^{a}OR⁵, —R^{a}NR⁶R⁷, or —R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁵, —R^{a}NR⁸R⁹, -Ay, -Het, —R^{a}Ay, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted 4-, 5-, or 6-membered heterocyclyl or heteroaryl group;
comprising the step of reacting a compound of formula (III) 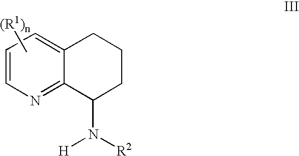
with a compound of formula (V) 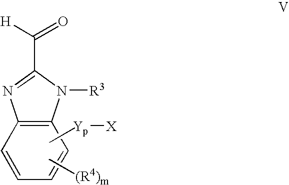
under reductive amination conditions to form a compound of formula (I).

65. A process for the preparation of a compound of formula (I) 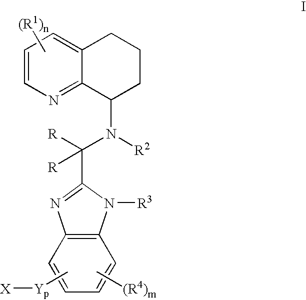
wherein
t is 1;
each R is H;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2, and, as shown, R¹ can be substituted throughout the depicted tetrahydroquinoline;
R² is selected from a group consisting of H, alkyl, haloalkyl, cycloalkyl, alkenyl, alkynyl, —R^{a}Ay, —R^{a}OR⁵, —R^{a}S(O)_{q}R⁵; wherein R² is not amine or alkylamine, or substituted with amine or alkylamine;
R³ is H, alkyl, haloalkyl, cycloalkyl, alkenyl, alkynyl, —R^{a}Ay, —R^{a}OR⁵, or —R^{a}S(O)_{q}R⁵;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰—C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, —S(O)_{q}NR¹⁰—, or —NR¹⁰S(O)_{q}—;
X is —N(R¹⁰)₂, —R^{a}N(R¹⁰)₂, -AyN(R¹⁰)₂, —R^{a}AyN(R¹⁰)₂, AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰))₂, -HetR^{a}Ay, or -HetR^{a}Het, wherein when p is 0 then X is not —N(R¹⁰)₂, or —R^{a}N(R¹⁰)₂;
each R^{a} independently is alkylene, cycloalkylene, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or —R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁵, —R^{a}NR⁸R⁹, -Ay, -Het, —R^{a}Ay, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted 4-, 5-, or 6-membered heterocyclyl or heteroaryl group;
comprising the steps of reacting a compound of formula (III) 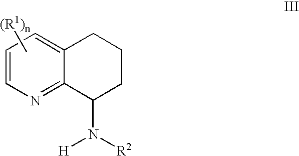
with a compound of formula (VI) 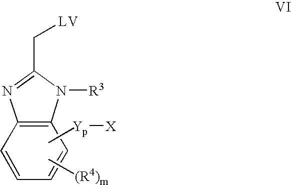
to form a compound of formula (I).

66. A process for the preparation of a compound of formula (I) 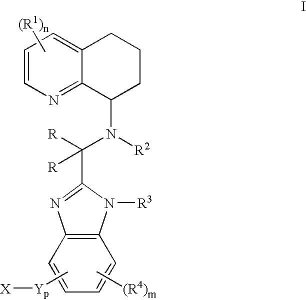
wherein
t is 1;
each R is H;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, R^{a}C(O)R¹⁰, —C(O)R¹⁰, —CO₂R¹⁰, R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2, and, as shown, R¹ can be substituted throughout the depicted tetrahydroquinoline;
R² is selected from a group consisting of H, alkyl, haloalkyl, cycloalkyl, alkenyl, alkynyl, —R^{a}Ay, —R^{a}OR⁵, —R^{a}S(O)_{q}R⁵; wherein R² is not amine or alkylamine, or substituted with amine or alkylamine;
R³ is H, alkyl, haloalkyl, cycloalkyl, alkenyl, alkynyl, —R^{a}Ay, —R^{a}OR⁵, or —R^{a}S(O)_{q}R⁵;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, —S(O)_{q}NR¹⁰—, or —NR¹⁰S(O)_{q}—;
X is —N(R¹⁰)₂, —R^{a}N(R¹⁰)₂, -AyN(R¹⁰)₂, R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het, wherein when p is 0 then X is not —N(R¹⁰)₂, or —R^{a}N(R¹⁰)₂;
each R^{a} independently is alkylene, cycloalkylene, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or —R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁵, —R^{a}NR⁸R⁹, -Ay, -Het, —R^{a}Ay —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted 4-, 5-, or 6-membered heterocyclyl or heteroaryl group;
comprising the steps of treating a compound of formula (XI) 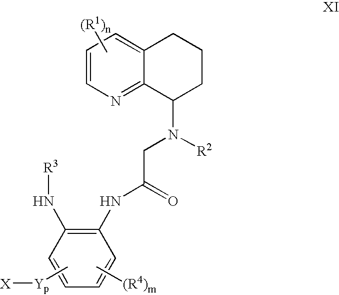
with an acid to form a compound of formula (I).

67. A process for the preparation of a compound of formula (I-B) 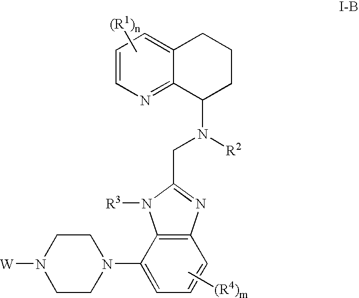
wherein
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2, and, as shown, R¹ can be substituted throughout the depicted tetrahydroquinoline;
R² is selected from a group consisting of H, alkyl, haloalkyl, cycloalkyl, alkenyl, alkynyl, —R^{a}, —R^{a}OR⁵, —R^{a}S(O)_{q}R⁵; wherein R² is not amine or alkylamine, or substituted with amine or alkylamine;
R³ is H, alkyl, haloalkyl, cycloalkyl, alkenyl, alkynyl, —R^{a}Ay, —R^{a}OR⁵, or —R^{a}S(O)_{q}R⁵;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, —S(O)_{q}NR¹⁰—, or —NR¹⁰S(O)_{q}—;
X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het, wherein when p is 0 then X is not —N(R¹⁰)₂, or —R^{a}N(R¹⁰)₂;
each R^{a} independently is alkylene, cycloalkylene, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or —R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁵, R^{a}NR⁸R⁹, -Ay, -Het —R^{a}Ay, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted 4-, 5-, or 6-membered heterocyclyl or heteroaryl group;
comprising the steps of reacting a compound of formula (IX) 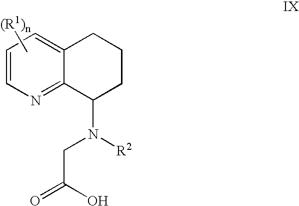
with a compound of formula (X-A) 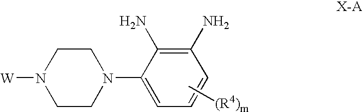
to form a compound of formula (I-B).

68. A process for the preparation of a compound of formula (I-B) 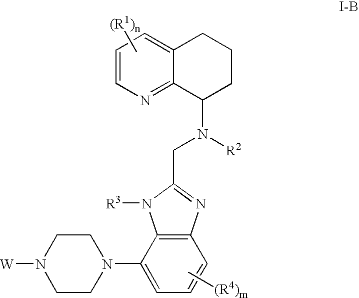
wherein
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2, and, as shown, R¹ can be substituted throughout the depicted tetrahydroquinoline;
R² is selected from a group consisting of H, alkyl, haloalkyl, cycloalkyl, alkenyl, alkynyl, —R^{a}Ay, —R^{a}OR⁵, —R^{a}S(O)_{q}R⁵; wherein R² is not amine or alkylamine, or substituted with amine or alkylamine;
R³ is H, alkyl, haloalkyl, cycloalkyl, alkenyl, alkynyl, —R^{a}Ay, —R^{a}OR⁵, or —R^{a}S(O)_{q}R⁵;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, —S(O)_{q}NR¹⁰—, or —NR¹⁰S(O)_{q}—;
X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR⁸Het, wherein when p is 0 then X is not —N(R¹⁰)₂, or R^{a}N(R¹⁰)₂;
each R^{a} independently is alkylene, cycloalkylene, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or —R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁵, —R^{a}NR⁸R⁹, -Ay, -Het, —R³Ay, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted 4-, 5-, or 6-membered heterocyclyl or heteroaryl group;
comprising the steps of reacting a compound of formula (XVI) 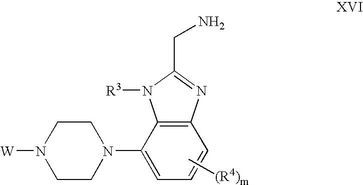
with a compound of formula (II) 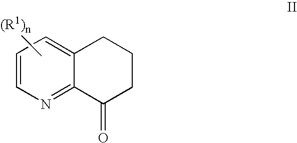
under reductive amination conditions to form a compound of formula (I-B).

69. A process for the preparation of a compound of formula (I-B) 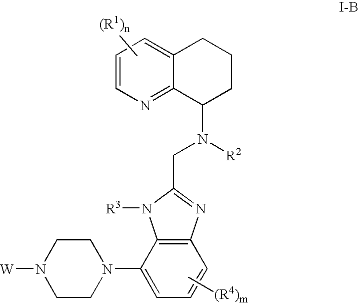
wherein
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido,
n is 0, 1, or 2, and, as shown, R¹ can be substituted throughout the depicted tetrahydroquinoline;
R² is selected from a group consisting of H, alkyl, haloalkyl, cycloalkyl, alkenyl, alkynyl, —R^{a}Ay, —R^{a}OR⁵, —R^{a}S(O)_{q}R⁵; wherein R² is not amine or alkylamine, or substituted with amine or alkylamine;
R³ is H;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —S—, —C(O)NR¹⁰—, —NR¹⁰C(O)—, —C(O)—, —C(O)O—, —NR¹⁰C(O)N(R¹⁰)₂—, —S(O)_{q}—, —S(O)_{q}NR¹⁰—, or —NR¹⁰S(O)_{q}—;
X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR³Ay, or -HetR^{a}Het, wherein when p is 0 then X is not —N(R¹⁰)₂, or —R^{a}N(R¹⁰)₂;
each R^{a} independently is alkylene, cycloalkylene, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or —R^{a}Het;
each of R⁶ and R⁷ independently are selected from H₁ alkyl, alkenyl, alkynyl, cycloalkyl, cycloalkenyl, —R^{a}cycloalkyl, —R^{a}OH, —R^{a}OR⁵, —R^{a}NR⁸OR⁹, -Ay, -Het, —R^{a}Ay, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted 4-, 5-, or 6-membered heterocyclyl or heteroaryl group;
comprising the step of reacting a compound of formula (XX) 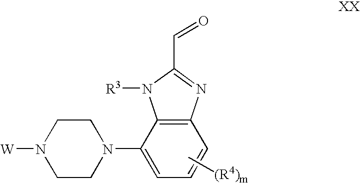
with a compound of formula (III) 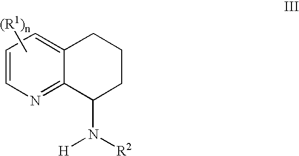
under reductive amination conditions to form a compound of formula (I-B).
